SEQUENCE LISTING (1204 sequences)

SEQ ID NO: 1 - RNA, 20 nt, synthetic construct
  misc_feature 1..20: note = Seynthetic
  source 1..20: mol_type = other RNA
aaaaaaacacaatgtgggga

SEQ ID NO: 2 - RNA, 20 nt, synthetic construct
  misc_feature 1..20: note = Seynthetic
  source 1..20: mol_type = other RNA
aaaaaaatacaatgtgggga

SEQ ID NO: 3 - RNA, 20 nt, synthetic construct
  misc_feature 1..20: note = Seynthetic
  source 1..20: mol_type = other RNA
aaaaaacacaatgtggggaa

SEQ ID NO: 4 - RNA, 20 nt, synthetic construct
  misc_feature 1..20: note = Seynthetic
  source 1..20: mol_type = other RNA
aaaaaatacaatgtggggaa

SEQ ID NO: 5 - RNA, 20 nt, synthetic construct
  misc_feature 1..20: note = Seynthetic
  source 1..20: mol_type = other RNA
aaaaacacaatgtggggaag

SEQ ID NO: 6 - RNA, 20 nt, synthetic construct
  misc_feature 1..20: note = Seynthetic
  source 1..20: mol_type = other RNA
aaaaacccacattgattatc

SEQ ID NO: 7 - RNA, 20 nt, synthetic construct
  misc_feature 1..20: note = Seynthetic
  source 1..20: mol_type = other RNA
aaaaagaaagtgttgctttg

SEQ ID NO: 8 - RNA, 20 nt, synthetic construct
  misc_feature 1..20: note = Seynthetic
  source 1..20: mol_type = other RNA
aaaaatacaatgtggggaag

SEQ ID NO: 9 - RNA, 20 nt, synthetic construct
  misc_feature 1..20: note = Seynthetic
  source 1..20: mol_type = other RNA
aaaacacaatgtggggaagt

SEQ ID NO: 10 - RNA, 20 nt, synthetic construct
  misc_feature 1..20: note = Seynthetic
  source 1..20: mol_type = other RNA
aaaacccacattgattatct

SEQ ID NO: 11 - RNA, 20 nt, synthetic construct
  misc_feature 1..20: note = Seynthetic
  source 1..20: mol_type = other RNA
aaaacccccgctccattaaa

SEQ ID NO: 12 - RNA, 20 nt, synthetic construct
  misc_feature 1..20: note = Seynthetic
  source 1..20: mol_type = other RNA
aaaagaaagtgttgctttga

SEQ ID NO: 13 - RNA, 20 nt, synthetic construct
  misc_feature 1..20: note = Seynthetic
  source 1..20: mol_type = other RNA
aaaagcagggggaacctcgt

SEQ ID NO: 14 - RNA, 20 nt, synthetic construct
  misc_feature 1..20: note = Seynthetic
  source 1..20: mol_type = other RNA
aaaagccgggggaacctcgt

SEQ ID NO: 15 - RNA, 20 nt, synthetic construct
  misc_feature 1..20: note = Seynthetic
  source 1..20: mol_type = other RNA
aaaatacaatgtggggaagt

SEQ ID NO: 16 - RNA, 20 nt, synthetic construct
  misc_feature 1..20: note = Seynthetic
  source 1..20: mol_type = other RNA
aaacacaatgtggggaagtc

SEQ ID NO: 17 - RNA, 20 nt, synthetic construct
  misc_feature 1..20: note = Seynthetic
  source 1..20: mol_type = other RNA
aaacaccctgggggactctg

SEQ ID NO: 18 - RNA, 20 nt, synthetic construct
  misc_feature 1..20: note = Seynthetic
  source 1..20: mol_type = other RNA
aaacaccctgggtgactctg

SEQ ID NO: 19 - RNA, 20 nt, synthetic construct
  misc_feature 1..20: note = Seynthetic
  source 1..20: mol_type = other RNA
aaacccacattgattatctg

SEQ ID NO: 20 - RNA, 20 nt, synthetic construct
  misc_feature 1..20: note = Seynthetic
  source 1..20: mol_type = other RNA
aaactaagaaaatagctgtg

SEQ ID NO: 21 - RNA, 20 nt, synthetic construct
  misc_feature 1..20: note = Seynthetic
  source 1..20: mol_type = other RNA
aaagactgcacgtgagaatg

SEQ ID NO: 22 - RNA, 20 nt, synthetic construct
  misc_feature 1..20: note = Seynthetic
  source 1..20: mol_type = other RNA
aaagatacagcgtcccccac

SEQ ID NO: 23 - RNA, 20 nt, synthetic construct
  misc_feature 1..20: note = Seynthetic
  source 1..20: mol_type = other RNA
aaagatacggcgtcccccac

SEQ ID NO: 24 - RNA, 20 nt, synthetic construct
  misc_feature 1..20: note = Seynthetic
  source 1..20: mol_type = other RNA
aaagcagggggaacctcgtg

SEQ ID NO: 25 - RNA, 20 nt, synthetic construct
  misc_feature 1..20: note = Seynthetic
  source 1..20: mol_type = other RNA
aaagccgggggaacctcgtg

SEQ ID NO: 26 - RNA, 20 nt, synthetic construct
  misc_feature 1..20: note = Seynthetic
  source 1..20: mol_type = other RNA
aaagggattcccaggaccca

SEQ ID NO: 27 - RNA, 20 nt, synthetic construct
  misc_feature 1..20: note = Seynthetic
  source 1..20: mol_type = other RNA
aaagttcagggatgtccgcc

SEQ ID NO: 28 - RNA, 20 nt, synthetic construct
  misc_feature 1..20: note = Seynthetic
  source 1..20: mol_type = other RNA
aaatacaatgtggggaagtc

SEQ ID NO: 29 - RNA, 20 nt, synthetic construct
  misc_feature 1..20: note = Seynthetic
  source 1..20: mol_type = other RNA
aaattcacgttcgtaaagtt

SEQ ID NO: 30 - RNA, 20 nt, synthetic construct
  misc_feature 1..20: note = Seynthetic
  source 1..20: mol_type = other RNA
aaatttgaatgcgattgtgc

SEQ ID NO: 31 - RNA, 20 nt, synthetic construct
  misc_feature 1..20: note = Seynthetic
  source 1..20: mol_type = other RNA
aacacaatgtggggaagtca

SEQ ID NO: 32 - RNA, 20 nt, synthetic construct
  misc_feature 1..20: note = Seynthetic
  source 1..20: mol_type = other RNA
aacaccctgggggactctgg

SEQ ID NO: 33 - RNA, 20 nt, synthetic construct
  misc_feature 1..20: note = Seynthetic
  source 1..20: mol_type = other RNA
aacaccctgggtgactctgg

SEQ ID NO: 34 - RNA, 20 nt, synthetic construct
  misc_feature 1..20: note = Seynthetic
  source 1..20: mol_type = other RNA
aacatcgtgggggagatggg

SEQ ID NO: 35 - RNA, 20 nt, synthetic construct
  misc_feature 1..20: note = Seynthetic
  source 1..20: mol_type = other RNA
aacatggtgggggagatggg

SEQ ID NO: 36 - RNA, 20 nt, synthetic construct
  misc_feature 1..20: note = Seynthetic
  source 1..20: mol_type = other RNA
aacccacattgattatctga

SEQ ID NO: 37 - RNA, 20 nt, synthetic construct
  misc_feature 1..20: note = Seynthetic
  source 1..20: mol_type = other RNA
aacccccgctccattaaaaa

SEQ ID NO: 38 - RNA, 20 nt, synthetic construct
  misc_feature 1..20: note = Seynthetic
  source 1..20: mol_type = other RNA
aacttcaccacacccagagt

SEQ ID NO: 39 - RNA, 20 nt, synthetic construct
  misc_feature 1..20: note = Seynthetic
  source 1..20: mol_type = other RNA
aactttacgaacgtgaattt

SEQ ID NO: 40 - RNA, 20 nt, synthetic construct
  misc_feature 1..20: note = Seynthetic
  source 1..20: mol_type = other RNA
aagaaaaaaacacaatgtgg

SEQ ID NO: 41 - RNA, 20 nt, synthetic construct
  misc_feature 1..20: note = Seynthetic
  source 1..20: mol_type = other RNA
aagaaagtgttgctttgaga

SEQ ID NO: 42 - RNA, 20 nt, synthetic construct
  misc_feature 1..20: note = Seynthetic
  source 1..20: mol_type = other RNA
aagaactttacgaacgtgaa

SEQ ID NO: 43 - RNA, 20 nt, synthetic construct
  misc_feature 1..20: note = Seynthetic
  source 1..20: mol_type = other RNA
aagaactttatgaacgtgaa

SEQ ID NO: 44 - RNA, 20 nt, synthetic construct
  misc_feature 1..20: note = Seynthetic
  source 1..20: mol_type = other RNA
aagactccagcctggcccag

SEQ ID NO: 45 - RNA, 20 nt, synthetic construct
  misc_feature 1..20: note = Seynthetic
  source 1..20: mol_type = other RNA
aagactgcacgtgagaatgt

SEQ ID NO: 46 - RNA, 20 nt, synthetic construct
  misc_feature 1..20: note = Seynthetic
  source 1..20: mol_type = other RNA
aagagaaaagcagggggaac

SEQ ID NO: 47 - RNA, 20 nt, synthetic construct
  misc_feature 1..20: note = Seynthetic
  source 1..20: mol_type = other RNA
aagagaaaagccgggggaac

SEQ ID NO: 48 - RNA, 20 nt, synthetic construct
  misc_feature 1..20: note = Seynthetic
  source 1..20: mol_type = other RNA
aagatacagcgtcccccacc

SEQ ID NO: 49 - RNA, 20 nt, synthetic construct
  misc_feature 1..20: note = Seynthetic
  source 1..20: mol_type = other RNA
aagatacggcgtcccccacc

SEQ ID NO: 50 - RNA, 20 nt, synthetic construct
  misc_feature 1..20: note = Seynthetic
  source 1..20: mol_type = other RNA
aagcagggggaacctcgtgt

SEQ ID NO: 51 - RNA, 20 nt, synthetic construct
  misc_feature 1..20: note = Seynthetic
  source 1..20: mol_type = other RNA
aagccacacctgaaggcgga

SEQ ID NO: 52 - RNA, 20 nt, synthetic construct
  misc_feature 1..20: note = Seynthetic
  source 1..20: mol_type = other RNA
aagccccagaggccacaggg

SEQ ID NO: 53 - RNA, 20 nt, synthetic construct
  misc_feature 1..20: note = Seynthetic
  source 1..20: mol_type = other RNA
aagccccttccaaagataca

SEQ ID NO: 54 - RNA, 20 nt, synthetic construct
  misc_feature 1..20: note = Seynthetic
  source 1..20: mol_type = other RNA
aagccccttccaaagatacg

SEQ ID NO: 55 - RNA, 20 nt, synthetic construct
  misc_feature 1..20: note = Seynthetic
  source 1..20: mol_type = other RNA
aagccgggggaacctcgtgt

SEQ ID NO: 56 - RNA, 20 nt, synthetic construct
  misc_feature 1..20: note = Seynthetic
  source 1..20: mol_type = other RNA
aaggcggacatccctgaact

SEQ ID NO: 57 - RNA, 20 nt, synthetic construct
  misc_feature 1..20: note = Seynthetic
  source 1..20: mol_type = other RNA
aagggattcccaggacccag

SEQ ID NO: 58 - RNA, 20 nt, synthetic construct
  misc_feature 1..20: note = Seynthetic
  source 1..20: mol_type = other RNA
aaggggctgccggagtcccc

SEQ ID NO: 59 - RNA, 20 nt, synthetic construct
  misc_feature 1..20: note = Seynthetic
  source 1..20: mol_type = other RNA
aagtgacttccccacattgt

SEQ ID NO: 60 - RNA, 20 nt, synthetic construct
  misc_feature 1..20: note = Seynthetic
  source 1..20: mol_type = other RNA
aagttcagggatgtccgcct

SEQ ID NO: 61 - RNA, 20 nt, synthetic construct
  misc_feature 1..20: note = Seynthetic
  source 1..20: mol_type = other RNA
aataaagactgcacgtgaga

SEQ ID NO: 62 - RNA, 20 nt, synthetic construct
  misc_feature 1..20: note = Seynthetic
  source 1..20: mol_type = other RNA
aataatgactgcacgtgaga

SEQ ID NO: 63 - RNA, 20 nt, synthetic construct
  misc_feature 1..20: note = Seynthetic
  source 1..20: mol_type = other RNA
aatacaatgtggggaagtca

SEQ ID NO: 64 - RNA, 20 nt, synthetic construct
  misc_feature 1..20: note = Seynthetic
  source 1..20: mol_type = other RNA
aatccatggtgcggggacca

SEQ ID NO: 65 - RNA, 20 nt, synthetic construct
  misc_feature 1..20: note = Seynthetic
  source 1..20: mol_type = other RNA
aatcccattcccgcagctgg

SEQ ID NO: 66 - RNA, 20 nt, synthetic construct
  misc_feature 1..20: note = Seynthetic
  source 1..20: mol_type = other RNA
aatccctttcccgcagctgg

SEQ ID NO: 67 - RNA, 20 nt, synthetic construct
  misc_feature 1..20: note = Seynthetic
  source 1..20: mol_type = other RNA
aatcgcattcaaatgtcaga

SEQ ID NO: 68 - RNA, 20 nt, synthetic construct
  misc_feature 1..20: note = Seynthetic
  source 1..20: mol_type = other RNA
aatcgcattcaaatttcaga

SEQ ID NO: 69 - RNA, 20 nt, synthetic construct
  misc_feature 1..20: note = Seynthetic
  source 1..20: mol_type = other RNA
aatgactgcacgtgagaatg

SEQ ID NO: 70 - RNA, 20 nt, synthetic construct
  misc_feature 1..20: note = Seynthetic
  source 1..20: mol_type = other RNA
aatgggattcccaggaccca

SEQ ID NO: 71 - RNA, 20 nt, synthetic construct
  misc_feature 1..20: note = Seynthetic
  source 1..20: mol_type = other RNA
aatgtcagataatcaatgtg

SEQ ID NO: 72 - RNA, 20 nt, synthetic construct
  misc_feature 1..20: note = Seynthetic
  source 1..20: mol_type = other RNA
aattcacgttcataaagttc

SEQ ID NO: 73 - RNA, 20 nt, synthetic construct
  misc_feature 1..20: note = Seynthetic
  source 1..20: mol_type = other RNA
aattcacgttcgtaaagttc

SEQ ID NO: 74 - RNA, 20 nt, synthetic construct
  misc_feature 1..20: note = Seynthetic
  source 1..20: mol_type = other RNA
aatttgaatgcgattgtgca

SEQ ID NO: 75 - RNA, 20 nt, synthetic construct
  misc_feature 1..20: note = Seynthetic
  source 1..20: mol_type = other RNA
acaatcgcattcaaatgtca

SEQ ID NO: 76 - RNA, 20 nt, synthetic construct
  misc_feature 1..20: note = Seynthetic
  source 1..20: mol_type = other RNA
acaatcgcattcaaatttca

SEQ ID NO: 77 - RNA, 20 nt, synthetic construct
  misc_feature 1..20: note = Seynthetic
  source 1..20: mol_type = other RNA
acacaatgtggggaagtcac

SEQ ID NO: 78 - RNA, 20 nt, synthetic construct
  misc_feature 1..20: note = Seynthetic
  source 1..20: mol_type = other RNA
acacacagctattttcttag

SEQ ID NO: 79 - RNA, 20 nt, synthetic construct
  misc_feature 1..20: note = Seynthetic
  source 1..20: mol_type = other RNA
acacccagagtcacccaggg

SEQ ID NO: 80 - RNA, 20 nt, synthetic construct
  misc_feature 1..20: note = Seynthetic
  source 1..20: mol_type = other RNA
acacccagagtcccccaggg

SEQ ID NO: 81 - RNA, 20 nt, synthetic construct
  misc_feature 1..20: note = Seynthetic
  source 1..20: mol_type = other RNA
acacccccttcctgcccacc

SEQ ID NO: 82 - RNA, 20 nt, synthetic construct
  misc_feature 1..20: note = Seynthetic
  source 1..20: mol_type = other RNA
acacccgctgtgaccataac

SEQ ID NO: 83 - RNA, 20 nt, synthetic construct
  misc_feature 1..20: note = Seynthetic
  source 1..20: mol_type = other RNA
acaccctgggggactctggg

SEQ ID NO: 84 - RNA, 20 nt, synthetic construct
  misc_feature 1..20: note = Seynthetic
  source 1..20: mol_type = other RNA
acaccctgggtgactctggg

SEQ ID NO: 85 - RNA, 20 nt, synthetic construct
  misc_feature 1..20: note = Seynthetic
  source 1..20: mol_type = other RNA
acacctgaaggcggacatcc

SEQ ID NO: 86 - RNA, 20 nt, synthetic construct
  misc_feature 1..20: note = Seynthetic
  source 1..20: mol_type = other RNA
acacctgctgtgaccataac

SEQ ID NO: 87 - RNA, 20 nt, synthetic construct
  misc_feature 1..20: note = Seynthetic
  source 1..20: mol_type = other RNA
acacctttgtctctgtccct

SEQ ID NO: 88 - RNA, 20 nt, synthetic construct
  misc_feature 1..20: note = Seynthetic
  source 1..20: mol_type = other RNA
acacgaggttcccccggctt

SEQ ID NO: 89 - RNA, 20 nt, synthetic construct
  misc_feature 1..20: note = Seynthetic
  source 1..20: mol_type = other RNA
acacgaggttccccctgctt

SEQ ID NO: 90 - RNA, 20 nt, synthetic construct
  misc_feature 1..20: note = Seynthetic
  source 1..20: mol_type = other RNA
acacggacggtcctccagcc

SEQ ID NO: 91 - RNA, 20 nt, synthetic construct
  misc_feature 1..20: note = Seynthetic
  source 1..20: mol_type = other RNA
acacttaataaagactgcac

SEQ ID NO: 92 - RNA, 20 nt, synthetic construct
  misc_feature 1..20: note = Seynthetic
  source 1..20: mol_type = other RNA
acacttaataatgactgcac

SEQ ID NO: 93 - RNA, 20 nt, synthetic construct
  misc_feature 1..20: note = Seynthetic
  source 1..20: mol_type = other RNA
acagcaggtgtagactccga

SEQ ID NO: 94 - RNA, 20 nt, synthetic construct
  misc_feature 1..20: note = Seynthetic
  source 1..20: mol_type = other RNA
acagcgggtgtagactccga

SEQ ID NO: 95 - RNA, 20 nt, synthetic construct
  misc_feature 1..20: note = Seynthetic
  source 1..20: mol_type = other RNA
acagcgtcccccacccctga

SEQ ID NO: 96 - RNA, 20 nt, synthetic construct
  misc_feature 1..20: note = Seynthetic
  source 1..20: mol_type = other RNA
acagggggactccggcagcc

SEQ ID NO: 97 - RNA, 20 nt, synthetic construct
  misc_feature 1..20: note = Seynthetic
  source 1..20: mol_type = other RNA
acatcgtgggggagatggga

SEQ ID NO: 98 - RNA, 20 nt, synthetic construct
  misc_feature 1..20: note = Seynthetic
  source 1..20: mol_type = other RNA
acatggtgggggagatggga

SEQ ID NO: 99 - RNA, 20 nt, synthetic construct
  misc_feature 1..20: note = Seynthetic
  source 1..20: mol_type = other RNA
acattctcacgtgcagtcat

SEQ ID NO: 100 - RNA, 20 nt, synthetic construct
  misc_feature 1..20: note = Seynthetic
  source 1..20: mol_type = other RNA
acattctcacgtgcagtctt

SEQ ID NO: 101 - RNA, 20 nt, synthetic construct
  misc_feature 1..20: note = Seynthetic
  source 1..20: mol_type = other RNA
acatttgaatgcgattgtgc

SEQ ID NO: 102 - RNA, 20 nt, synthetic construct
  misc_feature 1..20: note = Seynthetic
  source 1..20: mol_type = other RNA
accaaggggctgccggagtc

SEQ ID NO: 103 - RNA, 20 nt, synthetic construct
  misc_feature 1..20: note = Seynthetic
  source 1..20: mol_type = other RNA
accaaggggctgcctgagtc

SEQ ID NO: 104 - RNA, 20 nt, synthetic construct
  misc_feature 1..20: note = Seynthetic
  source 1..20: mol_type = other RNA
accacacccagagtcaccca

SEQ ID NO: 105 - RNA, 20 nt, synthetic construct
  misc_feature 1..20: note = Seynthetic
  source 1..20: mol_type = other RNA
accacacccagagtccccca

SEQ ID NO: 106 - RNA, 20 nt, synthetic construct
  misc_feature 1..20: note = Seynthetic
  source 1..20: mol_type = other RNA
accagggaactcctgccctg

SEQ ID NO: 107 - RNA, 20 nt, synthetic construct
  misc_feature 1..20: note = Seynthetic
  source 1..20: mol_type = other RNA
accatgttccagctgcaacg

SEQ ID NO: 108 - RNA, 20 nt, synthetic construct
  misc_feature 1..20: note = Seynthetic
  source 1..20: mol_type = other RNA
acccacattgattatctgaa

SEQ ID NO: 109 - RNA, 20 nt, synthetic construct
  misc_feature 1..20: note = Seynthetic
  source 1..20: mol_type = other RNA
acccacattgattatctgac

SEQ ID NO: 110 - RNA, 20 nt, synthetic construct
  misc_feature 1..20: note = Seynthetic
  source 1..20: mol_type = other RNA
acccagagtcacccagggtg

SEQ ID NO: 111 - RNA, 20 nt, synthetic construct
  misc_feature 1..20: note = Seynthetic
  source 1..20: mol_type = other RNA
acccagagtcccccagggtg

SEQ ID NO: 112 - RNA, 20 nt, synthetic construct
  misc_feature 1..20: note = Seynthetic
  source 1..20: mol_type = other RNA
acccagggtgtttcacaatc

SEQ ID NO: 113 - RNA, 20 nt, synthetic construct
  misc_feature 1..20: note = Seynthetic
  source 1..20: mol_type = other RNA
acccccgctccattaaaaaa

SEQ ID NO: 114 - RNA, 20 nt, synthetic construct
  misc_feature 1..20: note = Seynthetic
  source 1..20: mol_type = other RNA
acccccttcctgcccacctg

SEQ ID NO: 115 - RNA, 20 nt, synthetic construct
  misc_feature 1..20: note = Seynthetic
  source 1..20: mol_type = other RNA
acccgctgtgaccataacac

SEQ ID NO: 116 - RNA, 20 nt, synthetic construct
  misc_feature 1..20: note = Seynthetic
  source 1..20: mol_type = other RNA
accctgggggactctgggtg

SEQ ID NO: 117 - RNA, 20 nt, synthetic construct
  misc_feature 1..20: note = Seynthetic
  source 1..20: mol_type = other RNA
accctgggtgactctgggtg

SEQ ID NO: 118 - RNA, 20 nt, synthetic construct
  misc_feature 1..20: note = Seynthetic
  source 1..20: mol_type = other RNA
acctgaaggcggacatccct

SEQ ID NO: 119 - RNA, 20 nt, synthetic construct
  misc_feature 1..20: note = Seynthetic
  source 1..20: mol_type = other RNA
acctgctgtgaccataacac

SEQ ID NO: 120 - RNA, 20 nt, synthetic construct
  misc_feature 1..20: note = Seynthetic
  source 1..20: mol_type = other RNA
acctggaggggctggaggac

SEQ ID NO: 121 - RNA, 20 nt, synthetic construct
  misc_feature 1..20: note = Seynthetic
  source 1..20: mol_type = other RNA
acctggcggggctggaggac

SEQ ID NO: 122 - RNA, 20 nt, synthetic construct
  misc_feature 1..20: note = Seynthetic
  source 1..20: mol_type = other RNA
acctttgtctctgtccctgt

SEQ ID NO: 123 - RNA, 20 nt, synthetic construct
  misc_feature 1..20: note = Seynthetic
  source 1..20: mol_type = other RNA
acgaacgtgaatttatttca

SEQ ID NO: 124 - RNA, 20 nt, synthetic construct
  misc_feature 1..20: note = Seynthetic
  source 1..20: mol_type = other RNA
acgaggttcccccggctttt

SEQ ID NO: 125 - RNA, 20 nt, synthetic construct
  misc_feature 1..20: note = Seynthetic
  source 1..20: mol_type = other RNA
acgaggttccccctgctttt

SEQ ID NO: 126 - RNA, 20 nt, synthetic construct
  misc_feature 1..20: note = Seynthetic
  source 1..20: mol_type = other RNA
acgatgttccagctgcaacg

SEQ ID NO: 127 - RNA, 20 nt, synthetic construct
  misc_feature 1..20: note = Seynthetic
  source 1..20: mol_type = other RNA
acgccgtatctttggaaggg

SEQ ID NO: 128 - RNA, 20 nt, synthetic construct
  misc_feature 1..20: note = Seynthetic
  source 1..20: mol_type = other RNA
acgctgtatctttggaaggg

SEQ ID NO: 129 - RNA, 20 nt, synthetic construct
  misc_feature 1..20: note = Seynthetic
  source 1..20: mol_type = other RNA
acggacggtcctccagcccc

SEQ ID NO: 130 - RNA, 20 nt, synthetic construct
  misc_feature 1..20: note = Seynthetic
  source 1..20: mol_type = other RNA
acggcgtcccccacccctga

SEQ ID NO: 131 - RNA, 20 nt, synthetic construct
  misc_feature 1..20: note = Seynthetic
  source 1..20: mol_type = other RNA
acggtcctccagccccgcca

SEQ ID NO: 132 - RNA, 20 nt, synthetic construct
  misc_feature 1..20: note = Seynthetic
  source 1..20: mol_type = other RNA
acggtcctccagcccctcca

SEQ ID NO: 133 - RNA, 20 nt, synthetic construct
  misc_feature 1..20: note = Seynthetic
  source 1..20: mol_type = other RNA
acgtcaagactccagcctgg

SEQ ID NO: 134 - RNA, 20 nt, synthetic construct
  misc_feature 1..20: note = Seynthetic
  source 1..20: mol_type = other RNA
acgtcaggactccagcctgg

SEQ ID NO: 135 - RNA, 20 nt, synthetic construct
  misc_feature 1..20: note = Seynthetic
  source 1..20: mol_type = other RNA
acgtccccctcggagtctac

SEQ ID NO: 136 - RNA, 20 nt, synthetic construct
  misc_feature 1..20: note = Seynthetic
  source 1..20: mol_type = other RNA
acgtgcagtcattattaagt

SEQ ID NO: 137 - RNA, 20 nt, synthetic construct
  misc_feature 1..20: note = Seynthetic
  source 1..20: mol_type = other RNA
acgtgcagtctttattaagt

SEQ ID NO: 138 - RNA, 20 nt, synthetic construct
  misc_feature 1..20: note = Seynthetic
  source 1..20: mol_type = other RNA
acgttcataaagttcttagc

SEQ ID NO: 139 - RNA, 20 nt, synthetic construct
  misc_feature 1..20: note = Seynthetic
  source 1..20: mol_type = other RNA
acgttcgtaaagttcttagc

SEQ ID NO: 140 - RNA, 20 nt, synthetic construct
  misc_feature 1..20: note = Seynthetic
  source 1..20: mol_type = other RNA
acgttgcattaatccatggt

SEQ ID NO: 141 - RNA, 20 nt, synthetic construct
  misc_feature 1..20: note = Seynthetic
  source 1..20: mol_type = other RNA
actacattctcacgtgcagt

SEQ ID NO: 142 - RNA, 20 nt, synthetic construct
  misc_feature 1..20: note = Seynthetic
  source 1..20: mol_type = other RNA
actccggcagccccttggtc

SEQ ID NO: 143 - RNA, 20 nt, synthetic construct
  misc_feature 1..20: note = Seynthetic
  source 1..20: mol_type = other RNA
acttaataaagactgcacgt

SEQ ID NO: 144 - RNA, 20 nt, synthetic construct
  misc_feature 1..20: note = Seynthetic
  source 1..20: mol_type = other RNA
acttaataatgactgcacgt

SEQ ID NO: 145 - RNA, 20 nt, synthetic construct
  misc_feature 1..20: note = Seynthetic
  source 1..20: mol_type = other RNA
acttcaccacacccagagtc

SEQ ID NO: 146 - RNA, 20 nt, synthetic construct
  misc_feature 1..20: note = Seynthetic
  source 1..20: mol_type = other RNA
acttccccacattgtatttt

SEQ ID NO: 147 - RNA, 20 nt, synthetic construct
  misc_feature 1..20: note = Seynthetic
  source 1..20: mol_type = other RNA
acttccccacattgtgtttt

SEQ ID NO: 148 - RNA, 20 nt, synthetic construct
  misc_feature 1..20: note = Seynthetic
  source 1..20: mol_type = other RNA
acttgcctgtggtcacgtca

SEQ ID NO: 149 - RNA, 20 nt, synthetic construct
  misc_feature 1..20: note = Seynthetic
  source 1..20: mol_type = other RNA
actttacgaacgtgaattta

SEQ ID NO: 150 - RNA, 20 nt, synthetic construct
  misc_feature 1..20: note = Seynthetic
  source 1..20: mol_type = other RNA
actttcttagctaagaactt

SEQ ID NO: 151 - RNA, 20 nt, synthetic construct
  misc_feature 1..20: note = Seynthetic
  source 1..20: mol_type = other RNA
agaaaaaaacacaatgtggg

SEQ ID NO: 152 - RNA, 20 nt, synthetic construct
  misc_feature 1..20: note = Seynthetic
  source 1..20: mol_type = other RNA
agaaaagcagggggaacctc

SEQ ID NO: 153 - RNA, 20 nt, synthetic construct
  misc_feature 1..20: note = Seynthetic
  source 1..20: mol_type = other RNA
agaaaagccgggggaacctc

SEQ ID NO: 154 - RNA, 20 nt, synthetic construct
  misc_feature 1..20: note = Seynthetic
  source 1..20: mol_type = other RNA
agaaagtgttgctttgagag

SEQ ID NO: 155 - RNA, 20 nt, synthetic construct
  misc_feature 1..20: note = Seynthetic
  source 1..20: mol_type = other RNA
agaactttacgaacgtgaat

SEQ ID NO: 156 - RNA, 20 nt, synthetic construct
  misc_feature 1..20: note = Seynthetic
  source 1..20: mol_type = other RNA
agaactttatgaacgtgaat

SEQ ID NO: 157 - RNA, 20 nt, synthetic construct
  misc_feature 1..20: note = Seynthetic
  source 1..20: mol_type = other RNA
agaagtgacttccccacatt

SEQ ID NO: 158 - RNA, 20 nt, synthetic construct
  misc_feature 1..20: note = Seynthetic
  source 1..20: mol_type = other RNA
agaccaaggggctgccggag

SEQ ID NO: 159 - RNA, 20 nt, synthetic construct
  misc_feature 1..20: note = Seynthetic
  source 1..20: mol_type = other RNA
agactccagcctggcccagg

SEQ ID NO: 160 - RNA, 20 nt, synthetic construct
  misc_feature 1..20: note = Seynthetic
  source 1..20: mol_type = other RNA
agactgcacgtgagaatgta

SEQ ID NO: 161 - RNA, 20 nt, synthetic construct
  misc_feature 1..20: note = Seynthetic
  source 1..20: mol_type = other RNA
agagaaaagcagggggaacc

SEQ ID NO: 162 - RNA, 20 nt, synthetic construct
  misc_feature 1..20: note = Seynthetic
  source 1..20: mol_type = other RNA
agagaaaagccgggggaacc

SEQ ID NO: 163 - RNA, 20 nt, synthetic construct
  misc_feature 1..20: note = Seynthetic
  source 1..20: mol_type = other RNA
agagcctgggccaggctgga

SEQ ID NO: 164 - RNA, 20 nt, synthetic construct
  misc_feature 1..20: note = Seynthetic
  source 1..20: mol_type = other RNA
agaggccacagggacagaga

SEQ ID NO: 165 - RNA, 20 nt, synthetic construct
  misc_feature 1..20: note = Seynthetic
  source 1..20: mol_type = other RNA
agagtcacccagggtgtttc

SEQ ID NO: 166 - RNA, 20 nt, synthetic construct
  misc_feature 1..20: note = Seynthetic
  source 1..20: mol_type = other RNA
agagtcccccagggtgtttc

SEQ ID NO: 167 - RNA, 20 nt, synthetic construct
  misc_feature 1..20: note = Seynthetic
  source 1..20: mol_type = other RNA
agatacagcgtcccccaccc

SEQ ID NO: 168 - RNA, 20 nt, synthetic construct
  misc_feature 1..20: note = Seynthetic
  source 1..20: mol_type = other RNA
agatacggcgtcccccaccc

SEQ ID NO: 169 - RNA, 20 nt, synthetic construct
  misc_feature 1..20: note = Seynthetic
  source 1..20: mol_type = other RNA
agcacacgaggttcccccgg

SEQ ID NO: 170 - RNA, 20 nt, synthetic construct
  misc_feature 1..20: note = Seynthetic
  source 1..20: mol_type = other RNA
agcacacgaggttccccctg

SEQ ID NO: 171 - RNA, 20 nt, synthetic construct
  misc_feature 1..20: note = Seynthetic
  source 1..20: mol_type = other RNA
agcagcacacgaggttcccc

SEQ ID NO: 172 - RNA, 20 nt, synthetic construct
  misc_feature 1..20: note = Seynthetic
  source 1..20: mol_type = other RNA
agcagggggaacctcgtgtg

SEQ ID NO: 173 - RNA, 20 nt, synthetic construct
  misc_feature 1..20: note = Seynthetic
  source 1..20: mol_type = other RNA
agcaggtgtagactccgagg

SEQ ID NO: 174 - RNA, 20 nt, synthetic construct
  misc_feature 1..20: note = Seynthetic
  source 1..20: mol_type = other RNA
agccacacctgaaggcggac

SEQ ID NO: 175 - RNA, 20 nt, synthetic construct
  misc_feature 1..20: note = Seynthetic
  source 1..20: mol_type = other RNA
agccccagaggacacaggga

SEQ ID NO: 176 - RNA, 20 nt, synthetic construct
  misc_feature 1..20: note = Seynthetic
  source 1..20: mol_type = other RNA
agccccttccaaagatacag

SEQ ID NO: 177 - RNA, 20 nt, synthetic construct
  misc_feature 1..20: note = Seynthetic
  source 1..20: mol_type = other RNA
agccccttccaaagatacgg

SEQ ID NO: 178 - RNA, 20 nt, synthetic construct
  misc_feature 1..20: note = Seynthetic
  source 1..20: mol_type = other RNA
agccgggggaacctcgtgtg

SEQ ID NO: 179 - RNA, 20 nt, synthetic construct
  misc_feature 1..20: note = Seynthetic
  source 1..20: mol_type = other RNA
agcctgggccaggctggagt

SEQ ID NO: 180 - RNA, 20 nt, synthetic construct
  misc_feature 1..20: note = Seynthetic
  source 1..20: mol_type = other RNA
agcgggtgtagactccgagg

SEQ ID NO: 181 - RNA, 20 nt, synthetic construct
  misc_feature 1..20: note = Seynthetic
  source 1..20: mol_type = other RNA
agcgtcccccacccctgaaa

SEQ ID NO: 182 - RNA, 20 nt, synthetic construct
  misc_feature 1..20: note = Seynthetic
  source 1..20: mol_type = other RNA
agcgtcggcacacttaataa

SEQ ID NO: 183 - RNA, 20 nt, synthetic construct
  misc_feature 1..20: note = Seynthetic
  source 1..20: mol_type = other RNA
agctaagaactttacgaacg

SEQ ID NO: 184 - RNA, 20 nt, synthetic construct
  misc_feature 1..20: note = Seynthetic
  source 1..20: mol_type = other RNA
agctaagaactttatgaacg

SEQ ID NO: 185 - RNA, 20 nt, synthetic construct
  misc_feature 1..20: note = Seynthetic
  source 1..20: mol_type = other RNA
agctccctggtagaagcatg

SEQ ID NO: 186 - RNA, 20 nt, synthetic construct
  misc_feature 1..20: note = Seynthetic
  source 1..20: mol_type = other RNA
agctcctgccctgcctaccc

SEQ ID NO: 187 - RNA, 20 nt, synthetic construct
  misc_feature 1..20: note = Seynthetic
  source 1..20: mol_type = other RNA
agctgcgggaaagggattcc

SEQ ID NO: 188 - RNA, 20 nt, synthetic construct
  misc_feature 1..20: note = Seynthetic
  source 1..20: mol_type = other RNA
agctgcgggaatgggattcc

SEQ ID NO: 189 - RNA, 20 nt, synthetic construct
  misc_feature 1..20: note = Seynthetic
  source 1..20: mol_type = other RNA
agctggaacatcgtggggga

SEQ ID NO: 190 - RNA, 20 nt, synthetic construct
  misc_feature 1..20: note = Seynthetic
  source 1..20: mol_type = other RNA
agctggaacatggtggggga

SEQ ID NO: 191 - RNA, 20 nt, synthetic construct
  misc_feature 1..20: note = Seynthetic
  source 1..20: mol_type = other RNA
agctggagtcccagctgcgg

SEQ ID NO: 192 - RNA, 20 nt, synthetic construct
  misc_feature 1..20: note = Seynthetic
  source 1..20: mol_type = other RNA
aggactccagcctggcccag

SEQ ID NO: 193 - RNA, 20 nt, synthetic construct
  misc_feature 1..20: note = Seynthetic
  source 1..20: mol_type = other RNA
aggagctccctggtagaagc

SEQ ID NO: 194 - RNA, 20 nt, synthetic construct
  misc_feature 1..20: note = Seynthetic
  source 1..20: mol_type = other RNA
aggagttccctggtagaagc

SEQ ID NO: 195 - RNA, 20 nt, synthetic construct
  misc_feature 1..20: note = Seynthetic
  source 1..20: mol_type = other RNA
aggatgaaataaattcacgt

SEQ ID NO: 196 - RNA, 20 nt, synthetic construct
  misc_feature 1..20: note = Seynthetic
  source 1..20: mol_type = other RNA
aggatgcacaatcgcattca

SEQ ID NO: 197 - RNA, 20 nt, synthetic construct
  misc_feature 1..20: note = Seynthetic
  source 1..20: mol_type = other RNA
aggattgtgaaacaccctgg

SEQ ID NO: 198 - RNA, 20 nt, synthetic construct
  misc_feature 1..20: note = Seynthetic
  source 1..20: mol_type = other RNA
aggcagccccttggtctgca

SEQ ID NO: 199 - RNA, 20 nt, synthetic construct
  misc_feature 1..20: note = Seynthetic
  source 1..20: mol_type = other RNA
aggccacagggacagagaca

SEQ ID NO: 200 - RNA, 20 nt, synthetic construct
  misc_feature 1..20: note = Seynthetic
  source 1..20: mol_type = other RNA
aggcggacatccctgaactt

SEQ ID NO: 201 - RNA, 20 nt, synthetic construct
  misc_feature 1..20: note = Seynthetic
  source 1..20: mol_type = other RNA
aggctggagtcctgacgtga

SEQ ID NO: 202 - RNA, 20 nt, synthetic construct
  misc_feature 1..20: note = Seynthetic
  source 1..20: mol_type = other RNA
agggaactcctgccctgcct

SEQ ID NO: 203 - RNA, 20 nt, synthetic construct
  misc_feature 1..20: note = Seynthetic
  source 1..20: mol_type = other RNA
agggaccagaaggatgggga

SEQ ID NO: 204 - RNA, 20 nt, synthetic construct
  misc_feature 1..20: note = Seynthetic
  source 1..20: mol_type = other RNA
agggagctcctgccctgcct

SEQ ID NO: 205 - RNA, 20 nt, synthetic construct
  misc_feature 1..20: note = Seynthetic
  source 1..20: mol_type = other RNA
agggatgtccgccttcaggt

SEQ ID NO: 206 - RNA, 20 nt, synthetic construct
  misc_feature 1..20: note = Seynthetic
  source 1..20: mol_type = other RNA
agggattcccaggacccagc

SEQ ID NO: 207 - RNA, 20 nt, synthetic construct
  misc_feature 1..20: note = Seynthetic
  source 1..20: mol_type = other RNA
agggcaggagttccctggta

SEQ ID NO: 208 - RNA, 20 nt, synthetic construct
  misc_feature 1..20: note = Seynthetic
  source 1..20: mol_type = other RNA
aggggctggaggaccgtccg

SEQ ID NO: 209 - RNA, 20 nt, synthetic construct
  misc_feature 1..20: note = Seynthetic
  source 1..20: mol_type = other RNA
agggggaacctcgtgtgctg

SEQ ID NO: 210 - RNA, 20 nt, synthetic construct
  misc_feature 1..20: note = Seynthetic
  source 1..20: mol_type = other RNA
aggggtaggcagggcaggag

SEQ ID NO: 211 - RNA, 20 nt, synthetic construct
  misc_feature 1..20: note = Seynthetic
  source 1..20: mol_type = other RNA
aggggtgggggacgccgtat

SEQ ID NO: 212 - RNA, 20 nt, synthetic construct
  misc_feature 1..20: note = Seynthetic
  source 1..20: mol_type = other RNA
aggggtgggggacgctgtat

SEQ ID NO: 213 - RNA, 20 nt, synthetic construct
  misc_feature 1..20: note = Seynthetic
  source 1..20: mol_type = other RNA
aggggtgttatggtcacagc

SEQ ID NO: 214 - RNA, 20 nt, synthetic construct
  misc_feature 1..20: note = Seynthetic
  source 1..20: mol_type = other RNA
agggtgtcaagccccagagg

SEQ ID NO: 215 - RNA, 20 nt, synthetic construct
  misc_feature 1..20: note = Seynthetic
  source 1..20: mol_type = other RNA
aggtgtagactccgaggggg

SEQ ID NO: 216 - RNA, 20 nt, synthetic construct
  misc_feature 1..20: note = Seynthetic
  source 1..20: mol_type = other RNA
aggttcccccggcttttctc

SEQ ID NO: 217 - RNA, 20 nt, synthetic construct
  misc_feature 1..20: note = Seynthetic
  source 1..20: mol_type = other RNA
aggttccccctgcttttctc

SEQ ID NO: 218 - RNA, 20 nt, synthetic construct
  misc_feature 1..20: note = Seynthetic
  source 1..20: mol_type = other RNA
agtcacccagggtgtttcac

SEQ ID NO: 219 - RNA, 20 nt, synthetic construct
  misc_feature 1..20: note = Seynthetic
  source 1..20: mol_type = other RNA
agtcattattaagtgtgccg

SEQ ID NO: 220 - RNA, 20 nt, synthetic construct
  misc_feature 1..20: note = Seynthetic
  source 1..20: mol_type = other RNA
agtcccagctgcgggaaagg

SEQ ID NO: 221 - RNA, 20 nt, synthetic construct
  misc_feature 1..20: note = Seynthetic
  source 1..20: mol_type = other RNA
agtcccagctgcgggaatgg

SEQ ID NO: 222 - RNA, 20 nt, synthetic construct
  misc_feature 1..20: note = Seynthetic
  source 1..20: mol_type = other RNA
agtcccccagggtgtttcac

SEQ ID NO: 223 - RNA, 20 nt, synthetic construct
  misc_feature 1..20: note = Seynthetic
  source 1..20: mol_type = other RNA
agtcctgacgtgaccacagg

SEQ ID NO: 224 - RNA, 20 nt, synthetic construct
  misc_feature 1..20: note = Seynthetic
  source 1..20: mol_type = other RNA
agtctacacccgctgtgacc

SEQ ID NO: 225 - RNA, 20 nt, synthetic construct
  misc_feature 1..20: note = Seynthetic
  source 1..20: mol_type = other RNA
agtctacacctgctgtgacc

SEQ ID NO: 226 - RNA, 20 nt, synthetic construct
  misc_feature 1..20: note = Seynthetic
  source 1..20: mol_type = other RNA
agtcttgacgtgaccacagg

SEQ ID NO: 227 - RNA, 20 nt, synthetic construct
  misc_feature 1..20: note = Seynthetic
  source 1..20: mol_type = other RNA
agtctttattaagtgtgccg

SEQ ID NO: 228 - RNA, 20 nt, synthetic construct
  misc_feature 1..20: note = Seynthetic
  source 1..20: mol_type = other RNA
agtgacttccccacattgta

SEQ ID NO: 229 - RNA, 20 nt, synthetic construct
  misc_feature 1..20: note = Seynthetic
  source 1..20: mol_type = other RNA
agtgacttccccacattgtg

SEQ ID NO: 230 - RNA, 20 nt, synthetic construct
  misc_feature 1..20: note = Seynthetic
  source 1..20: mol_type = other RNA
agttcagggatgtccgcctt

SEQ ID NO: 231 - RNA, 20 nt, synthetic construct
  misc_feature 1..20: note = Seynthetic
  source 1..20: mol_type = other RNA
agttccctggtagaagcatg

SEQ ID NO: 232 - RNA, 20 nt, synthetic construct
  misc_feature 1..20: note = Seynthetic
  source 1..20: mol_type = other RNA
ataaagactgcacgtgagaa

SEQ ID NO: 233 - RNA, 20 nt, synthetic construct
  misc_feature 1..20: note = Seynthetic
  source 1..20: mol_type = other RNA
ataaattcacgttcgtaaag

SEQ ID NO: 234 - RNA, 20 nt, synthetic construct
  misc_feature 1..20: note = Seynthetic
  source 1..20: mol_type = other RNA
ataatgactgcacgtgagaa

SEQ ID NO: 235 - RNA, 20 nt, synthetic construct
  misc_feature 1..20: note = Seynthetic
  source 1..20: mol_type = other RNA
atacaatgtggggaagtcac

SEQ ID NO: 236 - RNA, 20 nt, synthetic construct
  misc_feature 1..20: note = Seynthetic
  source 1..20: mol_type = other RNA
atacagcgtcccccacccct

SEQ ID NO: 237 - RNA, 20 nt, synthetic construct
  misc_feature 1..20: note = Seynthetic
  source 1..20: mol_type = other RNA
atacggcgtcccccacccct

SEQ ID NO: 238 - RNA, 20 nt, synthetic construct
  misc_feature 1..20: note = Seynthetic
  source 1..20: mol_type = other RNA
atactacattctcacgtgca

SEQ ID NO: 239 - RNA, 20 nt, synthetic construct
  misc_feature 1..20: note = Seynthetic
  source 1..20: mol_type = other RNA
atatggaaagttcagggatg

SEQ ID NO: 240 - RNA, 20 nt, synthetic construct
  misc_feature 1..20: note = Seynthetic
  source 1..20: mol_type = other RNA
atccatggtgaagagaaaag

SEQ ID NO: 241 - RNA, 20 nt, synthetic construct
  misc_feature 1..20: note = Seynthetic
  source 1..20: mol_type = other RNA
atccatggtgcagggaccag

SEQ ID NO: 242 - RNA, 20 nt, synthetic construct
  misc_feature 1..20: note = Seynthetic
  source 1..20: mol_type = other RNA
atccatggtgcggggaccag

SEQ ID NO: 243 - RNA, 20 nt, synthetic construct
  misc_feature 1..20: note = Seynthetic
  source 1..20: mol_type = other RNA
atcccattcccgcagctggg

SEQ ID NO: 244 - RNA, 20 nt, synthetic construct
  misc_feature 1..20: note = Seynthetic
  source 1..20: mol_type = other RNA
atccccatccttctggtccc

SEQ ID NO: 245 - RNA, 20 nt, synthetic construct
  misc_feature 1..20: note = Seynthetic
  source 1..20: mol_type = other RNA
atccctttcccgcagctggg

SEQ ID NO: 246 - RNA, 20 nt, synthetic construct
  misc_feature 1..20: note = Seynthetic
  source 1..20: mol_type = other RNA
atccttctggtccccgcacc

SEQ ID NO: 247 - RNA, 20 nt, synthetic construct
  misc_feature 1..20: note = Seynthetic
  source 1..20: mol_type = other RNA
atccttctggtccctgcacc

SEQ ID NO: 248 - RNA, 20 nt, synthetic construct
  misc_feature 1..20: note = Seynthetic
  source 1..20: mol_type = other RNA
atcgcattcaaatgtcagat

SEQ ID NO: 249 - RNA, 20 nt, synthetic construct
  misc_feature 1..20: note = Seynthetic
  source 1..20: mol_type = other RNA
atcgtgggggagatgggaag

SEQ ID NO: 250 - RNA, 20 nt, synthetic construct
  misc_feature 1..20: note = Seynthetic
  source 1..20: mol_type = other RNA
atctcccccaccatgttcca

SEQ ID NO: 251 - RNA, 20 nt, synthetic construct
  misc_feature 1..20: note = Seynthetic
  source 1..20: mol_type = other RNA
atctcccccacgatgttcca

SEQ ID NO: 252 - RNA, 20 nt, synthetic construct
  misc_feature 1..20: note = Seynthetic
  source 1..20: mol_type = other RNA
atctgaaatttgaatgcgat

SEQ ID NO: 253 - RNA, 20 nt, synthetic construct
  misc_feature 1..20: note = Seynthetic
  source 1..20: mol_type = other RNA
atctgacatttgaatgcgat

SEQ ID NO: 254 - RNA, 20 nt, synthetic construct
  misc_feature 1..20: note = Seynthetic
  source 1..20: mol_type = other RNA
atgaaataaattcacgttcg

SEQ ID NO: 255 - RNA, 20 nt, synthetic construct
  misc_feature 1..20: note = Seynthetic
  source 1..20: mol_type = other RNA
atgactgcacgtgagaatgt

SEQ ID NO: 256 - RNA, 20 nt, synthetic construct
  misc_feature 1..20: note = Seynthetic
  source 1..20: mol_type = other RNA
atgcacaatcgcattcaaat

SEQ ID NO: 257 - RNA, 20 nt, synthetic construct
  misc_feature 1..20: note = Seynthetic
  source 1..20: mol_type = other RNA
atgccgccttcaggtgtggc

SEQ ID NO: 258 - RNA, 20 nt, synthetic construct
  misc_feature 1..20: note = Seynthetic
  source 1..20: mol_type = other RNA
atgcttctaccagggaactc

SEQ ID NO: 259 - RNA, 20 nt, synthetic construct
  misc_feature 1..20: note = Seynthetic
  source 1..20: mol_type = other RNA
atgcttctaccagggagctc

SEQ ID NO: 260 - RNA, 20 nt, synthetic construct
  misc_feature 1..20: note = Seynthetic
  source 1..20: mol_type = other RNA
atggaaagttcagggatgtc

SEQ ID NO: 261 - RNA, 20 nt, synthetic construct
  misc_feature 1..20: note = Seynthetic
  source 1..20: mol_type = other RNA
atgggattcccaggacccag

SEQ ID NO: 262 - RNA, 20 nt, synthetic construct
  misc_feature 1..20: note = Seynthetic
  source 1..20: mol_type = other RNA
atggtcacagcaggtgtaga

SEQ ID NO: 263 - RNA, 20 nt, synthetic construct
  misc_feature 1..20: note = Seynthetic
  source 1..20: mol_type = other RNA
atggtcacagcgggtgtaga

SEQ ID NO: 264 - RNA, 20 nt, synthetic construct
  misc_feature 1..20: note = Seynthetic
  source 1..20: mol_type = other RNA
atggtgaagagaaaagccgg

SEQ ID NO: 265 - RNA, 20 nt, synthetic construct
  misc_feature 1..20: note = Seynthetic
  source 1..20: mol_type = other RNA
atggtgcagggaccagaagg

SEQ ID NO: 266 - RNA, 20 nt, synthetic construct
  misc_feature 1..20: note = Seynthetic
  source 1..20: mol_type = other RNA
atggtgcggggaccagaagg

SEQ ID NO: 267 - RNA, 20 nt, synthetic construct
  misc_feature 1..20: note = Seynthetic
  source 1..20: mol_type = other RNA
atgtcagataatcaatgtgg

SEQ ID NO: 268 - RNA, 20 nt, synthetic construct
  misc_feature 1..20: note = Seynthetic
  source 1..20: mol_type = other RNA
atgtccgccttcaggtgtgg

SEQ ID NO: 269 - RNA, 20 nt, synthetic construct
  misc_feature 1..20: note = Seynthetic
  source 1..20: mol_type = other RNA
attaatccatggtgcaggga

SEQ ID NO: 270 - RNA, 20 nt, synthetic construct
  misc_feature 1..20: note = Seynthetic
  source 1..20: mol_type = other RNA
attaatccatggtgcgggga

SEQ ID NO: 271 - RNA, 20 nt, synthetic construct
  misc_feature 1..20: note = Seynthetic
  source 1..20: mol_type = other RNA
attatctgacatttgaatgc

SEQ ID NO: 272 - RNA, 20 nt, synthetic construct
  misc_feature 1..20: note = Seynthetic
  source 1..20: mol_type = other RNA
attattaagtgtgccgacgc

SEQ ID NO: 273 - RNA, 20 nt, synthetic construct
  misc_feature 1..20: note = Seynthetic
  source 1..20: mol_type = other RNA
attcacgttcataaagttct

SEQ ID NO: 274 - RNA, 20 nt, synthetic construct
  misc_feature 1..20: note = Seynthetic
  source 1..20: mol_type = other RNA
attcacgttcgtaaagttct

SEQ ID NO: 275 - RNA, 20 nt, synthetic construct
  misc_feature 1..20: note = Seynthetic
  source 1..20: mol_type = other RNA
attcccgcagctgggactcc

SEQ ID NO: 276 - RNA, 20 nt, synthetic construct
  misc_feature 1..20: note = Seynthetic
  source 1..20: mol_type = other RNA
attctcacgtgcagtcatta

SEQ ID NO: 277 - RNA, 20 nt, synthetic construct
  misc_feature 1..20: note = Seynthetic
  source 1..20: mol_type = other RNA
attctcacgtgcagtcttta

SEQ ID NO: 278 - RNA, 20 nt, synthetic construct
  misc_feature 1..20: note = Seynthetic
  source 1..20: mol_type = other RNA
attgctcatgcttctaccag

SEQ ID NO: 279 - RNA, 20 nt, synthetic construct
  misc_feature 1..20: note = Seynthetic
  source 1..20: mol_type = other RNA
attgtgaaacaccctggggg

SEQ ID NO: 280 - RNA, 20 nt, synthetic construct
  misc_feature 1..20: note = Seynthetic
  source 1..20: mol_type = other RNA
attgtgaaacaccctgggtg

SEQ ID NO: 281 - RNA, 20 nt, synthetic construct
  misc_feature 1..20: note = Seynthetic
  source 1..20: mol_type = other RNA
atttcagataatcaatgtgg

SEQ ID NO: 282 - RNA, 20 nt, synthetic construct
  misc_feature 1..20: note = Seynthetic
  source 1..20: mol_type = other RNA
atttcaggggtgggggacgc

SEQ ID NO: 283 - RNA, 20 nt, synthetic construct
  misc_feature 1..20: note = Seynthetic
  source 1..20: mol_type = other RNA
atttttttttaatggagcgg

SEQ ID NO: 284 - RNA, 20 nt, synthetic construct
  misc_feature 1..20: note = Seynthetic
  source 1..20: mol_type = other RNA
caaaacccccgctccattaa

SEQ ID NO: 285 - RNA, 20 nt, synthetic construct
  misc_feature 1..20: note = Seynthetic
  source 1..20: mol_type = other RNA
caaagatacagcgtccccca

SEQ ID NO: 286 - RNA, 20 nt, synthetic construct
  misc_feature 1..20: note = Seynthetic
  source 1..20: mol_type = other RNA
caaagatacggcgtccccca

SEQ ID NO: 287 - RNA, 20 nt, synthetic construct
  misc_feature 1..20: note = Seynthetic
  source 1..20: mol_type = other RNA
caaatgtcagataatcaatg

SEQ ID NO: 288 - RNA, 20 nt, synthetic construct
  misc_feature 1..20: note = Seynthetic
  source 1..20: mol_type = other RNA
caacttcaccacacccagag

SEQ ID NO: 289 - RNA, 20 nt, synthetic construct
  misc_feature 1..20: note = Seynthetic
  source 1..20: mol_type = other RNA
caagccacacctgaaggcgg

SEQ ID NO: 290 - RNA, 20 nt, synthetic construct
  misc_feature 1..20: note = Seynthetic
  source 1..20: mol_type = other RNA
caagccccagaggccacagg

SEQ ID NO: 291 - RNA, 20 nt, synthetic construct
  misc_feature 1..20: note = Seynthetic
  source 1..20: mol_type = other RNA
caagccccttccaaagatac

SEQ ID NO: 292 - RNA, 20 nt, synthetic construct
  misc_feature 1..20: note = Seynthetic
  source 1..20: mol_type = other RNA
caaggggctgccggagtccc

SEQ ID NO: 293 - RNA, 20 nt, synthetic construct
  misc_feature 1..20: note = Seynthetic
  source 1..20: mol_type = other RNA
caatcgcattcaaatgtcag

SEQ ID NO: 294 - RNA, 20 nt, synthetic construct
  misc_feature 1..20: note = Seynthetic
  source 1..20: mol_type = other RNA
caatcgcattcaaatttcag

SEQ ID NO: 295 - RNA, 20 nt, synthetic construct
  misc_feature 1..20: note = Seynthetic
  source 1..20: mol_type = other RNA
cacaatcgcattcaaatgtc

SEQ ID NO: 296 - RNA, 20 nt, synthetic construct
  misc_feature 1..20: note = Seynthetic
  source 1..20: mol_type = other RNA
cacaatcgcattcaaatttc

SEQ ID NO: 297 - RNA, 20 nt, synthetic construct
  misc_feature 1..20: note = Seynthetic
  source 1..20: mol_type = other RNA
cacaatgtggggaagtcact

SEQ ID NO: 298 - RNA, 20 nt, synthetic construct
  misc_feature 1..20: note = Seynthetic
  source 1..20: mol_type = other RNA
cacacacagctattttctta

SEQ ID NO: 299 - RNA, 20 nt, synthetic construct
  misc_feature 1..20: note = Seynthetic
  source 1..20: mol_type = other RNA
cacacccagagtcacccagg

SEQ ID NO: 300 - RNA, 20 nt, synthetic construct
  misc_feature 1..20: note = Seynthetic
  source 1..20: mol_type = other RNA
cacacccagagtcccccagg

SEQ ID NO: 301 - RNA, 20 nt, synthetic construct
  misc_feature 1..20: note = Seynthetic
  source 1..20: mol_type = other RNA
cacacctgaaggcggacatc

SEQ ID NO: 302 - RNA, 20 nt, synthetic construct
  misc_feature 1..20: note = Seynthetic
  source 1..20: mol_type = other RNA
cacacgaggttcccccggct

SEQ ID NO: 303 - RNA, 20 nt, synthetic construct
  misc_feature 1..20: note = Seynthetic
  source 1..20: mol_type = other RNA
cacacgaggttccccctgct

SEQ ID NO: 304 - RNA, 20 nt, synthetic construct
  misc_feature 1..20: note = Seynthetic
  source 1..20: mol_type = other RNA
cacacggacggtcctccagc

SEQ ID NO: 305 - RNA, 20 nt, synthetic construct
  misc_feature 1..20: note = Seynthetic
  source 1..20: mol_type = other RNA
cacacttaataaagactgca

SEQ ID NO: 306 - RNA, 20 nt, synthetic construct
  misc_feature 1..20: note = Seynthetic
  source 1..20: mol_type = other RNA
cacacttaataatgactgca

SEQ ID NO: 307 - RNA, 20 nt, synthetic construct
  misc_feature 1..20: note = Seynthetic
  source 1..20: mol_type = other RNA
cacagcaggtgtagactccg

SEQ ID NO: 308 - RNA, 20 nt, synthetic construct
  misc_feature 1..20: note = Seynthetic
  source 1..20: mol_type = other RNA
cacagcgggtgtagactccg

SEQ ID NO: 309 - RNA, 20 nt, synthetic construct
  misc_feature 1..20: note = Seynthetic
  source 1..20: mol_type = other RNA
cacagctattttcttagttt

SEQ ID NO: 310 - RNA, 20 nt, synthetic construct
  misc_feature 1..20: note = Seynthetic
  source 1..20: mol_type = other RNA
cacagggggactccggcagc

SEQ ID NO: 311 - RNA, 20 nt, synthetic construct
  misc_feature 1..20: note = Seynthetic
  source 1..20: mol_type = other RNA
cacattgattatctgacatt

SEQ ID NO: 312 - RNA, 20 nt, synthetic construct
  misc_feature 1..20: note = Seynthetic
  source 1..20: mol_type = other RNA
caccacacccagagtcaccc

SEQ ID NO: 313 - RNA, 20 nt, synthetic construct
  misc_feature 1..20: note = Seynthetic
  source 1..20: mol_type = other RNA
caccacacccagagtccccc

SEQ ID NO: 314 - RNA, 20 nt, synthetic construct
  misc_feature 1..20: note = Seynthetic
  source 1..20: mol_type = other RNA
caccatgttccagctgcaac

SEQ ID NO: 315 - RNA, 20 nt, synthetic construct
  misc_feature 1..20: note = Seynthetic
  source 1..20: mol_type = other RNA
cacccagagtcacccagggt

SEQ ID NO: 316 - RNA, 20 nt, synthetic construct
  misc_feature 1..20: note = Seynthetic
  source 1..20: mol_type = other RNA
cacccagagtcccccagggt

SEQ ID NO: 317 - RNA, 20 nt, synthetic construct
  misc_feature 1..20: note = Seynthetic
  source 1..20: mol_type = other RNA
cacccagggtgtttcacaat

SEQ ID NO: 318 - RNA, 20 nt, synthetic construct
  misc_feature 1..20: note = Seynthetic
  source 1..20: mol_type = other RNA
cacccccttcctgcccacct

SEQ ID NO: 319 - RNA, 20 nt, synthetic construct
  misc_feature 1..20: note = Seynthetic
  source 1..20: mol_type = other RNA
cacccgctgtgaccataaca

SEQ ID NO: 320 - RNA, 20 nt, synthetic construct
  misc_feature 1..20: note = Seynthetic
  source 1..20: mol_type = other RNA
caccctgggggactctgggt

SEQ ID NO: 321 - RNA, 20 nt, synthetic construct
  misc_feature 1..20: note = Seynthetic
  source 1..20: mol_type = other RNA
caccctgggtgactctgggt

SEQ ID NO: 322 - RNA, 20 nt, synthetic construct
  misc_feature 1..20: note = Seynthetic
  source 1..20: mol_type = other RNA
cacctgaaggcggacatccc

SEQ ID NO: 323 - RNA, 20 nt, synthetic construct
  misc_feature 1..20: note = Seynthetic
  source 1..20: mol_type = other RNA
cacctgctgtgaccataaca

SEQ ID NO: 324 - RNA, 20 nt, synthetic construct
  misc_feature 1..20: note = Seynthetic
  source 1..20: mol_type = other RNA
cacctggaggggctggagga

SEQ ID NO: 325 - RNA, 20 nt, synthetic construct
  misc_feature 1..20: note = Seynthetic
  source 1..20: mol_type = other RNA
cacctggcggggctggagga

SEQ ID NO: 326 - RNA, 20 nt, synthetic construct
  misc_feature 1..20: note = Seynthetic
  source 1..20: mol_type = other RNA
cacctttgtctctgtccctg

SEQ ID NO: 327 - RNA, 20 nt, synthetic construct
  misc_feature 1..20: note = Seynthetic
  source 1..20: mol_type = other RNA
cacgaggttcccccggcttt

SEQ ID NO: 328 - RNA, 20 nt, synthetic construct
  misc_feature 1..20: note = Seynthetic
  source 1..20: mol_type = other RNA
cacgaggttccccctgcttt

SEQ ID NO: 329 - RNA, 20 nt, synthetic construct
  misc_feature 1..20: note = Seynthetic
  source 1..20: mol_type = other RNA
cacgatgttccagctgcaac

SEQ ID NO: 330 - RNA, 20 nt, synthetic construct
  misc_feature 1..20: note = Seynthetic
  source 1..20: mol_type = other RNA
cacggacggtcctccagccc

SEQ ID NO: 331 - RNA, 20 nt, synthetic construct
  misc_feature 1..20: note = Seynthetic
  source 1..20: mol_type = other RNA
cacgtcaagactccagcctg

SEQ ID NO: 332 - RNA, 20 nt, synthetic construct
  misc_feature 1..20: note = Seynthetic
  source 1..20: mol_type = other RNA
cacgtcaggactccagcctg

SEQ ID NO: 333 - RNA, 20 nt, synthetic construct
  misc_feature 1..20: note = Seynthetic
  source 1..20: mol_type = other RNA
cacgtgcagtcattattaag

SEQ ID NO: 334 - RNA, 20 nt, synthetic construct
  misc_feature 1..20: note = Seynthetic
  source 1..20: mol_type = other RNA
cacgtgcagtctttattaag

SEQ ID NO: 335 - RNA, 20 nt, synthetic construct
  misc_feature 1..20: note = Seynthetic
  source 1..20: mol_type = other RNA
cacgttcataaagttcttag

SEQ ID NO: 336 - RNA, 20 nt, synthetic construct
  misc_feature 1..20: note = Seynthetic
  source 1..20: mol_type = other RNA
cacgttcgtaaagttcttag

SEQ ID NO: 337 - RNA, 20 nt, synthetic construct
  misc_feature 1..20: note = Seynthetic
  source 1..20: mol_type = other RNA
cacgttgcattaatccatgg

SEQ ID NO: 338 - RNA, 20 nt, synthetic construct
  misc_feature 1..20: note = Seynthetic
  source 1..20: mol_type = other RNA
cacttaataaagactgcacg

SEQ ID NO: 339 - RNA, 20 nt, synthetic construct
  misc_feature 1..20: note = Seynthetic
  source 1..20: mol_type = other RNA
cacttaataatgactgcacg

SEQ ID NO: 340 - RNA, 20 nt, synthetic construct
  misc_feature 1..20: note = Seynthetic
  source 1..20: mol_type = other RNA
cacttgcctgtggtcacgtc

SEQ ID NO: 341 - RNA, 20 nt, synthetic construct
  misc_feature 1..20: note = Seynthetic
  source 1..20: mol_type = other RNA
cactttcttagctaagaact

SEQ ID NO: 342 - RNA, 20 nt, synthetic construct
  misc_feature 1..20: note = Seynthetic
  source 1..20: mol_type = other RNA
cagaagtgacttccccacat

SEQ ID NO: 343 - RNA, 20 nt, synthetic construct
  misc_feature 1..20: note = Seynthetic
  source 1..20: mol_type = other RNA
cagaccaaggggctgccgga

SEQ ID NO: 344 - RNA, 20 nt, synthetic construct
  misc_feature 1..20: note = Seynthetic
  source 1..20: mol_type = other RNA
cagaccaaggggctgcctga

SEQ ID NO: 345 - RNA, 20 nt, synthetic construct
  misc_feature 1..20: note = Seynthetic
  source 1..20: mol_type = other RNA
cagaggacacagggacagag

SEQ ID NO: 346 - RNA, 20 nt, synthetic construct
  misc_feature 1..20: note = Seynthetic
  source 1..20: mol_type = other RNA
cagaggccacagggacagag

SEQ ID NO: 347 - RNA, 20 nt, synthetic construct
  misc_feature 1..20: note = Seynthetic
  source 1..20: mol_type = other RNA
cagagtcacccagggtgttt

SEQ ID NO: 348 - RNA, 20 nt, synthetic construct
  misc_feature 1..20: note = Seynthetic
  source 1..20: mol_type = other RNA
cagagtcccccagggtgttt

SEQ ID NO: 349 - RNA, 20 nt, synthetic construct
  misc_feature 1..20: note = Seynthetic
  source 1..20: mol_type = other RNA
cagcacacgaggttcccccg

SEQ ID NO: 350 - RNA, 20 nt, synthetic construct
  misc_feature 1..20: note = Seynthetic
  source 1..20: mol_type = other RNA
cagcacacgaggttccccct

SEQ ID NO: 351 - RNA, 20 nt, synthetic construct
  misc_feature 1..20: note = Seynthetic
  source 1..20: mol_type = other RNA
cagcaggtgtagactccgag

SEQ ID NO: 352 - RNA, 20 nt, synthetic construct
  misc_feature 1..20: note = Seynthetic
  source 1..20: mol_type = other RNA
cagcgggtgtagactccgag

SEQ ID NO: 353 - RNA, 20 nt, synthetic construct
  misc_feature 1..20: note = Seynthetic
  source 1..20: mol_type = other RNA
cagcgtcccccacccctgaa

SEQ ID NO: 354 - RNA, 20 nt, synthetic construct
  misc_feature 1..20: note = Seynthetic
  source 1..20: mol_type = other RNA
cagcgtcggcacacttaata

SEQ ID NO: 355 - RNA, 20 nt, synthetic construct
  misc_feature 1..20: note = Seynthetic
  source 1..20: mol_type = other RNA
cagctctcaaagcaacactt

SEQ ID NO: 356 - RNA, 20 nt, synthetic construct
  misc_feature 1..20: note = Seynthetic
  source 1..20: mol_type = other RNA
cagctgcgggaaagggattc

SEQ ID NO: 357 - RNA, 20 nt, synthetic construct
  misc_feature 1..20: note = Seynthetic
  source 1..20: mol_type = other RNA
cagctgcgggaatgggattc

SEQ ID NO: 358 - RNA, 20 nt, synthetic construct
  misc_feature 1..20: note = Seynthetic
  source 1..20: mol_type = other RNA
cagctggaacatcgtggggg

SEQ ID NO: 359 - RNA, 20 nt, synthetic construct
  misc_feature 1..20: note = Seynthetic
  source 1..20: mol_type = other RNA
cagctggaacatggtggggg

SEQ ID NO: 360 - RNA, 20 nt, synthetic construct
  misc_feature 1..20: note = Seynthetic
  source 1..20: mol_type = other RNA
caggactccagcctggccca

SEQ ID NO: 361 - RNA, 20 nt, synthetic construct
  misc_feature 1..20: note = Seynthetic
  source 1..20: mol_type = other RNA
caggagctccctggtagaag

SEQ ID NO: 362 - RNA, 20 nt, synthetic construct
  misc_feature 1..20: note = Seynthetic
  source 1..20: mol_type = other RNA
caggagttccctggtagaag

SEQ ID NO: 363 - RNA, 20 nt, synthetic construct
  misc_feature 1..20: note = Seynthetic
  source 1..20: mol_type = other RNA
caggctggagtcctgacgtg

SEQ ID NO: 364 - RNA, 20 nt, synthetic construct
  misc_feature 1..20: note = Seynthetic
  source 1..20: mol_type = other RNA
caggctggagtcttgacgtg

SEQ ID NO: 365 - RNA, 20 nt, synthetic construct
  misc_feature 1..20: note = Seynthetic
  source 1..20: mol_type = other RNA
cagggaactcctgccctgcc

SEQ ID NO: 366 - RNA, 20 nt, synthetic construct
  misc_feature 1..20: note = Seynthetic
  source 1..20: mol_type = other RNA
cagggaccagaaggatgggg

SEQ ID NO: 367 - RNA, 20 nt, synthetic construct
  misc_feature 1..20: note = Seynthetic
  source 1..20: mol_type = other RNA
cagggagctcctgccctgcc

SEQ ID NO: 368 - RNA, 20 nt, synthetic construct
  misc_feature 1..20: note = Seynthetic
  source 1..20: mol_type = other RNA
cagggatgtccgccttcagg

SEQ ID NO: 369 - RNA, 20 nt, synthetic construct
  misc_feature 1..20: note = Seynthetic
  source 1..20: mol_type = other RNA
cagggcaggagttccctggt

SEQ ID NO: 370 - RNA, 20 nt, synthetic construct
  misc_feature 1..20: note = Seynthetic
  source 1..20: mol_type = other RNA
cagggggaacctcgtgtgct

SEQ ID NO: 371 - RNA, 20 nt, synthetic construct
  misc_feature 1..20: note = Seynthetic
  source 1..20: mol_type = other RNA
caggggtgggggacgccgta

SEQ ID NO: 372 - RNA, 20 nt, synthetic construct
  misc_feature 1..20: note = Seynthetic
  source 1..20: mol_type = other RNA
caggggtgggggacgctgta

SEQ ID NO: 373 - RNA, 20 nt, synthetic construct
  misc_feature 1..20: note = Seynthetic
  source 1..20: mol_type = other RNA
cagggtgtcaagccccagag

SEQ ID NO: 374 - RNA, 20 nt, synthetic construct
  misc_feature 1..20: note = Seynthetic
  source 1..20: mol_type = other RNA
caggtgtagactccgagggg

SEQ ID NO: 375 - RNA, 20 nt, synthetic construct
  misc_feature 1..20: note = Seynthetic
  source 1..20: mol_type = other RNA
cagtcattattaagtgtgcc

SEQ ID NO: 376 - RNA, 20 nt, synthetic construct
  misc_feature 1..20: note = Seynthetic
  source 1..20: mol_type = other RNA
cagtctttattaagtgtgcc

SEQ ID NO: 377 - RNA, 20 nt, synthetic construct
  misc_feature 1..20: note = Seynthetic
  source 1..20: mol_type = other RNA
cataaagttcttagctaaga

SEQ ID NO: 378 - RNA, 20 nt, synthetic construct
  misc_feature 1..20: note = Seynthetic
  source 1..20: mol_type = other RNA
catccttctggtccccgcac

SEQ ID NO: 379 - RNA, 20 nt, synthetic construct
  misc_feature 1..20: note = Seynthetic
  source 1..20: mol_type = other RNA
catccttctggtccctgcac

SEQ ID NO: 380 - RNA, 20 nt, synthetic construct
  misc_feature 1..20: note = Seynthetic
  source 1..20: mol_type = other RNA
catcgtgggggagatgggaa

SEQ ID NO: 381 - RNA, 20 nt, synthetic construct
  misc_feature 1..20: note = Seynthetic
  source 1..20: mol_type = other RNA
catctcccccaccatgttcc

SEQ ID NO: 382 - RNA, 20 nt, synthetic construct
  misc_feature 1..20: note = Seynthetic
  source 1..20: mol_type = other RNA
catctcccccacgatgttcc

SEQ ID NO: 383 - RNA, 20 nt, synthetic construct
  misc_feature 1..20: note = Seynthetic
  source 1..20: mol_type = other RNA
catgcttctaccagggaact

SEQ ID NO: 384 - RNA, 20 nt, synthetic construct
  misc_feature 1..20: note = Seynthetic
  source 1..20: mol_type = other RNA
catgcttctaccagggagct

SEQ ID NO: 385 - RNA, 20 nt, synthetic construct
  misc_feature 1..20: note = Seynthetic
  source 1..20: mol_type = other RNA
catggtgaagagaaaagccg

SEQ ID NO: 386 - RNA, 20 nt, synthetic construct
  misc_feature 1..20: note = Seynthetic
  source 1..20: mol_type = other RNA
catggtgcagggaccagaag

SEQ ID NO: 387 - RNA, 20 nt, synthetic construct
  misc_feature 1..20: note = Seynthetic
  source 1..20: mol_type = other RNA
catggtgcggggaccagaag

SEQ ID NO: 388 - RNA, 20 nt, synthetic construct
  misc_feature 1..20: note = Seynthetic
  source 1..20: mol_type = other RNA
catgttccagctgcaacggc

SEQ ID NO: 389 - RNA, 20 nt, synthetic construct
  misc_feature 1..20: note = Seynthetic
  source 1..20: mol_type = other RNA
cattaatccatggtgcaggg

SEQ ID NO: 390 - RNA, 20 nt, synthetic construct
  misc_feature 1..20: note = Seynthetic
  source 1..20: mol_type = other RNA
cattaatccatggtgcgggg

SEQ ID NO: 391 - RNA, 20 nt, synthetic construct
  misc_feature 1..20: note = Seynthetic
  source 1..20: mol_type = other RNA
cattattaagtgtgccgacg

SEQ ID NO: 392 - RNA, 20 nt, synthetic construct
  misc_feature 1..20: note = Seynthetic
  source 1..20: mol_type = other RNA
cattcccgcagctgggactc

SEQ ID NO: 393 - RNA, 20 nt, synthetic construct
  misc_feature 1..20: note = Seynthetic
  source 1..20: mol_type = other RNA
cattctcacgtgcagtcatt

SEQ ID NO: 394 - RNA, 20 nt, synthetic construct
  misc_feature 1..20: note = Seynthetic
  source 1..20: mol_type = other RNA
cattctcacgtgcagtcttt

SEQ ID NO: 395 - RNA, 20 nt, synthetic construct
  misc_feature 1..20: note = Seynthetic
  source 1..20: mol_type = other RNA
catttgaatgcgattgtgca

SEQ ID NO: 396 - RNA, 20 nt, synthetic construct
  misc_feature 1..20: note = Seynthetic
  source 1..20: mol_type = other RNA
ccaaagatacagcgtccccc

SEQ ID NO: 397 - RNA, 20 nt, synthetic construct
  misc_feature 1..20: note = Seynthetic
  source 1..20: mol_type = other RNA
ccaaagatacggcgtccccc

SEQ ID NO: 398 - RNA, 20 nt, synthetic construct
  misc_feature 1..20: note = Seynthetic
  source 1..20: mol_type = other RNA
ccaacttcaccacacccaga

SEQ ID NO: 399 - RNA, 20 nt, synthetic construct
  misc_feature 1..20: note = Seynthetic
  source 1..20: mol_type = other RNA
ccaagccccttccaaagata

SEQ ID NO: 400 - RNA, 20 nt, synthetic construct
  misc_feature 1..20: note = Seynthetic
  source 1..20: mol_type = other RNA
ccaaggggctgccggagtcc

SEQ ID NO: 401 - RNA, 20 nt, synthetic construct
  misc_feature 1..20: note = Seynthetic
  source 1..20: mol_type = other RNA
ccacacccagagtcacccag

SEQ ID NO: 402 - RNA, 20 nt, synthetic construct
  misc_feature 1..20: note = Seynthetic
  source 1..20: mol_type = other RNA
ccacacccagagtcccccag

SEQ ID NO: 403 - RNA, 20 nt, synthetic construct
  misc_feature 1..20: note = Seynthetic
  source 1..20: mol_type = other RNA
ccacacctgaaggcggacat

SEQ ID NO: 404 - RNA, 20 nt, synthetic construct
  misc_feature 1..20: note = Seynthetic
  source 1..20: mol_type = other RNA
ccacagggacagagacaaag

SEQ ID NO: 405 - RNA, 20 nt, synthetic construct
  misc_feature 1..20: note = Seynthetic
  source 1..20: mol_type = other RNA
ccacagggggactccggcag

SEQ ID NO: 406 - RNA, 20 nt, synthetic construct
  misc_feature 1..20: note = Seynthetic
  source 1..20: mol_type = other RNA
ccacattgattatctgaaat

SEQ ID NO: 407 - RNA, 20 nt, synthetic construct
  misc_feature 1..20: note = Seynthetic
  source 1..20: mol_type = other RNA
ccacattgattatctgacat

SEQ ID NO: 408 - RNA, 20 nt, synthetic construct
  misc_feature 1..20: note = Seynthetic
  source 1..20: mol_type = other RNA
ccaccatgttccagctgcaa

SEQ ID NO: 409 - RNA, 20 nt, synthetic construct
  misc_feature 1..20: note = Seynthetic
  source 1..20: mol_type = other RNA
ccacctggaggggctggagg

SEQ ID NO: 410 - RNA, 20 nt, synthetic construct
  misc_feature 1..20: note = Seynthetic
  source 1..20: mol_type = other RNA
ccacgatgttccagctgcaa

SEQ ID NO: 411 - RNA, 20 nt, synthetic construct
  misc_feature 1..20: note = Seynthetic
  source 1..20: mol_type = other RNA
ccacttgcctgtggtcacgt

SEQ ID NO: 412 - RNA, 20 nt, synthetic construct
  misc_feature 1..20: note = Seynthetic
  source 1..20: mol_type = other RNA
ccagaggacacagggacaga

SEQ ID NO: 413 - RNA, 20 nt, synthetic construct
  misc_feature 1..20: note = Seynthetic
  source 1..20: mol_type = other RNA
ccagagtcacccagggtgtt

SEQ ID NO: 414 - RNA, 20 nt, synthetic construct
  misc_feature 1..20: note = Seynthetic
  source 1..20: mol_type = other RNA
ccagagtcccccagggtgtt

SEQ ID NO: 415 - RNA, 20 nt, synthetic construct
  misc_feature 1..20: note = Seynthetic
  source 1..20: mol_type = other RNA
ccagcgtcggcacacttaat

SEQ ID NO: 416 - RNA, 20 nt, synthetic construct
  misc_feature 1..20: note = Seynthetic
  source 1..20: mol_type = other RNA
ccagctctcaaagcaacact

SEQ ID NO: 417 - RNA, 20 nt, synthetic construct
  misc_feature 1..20: note = Seynthetic
  source 1..20: mol_type = other RNA
ccagctgcgggaaagggatt

SEQ ID NO: 418 - RNA, 20 nt, synthetic construct
  misc_feature 1..20: note = Seynthetic
  source 1..20: mol_type = other RNA
ccagctgcgggaatgggatt

SEQ ID NO: 419 - RNA, 20 nt, synthetic construct
  misc_feature 1..20: note = Seynthetic
  source 1..20: mol_type = other RNA
ccaggctggagtcctgacgt

SEQ ID NO: 420 - RNA, 20 nt, synthetic construct
  misc_feature 1..20: note = Seynthetic
  source 1..20: mol_type = other RNA
ccaggctggagtcttgacgt

SEQ ID NO: 421 - RNA, 20 nt, synthetic construct
  misc_feature 1..20: note = Seynthetic
  source 1..20: mol_type = other RNA
ccagggaactcctgccctgc

SEQ ID NO: 422 - RNA, 20 nt, synthetic construct
  misc_feature 1..20: note = Seynthetic
  source 1..20: mol_type = other RNA
ccatccttctggtccccgca

SEQ ID NO: 423 - RNA, 20 nt, synthetic construct
  misc_feature 1..20: note = Seynthetic
  source 1..20: mol_type = other RNA
ccatccttctggtccctgca

SEQ ID NO: 424 - RNA, 20 nt, synthetic construct
  misc_feature 1..20: note = Seynthetic
  source 1..20: mol_type = other RNA
ccatctcccccaccatgttc

SEQ ID NO: 425 - RNA, 20 nt, synthetic construct
  misc_feature 1..20: note = Seynthetic
  source 1..20: mol_type = other RNA
ccatctcccccacgatgttc

SEQ ID NO: 426 - RNA, 20 nt, synthetic construct
  misc_feature 1..20: note = Seynthetic
  source 1..20: mol_type = other RNA
ccatggtgaagagaaaagca

SEQ ID NO: 427 - RNA, 20 nt, synthetic construct
  misc_feature 1..20: note = Seynthetic
  source 1..20: mol_type = other RNA
ccatggtgaagagaaaagcc

SEQ ID NO: 428 - RNA, 20 nt, synthetic construct
  misc_feature 1..20: note = Seynthetic
  source 1..20: mol_type = other RNA
ccatggtgcagggaccagaa

SEQ ID NO: 429 - RNA, 20 nt, synthetic construct
  misc_feature 1..20: note = Seynthetic
  source 1..20: mol_type = other RNA
ccatggtgcggggaccagaa

SEQ ID NO: 430 - RNA, 20 nt, synthetic construct
  misc_feature 1..20: note = Seynthetic
  source 1..20: mol_type = other RNA
ccatgttccagctgcaacgg

SEQ ID NO: 431 - RNA, 20 nt, synthetic construct
  misc_feature 1..20: note = Seynthetic
  source 1..20: mol_type = other RNA
ccattcccgcagctgggact

SEQ ID NO: 432 - RNA, 20 nt, synthetic construct
  misc_feature 1..20: note = Seynthetic
  source 1..20: mol_type = other RNA
cccacattgattatctgaca

SEQ ID NO: 433 - RNA, 20 nt, synthetic construct
  misc_feature 1..20: note = Seynthetic
  source 1..20: mol_type = other RNA
cccaccatgttccagctgca

SEQ ID NO: 434 - RNA, 20 nt, synthetic construct
  misc_feature 1..20: note = Seynthetic
  source 1..20: mol_type = other RNA
cccacctggaggggctggag

SEQ ID NO: 435 - RNA, 20 nt, synthetic construct
  misc_feature 1..20: note = Seynthetic
  source 1..20: mol_type = other RNA
cccacgatgttccagctgca

SEQ ID NO: 436 - RNA, 20 nt, synthetic construct
  misc_feature 1..20: note = Seynthetic
  source 1..20: mol_type = other RNA
cccagaggacacagggacag

SEQ ID NO: 437 - RNA, 20 nt, synthetic construct
  misc_feature 1..20: note = Seynthetic
  source 1..20: mol_type = other RNA
cccagaggccacagggacag

SEQ ID NO: 438 - RNA, 20 nt, synthetic construct
  misc_feature 1..20: note = Seynthetic
  source 1..20: mol_type = other RNA
cccagagtcacccagggtgt

SEQ ID NO: 439 - RNA, 20 nt, synthetic construct
  misc_feature 1..20: note = Seynthetic
  source 1..20: mol_type = other RNA
cccagagtcccccagggtgt

SEQ ID NO: 440 - RNA, 20 nt, synthetic construct
  misc_feature 1..20: note = Seynthetic
  source 1..20: mol_type = other RNA
cccagcgtcggcacacttaa

SEQ ID NO: 441 - RNA, 20 nt, synthetic construct
  misc_feature 1..20: note = Seynthetic
  source 1..20: mol_type = other RNA
cccagctgcgggaaagggat

SEQ ID NO: 442 - RNA, 20 nt, synthetic construct
  misc_feature 1..20: note = Seynthetic
  source 1..20: mol_type = other RNA
cccagctgcgggaatgggat

SEQ ID NO: 443 - RNA, 20 nt, synthetic construct
  misc_feature 1..20: note = Seynthetic
  source 1..20: mol_type = other RNA
cccatccttctggtccccgc

SEQ ID NO: 444 - RNA, 20 nt, synthetic construct
  misc_feature 1..20: note = Seynthetic
  source 1..20: mol_type = other RNA
cccatccttctggtccctgc

SEQ ID NO: 445 - RNA, 20 nt, synthetic construct
  misc_feature 1..20: note = Seynthetic
  source 1..20: mol_type = other RNA
cccatctcccccaccatgtt

SEQ ID NO: 446 - RNA, 20 nt, synthetic construct
  misc_feature 1..20: note = Seynthetic
  source 1..20: mol_type = other RNA
cccatctcccccacgatgtt

SEQ ID NO: 447 - RNA, 20 nt, synthetic construct
  misc_feature 1..20: note = Seynthetic
  source 1..20: mol_type = other RNA
cccattcccgcagctgggac

SEQ ID NO: 448 - RNA, 20 nt, synthetic construct
  misc_feature 1..20: note = Seynthetic
  source 1..20: mol_type = other RNA
ccccacattgtgtttttttc

SEQ ID NO: 449 - RNA, 20 nt, synthetic construct
  misc_feature 1..20: note = Seynthetic
  source 1..20: mol_type = other RNA
ccccaccatgttccagctgc

SEQ ID NO: 450 - RNA, 20 nt, synthetic construct
  misc_feature 1..20: note = Seynthetic
  source 1..20: mol_type = other RNA
ccccacgatgttccagctgc

SEQ ID NO: 451 - RNA, 20 nt, synthetic construct
  misc_feature 1..20: note = Seynthetic
  source 1..20: mol_type = other RNA
ccccagaggacacagggaca

SEQ ID NO: 452 - RNA, 20 nt, synthetic construct
  misc_feature 1..20: note = Seynthetic
  source 1..20: mol_type = other RNA
ccccagaggccacagggaca

SEQ ID NO: 453 - RNA, 20 nt, synthetic construct
  misc_feature 1..20: note = Seynthetic
  source 1..20: mol_type = other RNA
ccccagggtgtttcacaatc

SEQ ID NO: 454 - RNA, 20 nt, synthetic construct
  misc_feature 1..20: note = Seynthetic
  source 1..20: mol_type = other RNA
ccccatccttctggtccccg

SEQ ID NO: 455 - RNA, 20 nt, synthetic construct
  misc_feature 1..20: note = Seynthetic
  source 1..20: mol_type = other RNA
ccccatccttctggtccctg

SEQ ID NO: 456 - RNA, 20 nt, synthetic construct
  misc_feature 1..20: note = Seynthetic
  source 1..20: mol_type = other RNA
cccccaccatgttccagctg

SEQ ID NO: 457 - RNA, 20 nt, synthetic construct
  misc_feature 1..20: note = Seynthetic
  source 1..20: mol_type = other RNA
cccccacgatgttccagctg

SEQ ID NO: 458 - RNA, 20 nt, synthetic construct
  misc_feature 1..20: note = Seynthetic
  source 1..20: mol_type = other RNA
cccccagggtgtttcacaat

SEQ ID NO: 459 - RNA, 20 nt, synthetic construct
  misc_feature 1..20: note = Seynthetic
  source 1..20: mol_type = other RNA
cccccgctccattaaaaaaa

SEQ ID NO: 460 - RNA, 20 nt, synthetic construct
  misc_feature 1..20: note = Seynthetic
  source 1..20: mol_type = other RNA
cccccggcttttctcttcac

SEQ ID NO: 461 - RNA, 20 nt, synthetic construct
  misc_feature 1..20: note = Seynthetic
  source 1..20: mol_type = other RNA
ccccctcggagtctacaccc

SEQ ID NO: 462 - RNA, 20 nt, synthetic construct
  misc_feature 1..20: note = Seynthetic
  source 1..20: mol_type = other RNA
ccccctcggagtctacacct

SEQ ID NO: 463 - RNA, 20 nt, synthetic construct
  misc_feature 1..20: note = Seynthetic
  source 1..20: mol_type = other RNA
cccccttcctgcccacctgg

SEQ ID NO: 464 - RNA, 20 nt, synthetic construct
  misc_feature 1..20: note = Seynthetic
  source 1..20: mol_type = other RNA
ccccgcaccatggattaatg

SEQ ID NO: 465 - RNA, 20 nt, synthetic construct
  misc_feature 1..20: note = Seynthetic
  source 1..20: mol_type = other RNA
ccccggcttttctcttcacc

SEQ ID NO: 466 - RNA, 20 nt, synthetic construct
  misc_feature 1..20: note = Seynthetic
  source 1..20: mol_type = other RNA
cccctcggagtctacacccg

SEQ ID NO: 467 - RNA, 20 nt, synthetic construct
  misc_feature 1..20: note = Seynthetic
  source 1..20: mol_type = other RNA
cccctcggagtctacacctg

SEQ ID NO: 468 - RNA, 20 nt, synthetic construct
  misc_feature 1..20: note = Seynthetic
  source 1..20: mol_type = other RNA
cccctgcttttctcttcacc

SEQ ID NO: 469 - RNA, 20 nt, synthetic construct
  misc_feature 1..20: note = Seynthetic
  source 1..20: mol_type = other RNA
ccccttccaaagatacagcg

SEQ ID NO: 470 - RNA, 20 nt, synthetic construct
  misc_feature 1..20: note = Seynthetic
  source 1..20: mol_type = other RNA
ccccttccaaagatacggcg

SEQ ID NO: 471 - RNA, 20 nt, synthetic construct
  misc_feature 1..20: note = Seynthetic
  source 1..20: mol_type = other RNA
cccgcaccatggattaatgc

SEQ ID NO: 472 - RNA, 20 nt, synthetic construct
  misc_feature 1..20: note = Seynthetic
  source 1..20: mol_type = other RNA
cccgccaggtgggcaggaag

SEQ ID NO: 473 - RNA, 20 nt, synthetic construct
  misc_feature 1..20: note = Seynthetic
  source 1..20: mol_type = other RNA
cccgctccattaaaaaaaaa

SEQ ID NO: 474 - RNA, 20 nt, synthetic construct
  misc_feature 1..20: note = Seynthetic
  source 1..20: mol_type = other RNA
cccgctgtgaccataacacc

SEQ ID NO: 475 - RNA, 20 nt, synthetic construct
  misc_feature 1..20: note = Seynthetic
  source 1..20: mol_type = other RNA
cccggccgttgcagctggaa

SEQ ID NO: 476 - RNA, 20 nt, synthetic construct
  misc_feature 1..20: note = Seynthetic
  source 1..20: mol_type = other RNA
cccggcttttctcttcacca

SEQ ID NO: 477 - RNA, 20 nt, synthetic construct
  misc_feature 1..20: note = Seynthetic
  source 1..20: mol_type = other RNA
ccctcggagtctacacccgc

SEQ ID NO: 478 - RNA, 20 nt, synthetic construct
  misc_feature 1..20: note = Seynthetic
  source 1..20: mol_type = other RNA
ccctcggagtctacacctgc

SEQ ID NO: 479 - RNA, 20 nt, synthetic construct
  misc_feature 1..20: note = Seynthetic
  source 1..20: mol_type = other RNA
ccctgcaccatggattaatg

SEQ ID NO: 480 - RNA, 20 nt, synthetic construct
  misc_feature 1..20: note = Seynthetic
  source 1..20: mol_type = other RNA
ccctgcttttctcttcacca

SEQ ID NO: 481 - RNA, 20 nt, synthetic construct
  misc_feature 1..20: note = Seynthetic
  source 1..20: mol_type = other RNA
ccctgggggactctgggtgt

SEQ ID NO: 482 - RNA, 20 nt, synthetic construct
  misc_feature 1..20: note = Seynthetic
  source 1..20: mol_type = other RNA
ccctgggtgactctgggtgt

SEQ ID NO: 483 - RNA, 20 nt, synthetic construct
  misc_feature 1..20: note = Seynthetic
  source 1..20: mol_type = other RNA
ccctgtggcctctggggctt

SEQ ID NO: 484 - RNA, 20 nt, synthetic construct
  misc_feature 1..20: note = Seynthetic
  source 1..20: mol_type = other RNA
ccctgtgtcctctggggctt

SEQ ID NO: 485 - RNA, 20 nt, synthetic construct
  misc_feature 1..20: note = Seynthetic
  source 1..20: mol_type = other RNA
cccttccaaagatacagcgt

SEQ ID NO: 486 - RNA, 20 nt, synthetic construct
  misc_feature 1..20: note = Seynthetic
  source 1..20: mol_type = other RNA
cccttccaaagatacggcgt

SEQ ID NO: 487 - RNA, 20 nt, synthetic construct
  misc_feature 1..20: note = Seynthetic
  source 1..20: mol_type = other RNA
cccttcctgcccacctggag

SEQ ID NO: 488 - RNA, 20 nt, synthetic construct
  misc_feature 1..20: note = Seynthetic
  source 1..20: mol_type = other RNA
ccctttcccgcagctgggac

SEQ ID NO: 489 - RNA, 20 nt, synthetic construct
  misc_feature 1..20: note = Seynthetic
  source 1..20: mol_type = other RNA
ccgcaccatggattaatgca

SEQ ID NO: 490 - RNA, 20 nt, synthetic construct
  misc_feature 1..20: note = Seynthetic
  source 1..20: mol_type = other RNA
ccgccaggtgggcaggaagg

SEQ ID NO: 491 - RNA, 20 nt, synthetic construct
  misc_feature 1..20: note = Seynthetic
  source 1..20: mol_type = other RNA
ccgctgtgaccataacaccc

SEQ ID NO: 492 - RNA, 20 nt, synthetic construct
  misc_feature 1..20: note = Seynthetic
  source 1..20: mol_type = other RNA
ccggagtccccctgtggagg

SEQ ID NO: 493 - RNA, 20 nt, synthetic construct
  misc_feature 1..20: note = Seynthetic
  source 1..20: mol_type = other RNA
ccggcagccccttggtctgc

SEQ ID NO: 494 - RNA, 20 nt, synthetic construct
  misc_feature 1..20: note = Seynthetic
  source 1..20: mol_type = other RNA
ccggccgttgcagctggaac

SEQ ID NO: 495 - RNA, 20 nt, synthetic construct
  misc_feature 1..20: note = Seynthetic
  source 1..20: mol_type = other RNA
ccggcttttctcttcaccat

SEQ ID NO: 496 - RNA, 20 nt, synthetic construct
  misc_feature 1..20: note = Seynthetic
  source 1..20: mol_type = other RNA
ccgggggaacctcgtgtgct

SEQ ID NO: 497 - RNA, 20 nt, synthetic construct
  misc_feature 1..20: note = Seynthetic
  source 1..20: mol_type = other RNA
ccgtatctttggaaggggct

SEQ ID NO: 498 - RNA, 20 nt, synthetic construct
  misc_feature 1..20: note = Seynthetic
  source 1..20: mol_type = other RNA
ccgttgcagaccaaggggct

SEQ ID NO: 499 - RNA, 20 nt, synthetic construct
  misc_feature 1..20: note = Seynthetic
  source 1..20: mol_type = other RNA
ccgttgcagctggaacatcg

SEQ ID NO: 500 - RNA, 20 nt, synthetic construct
  misc_feature 1..20: note = Seynthetic
  source 1..20: mol_type = other RNA
ccgttgcagctggaacatgg

SEQ ID NO: 501 - RNA, 20 nt, synthetic construct
  misc_feature 1..20: note = Seynthetic
  source 1..20: mol_type = other RNA
cctccacagggggactcagg

SEQ ID NO: 502 - RNA, 20 nt, synthetic construct
  misc_feature 1..20: note = Seynthetic
  source 1..20: mol_type = other RNA
cctccacagggggactccgg

SEQ ID NO: 503 - RNA, 20 nt, synthetic construct
  misc_feature 1..20: note = Seynthetic
  source 1..20: mol_type = other RNA
cctccagcccctccaggtgg

SEQ ID NO: 504 - RNA, 20 nt, synthetic construct
  misc_feature 1..20: note = Seynthetic
  source 1..20: mol_type = other RNA
cctcggagtctacacccgct

SEQ ID NO: 505 - RNA, 20 nt, synthetic construct
  misc_feature 1..20: note = Seynthetic
  source 1..20: mol_type = other RNA
cctcggagtctacacctgct

SEQ ID NO: 506 - RNA, 20 nt, synthetic construct
  misc_feature 1..20: note = Seynthetic
  source 1..20: mol_type = other RNA
cctcttcccatctcccccac

SEQ ID NO: 507 - RNA, 20 nt, synthetic construct
  misc_feature 1..20: note = Seynthetic
  source 1..20: mol_type = other RNA
cctgaaggcggacatccctg

SEQ ID NO: 508 - RNA, 20 nt, synthetic construct
  misc_feature 1..20: note = Seynthetic
  source 1..20: mol_type = other RNA
cctgacgtgaccacaggcaa

SEQ ID NO: 509 - RNA, 20 nt, synthetic construct
  misc_feature 1..20: note = Seynthetic
  source 1..20: mol_type = other RNA
cctgagtccccctgtggagg

SEQ ID NO: 510 - RNA, 20 nt, synthetic construct
  misc_feature 1..20: note = Seynthetic
  source 1..20: mol_type = other RNA
cctgcaccatggattaatgc

SEQ ID NO: 511 - RNA, 20 nt, synthetic construct
  misc_feature 1..20: note = Seynthetic
  source 1..20: mol_type = other RNA
cctgcttttctcttcaccat

SEQ ID NO: 512 - RNA, 20 nt, synthetic construct
  misc_feature 1..20: note = Seynthetic
  source 1..20: mol_type = other RNA
cctggaggggctggaggacc

SEQ ID NO: 513 - RNA, 20 nt, synthetic construct
  misc_feature 1..20: note = Seynthetic
  source 1..20: mol_type = other RNA
cctgggaatcccattcccgc

SEQ ID NO: 514 - RNA, 20 nt, synthetic construct
  misc_feature 1..20: note = Seynthetic
  source 1..20: mol_type = other RNA
cctgggaatccctttcccgc

SEQ ID NO: 515 - RNA, 20 nt, synthetic construct
  misc_feature 1..20: note = Seynthetic
  source 1..20: mol_type = other RNA
cctgggggactctgggtgtg

SEQ ID NO: 516 - RNA, 20 nt, synthetic construct
  misc_feature 1..20: note = Seynthetic
  source 1..20: mol_type = other RNA
cctgggtgactctgggtgtg

SEQ ID NO: 517 - RNA, 20 nt, synthetic construct
  misc_feature 1..20: note = Seynthetic
  source 1..20: mol_type = other RNA
cctgtggcctctggggcttg

SEQ ID NO: 518 - RNA, 20 nt, synthetic construct
  misc_feature 1..20: note = Seynthetic
  source 1..20: mol_type = other RNA
cctgtggtcacgtcaagact

SEQ ID NO: 519 - RNA, 20 nt, synthetic construct
  misc_feature 1..20: note = Seynthetic
  source 1..20: mol_type = other RNA
cctgtggtcacgtcaggact

SEQ ID NO: 520 - RNA, 20 nt, synthetic construct
  misc_feature 1..20: note = Seynthetic
  source 1..20: mol_type = other RNA
cctgtgtcctctggggcttg

SEQ ID NO: 521 - RNA, 20 nt, synthetic construct
  misc_feature 1..20: note = Seynthetic
  source 1..20: mol_type = other RNA
ccttccaaagatacagcgtc

SEQ ID NO: 522 - RNA, 20 nt, synthetic construct
  misc_feature 1..20: note = Seynthetic
  source 1..20: mol_type = other RNA
ccttccaaagatacggcgtc

SEQ ID NO: 523 - RNA, 20 nt, synthetic construct
  misc_feature 1..20: note = Seynthetic
  source 1..20: mol_type = other RNA
ccttctggtccccgcaccat

SEQ ID NO: 524 - RNA, 20 nt, synthetic construct
  misc_feature 1..20: note = Seynthetic
  source 1..20: mol_type = other RNA
ccttctggtccctgcaccat

SEQ ID NO: 525 - RNA, 20 nt, synthetic construct
  misc_feature 1..20: note = Seynthetic
  source 1..20: mol_type = other RNA
cctttgtctctgtccctgtg

SEQ ID NO: 526 - RNA, 20 nt, synthetic construct
  misc_feature 1..20: note = Seynthetic
  source 1..20: mol_type = other RNA
cgaacgtgaatttatttcat

SEQ ID NO: 527 - RNA, 20 nt, synthetic construct
  misc_feature 1..20: note = Seynthetic
  source 1..20: mol_type = other RNA
cgaggttcccccggcttttc

SEQ ID NO: 528 - RNA, 20 nt, synthetic construct
  misc_feature 1..20: note = Seynthetic
  source 1..20: mol_type = other RNA
cgaggttccccctgcttttc

SEQ ID NO: 529 - RNA, 20 nt, synthetic construct
  misc_feature 1..20: note = Seynthetic
  source 1..20: mol_type = other RNA
cgatgttccagctgcaacgg

SEQ ID NO: 530 - RNA, 20 nt, synthetic construct
  misc_feature 1..20: note = Seynthetic
  source 1..20: mol_type = other RNA
cgcaccatggattaatgcaa

SEQ ID NO: 531 - RNA, 20 nt, synthetic construct
  misc_feature 1..20: note = Seynthetic
  source 1..20: mol_type = other RNA
cgcattcaaatgtcagataa

SEQ ID NO: 532 - RNA, 20 nt, synthetic construct
  misc_feature 1..20: note = Seynthetic
  source 1..20: mol_type = other RNA
cgccgtatctttggaagggg

SEQ ID NO: 533 - RNA, 20 nt, synthetic construct
  misc_feature 1..20: note = Seynthetic
  source 1..20: mol_type = other RNA
cgctgtgaccataacacccc

SEQ ID NO: 534 - RNA, 20 nt, synthetic construct
  misc_feature 1..20: note = Seynthetic
  source 1..20: mol_type = other RNA
cggacatccctgaactttcc

SEQ ID NO: 535 - RNA, 20 nt, synthetic construct
  misc_feature 1..20: note = Seynthetic
  source 1..20: mol_type = other RNA
cggacggtcctccagcccct

SEQ ID NO: 536 - RNA, 20 nt, synthetic construct
  misc_feature 1..20: note = Seynthetic
  source 1..20: mol_type = other RNA
cggagtccccctgtggaggc

SEQ ID NO: 537 - RNA, 20 nt, synthetic construct
  misc_feature 1..20: note = Seynthetic
  source 1..20: mol_type = other RNA
cggagtctacacccgctgtg

SEQ ID NO: 538 - RNA, 20 nt, synthetic construct
  misc_feature 1..20: note = Seynthetic
  source 1..20: mol_type = other RNA
cggagtctacacctgctgtg

SEQ ID NO: 539 - RNA, 20 nt, synthetic construct
  misc_feature 1..20: note = Seynthetic
  source 1..20: mol_type = other RNA
cggcacacttaataaagact

SEQ ID NO: 540 - RNA, 20 nt, synthetic construct
  misc_feature 1..20: note = Seynthetic
  source 1..20: mol_type = other RNA
cggcacacttaataatgact

SEQ ID NO: 541 - RNA, 20 nt, synthetic construct
  misc_feature 1..20: note = Seynthetic
  source 1..20: mol_type = other RNA
cggcagccccttggtctgca

SEQ ID NO: 542 - RNA, 20 nt, synthetic construct
  misc_feature 1..20: note = Seynthetic
  source 1..20: mol_type = other RNA
cggccgttgcagctggaaca

SEQ ID NO: 543 - RNA, 20 nt, synthetic construct
  misc_feature 1..20: note = Seynthetic
  source 1..20: mol_type = other RNA
cggcgtcccccacccctgaa

SEQ ID NO: 544 - RNA, 20 nt, synthetic construct
  misc_feature 1..20: note = Seynthetic
  source 1..20: mol_type = other RNA
cggcttttctcttcaccatg

SEQ ID NO: 545 - RNA, 20 nt, synthetic construct
  misc_feature 1..20: note = Seynthetic
  source 1..20: mol_type = other RNA
cgggaaagggattcccagga

SEQ ID NO: 546 - RNA, 20 nt, synthetic construct
  misc_feature 1..20: note = Seynthetic
  source 1..20: mol_type = other RNA
cgggaatgggattcccagga

SEQ ID NO: 547 - RNA, 20 nt, synthetic construct
  misc_feature 1..20: note = Seynthetic
  source 1..20: mol_type = other RNA
cggggaccagaaggatgggg

SEQ ID NO: 548 - RNA, 20 nt, synthetic construct
  misc_feature 1..20: note = Seynthetic
  source 1..20: mol_type = other RNA
cggggctgggtcctgggaat

SEQ ID NO: 549 - RNA, 20 nt, synthetic construct
  misc_feature 1..20: note = Seynthetic
  source 1..20: mol_type = other RNA
cgggggaacctcgtgtgctg

SEQ ID NO: 550 - RNA, 20 nt, synthetic construct
  misc_feature 1..20: note = Seynthetic
  source 1..20: mol_type = other RNA
cgggtgtagactccgagggg

SEQ ID NO: 551 - RNA, 20 nt, synthetic construct
  misc_feature 1..20: note = Seynthetic
  source 1..20: mol_type = other RNA
cggtcctccagcccctccag

SEQ ID NO: 552 - RNA, 20 nt, synthetic construct
  misc_feature 1..20: note = Seynthetic
  source 1..20: mol_type = other RNA
cgtaaagttcttagctaaga

SEQ ID NO: 553 - RNA, 20 nt, synthetic construct
  misc_feature 1..20: note = Seynthetic
  source 1..20: mol_type = other RNA
cgtatctttggaaggggctt

SEQ ID NO: 554 - RNA, 20 nt, synthetic construct
  misc_feature 1..20: note = Seynthetic
  source 1..20: mol_type = other RNA
cgtcaagactccagcctggc

SEQ ID NO: 555 - RNA, 20 nt, synthetic construct
  misc_feature 1..20: note = Seynthetic
  source 1..20: mol_type = other RNA
cgtcaggactccagcctggc

SEQ ID NO: 556 - RNA, 20 nt, synthetic construct
  misc_feature 1..20: note = Seynthetic
  source 1..20: mol_type = other RNA
cgtccccctcggagtctaca

SEQ ID NO: 557 - RNA, 20 nt, synthetic construct
  misc_feature 1..20: note = Seynthetic
  source 1..20: mol_type = other RNA
cgtcggcacacttaataaag

SEQ ID NO: 558 - RNA, 20 nt, synthetic construct
  misc_feature 1..20: note = Seynthetic
  source 1..20: mol_type = other RNA
cgtcggcacacttaataatg

SEQ ID NO: 559 - RNA, 20 nt, synthetic construct
  misc_feature 1..20: note = Seynthetic
  source 1..20: mol_type = other RNA
cgtgcagtcattattaagtg

SEQ ID NO: 560 - RNA, 20 nt, synthetic construct
  misc_feature 1..20: note = Seynthetic
  source 1..20: mol_type = other RNA
cgtgcagtctttattaagtg

SEQ ID NO: 561 - RNA, 20 nt, synthetic construct
  misc_feature 1..20: note = Seynthetic
  source 1..20: mol_type = other RNA
cgtgggggagatgggaagag

SEQ ID NO: 562 - RNA, 20 nt, synthetic construct
  misc_feature 1..20: note = Seynthetic
  source 1..20: mol_type = other RNA
cgttcgtaaagttcttagct

SEQ ID NO: 563 - RNA, 20 nt, synthetic construct
  misc_feature 1..20: note = Seynthetic
  source 1..20: mol_type = other RNA
cgttgcagaccaaggggctg

SEQ ID NO: 564 - RNA, 20 nt, synthetic construct
  misc_feature 1..20: note = Seynthetic
  source 1..20: mol_type = other RNA
cgttgcagctggaacatcgt

SEQ ID NO: 565 - RNA, 20 nt, synthetic construct
  misc_feature 1..20: note = Seynthetic
  source 1..20: mol_type = other RNA
cgttgcagctggaacatggt

SEQ ID NO: 566 - RNA, 20 nt, synthetic construct
  misc_feature 1..20: note = Seynthetic
  source 1..20: mol_type = other RNA
cgttgcattaatccatggtg

SEQ ID NO: 567 - RNA, 20 nt, synthetic construct
  misc_feature 1..20: note = Seynthetic
  source 1..20: mol_type = other RNA
ctaagaactttacgaacgtg

SEQ ID NO: 568 - RNA, 20 nt, synthetic construct
  misc_feature 1..20: note = Seynthetic
  source 1..20: mol_type = other RNA
ctaagaactttatgaacgtg

SEQ ID NO: 569 - RNA, 20 nt, synthetic construct
  misc_feature 1..20: note = Seynthetic
  source 1..20: mol_type = other RNA
ctacacccgctgtgaccata

SEQ ID NO: 570 - RNA, 20 nt, synthetic construct
  misc_feature 1..20: note = Seynthetic
  source 1..20: mol_type = other RNA
ctacacctgctgtgaccata

SEQ ID NO: 571 - RNA, 20 nt, synthetic construct
  misc_feature 1..20: note = Seynthetic
  source 1..20: mol_type = other RNA
ctacattctcacgtgcagtc

SEQ ID NO: 572 - RNA, 20 nt, synthetic construct
  misc_feature 1..20: note = Seynthetic
  source 1..20: mol_type = other RNA
ctaggattgtgaaacaccct

SEQ ID NO: 573 - RNA, 20 nt, synthetic construct
  misc_feature 1..20: note = Seynthetic
  source 1..20: mol_type = other RNA
ctcacgtgcagtcattatta

SEQ ID NO: 574 - RNA, 20 nt, synthetic construct
  misc_feature 1..20: note = Seynthetic
  source 1..20: mol_type = other RNA
ctcacgtgcagtctttatta

SEQ ID NO: 575 - RNA, 20 nt, synthetic construct
  misc_feature 1..20: note = Seynthetic
  source 1..20: mol_type = other RNA
ctcatgcttctaccagggaa

SEQ ID NO: 576 - RNA, 20 nt, synthetic construct
  misc_feature 1..20: note = Seynthetic
  source 1..20: mol_type = other RNA
ctcatgcttctaccagggag

SEQ ID NO: 577 - RNA, 20 nt, synthetic construct
  misc_feature 1..20: note = Seynthetic
  source 1..20: mol_type = other RNA
ctccacagggggactccggc

SEQ ID NO: 578 - RNA, 20 nt, synthetic construct
  misc_feature 1..20: note = Seynthetic
  source 1..20: mol_type = other RNA
ctccaggtgggcaggaaggg

SEQ ID NO: 579 - RNA, 20 nt, synthetic construct
  misc_feature 1..20: note = Seynthetic
  source 1..20: mol_type = other RNA
ctcccccaccatgttccagc

SEQ ID NO: 580 - RNA, 20 nt, synthetic construct
  misc_feature 1..20: note = Seynthetic
  source 1..20: mol_type = other RNA
ctcccccacgatgttccagc

SEQ ID NO: 581 - RNA, 20 nt, synthetic construct
  misc_feature 1..20: note = Seynthetic
  source 1..20: mol_type = other RNA
ctccctggtagaagcatgag

SEQ ID NO: 582 - RNA, 20 nt, synthetic construct
  misc_feature 1..20: note = Seynthetic
  source 1..20: mol_type = other RNA
ctccggcagccccttggtct

SEQ ID NO: 583 - RNA, 20 nt, synthetic construct
  misc_feature 1..20: note = Seynthetic
  source 1..20: mol_type = other RNA
ctcggagtctacacccgctg

SEQ ID NO: 584 - RNA, 20 nt, synthetic construct
  misc_feature 1..20: note = Seynthetic
  source 1..20: mol_type = other RNA
ctcggagtctacacctgctg

SEQ ID NO: 585 - RNA, 20 nt, synthetic construct
  misc_feature 1..20: note = Seynthetic
  source 1..20: mol_type = other RNA
ctctcaaagcaacactttct

SEQ ID NO: 586 - RNA, 20 nt, synthetic construct
  misc_feature 1..20: note = Seynthetic
  source 1..20: mol_type = other RNA
ctcttcccatctcccccacc

SEQ ID NO: 587 - RNA, 20 nt, synthetic construct
  misc_feature 1..20: note = Seynthetic
  source 1..20: mol_type = other RNA
ctcttcccatctcccccacg

SEQ ID NO: 588 - RNA, 20 nt, synthetic construct
  misc_feature 1..20: note = Seynthetic
  source 1..20: mol_type = other RNA
ctgaaatttgaatgcgattg

SEQ ID NO: 589 - RNA, 20 nt, synthetic construct
  misc_feature 1..20: note = Seynthetic
  source 1..20: mol_type = other RNA
ctgaaggcggacatccctga

SEQ ID NO: 590 - RNA, 20 nt, synthetic construct
  misc_feature 1..20: note = Seynthetic
  source 1..20: mol_type = other RNA
ctgacatttgaatgcgattg

SEQ ID NO: 591 - RNA, 20 nt, synthetic construct
  misc_feature 1..20: note = Seynthetic
  source 1..20: mol_type = other RNA
ctgacgtgaccacaggcaag

SEQ ID NO: 592 - RNA, 20 nt, synthetic construct
  misc_feature 1..20: note = Seynthetic
  source 1..20: mol_type = other RNA
ctgagtccccctgtggaggc

SEQ ID NO: 593 - RNA, 20 nt, synthetic construct
  misc_feature 1..20: note = Seynthetic
  source 1..20: mol_type = other RNA
ctgcaccatggattaatgca

SEQ ID NO: 594 - RNA, 20 nt, synthetic construct
  misc_feature 1..20: note = Seynthetic
  source 1..20: mol_type = other RNA
ctgcccacctggaggggctg

SEQ ID NO: 595 - RNA, 20 nt, synthetic construct
  misc_feature 1..20: note = Seynthetic
  source 1..20: mol_type = other RNA
ctgccggagtccccctgtgg

SEQ ID NO: 596 - RNA, 20 nt, synthetic construct
  misc_feature 1..20: note = Seynthetic
  source 1..20: mol_type = other RNA
ctgcctccacagggggactc

SEQ ID NO: 597 - RNA, 20 nt, synthetic construct
  misc_feature 1..20: note = Seynthetic
  source 1..20: mol_type = other RNA
ctgcctgagtccccctgtgg

SEQ ID NO: 598 - RNA, 20 nt, synthetic construct
  misc_feature 1..20: note = Seynthetic
  source 1..20: mol_type = other RNA
ctgcgggaaagggattccca

SEQ ID NO: 599 - RNA, 20 nt, synthetic construct
  misc_feature 1..20: note = Seynthetic
  source 1..20: mol_type = other RNA
ctgcgggaatgggattccca

SEQ ID NO: 600 - RNA, 20 nt, synthetic construct
  misc_feature 1..20: note = Seynthetic
  source 1..20: mol_type = other RNA
ctgcttttctcttcaccatg

SEQ ID NO: 601 - RNA, 20 nt, synthetic construct
  misc_feature 1..20: note = Seynthetic
  source 1..20: mol_type = other RNA
ctggaacatcgtgggggaga

SEQ ID NO: 602 - RNA, 20 nt, synthetic construct
  misc_feature 1..20: note = Seynthetic
  source 1..20: mol_type = other RNA
ctggaacatggtgggggaga

SEQ ID NO: 603 - RNA, 20 nt, synthetic construct
  misc_feature 1..20: note = Seynthetic
  source 1..20: mol_type = other RNA
ctggaggggctggaggaccg

SEQ ID NO: 604 - RNA, 20 nt, synthetic construct
  misc_feature 1..20: note = Seynthetic
  source 1..20: mol_type = other RNA
ctggagtcccagctgcggga

SEQ ID NO: 605 - RNA, 20 nt, synthetic construct
  misc_feature 1..20: note = Seynthetic
  source 1..20: mol_type = other RNA
ctggagtcctgacgtgacca

SEQ ID NO: 606 - RNA, 20 nt, synthetic construct
  misc_feature 1..20: note = Seynthetic
  source 1..20: mol_type = other RNA
ctggagtcttgacgtgacca

SEQ ID NO: 607 - RNA, 20 nt, synthetic construct
  misc_feature 1..20: note = Seynthetic
  source 1..20: mol_type = other RNA
ctgggaatcccattcccgca

SEQ ID NO: 608 - RNA, 20 nt, synthetic construct
  misc_feature 1..20: note = Seynthetic
  source 1..20: mol_type = other RNA
ctgggaatccctttcccgca

SEQ ID NO: 609 - RNA, 20 nt, synthetic construct
  misc_feature 1..20: note = Seynthetic
  source 1..20: mol_type = other RNA
ctgggccaggctggagtcct

SEQ ID NO: 610 - RNA, 20 nt, synthetic construct
  misc_feature 1..20: note = Seynthetic
  source 1..20: mol_type = other RNA
ctgggccaggctggagtctt

SEQ ID NO: 611 - RNA, 20 nt, synthetic construct
  misc_feature 1..20: note = Seynthetic
  source 1..20: mol_type = other RNA
ctgggggactctgggtgtgg

SEQ ID NO: 612 - RNA, 20 nt, synthetic construct
  misc_feature 1..20: note = Seynthetic
  source 1..20: mol_type = other RNA
ctgggtcctgggaatcccat

SEQ ID NO: 613 - RNA, 20 nt, synthetic construct
  misc_feature 1..20: note = Seynthetic
  source 1..20: mol_type = other RNA
ctgggtcctgggaatccctt

SEQ ID NO: 614 - RNA, 20 nt, synthetic construct
  misc_feature 1..20: note = Seynthetic
  source 1..20: mol_type = other RNA
ctggtccccgcaccatggat

SEQ ID NO: 615 - RNA, 20 nt, synthetic construct
  misc_feature 1..20: note = Seynthetic
  source 1..20: mol_type = other RNA
ctggtccctgcaccatggat

SEQ ID NO: 616 - RNA, 20 nt, synthetic construct
  misc_feature 1..20: note = Seynthetic
  source 1..20: mol_type = other RNA
ctgtatctttggaaggggct

SEQ ID NO: 617 - RNA, 20 nt, synthetic construct
  misc_feature 1..20: note = Seynthetic
  source 1..20: mol_type = other RNA
ctgtccctgtggcctctggg

SEQ ID NO: 618 - RNA, 20 nt, synthetic construct
  misc_feature 1..20: note = Seynthetic
  source 1..20: mol_type = other RNA
ctgtggcctctggggcttga

SEQ ID NO: 619 - RNA, 20 nt, synthetic construct
  misc_feature 1..20: note = Seynthetic
  source 1..20: mol_type = other RNA
ctgtggtcacgtcaagactc

SEQ ID NO: 620 - RNA, 20 nt, synthetic construct
  misc_feature 1..20: note = Seynthetic
  source 1..20: mol_type = other RNA
ctgtggtcacgtcaggactc

SEQ ID NO: 621 - RNA, 20 nt, synthetic construct
  misc_feature 1..20: note = Seynthetic
  source 1..20: mol_type = other RNA
ctgtgtcctctggggcttga

SEQ ID NO: 622 - RNA, 20 nt, synthetic construct
  misc_feature 1..20: note = Seynthetic
  source 1..20: mol_type = other RNA
cttaataaagactgcacgtg

SEQ ID NO: 623 - RNA, 20 nt, synthetic construct
  misc_feature 1..20: note = Seynthetic
  source 1..20: mol_type = other RNA
cttaataatgactgcacgtg

SEQ ID NO: 624 - RNA, 20 nt, synthetic construct
  misc_feature 1..20: note = Seynthetic
  source 1..20: mol_type = other RNA
cttagctaagaactttacga

SEQ ID NO: 625 - RNA, 20 nt, synthetic construct
  misc_feature 1..20: note = Seynthetic
  source 1..20: mol_type = other RNA
cttagctaagaactttatga

SEQ ID NO: 626 - RNA, 20 nt, synthetic construct
  misc_feature 1..20: note = Seynthetic
  source 1..20: mol_type = other RNA
cttcaccacacccagagtca

SEQ ID NO: 627 - RNA, 20 nt, synthetic construct
  misc_feature 1..20: note = Seynthetic
  source 1..20: mol_type = other RNA
cttcaccacacccagagtcc

SEQ ID NO: 628 - RNA, 20 nt, synthetic construct
  misc_feature 1..20: note = Seynthetic
  source 1..20: mol_type = other RNA
cttccaaagatacagcgtcc

SEQ ID NO: 629 - RNA, 20 nt, synthetic construct
  misc_feature 1..20: note = Seynthetic
  source 1..20: mol_type = other RNA
cttccaaagatacggcgtcc

SEQ ID NO: 630 - RNA, 20 nt, synthetic construct
  misc_feature 1..20: note = Seynthetic
  source 1..20: mol_type = other RNA
cttcccatctcccccacgat

SEQ ID NO: 631 - RNA, 20 nt, synthetic construct
  misc_feature 1..20: note = Seynthetic
  source 1..20: mol_type = other RNA
cttccccacattgtgttttt

SEQ ID NO: 632 - RNA, 20 nt, synthetic construct
  misc_feature 1..20: note = Seynthetic
  source 1..20: mol_type = other RNA
cttctaccagggaactcctg

SEQ ID NO: 633 - RNA, 20 nt, synthetic construct
  misc_feature 1..20: note = Seynthetic
  source 1..20: mol_type = other RNA
cttctggtccccgcaccatg

SEQ ID NO: 634 - RNA, 20 nt, synthetic construct
  misc_feature 1..20: note = Seynthetic
  source 1..20: mol_type = other RNA
cttgacgtgaccacaggcaa

SEQ ID NO: 635 - RNA, 20 nt, synthetic construct
  misc_feature 1..20: note = Seynthetic
  source 1..20: mol_type = other RNA
cttgcctgtggtcacgtcaa

SEQ ID NO: 636 - RNA, 20 nt, synthetic construct
  misc_feature 1..20: note = Seynthetic
  source 1..20: mol_type = other RNA
cttgcctgtggtcacgtcag

SEQ ID NO: 637 - RNA, 20 nt, synthetic construct
  misc_feature 1..20: note = Seynthetic
  source 1..20: mol_type = other RNA
ctttacgaacgtgaatttat

SEQ ID NO: 638 - RNA, 20 nt, synthetic construct
  misc_feature 1..20: note = Seynthetic
  source 1..20: mol_type = other RNA
ctttattaagtgtgccgacg

SEQ ID NO: 639 - RNA, 20 nt, synthetic construct
  misc_feature 1..20: note = Seynthetic
  source 1..20: mol_type = other RNA
ctttcccgcagctgggactc

SEQ ID NO: 640 - RNA, 20 nt, synthetic construct
  misc_feature 1..20: note = Seynthetic
  source 1..20: mol_type = other RNA
ctttcttagctaagaacttt

SEQ ID NO: 641 - RNA, 20 nt, synthetic construct
  misc_feature 1..20: note = Seynthetic
  source 1..20: mol_type = other RNA
ctttgtctctgtccctgtgg

SEQ ID NO: 642 - RNA, 20 nt, synthetic construct
  misc_feature 1..20: note = Seynthetic
  source 1..20: mol_type = other RNA
gaaaaaaacacaatgtgggg

SEQ ID NO: 643 - RNA, 20 nt, synthetic construct
  misc_feature 1..20: note = Seynthetic
  source 1..20: mol_type = other RNA
gaaaaaaatacaatgtgggg

SEQ ID NO: 644 - RNA, 20 nt, synthetic construct
  misc_feature 1..20: note = Seynthetic
  source 1..20: mol_type = other RNA
gaaaagcagggggaacctcg

SEQ ID NO: 645 - RNA, 20 nt, synthetic construct
  misc_feature 1..20: note = Seynthetic
  source 1..20: mol_type = other RNA
gaaaagccgggggaacctcg

SEQ ID NO: 646 - RNA, 20 nt, synthetic construct
  misc_feature 1..20: note = Seynthetic
  source 1..20: mol_type = other RNA
gaaacaccctgggggactct

SEQ ID NO: 647 - RNA, 20 nt, synthetic construct
  misc_feature 1..20: note = Seynthetic
  source 1..20: mol_type = other RNA
gaaacaccctgggtgactct

SEQ ID NO: 648 - RNA, 20 nt, synthetic construct
  misc_feature 1..20: note = Seynthetic
  source 1..20: mol_type = other RNA
gaaagggattcccaggaccc

SEQ ID NO: 649 - RNA, 20 nt, synthetic construct
  misc_feature 1..20: note = Seynthetic
  source 1..20: mol_type = other RNA
gaaagttcagggatgtccgc

SEQ ID NO: 650 - RNA, 20 nt, synthetic construct
  misc_feature 1..20: note = Seynthetic
  source 1..20: mol_type = other RNA
gaaataaattcacgttcgta

SEQ ID NO: 651 - RNA, 20 nt, synthetic construct
  misc_feature 1..20: note = Seynthetic
  source 1..20: mol_type = other RNA
gaaatttgaatgcgattgtg

SEQ ID NO: 652 - RNA, 20 nt, synthetic construct
  misc_feature 1..20: note = Seynthetic
  source 1..20: mol_type = other RNA
gaacatcgtgggggagatgg

SEQ ID NO: 653 - RNA, 20 nt, synthetic construct
  misc_feature 1..20: note = Seynthetic
  source 1..20: mol_type = other RNA
gaactttacgaacgtgaatt

SEQ ID NO: 654 - RNA, 20 nt, synthetic construct
  misc_feature 1..20: note = Seynthetic
  source 1..20: mol_type = other RNA
gaactttatgaacgtgaatt

SEQ ID NO: 655 - RNA, 20 nt, synthetic construct
  misc_feature 1..20: note = Seynthetic
  source 1..20: mol_type = other RNA
gaagagaaaagccgggggaa

SEQ ID NO: 656 - RNA, 20 nt, synthetic construct
  misc_feature 1..20: note = Seynthetic
  source 1..20: mol_type = other RNA
gaaggcggacatccctgaac

SEQ ID NO: 657 - RNA, 20 nt, synthetic construct
  misc_feature 1..20: note = Seynthetic
  source 1..20: mol_type = other RNA
gaagtgacttccccacattg

SEQ ID NO: 658 - RNA, 20 nt, synthetic construct
  misc_feature 1..20: note = Seynthetic
  source 1..20: mol_type = other RNA
gaatcccattcccgcagctg

SEQ ID NO: 659 - RNA, 20 nt, synthetic construct
  misc_feature 1..20: note = Seynthetic
  source 1..20: mol_type = other RNA
gaatccctttcccgcagctg

SEQ ID NO: 660 - RNA, 20 nt, synthetic construct
  misc_feature 1..20: note = Seynthetic
  source 1..20: mol_type = other RNA
gaatgggattcccaggaccc

SEQ ID NO: 661 - RNA, 20 nt, synthetic construct
  misc_feature 1..20: note = Seynthetic
  source 1..20: mol_type = other RNA
gacacagggacagagacaaa

SEQ ID NO: 662 - RNA, 20 nt, synthetic construct
  misc_feature 1..20: note = Seynthetic
  source 1..20: mol_type = other RNA
gacatccctgaactttccat

SEQ ID NO: 663 - RNA, 20 nt, synthetic construct
  misc_feature 1..20: note = Seynthetic
  source 1..20: mol_type = other RNA
gaccaaggggctgccggagt

SEQ ID NO: 664 - RNA, 20 nt, synthetic construct
  misc_feature 1..20: note = Seynthetic
  source 1..20: mol_type = other RNA
gacgccgtatctttggaagg

SEQ ID NO: 665 - RNA, 20 nt, synthetic construct
  misc_feature 1..20: note = Seynthetic
  source 1..20: mol_type = other RNA
gacgctgtatctttggaagg

SEQ ID NO: 666 - RNA, 20 nt, synthetic construct
  misc_feature 1..20: note = Seynthetic
  source 1..20: mol_type = other RNA
gacggtcctccagcccctcc

SEQ ID NO: 667 - RNA, 20 nt, synthetic construct
  misc_feature 1..20: note = Seynthetic
  source 1..20: mol_type = other RNA
gactccggcagccccttggt

SEQ ID NO: 668 - RNA, 20 nt, synthetic construct
  misc_feature 1..20: note = Seynthetic
  source 1..20: mol_type = other RNA
gacttccccacattgtattt

SEQ ID NO: 669 - RNA, 20 nt, synthetic construct
  misc_feature 1..20: note = Seynthetic
  source 1..20: mol_type = other RNA
gacttccccacattgtgttt

SEQ ID NO: 670 - RNA, 20 nt, synthetic construct
  misc_feature 1..20: note = Seynthetic
  source 1..20: mol_type = other RNA
gagaaaagcagggggaacct

SEQ ID NO: 671 - RNA, 20 nt, synthetic construct
  misc_feature 1..20: note = Seynthetic
  source 1..20: mol_type = other RNA
gagaaaagccgggggaacct

SEQ ID NO: 672 - RNA, 20 nt, synthetic construct
  misc_feature 1..20: note = Seynthetic
  source 1..20: mol_type = other RNA
gagcagcacacgaggttccc

SEQ ID NO: 673 - RNA, 20 nt, synthetic construct
  misc_feature 1..20: note = Seynthetic
  source 1..20: mol_type = other RNA
gagcctgggccaggctggag

SEQ ID NO: 674 - RNA, 20 nt, synthetic construct
  misc_feature 1..20: note = Seynthetic
  source 1..20: mol_type = other RNA
gagctccctggtagaagcat

SEQ ID NO: 675 - RNA, 20 nt, synthetic construct
  misc_feature 1..20: note = Seynthetic
  source 1..20: mol_type = other RNA
gaggatgaaataaattcacg

SEQ ID NO: 676 - RNA, 20 nt, synthetic construct
  misc_feature 1..20: note = Seynthetic
  source 1..20: mol_type = other RNA
gaggccacagggacagagac

SEQ ID NO: 677 - RNA, 20 nt, synthetic construct
  misc_feature 1..20: note = Seynthetic
  source 1..20: mol_type = other RNA
gaggggctggaggaccgtcc

SEQ ID NO: 678 - RNA, 20 nt, synthetic construct
  misc_feature 1..20: note = Seynthetic
  source 1..20: mol_type = other RNA
gaggttcccccggcttttct

SEQ ID NO: 679 - RNA, 20 nt, synthetic construct
  misc_feature 1..20: note = Seynthetic
  source 1..20: mol_type = other RNA
gagtcacccagggtgtttca

SEQ ID NO: 680 - RNA, 20 nt, synthetic construct
  misc_feature 1..20: note = Seynthetic
  source 1..20: mol_type = other RNA
gagtcccagctgcgggaaag

SEQ ID NO: 681 - RNA, 20 nt, synthetic construct
  misc_feature 1..20: note = Seynthetic
  source 1..20: mol_type = other RNA
gagtcccagctgcgggaatg

SEQ ID NO: 682 - RNA, 20 nt, synthetic construct
  misc_feature 1..20: note = Seynthetic
  source 1..20: mol_type = other RNA
gagtcccccagggtgtttca

SEQ ID NO: 683 - RNA, 20 nt, synthetic construct
  misc_feature 1..20: note = Seynthetic
  source 1..20: mol_type = other RNA
gagtcctgacgtgaccacag

SEQ ID NO: 684 - RNA, 20 nt, synthetic construct
  misc_feature 1..20: note = Seynthetic
  source 1..20: mol_type = other RNA
gagtctacacccgctgtgac

SEQ ID NO: 685 - RNA, 20 nt, synthetic construct
  misc_feature 1..20: note = Seynthetic
  source 1..20: mol_type = other RNA
gagtctacacctgctgtgac

SEQ ID NO: 686 - RNA, 20 nt, synthetic construct
  misc_feature 1..20: note = Seynthetic
  source 1..20: mol_type = other RNA
gagtcttgacgtgaccacag

SEQ ID NO: 687 - RNA, 20 nt, synthetic construct
  misc_feature 1..20: note = Seynthetic
  source 1..20: mol_type = other RNA
gagttccctggtagaagcat

SEQ ID NO: 688 - RNA, 20 nt, synthetic construct
  misc_feature 1..20: note = Seynthetic
  source 1..20: mol_type = other RNA
gatacagcgtcccccacccc

SEQ ID NO: 689 - RNA, 20 nt, synthetic construct
  misc_feature 1..20: note = Seynthetic
  source 1..20: mol_type = other RNA
gatacggcgtcccccacccc

SEQ ID NO: 690 - RNA, 20 nt, synthetic construct
  misc_feature 1..20: note = Seynthetic
  source 1..20: mol_type = other RNA
gatccatggtgaagagaaaa

SEQ ID NO: 691 - RNA, 20 nt, synthetic construct
  misc_feature 1..20: note = Seynthetic
  source 1..20: mol_type = other RNA
gatgaaataaattcacgttc

SEQ ID NO: 692 - RNA, 20 nt, synthetic construct
  misc_feature 1..20: note = Seynthetic
  source 1..20: mol_type = other RNA
gatgcacaatcgcattcaaa

SEQ ID NO: 693 - RNA, 20 nt, synthetic construct
  misc_feature 1..20: note = Seynthetic
  source 1..20: mol_type = other RNA
gatgtccgccttcaggtgtg

SEQ ID NO: 694 - RNA, 20 nt, synthetic construct
  misc_feature 1..20: note = Seynthetic
  source 1..20: mol_type = other RNA
gatgttccagctgcaacggc

SEQ ID NO: 695 - RNA, 20 nt, synthetic construct
  misc_feature 1..20: note = Seynthetic
  source 1..20: mol_type = other RNA
gattatctgacatttgaatg

SEQ ID NO: 696 - RNA, 20 nt, synthetic construct
  misc_feature 1..20: note = Seynthetic
  source 1..20: mol_type = other RNA
gattgtgaaacaccctgggg

SEQ ID NO: 697 - RNA, 20 nt, synthetic construct
  misc_feature 1..20: note = Seynthetic
  source 1..20: mol_type = other RNA
gattgtgaaacaccctgggt

SEQ ID NO: 698 - RNA, 20 nt, synthetic construct
  misc_feature 1..20: note = Seynthetic
  source 1..20: mol_type = other RNA
gatttcaggggtgggggacg

SEQ ID NO: 699 - RNA, 20 nt, synthetic construct
  misc_feature 1..20: note = Seynthetic
  source 1..20: mol_type = other RNA
gcaaaacccccgctccatta

SEQ ID NO: 700 - RNA, 20 nt, synthetic construct
  misc_feature 1..20: note = Seynthetic
  source 1..20: mol_type = other RNA
gcacaatcgcattcaaatgt

SEQ ID NO: 701 - RNA, 20 nt, synthetic construct
  misc_feature 1..20: note = Seynthetic
  source 1..20: mol_type = other RNA
gcacaatcgcattcaaattt

SEQ ID NO: 702 - RNA, 20 nt, synthetic construct
  misc_feature 1..20: note = Seynthetic
  source 1..20: mol_type = other RNA
gcacacgaggttcccccggc

SEQ ID NO: 703 - RNA, 20 nt, synthetic construct
  misc_feature 1..20: note = Seynthetic
  source 1..20: mol_type = other RNA
gcacacgaggttccccctgc

SEQ ID NO: 704 - RNA, 20 nt, synthetic construct
  misc_feature 1..20: note = Seynthetic
  source 1..20: mol_type = other RNA
gcacacttaataaagactgc

SEQ ID NO: 705 - RNA, 20 nt, synthetic construct
  misc_feature 1..20: note = Seynthetic
  source 1..20: mol_type = other RNA
gcacacttaataatgactgc

SEQ ID NO: 706 - RNA, 20 nt, synthetic construct
  misc_feature 1..20: note = Seynthetic
  source 1..20: mol_type = other RNA
gcagaccaaggggctgccgg

SEQ ID NO: 707 - RNA, 20 nt, synthetic construct
  misc_feature 1..20: note = Seynthetic
  source 1..20: mol_type = other RNA
gcagcacacgaggttccccc

SEQ ID NO: 708 - RNA, 20 nt, synthetic construct
  misc_feature 1..20: note = Seynthetic
  source 1..20: mol_type = other RNA
gcagctggaacatcgtgggg

SEQ ID NO: 709 - RNA, 20 nt, synthetic construct
  misc_feature 1..20: note = Seynthetic
  source 1..20: mol_type = other RNA
gcagctggaacatggtgggg

SEQ ID NO: 710 - RNA, 20 nt, synthetic construct
  misc_feature 1..20: note = Seynthetic
  source 1..20: mol_type = other RNA
gcaggagctccctggtagaa

SEQ ID NO: 711 - RNA, 20 nt, synthetic construct
  misc_feature 1..20: note = Seynthetic
  source 1..20: mol_type = other RNA
gcaggagttccctggtagaa

SEQ ID NO: 712 - RNA, 20 nt, synthetic construct
  misc_feature 1..20: note = Seynthetic
  source 1..20: mol_type = other RNA
gcagggaccagaaggatggg

SEQ ID NO: 713 - RNA, 20 nt, synthetic construct
  misc_feature 1..20: note = Seynthetic
  source 1..20: mol_type = other RNA
gcagggggaacctcgtgtgc

SEQ ID NO: 714 - RNA, 20 nt, synthetic construct
  misc_feature 1..20: note = Seynthetic
  source 1..20: mol_type = other RNA
gcaggtgtagactccgaggg

SEQ ID NO: 715 - RNA, 20 nt, synthetic construct
  misc_feature 1..20: note = Seynthetic
  source 1..20: mol_type = other RNA
gcagtcattattaagtgtgc

SEQ ID NO: 716 - RNA, 20 nt, synthetic construct
  misc_feature 1..20: note = Seynthetic
  source 1..20: mol_type = other RNA
gcagtctttattaagtgtgc

SEQ ID NO: 717 - RNA, 20 nt, synthetic construct
  misc_feature 1..20: note = Seynthetic
  source 1..20: mol_type = other RNA
gcatccctgaactttccata

SEQ ID NO: 718 - RNA, 20 nt, synthetic construct
  misc_feature 1..20: note = Seynthetic
  source 1..20: mol_type = other RNA
gcattaatccatggtgcagg

SEQ ID NO: 719 - RNA, 20 nt, synthetic construct
  misc_feature 1..20: note = Seynthetic
  source 1..20: mol_type = other RNA
gcattaatccatggtgcggg

SEQ ID NO: 720 - RNA, 20 nt, synthetic construct
  misc_feature 1..20: note = Seynthetic
  source 1..20: mol_type = other RNA
gccaagccccttccaaagat

SEQ ID NO: 721 - RNA, 20 nt, synthetic construct
  misc_feature 1..20: note = Seynthetic
  source 1..20: mol_type = other RNA
gccacacctgaaggcggaca

SEQ ID NO: 722 - RNA, 20 nt, synthetic construct
  misc_feature 1..20: note = Seynthetic
  source 1..20: mol_type = other RNA
gccacagggacagagacaaa

SEQ ID NO: 723 - RNA, 20 nt, synthetic construct
  misc_feature 1..20: note = Seynthetic
  source 1..20: mol_type = other RNA
gccaggctggagtcctgacg

SEQ ID NO: 724 - RNA, 20 nt, synthetic construct
  misc_feature 1..20: note = Seynthetic
  source 1..20: mol_type = other RNA
gccaggctggagtcttgacg

SEQ ID NO: 725 - RNA, 20 nt, synthetic construct
  misc_feature 1..20: note = Seynthetic
  source 1..20: mol_type = other RNA
gccaggtgggcaggaagggg

SEQ ID NO: 726 - RNA, 20 nt, synthetic construct
  misc_feature 1..20: note = Seynthetic
  source 1..20: mol_type = other RNA
gcccctccaggtgggcagga

SEQ ID NO: 727 - RNA, 20 nt, synthetic construct
  misc_feature 1..20: note = Seynthetic
  source 1..20: mol_type = other RNA
gccccttccaaagatacagc

SEQ ID NO: 728 - RNA, 20 nt, synthetic construct
  misc_feature 1..20: note = Seynthetic
  source 1..20: mol_type = other RNA
gccccttccaaagatacggc

SEQ ID NO: 729 - RNA, 20 nt, synthetic construct
  misc_feature 1..20: note = Seynthetic
  source 1..20: mol_type = other RNA
gccggagtccccctgtggag

SEQ ID NO: 730 - RNA, 20 nt, synthetic construct
  misc_feature 1..20: note = Seynthetic
  source 1..20: mol_type = other RNA
gccgggggaacctcgtgtgc

SEQ ID NO: 731 - RNA, 20 nt, synthetic construct
  misc_feature 1..20: note = Seynthetic
  source 1..20: mol_type = other RNA
gccgtatctttggaaggggc

SEQ ID NO: 732 - RNA, 20 nt, synthetic construct
  misc_feature 1..20: note = Seynthetic
  source 1..20: mol_type = other RNA
gccgttgcagctggaacatc

SEQ ID NO: 733 - RNA, 20 nt, synthetic construct
  misc_feature 1..20: note = Seynthetic
  source 1..20: mol_type = other RNA
gccgttgcagctggaacatg

SEQ ID NO: 734 - RNA, 20 nt, synthetic construct
  misc_feature 1..20: note = Seynthetic
  source 1..20: mol_type = other RNA
gcctccacagggggactcag

SEQ ID NO: 735 - RNA, 20 nt, synthetic construct
  misc_feature 1..20: note = Seynthetic
  source 1..20: mol_type = other RNA
gcctccacagggggactccg

SEQ ID NO: 736 - RNA, 20 nt, synthetic construct
  misc_feature 1..20: note = Seynthetic
  source 1..20: mol_type = other RNA
gcctctggggcttgacaccc

SEQ ID NO: 737 - RNA, 20 nt, synthetic construct
  misc_feature 1..20: note = Seynthetic
  source 1..20: mol_type = other RNA
gcctgagtccccctgtggag

SEQ ID NO: 738 - RNA, 20 nt, synthetic construct
  misc_feature 1..20: note = Seynthetic
  source 1..20: mol_type = other RNA
gcctgggccaggctggagtc

SEQ ID NO: 739 - RNA, 20 nt, synthetic construct
  misc_feature 1..20: note = Seynthetic
  source 1..20: mol_type = other RNA
gcctgtggtcacgtcaagac

SEQ ID NO: 740 - RNA, 20 nt, synthetic construct
  misc_feature 1..20: note = Seynthetic
  source 1..20: mol_type = other RNA
gcctgtggtcacgtcaggac

SEQ ID NO: 741 - RNA, 20 nt, synthetic construct
  misc_feature 1..20: note = Seynthetic
  source 1..20: mol_type = other RNA
gcggacatccctgaactttc

SEQ ID NO: 742 - RNA, 20 nt, synthetic construct
  misc_feature 1..20: note = Seynthetic
  source 1..20: mol_type = other RNA
gcgggaaagggattcccagg

SEQ ID NO: 743 - RNA, 20 nt, synthetic construct
  misc_feature 1..20: note = Seynthetic
  source 1..20: mol_type = other RNA
gcgggaatgggattcccagg

SEQ ID NO: 744 - RNA, 20 nt, synthetic construct
  misc_feature 1..20: note = Seynthetic
  source 1..20: mol_type = other RNA
gcggggaccagaaggatggg

SEQ ID NO: 745 - RNA, 20 nt, synthetic construct
  misc_feature 1..20: note = Seynthetic
  source 1..20: mol_type = other RNA
gcgggtgtagactccgaggg

SEQ ID NO: 746 - RNA, 20 nt, synthetic construct
  misc_feature 1..20: note = Seynthetic
  source 1..20: mol_type = other RNA
gcgtcggcacacttaataaa

SEQ ID NO: 747 - RNA, 20 nt, synthetic construct
  misc_feature 1..20: note = Seynthetic
  source 1..20: mol_type = other RNA
gcgtcggcacacttaataat

SEQ ID NO: 748 - RNA, 20 nt, synthetic construct
  misc_feature 1..20: note = Seynthetic
  source 1..20: mol_type = other RNA
gctaagaactttacgaacgt

SEQ ID NO: 749 - RNA, 20 nt, synthetic construct
  misc_feature 1..20: note = Seynthetic
  source 1..20: mol_type = other RNA
gctaagaactttatgaacgt

SEQ ID NO: 750 - RNA, 20 nt, synthetic construct
  misc_feature 1..20: note = Seynthetic
  source 1..20: mol_type = other RNA
gctcatgcttctaccaggga

SEQ ID NO: 751 - RNA, 20 nt, synthetic construct
  misc_feature 1..20: note = Seynthetic
  source 1..20: mol_type = other RNA
gctccctggtagaagcatga

SEQ ID NO: 752 - RNA, 20 nt, synthetic construct
  misc_feature 1..20: note = Seynthetic
  source 1..20: mol_type = other RNA
gctctcaaagcaacactttc

SEQ ID NO: 753 - RNA, 20 nt, synthetic construct
  misc_feature 1..20: note = Seynthetic
  source 1..20: mol_type = other RNA
gctgccggagtccccctgtg

SEQ ID NO: 754 - RNA, 20 nt, synthetic construct
  misc_feature 1..20: note = Seynthetic
  source 1..20: mol_type = other RNA
gctgcctgagtccccctgtg

SEQ ID NO: 755 - RNA, 20 nt, synthetic construct
  misc_feature 1..20: note = Seynthetic
  source 1..20: mol_type = other RNA
gctgcgggaaagggattccc

SEQ ID NO: 756 - RNA, 20 nt, synthetic construct
  misc_feature 1..20: note = Seynthetic
  source 1..20: mol_type = other RNA
gctgcgggaatgggattccc

SEQ ID NO: 757 - RNA, 20 nt, synthetic construct
  misc_feature 1..20: note = Seynthetic
  source 1..20: mol_type = other RNA
gctggaacatcgtgggggag

SEQ ID NO: 758 - RNA, 20 nt, synthetic construct
  misc_feature 1..20: note = Seynthetic
  source 1..20: mol_type = other RNA
gctggaacatggtgggggag

SEQ ID NO: 759 - RNA, 20 nt, synthetic construct
  misc_feature 1..20: note = Seynthetic
  source 1..20: mol_type = other RNA
gctggagtcccagctgcggg

SEQ ID NO: 760 - RNA, 20 nt, synthetic construct
  misc_feature 1..20: note = Seynthetic
  source 1..20: mol_type = other RNA
gctggagtcctgacgtgacc

SEQ ID NO: 761 - RNA, 20 nt, synthetic construct
  misc_feature 1..20: note = Seynthetic
  source 1..20: mol_type = other RNA
gctggagtcttgacgtgacc

SEQ ID NO: 762 - RNA, 20 nt, synthetic construct
  misc_feature 1..20: note = Seynthetic
  source 1..20: mol_type = other RNA
gctgggtcctgggaatccca

SEQ ID NO: 763 - RNA, 20 nt, synthetic construct
  misc_feature 1..20: note = Seynthetic
  source 1..20: mol_type = other RNA
gctgggtcctgggaatccct

SEQ ID NO: 764 - RNA, 20 nt, synthetic construct
  misc_feature 1..20: note = Seynthetic
  source 1..20: mol_type = other RNA
gctgtatctttggaaggggc

SEQ ID NO: 765 - RNA, 20 nt, synthetic construct
  misc_feature 1..20: note = Seynthetic
  source 1..20: mol_type = other RNA
gcttctaccagggaactcct

SEQ ID NO: 766 - RNA, 20 nt, synthetic construct
  misc_feature 1..20: note = Seynthetic
  source 1..20: mol_type = other RNA
gcttctaccagggagctcct

SEQ ID NO: 767 - RNA, 20 nt, synthetic construct
  misc_feature 1..20: note = Seynthetic
  source 1..20: mol_type = other RNA
ggaaagggattcccaggacc

SEQ ID NO: 768 - RNA, 20 nt, synthetic construct
  misc_feature 1..20: note = Seynthetic
  source 1..20: mol_type = other RNA
ggaaagttcagggatgtccg

SEQ ID NO: 769 - RNA, 20 nt, synthetic construct
  misc_feature 1..20: note = Seynthetic
  source 1..20: mol_type = other RNA
ggaacatcgtgggggagatg

SEQ ID NO: 770 - RNA, 20 nt, synthetic construct
  misc_feature 1..20: note = Seynthetic
  source 1..20: mol_type = other RNA
ggaacatggtgggggagatg

SEQ ID NO: 771 - RNA, 20 nt, synthetic construct
  misc_feature 1..20: note = Seynthetic
  source 1..20: mol_type = other RNA
ggaatcccattcccgcagct

SEQ ID NO: 772 - RNA, 20 nt, synthetic construct
  misc_feature 1..20: note = Seynthetic
  source 1..20: mol_type = other RNA
ggaatccctttcccgcagct

SEQ ID NO: 773 - RNA, 20 nt, synthetic construct
  misc_feature 1..20: note = Seynthetic
  source 1..20: mol_type = other RNA
ggaatgggattcccaggacc

SEQ ID NO: 774 - RNA, 20 nt, synthetic construct
  misc_feature 1..20: note = Seynthetic
  source 1..20: mol_type = other RNA
ggacatccctgaactttcca

SEQ ID NO: 775 - RNA, 20 nt, synthetic construct
  misc_feature 1..20: note = Seynthetic
  source 1..20: mol_type = other RNA
ggacgccgtatctttggaag

SEQ ID NO: 776 - RNA, 20 nt, synthetic construct
  misc_feature 1..20: note = Seynthetic
  source 1..20: mol_type = other RNA
ggacgctgtatctttggaag

SEQ ID NO: 777 - RNA, 20 nt, synthetic construct
  misc_feature 1..20: note = Seynthetic
  source 1..20: mol_type = other RNA
ggacggtcctccagcccctc

SEQ ID NO: 778 - RNA, 20 nt, synthetic construct
  misc_feature 1..20: note = Seynthetic
  source 1..20: mol_type = other RNA
ggactcaggcagccccttgg

SEQ ID NO: 779 - RNA, 20 nt, synthetic construct
  misc_feature 1..20: note = Seynthetic
  source 1..20: mol_type = other RNA
ggactccagcctggcccagg

SEQ ID NO: 780 - RNA, 20 nt, synthetic construct
  misc_feature 1..20: note = Seynthetic
  source 1..20: mol_type = other RNA
ggactccggcagccccttgg

SEQ ID NO: 781 - RNA, 20 nt, synthetic construct
  misc_feature 1..20: note = Seynthetic
  source 1..20: mol_type = other RNA
ggagcagcacacgaggttcc

SEQ ID NO: 782 - RNA, 20 nt, synthetic construct
  misc_feature 1..20: note = Seynthetic
  source 1..20: mol_type = other RNA
ggagctccctggtagaagca

SEQ ID NO: 783 - RNA, 20 nt, synthetic construct
  misc_feature 1..20: note = Seynthetic
  source 1..20: mol_type = other RNA
ggagctcctgccctgcctac

SEQ ID NO: 784 - RNA, 20 nt, synthetic construct
  misc_feature 1..20: note = Seynthetic
  source 1..20: mol_type = other RNA
ggaggggctggaggaccgtc

SEQ ID NO: 785 - RNA, 20 nt, synthetic construct
  misc_feature 1..20: note = Seynthetic
  source 1..20: mol_type = other RNA
ggaggggtaggcagggcagg

SEQ ID NO: 786 - RNA, 20 nt, synthetic construct
  misc_feature 1..20: note = Seynthetic
  source 1..20: mol_type = other RNA
ggagtcccagctgcgggaaa

SEQ ID NO: 787 - RNA, 20 nt, synthetic construct
  misc_feature 1..20: note = Seynthetic
  source 1..20: mol_type = other RNA
ggagtcccagctgcgggaat

SEQ ID NO: 788 - RNA, 20 nt, synthetic construct
  misc_feature 1..20: note = Seynthetic
  source 1..20: mol_type = other RNA
ggagtccccctgtggaggca

SEQ ID NO: 789 - RNA, 20 nt, synthetic construct
  misc_feature 1..20: note = Seynthetic
  source 1..20: mol_type = other RNA
ggagtcctgacgtgaccaca

SEQ ID NO: 790 - RNA, 20 nt, synthetic construct
  misc_feature 1..20: note = Seynthetic
  source 1..20: mol_type = other RNA
ggagtctacacccgctgtga

SEQ ID NO: 791 - RNA, 20 nt, synthetic construct
  misc_feature 1..20: note = Seynthetic
  source 1..20: mol_type = other RNA
ggagtctacacctgctgtga

SEQ ID NO: 792 - RNA, 20 nt, synthetic construct
  misc_feature 1..20: note = Seynthetic
  source 1..20: mol_type = other RNA
ggagtcttgacgtgaccaca

SEQ ID NO: 793 - RNA, 20 nt, synthetic construct
  misc_feature 1..20: note = Seynthetic
  source 1..20: mol_type = other RNA
ggagttccctggtagaagca

SEQ ID NO: 794 - RNA, 20 nt, synthetic construct
  misc_feature 1..20: note = Seynthetic
  source 1..20: mol_type = other RNA
ggatgaaataaattcacgtt

SEQ ID NO: 795 - RNA, 20 nt, synthetic construct
  misc_feature 1..20: note = Seynthetic
  source 1..20: mol_type = other RNA
ggatgcacaatcgcattcaa

SEQ ID NO: 796 - RNA, 20 nt, synthetic construct
  misc_feature 1..20: note = Seynthetic
  source 1..20: mol_type = other RNA
ggatgtccgccttcaggtgt

SEQ ID NO: 797 - RNA, 20 nt, synthetic construct
  misc_feature 1..20: note = Seynthetic
  source 1..20: mol_type = other RNA
ggattgtgaaacaccctggg

SEQ ID NO: 798 - RNA, 20 nt, synthetic construct
  misc_feature 1..20: note = Seynthetic
  source 1..20: mol_type = other RNA
ggatttcaggggtgggggac

SEQ ID NO: 799 - RNA, 20 nt, synthetic construct
  misc_feature 1..20: note = Seynthetic
  source 1..20: mol_type = other RNA
ggcaaaacccccgctccatt

SEQ ID NO: 800 - RNA, 20 nt, synthetic construct
  misc_feature 1..20: note = Seynthetic
  source 1..20: mol_type = other RNA
ggcacacttaataaagactg

SEQ ID NO: 801 - RNA, 20 nt, synthetic construct
  misc_feature 1..20: note = Seynthetic
  source 1..20: mol_type = other RNA
ggcacacttaataatgactg

SEQ ID NO: 802 - RNA, 20 nt, synthetic construct
  misc_feature 1..20: note = Seynthetic
  source 1..20: mol_type = other RNA
ggcaggagctccctggtaga

SEQ ID NO: 803 - RNA, 20 nt, synthetic construct
  misc_feature 1..20: note = Seynthetic
  source 1..20: mol_type = other RNA
ggcaggagttccctggtaga

SEQ ID NO: 804 - RNA, 20 nt, synthetic construct
  misc_feature 1..20: note = Seynthetic
  source 1..20: mol_type = other RNA
ggcatccctgaactttccat

SEQ ID NO: 805 - RNA, 20 nt, synthetic construct
  misc_feature 1..20: note = Seynthetic
  source 1..20: mol_type = other RNA
ggccaagccccttccaaaga

SEQ ID NO: 806 - RNA, 20 nt, synthetic construct
  misc_feature 1..20: note = Seynthetic
  source 1..20: mol_type = other RNA
ggccacagggacagagacaa

SEQ ID NO: 807 - RNA, 20 nt, synthetic construct
  misc_feature 1..20: note = Seynthetic
  source 1..20: mol_type = other RNA
ggccaggctggagtcctgac

SEQ ID NO: 808 - RNA, 20 nt, synthetic construct
  misc_feature 1..20: note = Seynthetic
  source 1..20: mol_type = other RNA
ggccaggctggagtcttgac

SEQ ID NO: 809 - RNA, 20 nt, synthetic construct
  misc_feature 1..20: note = Seynthetic
  source 1..20: mol_type = other RNA
ggccgttgcagctggaacat

SEQ ID NO: 810 - RNA, 20 nt, synthetic construct
  misc_feature 1..20: note = Seynthetic
  source 1..20: mol_type = other RNA
ggcggacatccctgaacttt

SEQ ID NO: 811 - RNA, 20 nt, synthetic construct
  misc_feature 1..20: note = Seynthetic
  source 1..20: mol_type = other RNA
ggcgtcccccacccctgaaa

SEQ ID NO: 812 - RNA, 20 nt, synthetic construct
  misc_feature 1..20: note = Seynthetic
  source 1..20: mol_type = other RNA
ggctgccggagtccccctgt

SEQ ID NO: 813 - RNA, 20 nt, synthetic construct
  misc_feature 1..20: note = Seynthetic
  source 1..20: mol_type = other RNA
ggctggagtcctgacgtgac

SEQ ID NO: 814 - RNA, 20 nt, synthetic construct
  misc_feature 1..20: note = Seynthetic
  source 1..20: mol_type = other RNA
ggctggagtcttgacgtgac

SEQ ID NO: 815 - RNA, 20 nt, synthetic construct
  misc_feature 1..20: note = Seynthetic
  source 1..20: mol_type = other RNA
ggctgggtcctgggaatccc

SEQ ID NO: 816 - RNA, 20 nt, synthetic construct
  misc_feature 1..20: note = Seynthetic
  source 1..20: mol_type = other RNA
gggaaagggattcccaggac

SEQ ID NO: 817 - RNA, 20 nt, synthetic construct
  misc_feature 1..20: note = Seynthetic
  source 1..20: mol_type = other RNA
gggaactcctgccctgccta

SEQ ID NO: 818 - RNA, 20 nt, synthetic construct
  misc_feature 1..20: note = Seynthetic
  source 1..20: mol_type = other RNA
gggaatcccattcccgcagc

SEQ ID NO: 819 - RNA, 20 nt, synthetic construct
  misc_feature 1..20: note = Seynthetic
  source 1..20: mol_type = other RNA
gggaatccctttcccgcagc

SEQ ID NO: 820 - RNA, 20 nt, synthetic construct
  misc_feature 1..20: note = Seynthetic
  source 1..20: mol_type = other RNA
gggaatgggattcccaggac

SEQ ID NO: 821 - RNA, 20 nt, synthetic construct
  misc_feature 1..20: note = Seynthetic
  source 1..20: mol_type = other RNA
gggacgccgtatctttggaa

SEQ ID NO: 822 - RNA, 20 nt, synthetic construct
  misc_feature 1..20: note = Seynthetic
  source 1..20: mol_type = other RNA
gggacgctgtatctttggaa

SEQ ID NO: 823 - RNA, 20 nt, synthetic construct
  misc_feature 1..20: note = Seynthetic
  source 1..20: mol_type = other RNA
gggactccggcagccccttg

SEQ ID NO: 824 - RNA, 20 nt, synthetic construct
  misc_feature 1..20: note = Seynthetic
  source 1..20: mol_type = other RNA
gggagctcctgccctgccta

SEQ ID NO: 825 - RNA, 20 nt, synthetic construct
  misc_feature 1..20: note = Seynthetic
  source 1..20: mol_type = other RNA
gggatgtccgccttcaggtg

SEQ ID NO: 826 - RNA, 20 nt, synthetic construct
  misc_feature 1..20: note = Seynthetic
  source 1..20: mol_type = other RNA
gggatttcaggggtggggga

SEQ ID NO: 827 - RNA, 20 nt, synthetic construct
  misc_feature 1..20: note = Seynthetic
  source 1..20: mol_type = other RNA
gggcaggagctccctggtag

SEQ ID NO: 828 - RNA, 20 nt, synthetic construct
  misc_feature 1..20: note = Seynthetic
  source 1..20: mol_type = other RNA
gggcaggagttccctggtag

SEQ ID NO: 829 - RNA, 20 nt, synthetic construct
  misc_feature 1..20: note = Seynthetic
  source 1..20: mol_type = other RNA
gggccaggctggagtcctga

SEQ ID NO: 830 - RNA, 20 nt, synthetic construct
  misc_feature 1..20: note = Seynthetic
  source 1..20: mol_type = other RNA
gggccaggctggagtcttga

SEQ ID NO: 831 - RNA, 20 nt, synthetic construct
  misc_feature 1..20: note = Seynthetic
  source 1..20: mol_type = other RNA
gggctgggtcctgggaatcc

SEQ ID NO: 832 - RNA, 20 nt, synthetic construct
  misc_feature 1..20: note = Seynthetic
  source 1..20: mol_type = other RNA
ggggaccagaaggatgggga

SEQ ID NO: 833 - RNA, 20 nt, synthetic construct
  misc_feature 1..20: note = Seynthetic
  source 1..20: mol_type = other RNA
ggggacgccgtatctttgga

SEQ ID NO: 834 - RNA, 20 nt, synthetic construct
  misc_feature 1..20: note = Seynthetic
  source 1..20: mol_type = other RNA
ggggacgctgtatctttgga

SEQ ID NO: 835 - RNA, 20 nt, synthetic construct
  misc_feature 1..20: note = Seynthetic
  source 1..20: mol_type = other RNA
ggggactccggcagcccctt

SEQ ID NO: 836 - RNA, 20 nt, synthetic construct
  misc_feature 1..20: note = Seynthetic
  source 1..20: mol_type = other RNA
ggggctgggtcctgggaatc

SEQ ID NO: 837 - RNA, 20 nt, synthetic construct
  misc_feature 1..20: note = Seynthetic
  source 1..20: mol_type = other RNA
gggggacgccgtatctttgg

SEQ ID NO: 838 - RNA, 20 nt, synthetic construct
  misc_feature 1..20: note = Seynthetic
  source 1..20: mol_type = other RNA
gggggacgctgtatctttgg

SEQ ID NO: 839 - RNA, 20 nt, synthetic construct
  misc_feature 1..20: note = Seynthetic
  source 1..20: mol_type = other RNA
gggggactcaggcagcccct

SEQ ID NO: 840 - RNA, 20 nt, synthetic construct
  misc_feature 1..20: note = Seynthetic
  source 1..20: mol_type = other RNA
gggggactctgggtgtggtg

SEQ ID NO: 841 - RNA, 20 nt, synthetic construct
  misc_feature 1..20: note = Seynthetic
  source 1..20: mol_type = other RNA
ggggtaggcagggcaggagc

SEQ ID NO: 842 - RNA, 20 nt, synthetic construct
  misc_feature 1..20: note = Seynthetic
  source 1..20: mol_type = other RNA
ggggtaggcagggcaggagt

SEQ ID NO: 843 - RNA, 20 nt, synthetic construct
  misc_feature 1..20: note = Seynthetic
  source 1..20: mol_type = other RNA
ggggtgggggacgccgtatc

SEQ ID NO: 844 - RNA, 20 nt, synthetic construct
  misc_feature 1..20: note = Seynthetic
  source 1..20: mol_type = other RNA
ggggtgggggacgctgtatc

SEQ ID NO: 845 - RNA, 20 nt, synthetic construct
  misc_feature 1..20: note = Seynthetic
  source 1..20: mol_type = other RNA
ggggtgttatggtcacagca

SEQ ID NO: 846 - RNA, 20 nt, synthetic construct
  misc_feature 1..20: note = Seynthetic
  source 1..20: mol_type = other RNA
ggggtgttatggtcacagcg

SEQ ID NO: 847 - RNA, 20 nt, synthetic construct
  misc_feature 1..20: note = Seynthetic
  source 1..20: mol_type = other RNA
gggtaggcagggcaggagct

SEQ ID NO: 848 - RNA, 20 nt, synthetic construct
  misc_feature 1..20: note = Seynthetic
  source 1..20: mol_type = other RNA
gggtaggcagggcaggagtt

SEQ ID NO: 849 - RNA, 20 nt, synthetic construct
  misc_feature 1..20: note = Seynthetic
  source 1..20: mol_type = other RNA
gggtcctgggaatcccattc

SEQ ID NO: 850 - RNA, 20 nt, synthetic construct
  misc_feature 1..20: note = Seynthetic
  source 1..20: mol_type = other RNA
gggtcctgggaatccctttc

SEQ ID NO: 851 - RNA, 20 nt, synthetic construct
  misc_feature 1..20: note = Seynthetic
  source 1..20: mol_type = other RNA
gggtgactctgggtgtggtg

SEQ ID NO: 852 - RNA, 20 nt, synthetic construct
  misc_feature 1..20: note = Seynthetic
  source 1..20: mol_type = other RNA
gggtgggggacgccgtatct

SEQ ID NO: 853 - RNA, 20 nt, synthetic construct
  misc_feature 1..20: note = Seynthetic
  source 1..20: mol_type = other RNA
gggtgggggacgctgtatct

SEQ ID NO: 854 - RNA, 20 nt, synthetic construct
  misc_feature 1..20: note = Seynthetic
  source 1..20: mol_type = other RNA
gggtgtagactccgaggggg

SEQ ID NO: 855 - RNA, 20 nt, synthetic construct
  misc_feature 1..20: note = Seynthetic
  source 1..20: mol_type = other RNA
gggtgtcaagccccagagga

SEQ ID NO: 856 - RNA, 20 nt, synthetic construct
  misc_feature 1..20: note = Seynthetic
  source 1..20: mol_type = other RNA
gggtgtcaagccccagaggc

SEQ ID NO: 857 - RNA, 20 nt, synthetic construct
  misc_feature 1..20: note = Seynthetic
  source 1..20: mol_type = other RNA
gggtgttatggtcacagcag

SEQ ID NO: 858 - RNA, 20 nt, synthetic construct
  misc_feature 1..20: note = Seynthetic
  source 1..20: mol_type = other RNA
gggtgttatggtcacagcgg

SEQ ID NO: 859 - RNA, 20 nt, synthetic construct
  misc_feature 1..20: note = Seynthetic
  source 1..20: mol_type = other RNA
ggtaggcagggcaggagctc

SEQ ID NO: 860 - RNA, 20 nt, synthetic construct
  misc_feature 1..20: note = Seynthetic
  source 1..20: mol_type = other RNA
ggtaggcagggcaggagttc

SEQ ID NO: 861 - RNA, 20 nt, synthetic construct
  misc_feature 1..20: note = Seynthetic
  source 1..20: mol_type = other RNA
ggtaggggtgttatggtcac

SEQ ID NO: 862 - RNA, 20 nt, synthetic construct
  misc_feature 1..20: note = Seynthetic
  source 1..20: mol_type = other RNA
ggtatccccatccttctggt

SEQ ID NO: 863 - RNA, 20 nt, synthetic construct
  misc_feature 1..20: note = Seynthetic
  source 1..20: mol_type = other RNA
ggtcacagcaggtgtagact

SEQ ID NO: 864 - RNA, 20 nt, synthetic construct
  misc_feature 1..20: note = Seynthetic
  source 1..20: mol_type = other RNA
ggtcacagcgggtgtagact

SEQ ID NO: 865 - RNA, 20 nt, synthetic construct
  misc_feature 1..20: note = Seynthetic
  source 1..20: mol_type = other RNA
ggtcacgtcaagactccagc

SEQ ID NO: 866 - RNA, 20 nt, synthetic construct
  misc_feature 1..20: note = Seynthetic
  source 1..20: mol_type = other RNA
ggtcacgtcaggactccagc

SEQ ID NO: 867 - RNA, 20 nt, synthetic construct
  misc_feature 1..20: note = Seynthetic
  source 1..20: mol_type = other RNA
ggtccccgcaccatggatta

SEQ ID NO: 868 - RNA, 20 nt, synthetic construct
  misc_feature 1..20: note = Seynthetic
  source 1..20: mol_type = other RNA
ggtccctgcaccatggatta

SEQ ID NO: 869 - RNA, 20 nt, synthetic construct
  misc_feature 1..20: note = Seynthetic
  source 1..20: mol_type = other RNA
ggtcctccagcccctccagg

SEQ ID NO: 870 - RNA, 20 nt, synthetic construct
  misc_feature 1..20: note = Seynthetic
  source 1..20: mol_type = other RNA
ggtcctgggaatcccattcc

SEQ ID NO: 871 - RNA, 20 nt, synthetic construct
  misc_feature 1..20: note = Seynthetic
  source 1..20: mol_type = other RNA
ggtcctgggaatccctttcc

SEQ ID NO: 872 - RNA, 20 nt, synthetic construct
  misc_feature 1..20: note = Seynthetic
  source 1..20: mol_type = other RNA
ggtgaagagaaaagccgggg

SEQ ID NO: 873 - RNA, 20 nt, synthetic construct
  misc_feature 1..20: note = Seynthetic
  source 1..20: mol_type = other RNA
ggtgactctgggtgtggtga

SEQ ID NO: 874 - RNA, 20 nt, synthetic construct
  misc_feature 1..20: note = Seynthetic
  source 1..20: mol_type = other RNA
ggtgcagggaccagaaggat

SEQ ID NO: 875 - RNA, 20 nt, synthetic construct
  misc_feature 1..20: note = Seynthetic
  source 1..20: mol_type = other RNA
ggtgcggggaccagaaggat

SEQ ID NO: 876 - RNA, 20 nt, synthetic construct
  misc_feature 1..20: note = Seynthetic
  source 1..20: mol_type = other RNA
ggtgggggacgccgtatctt

SEQ ID NO: 877 - RNA, 20 nt, synthetic construct
  misc_feature 1..20: note = Seynthetic
  source 1..20: mol_type = other RNA
ggtgggggacgctgtatctt

SEQ ID NO: 878 - RNA, 20 nt, synthetic construct
  misc_feature 1..20: note = Seynthetic
  source 1..20: mol_type = other RNA
ggtgtcaagccccagaggac

SEQ ID NO: 879 - RNA, 20 nt, synthetic construct
  misc_feature 1..20: note = Seynthetic
  source 1..20: mol_type = other RNA
ggtgtcaagccccagaggcc

SEQ ID NO: 880 - RNA, 20 nt, synthetic construct
  misc_feature 1..20: note = Seynthetic
  source 1..20: mol_type = other RNA
ggtgttatggtcacagcagg

SEQ ID NO: 881 - RNA, 20 nt, synthetic construct
  misc_feature 1..20: note = Seynthetic
  source 1..20: mol_type = other RNA
ggtgttatggtcacagcggg

SEQ ID NO: 882 - RNA, 20 nt, synthetic construct
  misc_feature 1..20: note = Seynthetic
  source 1..20: mol_type = other RNA
ggttcccccggcttttctct

SEQ ID NO: 883 - RNA, 20 nt, synthetic construct
  misc_feature 1..20: note = Seynthetic
  source 1..20: mol_type = other RNA
ggttccccctgcttttctct

SEQ ID NO: 884 - RNA, 20 nt, synthetic construct
  misc_feature 1..20: note = Seynthetic
  source 1..20: mol_type = other RNA
gtaaagttcttagctaagaa

SEQ ID NO: 885 - RNA, 20 nt, synthetic construct
  misc_feature 1..20: note = Seynthetic
  source 1..20: mol_type = other RNA
gtaggcagggcaggagttcc

SEQ ID NO: 886 - RNA, 20 nt, synthetic construct
  misc_feature 1..20: note = Seynthetic
  source 1..20: mol_type = other RNA
gtaggggtgttatggtcaca

SEQ ID NO: 887 - RNA, 20 nt, synthetic construct
  misc_feature 1..20: note = Seynthetic
  source 1..20: mol_type = other RNA
gtatccccatccttctggtc

SEQ ID NO: 888 - RNA, 20 nt, synthetic construct
  misc_feature 1..20: note = Seynthetic
  source 1..20: mol_type = other RNA
gtcaagccccagaggacaca

SEQ ID NO: 889 - RNA, 20 nt, synthetic construct
  misc_feature 1..20: note = Seynthetic
  source 1..20: mol_type = other RNA
gtcaagccccagaggccaca

SEQ ID NO: 890 - RNA, 20 nt, synthetic construct
  misc_feature 1..20: note = Seynthetic
  source 1..20: mol_type = other RNA
gtcacagcaggtgtagactc

SEQ ID NO: 891 - RNA, 20 nt, synthetic construct
  misc_feature 1..20: note = Seynthetic
  source 1..20: mol_type = other RNA
gtcacagcgggtgtagactc

SEQ ID NO: 892 - RNA, 20 nt, synthetic construct
  misc_feature 1..20: note = Seynthetic
  source 1..20: mol_type = other RNA
gtcacccagggtgtttcaca

SEQ ID NO: 893 - RNA, 20 nt, synthetic construct
  misc_feature 1..20: note = Seynthetic
  source 1..20: mol_type = other RNA
gtcacgtcaagactccagcc

SEQ ID NO: 894 - RNA, 20 nt, synthetic construct
  misc_feature 1..20: note = Seynthetic
  source 1..20: mol_type = other RNA
gtcacgtcaggactccagcc

SEQ ID NO: 895 - RNA, 20 nt, synthetic construct
  misc_feature 1..20: note = Seynthetic
  source 1..20: mol_type = other RNA
gtcagataatcaatgtgggt

SEQ ID NO: 896 - RNA, 20 nt, synthetic construct
  misc_feature 1..20: note = Seynthetic
  source 1..20: mol_type = other RNA
gtcagggtgtcaagccccag

SEQ ID NO: 897 - RNA, 20 nt, synthetic construct
  misc_feature 1..20: note = Seynthetic
  source 1..20: mol_type = other RNA
gtcattattaagtgtgccga

SEQ ID NO: 898 - RNA, 20 nt, synthetic construct
  misc_feature 1..20: note = Seynthetic
  source 1..20: mol_type = other RNA
gtcccagctgcgggaaaggg

SEQ ID NO: 899 - RNA, 20 nt, synthetic construct
  misc_feature 1..20: note = Seynthetic
  source 1..20: mol_type = other RNA
gtcccagctgcgggaatggg

SEQ ID NO: 900 - RNA, 20 nt, synthetic construct
  misc_feature 1..20: note = Seynthetic
  source 1..20: mol_type = other RNA
gtcccccagggtgtttcaca

SEQ ID NO: 901 - RNA, 20 nt, synthetic construct
  misc_feature 1..20: note = Seynthetic
  source 1..20: mol_type = other RNA
gtccccctcggagtctacac

SEQ ID NO: 902 - RNA, 20 nt, synthetic construct
  misc_feature 1..20: note = Seynthetic
  source 1..20: mol_type = other RNA
gtccctgcaccatggattaa

SEQ ID NO: 903 - RNA, 20 nt, synthetic construct
  misc_feature 1..20: note = Seynthetic
  source 1..20: mol_type = other RNA
gtccctgtggcctctggggc

SEQ ID NO: 904 - RNA, 20 nt, synthetic construct
  misc_feature 1..20: note = Seynthetic
  source 1..20: mol_type = other RNA
gtcctccagccccgccaggt

SEQ ID NO: 905 - RNA, 20 nt, synthetic construct
  misc_feature 1..20: note = Seynthetic
  source 1..20: mol_type = other RNA
gtcctctggggcttgacacc

SEQ ID NO: 906 - RNA, 20 nt, synthetic construct
  misc_feature 1..20: note = Seynthetic
  source 1..20: mol_type = other RNA
gtcctgacgtgaccacaggc

SEQ ID NO: 907 - RNA, 20 nt, synthetic construct
  misc_feature 1..20: note = Seynthetic
  source 1..20: mol_type = other RNA
gtcctgggaatcccattccc

SEQ ID NO: 908 - RNA, 20 nt, synthetic construct
  misc_feature 1..20: note = Seynthetic
  source 1..20: mol_type = other RNA
gtcctgggaatccctttccc

SEQ ID NO: 909 - RNA, 20 nt, synthetic construct
  misc_feature 1..20: note = Seynthetic
  source 1..20: mol_type = other RNA
gtcggcacacttaataaaga

SEQ ID NO: 910 - RNA, 20 nt, synthetic construct
  misc_feature 1..20: note = Seynthetic
  source 1..20: mol_type = other RNA
gtcggcacacttaataatga

SEQ ID NO: 911 - RNA, 20 nt, synthetic construct
  misc_feature 1..20: note = Seynthetic
  source 1..20: mol_type = other RNA
gtctacacccgctgtgacca

SEQ ID NO: 912 - RNA, 20 nt, synthetic construct
  misc_feature 1..20: note = Seynthetic
  source 1..20: mol_type = other RNA
gtctacacctgctgtgacca

SEQ ID NO: 913 - RNA, 20 nt, synthetic construct
  misc_feature 1..20: note = Seynthetic
  source 1..20: mol_type = other RNA
gtctctgtccctgtggcctc

SEQ ID NO: 914 - RNA, 20 nt, synthetic construct
  misc_feature 1..20: note = Seynthetic
  source 1..20: mol_type = other RNA
gtctctgtccctgtgtcctc

SEQ ID NO: 915 - RNA, 20 nt, synthetic construct
  misc_feature 1..20: note = Seynthetic
  source 1..20: mol_type = other RNA
gtctgcctccacagggggac

SEQ ID NO: 916 - RNA, 20 nt, synthetic construct
  misc_feature 1..20: note = Seynthetic
  source 1..20: mol_type = other RNA
gtcttgacgtgaccacaggc

SEQ ID NO: 917 - RNA, 20 nt, synthetic construct
  misc_feature 1..20: note = Seynthetic
  source 1..20: mol_type = other RNA
gtctttattaagtgtgccga

SEQ ID NO: 918 - RNA, 20 nt, synthetic construct
  misc_feature 1..20: note = Seynthetic
  source 1..20: mol_type = other RNA
gtgaaacaccctgggggact

SEQ ID NO: 919 - RNA, 20 nt, synthetic construct
  misc_feature 1..20: note = Seynthetic
  source 1..20: mol_type = other RNA
gtgaaacaccctgggtgact

SEQ ID NO: 920 - RNA, 20 nt, synthetic construct
  misc_feature 1..20: note = Seynthetic
  source 1..20: mol_type = other RNA
gtgaagagaaaagcaggggg

SEQ ID NO: 921 - RNA, 20 nt, synthetic construct
  misc_feature 1..20: note = Seynthetic
  source 1..20: mol_type = other RNA
gtgaagagaaaagccggggg

SEQ ID NO: 922 - RNA, 20 nt, synthetic construct
  misc_feature 1..20: note = Seynthetic
  source 1..20: mol_type = other RNA
gtgactctgggtgtggtgaa

SEQ ID NO: 923 - RNA, 20 nt, synthetic construct
  misc_feature 1..20: note = Seynthetic
  source 1..20: mol_type = other RNA
gtgacttccccacattgtat

SEQ ID NO: 924 - RNA, 20 nt, synthetic construct
  misc_feature 1..20: note = Seynthetic
  source 1..20: mol_type = other RNA
gtgacttccccacattgtgt

SEQ ID NO: 925 - RNA, 20 nt, synthetic construct
  misc_feature 1..20: note = Seynthetic
  source 1..20: mol_type = other RNA
gtgcagggaccagaaggatg

SEQ ID NO: 926 - RNA, 20 nt, synthetic construct
  misc_feature 1..20: note = Seynthetic
  source 1..20: mol_type = other RNA
gtgcagtcattattaagtgt

SEQ ID NO: 927 - RNA, 20 nt, synthetic construct
  misc_feature 1..20: note = Seynthetic
  source 1..20: mol_type = other RNA
gtgcagtctttattaagtgt

SEQ ID NO: 928 - RNA, 20 nt, synthetic construct
  misc_feature 1..20: note = Seynthetic
  source 1..20: mol_type = other RNA
gtgcggggaccagaaggatg

SEQ ID NO: 929 - RNA, 20 nt, synthetic construct
  misc_feature 1..20: note = Seynthetic
  source 1..20: mol_type = other RNA
gtggcctctggggcttgaca

SEQ ID NO: 930 - RNA, 20 nt, synthetic construct
  misc_feature 1..20: note = Seynthetic
  source 1..20: mol_type = other RNA
gtgggggacgccgtatcttt

SEQ ID NO: 931 - RNA, 20 nt, synthetic construct
  misc_feature 1..20: note = Seynthetic
  source 1..20: mol_type = other RNA
gtgggggacgctgtatcttt

SEQ ID NO: 932 - RNA, 20 nt, synthetic construct
  misc_feature 1..20: note = Seynthetic
  source 1..20: mol_type = other RNA
gtggtcacgtcaggactcca

SEQ ID NO: 933 - RNA, 20 nt, synthetic construct
  misc_feature 1..20: note = Seynthetic
  source 1..20: mol_type = other RNA
gtgtcaagccccagaggaca

SEQ ID NO: 934 - RNA, 20 nt, synthetic construct
  misc_feature 1..20: note = Seynthetic
  source 1..20: mol_type = other RNA
gtgtcaagccccagaggcca

SEQ ID NO: 935 - RNA, 20 nt, synthetic construct
  misc_feature 1..20: note = Seynthetic
  source 1..20: mol_type = other RNA
gtgtcctctggggcttgaca

SEQ ID NO: 936 - RNA, 20 nt, synthetic construct
  misc_feature 1..20: note = Seynthetic
  source 1..20: mol_type = other RNA
gtgttatggtcacagcaggt

SEQ ID NO: 937 - RNA, 20 nt, synthetic construct
  misc_feature 1..20: note = Seynthetic
  source 1..20: mol_type = other RNA
gtgttatggtcacagcgggt

SEQ ID NO: 938 - RNA, 20 nt, synthetic construct
  misc_feature 1..20: note = Seynthetic
  source 1..20: mol_type = other RNA
gttatatggaaagttcaggg

SEQ ID NO: 939 - RNA, 20 nt, synthetic construct
  misc_feature 1..20: note = Seynthetic
  source 1..20: mol_type = other RNA
gttatggtcacagcaggtgt

SEQ ID NO: 940 - RNA, 20 nt, synthetic construct
  misc_feature 1..20: note = Seynthetic
  source 1..20: mol_type = other RNA
gttatggtcacagcgggtgt

SEQ ID NO: 941 - RNA, 20 nt, synthetic construct
  misc_feature 1..20: note = Seynthetic
  source 1..20: mol_type = other RNA
gttcagggatgtccgccttc

SEQ ID NO: 942 - RNA, 20 nt, synthetic construct
  misc_feature 1..20: note = Seynthetic
  source 1..20: mol_type = other RNA
gttcataaagttcttagcta

SEQ ID NO: 943 - RNA, 20 nt, synthetic construct
  misc_feature 1..20: note = Seynthetic
  source 1..20: mol_type = other RNA
gttcccccggcttttctctt

SEQ ID NO: 944 - RNA, 20 nt, synthetic construct
  misc_feature 1..20: note = Seynthetic
  source 1..20: mol_type = other RNA
gttccctggtagaagcatga

SEQ ID NO: 945 - RNA, 20 nt, synthetic construct
  misc_feature 1..20: note = Seynthetic
  source 1..20: mol_type = other RNA
gttcctcttcccatctcccc

SEQ ID NO: 946 - RNA, 20 nt, synthetic construct
  misc_feature 1..20: note = Seynthetic
  source 1..20: mol_type = other RNA
gttcgtaaagttcttagcta

SEQ ID NO: 947 - RNA, 20 nt, synthetic construct
  misc_feature 1..20: note = Seynthetic
  source 1..20: mol_type = other RNA
gttgcagaccaaggggctgc

SEQ ID NO: 948 - RNA, 20 nt, synthetic construct
  misc_feature 1..20: note = Seynthetic
  source 1..20: mol_type = other RNA
gttgcagctggaacatcgtg

SEQ ID NO: 949 - RNA, 20 nt, synthetic construct
  misc_feature 1..20: note = Seynthetic
  source 1..20: mol_type = other RNA
gttgcagctggaacatggtg

SEQ ID NO: 950 - RNA, 20 nt, synthetic construct
  misc_feature 1..20: note = Seynthetic
  source 1..20: mol_type = other RNA
gttgcattaatccatggtgc

SEQ ID NO: 951 - RNA, 20 nt, synthetic construct
  misc_feature 1..20: note = Seynthetic
  source 1..20: mol_type = other RNA
taaagactgcacgtgagaat

SEQ ID NO: 952 - RNA, 20 nt, synthetic construct
  misc_feature 1..20: note = Seynthetic
  source 1..20: mol_type = other RNA
taaattcacgttcgtaaagt

SEQ ID NO: 953 - RNA, 20 nt, synthetic construct
  misc_feature 1..20: note = Seynthetic
  source 1..20: mol_type = other RNA
taagaactttacgaacgtga

SEQ ID NO: 954 - RNA, 20 nt, synthetic construct
  misc_feature 1..20: note = Seynthetic
  source 1..20: mol_type = other RNA
taagaactttatgaacgtga

SEQ ID NO: 955 - RNA, 20 nt, synthetic construct
  misc_feature 1..20: note = Seynthetic
  source 1..20: mol_type = other RNA
taataaagactgcacgtgag

SEQ ID NO: 956 - RNA, 20 nt, synthetic construct
  misc_feature 1..20: note = Seynthetic
  source 1..20: mol_type = other RNA
taataatgactgcacgtgag

SEQ ID NO: 957 - RNA, 20 nt, synthetic construct
  misc_feature 1..20: note = Seynthetic
  source 1..20: mol_type = other RNA
taatccatggtgcggggacc

SEQ ID NO: 958 - RNA, 20 nt, synthetic construct
  misc_feature 1..20: note = Seynthetic
  source 1..20: mol_type = other RNA
taatgactgcacgtgagaat

SEQ ID NO: 959 - RNA, 20 nt, synthetic construct
  misc_feature 1..20: note = Seynthetic
  source 1..20: mol_type = other RNA
tacaatgtggggaagtcact

SEQ ID NO: 960 - RNA, 20 nt, synthetic construct
  misc_feature 1..20: note = Seynthetic
  source 1..20: mol_type = other RNA
tacacccgctgtgaccataa

SEQ ID NO: 961 - RNA, 20 nt, synthetic construct
  misc_feature 1..20: note = Seynthetic
  source 1..20: mol_type = other RNA
tacacctgctgtgaccataa

SEQ ID NO: 962 - RNA, 20 nt, synthetic construct
  misc_feature 1..20: note = Seynthetic
  source 1..20: mol_type = other RNA
tacagcgtcccccacccctg

SEQ ID NO: 963 - RNA, 20 nt, synthetic construct
  misc_feature 1..20: note = Seynthetic
  source 1..20: mol_type = other RNA
tacattctcacgtgcagtca

SEQ ID NO: 964 - RNA, 20 nt, synthetic construct
  misc_feature 1..20: note = Seynthetic
  source 1..20: mol_type = other RNA
tacattctcacgtgcagtct

SEQ ID NO: 965 - RNA, 20 nt, synthetic construct
  misc_feature 1..20: note = Seynthetic
  source 1..20: mol_type = other RNA
tacgaacgtgaatttatttc

SEQ ID NO: 966 - RNA, 20 nt, synthetic construct
  misc_feature 1..20: note = Seynthetic
  source 1..20: mol_type = other RNA
tacggcgtcccccacccctg

SEQ ID NO: 967 - RNA, 20 nt, synthetic construct
  misc_feature 1..20: note = Seynthetic
  source 1..20: mol_type = other RNA
tactacattctcacgtgcag

SEQ ID NO: 968 - RNA, 20 nt, synthetic construct
  misc_feature 1..20: note = Seynthetic
  source 1..20: mol_type = other RNA
tagctaagaactttacgaac

SEQ ID NO: 969 - RNA, 20 nt, synthetic construct
  misc_feature 1..20: note = Seynthetic
  source 1..20: mol_type = other RNA
tagctaagaactttatgaac

SEQ ID NO: 970 - RNA, 20 nt, synthetic construct
  misc_feature 1..20: note = Seynthetic
  source 1..20: mol_type = other RNA
taggattgtgaaacaccctg

SEQ ID NO: 971 - RNA, 20 nt, synthetic construct
  misc_feature 1..20: note = Seynthetic
  source 1..20: mol_type = other RNA
taggcagggcaggagttccc

SEQ ID NO: 972 - RNA, 20 nt, synthetic construct
  misc_feature 1..20: note = Seynthetic
  source 1..20: mol_type = other RNA
taggggtgttatggtcacag

SEQ ID NO: 973 - RNA, 20 nt, synthetic construct
  misc_feature 1..20: note = Seynthetic
  source 1..20: mol_type = other RNA
tatatggaaagttcagggat

SEQ ID NO: 974 - RNA, 20 nt, synthetic construct
  misc_feature 1..20: note = Seynthetic
  source 1..20: mol_type = other RNA
tatccccatccttctggtcc

SEQ ID NO: 975 - RNA, 20 nt, synthetic construct
  misc_feature 1..20: note = Seynthetic
  source 1..20: mol_type = other RNA
tatctgaaatttgaatgcga

SEQ ID NO: 976 - RNA, 20 nt, synthetic construct
  misc_feature 1..20: note = Seynthetic
  source 1..20: mol_type = other RNA
tatctgacatttgaatgcga

SEQ ID NO: 977 - RNA, 20 nt, synthetic construct
  misc_feature 1..20: note = Seynthetic
  source 1..20: mol_type = other RNA
tatggaaagttcagggatgt

SEQ ID NO: 978 - RNA, 20 nt, synthetic construct
  misc_feature 1..20: note = Seynthetic
  source 1..20: mol_type = other RNA
tatggtcacagcaggtgtag

SEQ ID NO: 979 - RNA, 20 nt, synthetic construct
  misc_feature 1..20: note = Seynthetic
  source 1..20: mol_type = other RNA
tatggtcacagcgggtgtag

SEQ ID NO: 980 - RNA, 20 nt, synthetic construct
  misc_feature 1..20: note = Seynthetic
  source 1..20: mol_type = other RNA
tcaaagcaacactttctttt

SEQ ID NO: 981 - RNA, 20 nt, synthetic construct
  misc_feature 1..20: note = Seynthetic
  source 1..20: mol_type = other RNA
tcaagccacacctgaaggcg

SEQ ID NO: 982 - RNA, 20 nt, synthetic construct
  misc_feature 1..20: note = Seynthetic
  source 1..20: mol_type = other RNA
tcaagccccagaggccacag

SEQ ID NO: 983 - RNA, 20 nt, synthetic construct
  misc_feature 1..20: note = Seynthetic
  source 1..20: mol_type = other RNA
tcacagcaggtgtagactcc

SEQ ID NO: 984 - RNA, 20 nt, synthetic construct
  misc_feature 1..20: note = Seynthetic
  source 1..20: mol_type = other RNA
tcacagcgggtgtagactcc

SEQ ID NO: 985 - RNA, 20 nt, synthetic construct
  misc_feature 1..20: note = Seynthetic
  source 1..20: mol_type = other RNA
tcaccacacccagagtcacc

SEQ ID NO: 986 - RNA, 20 nt, synthetic construct
  misc_feature 1..20: note = Seynthetic
  source 1..20: mol_type = other RNA
tcaccacacccagagtcccc

SEQ ID NO: 987 - RNA, 20 nt, synthetic construct
  misc_feature 1..20: note = Seynthetic
  source 1..20: mol_type = other RNA
tcacccagggtgtttcacaa

SEQ ID NO: 988 - RNA, 20 nt, synthetic construct
  misc_feature 1..20: note = Seynthetic
  source 1..20: mol_type = other RNA
tcacgtcaagactccagcct

SEQ ID NO: 989 - RNA, 20 nt, synthetic construct
  misc_feature 1..20: note = Seynthetic
  source 1..20: mol_type = other RNA
tcacgtcaggactccagcct

SEQ ID NO: 990 - RNA, 20 nt, synthetic construct
  misc_feature 1..20: note = Seynthetic
  source 1..20: mol_type = other RNA
tcacgtgcagtcattattaa

SEQ ID NO: 991 - RNA, 20 nt, synthetic construct
  misc_feature 1..20: note = Seynthetic
  source 1..20: mol_type = other RNA
tcacgtgcagtctttattaa

SEQ ID NO: 992 - RNA, 20 nt, synthetic construct
  misc_feature 1..20: note = Seynthetic
  source 1..20: mol_type = other RNA
tcacgttcataaagttctta

SEQ ID NO: 993 - RNA, 20 nt, synthetic construct
  misc_feature 1..20: note = Seynthetic
  source 1..20: mol_type = other RNA
tcacgttcgtaaagttctta

SEQ ID NO: 994 - RNA, 20 nt, synthetic construct
  misc_feature 1..20: note = Seynthetic
  source 1..20: mol_type = other RNA
tcaggactccagcctggccc

SEQ ID NO: 995 - RNA, 20 nt, synthetic construct
  misc_feature 1..20: note = Seynthetic
  source 1..20: mol_type = other RNA
tcagggatgtccgccttcag

SEQ ID NO: 996 - RNA, 20 nt, synthetic construct
  misc_feature 1..20: note = Seynthetic
  source 1..20: mol_type = other RNA
tcaggggtgggggacgccgt

SEQ ID NO: 997 - RNA, 20 nt, synthetic construct
  misc_feature 1..20: note = Seynthetic
  source 1..20: mol_type = other RNA
tcaggggtgggggacgctgt

SEQ ID NO: 998 - RNA, 20 nt, synthetic construct
  misc_feature 1..20: note = Seynthetic
  source 1..20: mol_type = other RNA
tcagggtgtcaagccccaga

SEQ ID NO: 999 - RNA, 20 nt, synthetic construct
  misc_feature 1..20: note = Seynthetic
  source 1..20: mol_type = other RNA
tcatgcttctaccagggaac

SEQ ID NO: 1000 - RNA, 20 nt, synthetic construct
  misc_feature 1..20: note = Seynthetic
  source 1..20: mol_type = other RNA
tcatgcttctaccagggagc

SEQ ID NO: 1001 - RNA, 20 nt, synthetic construct
  misc_feature 1..20: note = Seynthetic
  source 1..20: mol_type = other RNA
tcattattaagtgtgccgac

SEQ ID NO: 1002 - RNA, 20 nt, synthetic construct
  misc_feature 1..20: note = Seynthetic
  source 1..20: mol_type = other RNA
tccaaagatacagcgtcccc

SEQ ID NO: 1003 - RNA, 20 nt, synthetic construct
  misc_feature 1..20: note = Seynthetic
  source 1..20: mol_type = other RNA
tccaaagatacggcgtcccc

SEQ ID NO: 1004 - RNA, 20 nt, synthetic construct
  misc_feature 1..20: note = Seynthetic
  source 1..20: mol_type = other RNA
tccacagggggactccggca

SEQ ID NO: 1005 - RNA, 20 nt, synthetic construct
  misc_feature 1..20: note = Seynthetic
  source 1..20: mol_type = other RNA
tccacttgcctgtggtcacg

SEQ ID NO: 1006 - RNA, 20 nt, synthetic construct
  misc_feature 1..20: note = Seynthetic
  source 1..20: mol_type = other RNA
tccatggtgaagagaaaagc

SEQ ID NO: 1007 - RNA, 20 nt, synthetic construct
  misc_feature 1..20: note = Seynthetic
  source 1..20: mol_type = other RNA
tccatggtgcagggaccaga

SEQ ID NO: 1008 - RNA, 20 nt, synthetic construct
  misc_feature 1..20: note = Seynthetic
  source 1..20: mol_type = other RNA
tccatggtgcggggaccaga

SEQ ID NO: 1009 - RNA, 20 nt, synthetic construct
  misc_feature 1..20: note = Seynthetic
  source 1..20: mol_type = other RNA
tcccagctgcgggaaaggga

SEQ ID NO: 1010 - RNA, 20 nt, synthetic construct
  misc_feature 1..20: note = Seynthetic
  source 1..20: mol_type = other RNA
tcccagctgcgggaatggga

SEQ ID NO: 1011 - RNA, 20 nt, synthetic construct
  misc_feature 1..20: note = Seynthetic
  source 1..20: mol_type = other RNA
tcccatctcccccaccatgt

SEQ ID NO: 1012 - RNA, 20 nt, synthetic construct
  misc_feature 1..20: note = Seynthetic
  source 1..20: mol_type = other RNA
tcccatctcccccacgatgt

SEQ ID NO: 1013 - RNA, 20 nt, synthetic construct
  misc_feature 1..20: note = Seynthetic
  source 1..20: mol_type = other RNA
tcccattcccgcagctggga

SEQ ID NO: 1014 - RNA, 20 nt, synthetic construct
  misc_feature 1..20: note = Seynthetic
  source 1..20: mol_type = other RNA
tccccatccttctggtcccc

SEQ ID NO: 1015 - RNA, 20 nt, synthetic construct
  misc_feature 1..20: note = Seynthetic
  source 1..20: mol_type = other RNA
tccccatccttctggtccct

SEQ ID NO: 1016 - RNA, 20 nt, synthetic construct
  misc_feature 1..20: note = Seynthetic
  source 1..20: mol_type = other RNA
tcccccagggtgtttcacaa

SEQ ID NO: 1017 - RNA, 20 nt, synthetic construct
  misc_feature 1..20: note = Seynthetic
  source 1..20: mol_type = other RNA
tcccccggcttttctcttca

SEQ ID NO: 1018 - RNA, 20 nt, synthetic construct
  misc_feature 1..20: note = Seynthetic
  source 1..20: mol_type = other RNA
tccccctcggagtctacacc

SEQ ID NO: 1019 - RNA, 20 nt, synthetic construct
  misc_feature 1..20: note = Seynthetic
  source 1..20: mol_type = other RNA
tccccgcaccatggattaat

SEQ ID NO: 1020 - RNA, 20 nt, synthetic construct
  misc_feature 1..20: note = Seynthetic
  source 1..20: mol_type = other RNA
tccctgcaccatggattaat

SEQ ID NO: 1021 - RNA, 20 nt, synthetic construct
  misc_feature 1..20: note = Seynthetic
  source 1..20: mol_type = other RNA
tccctgtggcctctggggct

SEQ ID NO: 1022 - RNA, 20 nt, synthetic construct
  misc_feature 1..20: note = Seynthetic
  source 1..20: mol_type = other RNA
tccctgtgtcctctggggct

SEQ ID NO: 1023 - RNA, 20 nt, synthetic construct
  misc_feature 1..20: note = Seynthetic
  source 1..20: mol_type = other RNA
tccctttcccgcagctggga

SEQ ID NO: 1024 - RNA, 20 nt, synthetic construct
  misc_feature 1..20: note = Seynthetic
  source 1..20: mol_type = other RNA
tccggcagccccttggtctg

SEQ ID NO: 1025 - RNA, 20 nt, synthetic construct
  misc_feature 1..20: note = Seynthetic
  source 1..20: mol_type = other RNA
tcctccagccccgccaggtg

SEQ ID NO: 1026 - RNA, 20 nt, synthetic construct
  misc_feature 1..20: note = Seynthetic
  source 1..20: mol_type = other RNA
tcctctggggcttgacaccc

SEQ ID NO: 1027 - RNA, 20 nt, synthetic construct
  misc_feature 1..20: note = Seynthetic
  source 1..20: mol_type = other RNA
tcctcttcccatctccccca

SEQ ID NO: 1028 - RNA, 20 nt, synthetic construct
  misc_feature 1..20: note = Seynthetic
  source 1..20: mol_type = other RNA
tcctgacgtgaccacaggca

SEQ ID NO: 1029 - RNA, 20 nt, synthetic construct
  misc_feature 1..20: note = Seynthetic
  source 1..20: mol_type = other RNA
tcctgggaatcccattcccg

SEQ ID NO: 1030 - RNA, 20 nt, synthetic construct
  misc_feature 1..20: note = Seynthetic
  source 1..20: mol_type = other RNA
tcctgggaatccctttcccg

SEQ ID NO: 1031 - RNA, 20 nt, synthetic construct
  misc_feature 1..20: note = Seynthetic
  source 1..20: mol_type = other RNA
tccttctggtccccgcacca

SEQ ID NO: 1032 - RNA, 20 nt, synthetic construct
  misc_feature 1..20: note = Seynthetic
  source 1..20: mol_type = other RNA
tccttctggtccctgcacca

SEQ ID NO: 1033 - RNA, 20 nt, synthetic construct
  misc_feature 1..20: note = Seynthetic
  source 1..20: mol_type = other RNA
tcgcattcaaatgtcagata

SEQ ID NO: 1034 - RNA, 20 nt, synthetic construct
  misc_feature 1..20: note = Seynthetic
  source 1..20: mol_type = other RNA
tcggagtctacacccgctgt

SEQ ID NO: 1035 - RNA, 20 nt, synthetic construct
  misc_feature 1..20: note = Seynthetic
  source 1..20: mol_type = other RNA
tcggagtctacacctgctgt

SEQ ID NO: 1036 - RNA, 20 nt, synthetic construct
  misc_feature 1..20: note = Seynthetic
  source 1..20: mol_type = other RNA
tcggcacacttaataaagac

SEQ ID NO: 1037 - RNA, 20 nt, synthetic construct
  misc_feature 1..20: note = Seynthetic
  source 1..20: mol_type = other RNA
tcggcacacttaataatgac

SEQ ID NO: 1038 - RNA, 20 nt, synthetic construct
  misc_feature 1..20: note = Seynthetic
  source 1..20: mol_type = other RNA
tcgtaaagttcttagctaag

SEQ ID NO: 1039 - RNA, 20 nt, synthetic construct
  misc_feature 1..20: note = Seynthetic
  source 1..20: mol_type = other RNA
tcgtgggggagatgggaaga

SEQ ID NO: 1040 - RNA, 20 nt, synthetic construct
  misc_feature 1..20: note = Seynthetic
  source 1..20: mol_type = other RNA
tctacacccgctgtgaccat

SEQ ID NO: 1041 - RNA, 20 nt, synthetic construct
  misc_feature 1..20: note = Seynthetic
  source 1..20: mol_type = other RNA
tctcaaagcaacactttctt

SEQ ID NO: 1042 - RNA, 20 nt, synthetic construct
  misc_feature 1..20: note = Seynthetic
  source 1..20: mol_type = other RNA
tctcacgtgcagtcattatt

SEQ ID NO: 1043 - RNA, 20 nt, synthetic construct
  misc_feature 1..20: note = Seynthetic
  source 1..20: mol_type = other RNA
tctcacgtgcagtctttatt

SEQ ID NO: 1044 - RNA, 20 nt, synthetic construct
  misc_feature 1..20: note = Seynthetic
  source 1..20: mol_type = other RNA
tctcccccaccatgttccag

SEQ ID NO: 1045 - RNA, 20 nt, synthetic construct
  misc_feature 1..20: note = Seynthetic
  source 1..20: mol_type = other RNA
tctcccccacgatgttccag

SEQ ID NO: 1046 - RNA, 20 nt, synthetic construct
  misc_feature 1..20: note = Seynthetic
  source 1..20: mol_type = other RNA
tctctgtccctgtggcctct

SEQ ID NO: 1047 - RNA, 20 nt, synthetic construct
  misc_feature 1..20: note = Seynthetic
  source 1..20: mol_type = other RNA
tctctgtccctgtgtcctct

SEQ ID NO: 1048 - RNA, 20 nt, synthetic construct
  misc_feature 1..20: note = Seynthetic
  source 1..20: mol_type = other RNA
tctgaaatttgaatgcgatt

SEQ ID NO: 1049 - RNA, 20 nt, synthetic construct
  misc_feature 1..20: note = Seynthetic
  source 1..20: mol_type = other RNA
tctgacatttgaatgcgatt

SEQ ID NO: 1050 - RNA, 20 nt, synthetic construct
  misc_feature 1..20: note = Seynthetic
  source 1..20: mol_type = other RNA
tctgcctccacagggggact

SEQ ID NO: 1051 - RNA, 20 nt, synthetic construct
  misc_feature 1..20: note = Seynthetic
  source 1..20: mol_type = other RNA
tctggtccccgcaccatgga

SEQ ID NO: 1052 - RNA, 20 nt, synthetic construct
  misc_feature 1..20: note = Seynthetic
  source 1..20: mol_type = other RNA
tctggtccctgcaccatgga

SEQ ID NO: 1053 - RNA, 20 nt, synthetic construct
  misc_feature 1..20: note = Seynthetic
  source 1..20: mol_type = other RNA
tctgtccctgtgtcctctgg

SEQ ID NO: 1054 - RNA, 20 nt, synthetic construct
  misc_feature 1..20: note = Seynthetic
  source 1..20: mol_type = other RNA
tcttagctaagaactttacg

SEQ ID NO: 1055 - RNA, 20 nt, synthetic construct
  misc_feature 1..20: note = Seynthetic
  source 1..20: mol_type = other RNA
tcttcccatctcccccacca

SEQ ID NO: 1056 - RNA, 20 nt, synthetic construct
  misc_feature 1..20: note = Seynthetic
  source 1..20: mol_type = other RNA
tcttcccatctcccccacga

SEQ ID NO: 1057 - RNA, 20 nt, synthetic construct
  misc_feature 1..20: note = Seynthetic
  source 1..20: mol_type = other RNA
tcttgacgtgaccacaggca

SEQ ID NO: 1058 - RNA, 20 nt, synthetic construct
  misc_feature 1..20: note = Seynthetic
  source 1..20: mol_type = other RNA
tctttattaagtgtgccgac

SEQ ID NO: 1059 - RNA, 20 nt, synthetic construct
  misc_feature 1..20: note = Seynthetic
  source 1..20: mol_type = other RNA
tgaaacaccctgggggactc

SEQ ID NO: 1060 - RNA, 20 nt, synthetic construct
  misc_feature 1..20: note = Seynthetic
  source 1..20: mol_type = other RNA
tgaaacaccctgggtgactc

SEQ ID NO: 1061 - RNA, 20 nt, synthetic construct
  misc_feature 1..20: note = Seynthetic
  source 1..20: mol_type = other RNA
tgaaataaattcacgttcgt

SEQ ID NO: 1062 - RNA, 20 nt, synthetic construct
  misc_feature 1..20: note = Seynthetic
  source 1..20: mol_type = other RNA
tgaaatttgaatgcgattgt

SEQ ID NO: 1063 - RNA, 20 nt, synthetic construct
  misc_feature 1..20: note = Seynthetic
  source 1..20: mol_type = other RNA
tgaagagaaaagcaggggga

SEQ ID NO: 1064 - RNA, 20 nt, synthetic construct
  misc_feature 1..20: note = Seynthetic
  source 1..20: mol_type = other RNA
tgaagagaaaagccggggga

SEQ ID NO: 1065 - RNA, 20 nt, synthetic construct
  misc_feature 1..20: note = Seynthetic
  source 1..20: mol_type = other RNA
tgaaggcggacatccctgaa

SEQ ID NO: 1066 - RNA, 20 nt, synthetic construct
  misc_feature 1..20: note = Seynthetic
  source 1..20: mol_type = other RNA
tgactctgggtgtggtgaag

SEQ ID NO: 1067 - RNA, 20 nt, synthetic construct
  misc_feature 1..20: note = Seynthetic
  source 1..20: mol_type = other RNA
tgacttccccacattgtatt

SEQ ID NO: 1068 - RNA, 20 nt, synthetic construct
  misc_feature 1..20: note = Seynthetic
  source 1..20: mol_type = other RNA
tgacttccccacattgtgtt

SEQ ID NO: 1069 - RNA, 20 nt, synthetic construct
  misc_feature 1..20: note = Seynthetic
  source 1..20: mol_type = other RNA
tgagtccccctgtggaggca

SEQ ID NO: 1070 - RNA, 20 nt, synthetic construct
  misc_feature 1..20: note = Seynthetic
  source 1..20: mol_type = other RNA
tgcacaatcgcattcaaatg

SEQ ID NO: 1071 - RNA, 20 nt, synthetic construct
  misc_feature 1..20: note = Seynthetic
  source 1..20: mol_type = other RNA
tgcacaatcgcattcaaatt

SEQ ID NO: 1072 - RNA, 20 nt, synthetic construct
  misc_feature 1..20: note = Seynthetic
  source 1..20: mol_type = other RNA
tgcaccatggattaatgcaa

SEQ ID NO: 1073 - RNA, 20 nt, synthetic construct
  misc_feature 1..20: note = Seynthetic
  source 1..20: mol_type = other RNA
tgcagaccaaggggctgccg

SEQ ID NO: 1074 - RNA, 20 nt, synthetic construct
  misc_feature 1..20: note = Seynthetic
  source 1..20: mol_type = other RNA
tgcagctggaacatcgtggg

SEQ ID NO: 1075 - RNA, 20 nt, synthetic construct
  misc_feature 1..20: note = Seynthetic
  source 1..20: mol_type = other RNA
tgcagctggaacatggtggg

SEQ ID NO: 1076 - RNA, 20 nt, synthetic construct
  misc_feature 1..20: note = Seynthetic
  source 1..20: mol_type = other RNA
tgcagggaccagaaggatgg

SEQ ID NO: 1077 - RNA, 20 nt, synthetic construct
  misc_feature 1..20: note = Seynthetic
  source 1..20: mol_type = other RNA
tgcagtcattattaagtgtg

SEQ ID NO: 1078 - RNA, 20 nt, synthetic construct
  misc_feature 1..20: note = Seynthetic
  source 1..20: mol_type = other RNA
tgcagtctttattaagtgtg

SEQ ID NO: 1079 - RNA, 20 nt, synthetic construct
  misc_feature 1..20: note = Seynthetic
  source 1..20: mol_type = other RNA
tgcattaatccatggtgcag

SEQ ID NO: 1080 - RNA, 20 nt, synthetic construct
  misc_feature 1..20: note = Seynthetic
  source 1..20: mol_type = other RNA
tgcctgtggtcacgtcaaga

SEQ ID NO: 1081 - RNA, 20 nt, synthetic construct
  misc_feature 1..20: note = Seynthetic
  source 1..20: mol_type = other RNA
tgcctgtggtcacgtcagga

SEQ ID NO: 1082 - RNA, 20 nt, synthetic construct
  misc_feature 1..20: note = Seynthetic
  source 1..20: mol_type = other RNA
tgcgggaaagggattcccag

SEQ ID NO: 1083 - RNA, 20 nt, synthetic construct
  misc_feature 1..20: note = Seynthetic
  source 1..20: mol_type = other RNA
tgcgggaatgggattcccag

SEQ ID NO: 1084 - RNA, 20 nt, synthetic construct
  misc_feature 1..20: note = Seynthetic
  source 1..20: mol_type = other RNA
tgcggggaccagaaggatgg

SEQ ID NO: 1085 - RNA, 20 nt, synthetic construct
  misc_feature 1..20: note = Seynthetic
  source 1..20: mol_type = other RNA
tgctcatgcttctaccaggg

SEQ ID NO: 1086 - RNA, 20 nt, synthetic construct
  misc_feature 1..20: note = Seynthetic
  source 1..20: mol_type = other RNA
tgctgtgaccataacacccc

SEQ ID NO: 1087 - RNA, 20 nt, synthetic construct
  misc_feature 1..20: note = Seynthetic
  source 1..20: mol_type = other RNA
tgcttctaccagggaactcc

SEQ ID NO: 1088 - RNA, 20 nt, synthetic construct
  misc_feature 1..20: note = Seynthetic
  source 1..20: mol_type = other RNA
tgcttctaccagggagctcc

SEQ ID NO: 1089 - RNA, 20 nt, synthetic construct
  misc_feature 1..20: note = Seynthetic
  source 1..20: mol_type = other RNA
tggaaagttcagggatgtcc

SEQ ID NO: 1090 - RNA, 20 nt, synthetic construct
  misc_feature 1..20: note = Seynthetic
  source 1..20: mol_type = other RNA
tggaacatcgtgggggagat

SEQ ID NO: 1091 - RNA, 20 nt, synthetic construct
  misc_feature 1..20: note = Seynthetic
  source 1..20: mol_type = other RNA
tggaacatggtgggggagat

SEQ ID NO: 1092 - RNA, 20 nt, synthetic construct
  misc_feature 1..20: note = Seynthetic
  source 1..20: mol_type = other RNA
tggaggggctggaggaccgt

SEQ ID NO: 1093 - RNA, 20 nt, synthetic construct
  misc_feature 1..20: note = Seynthetic
  source 1..20: mol_type = other RNA
tggagtcccagctgcgggaa

SEQ ID NO: 1094 - RNA, 20 nt, synthetic construct
  misc_feature 1..20: note = Seynthetic
  source 1..20: mol_type = other RNA
tggagtcctgacgtgaccac

SEQ ID NO: 1095 - RNA, 20 nt, synthetic construct
  misc_feature 1..20: note = Seynthetic
  source 1..20: mol_type = other RNA
tggagtcttgacgtgaccac

SEQ ID NO: 1096 - RNA, 20 nt, synthetic construct
  misc_feature 1..20: note = Seynthetic
  source 1..20: mol_type = other RNA
tggattctagtttgttgttt

SEQ ID NO: 1097 - RNA, 20 nt, synthetic construct
  misc_feature 1..20: note = Seynthetic
  source 1..20: mol_type = other RNA
tggcctctggggcttgacac

SEQ ID NO: 1098 - RNA, 20 nt, synthetic construct
  misc_feature 1..20: note = Seynthetic
  source 1..20: mol_type = other RNA
tggcggggctggaggaccgt

SEQ ID NO: 1099 - RNA, 20 nt, synthetic construct
  misc_feature 1..20: note = Seynthetic
  source 1..20: mol_type = other RNA
tgggaatcccattcccgcag

SEQ ID NO: 1100 - RNA, 20 nt, synthetic construct
  misc_feature 1..20: note = Seynthetic
  source 1..20: mol_type = other RNA
tgggaatccctttcccgcag

SEQ ID NO: 1101 - RNA, 20 nt, synthetic construct
  misc_feature 1..20: note = Seynthetic
  source 1..20: mol_type = other RNA
tgggattcccaggacccagc

SEQ ID NO: 1102 - RNA, 20 nt, synthetic construct
  misc_feature 1..20: note = Seynthetic
  source 1..20: mol_type = other RNA
tgggccaggctggagtcctg

SEQ ID NO: 1103 - RNA, 20 nt, synthetic construct
  misc_feature 1..20: note = Seynthetic
  source 1..20: mol_type = other RNA
tgggccaggctggagtcttg

SEQ ID NO: 1104 - RNA, 20 nt, synthetic construct
  misc_feature 1..20: note = Seynthetic
  source 1..20: mol_type = other RNA
tgggggacgccgtatctttg

SEQ ID NO: 1105 - RNA, 20 nt, synthetic construct
  misc_feature 1..20: note = Seynthetic
  source 1..20: mol_type = other RNA
tgggggacgctgtatctttg

SEQ ID NO: 1106 - RNA, 20 nt, synthetic construct
  misc_feature 1..20: note = Seynthetic
  source 1..20: mol_type = other RNA
tgggggactctgggtgtggt

SEQ ID NO: 1107 - RNA, 20 nt, synthetic construct
  misc_feature 1..20: note = Seynthetic
  source 1..20: mol_type = other RNA
tgggtcctgggaatcccatt

SEQ ID NO: 1108 - RNA, 20 nt, synthetic construct
  misc_feature 1..20: note = Seynthetic
  source 1..20: mol_type = other RNA
tgggtcctgggaatcccttt

SEQ ID NO: 1109 - RNA, 20 nt, synthetic construct
  misc_feature 1..20: note = Seynthetic
  source 1..20: mol_type = other RNA
tgggtgactctgggtgtggt

SEQ ID NO: 1110 - RNA, 20 nt, synthetic construct
  misc_feature 1..20: note = Seynthetic
  source 1..20: mol_type = other RNA
tggtcacagcaggtgtagac

SEQ ID NO: 1111 - RNA, 20 nt, synthetic construct
  misc_feature 1..20: note = Seynthetic
  source 1..20: mol_type = other RNA
tggtcacagcgggtgtagac

SEQ ID NO: 1112 - RNA, 20 nt, synthetic construct
  misc_feature 1..20: note = Seynthetic
  source 1..20: mol_type = other RNA
tggtcacgtcaagactccag

SEQ ID NO: 1113 - RNA, 20 nt, synthetic construct
  misc_feature 1..20: note = Seynthetic
  source 1..20: mol_type = other RNA
tggtcacgtcaggactccag

SEQ ID NO: 1114 - RNA, 20 nt, synthetic construct
  misc_feature 1..20: note = Seynthetic
  source 1..20: mol_type = other RNA
tggtccccgcaccatggatt

SEQ ID NO: 1115 - RNA, 20 nt, synthetic construct
  misc_feature 1..20: note = Seynthetic
  source 1..20: mol_type = other RNA
tggtccctgcaccatggatt

SEQ ID NO: 1116 - RNA, 20 nt, synthetic construct
  misc_feature 1..20: note = Seynthetic
  source 1..20: mol_type = other RNA
tggtgaagagaaaagcaggg

SEQ ID NO: 1117 - RNA, 20 nt, synthetic construct
  misc_feature 1..20: note = Seynthetic
  source 1..20: mol_type = other RNA
tggtgaagagaaaagccggg

SEQ ID NO: 1118 - RNA, 20 nt, synthetic construct
  misc_feature 1..20: note = Seynthetic
  source 1..20: mol_type = other RNA
tggtgcagggaccagaagga

SEQ ID NO: 1119 - RNA, 20 nt, synthetic construct
  misc_feature 1..20: note = Seynthetic
  source 1..20: mol_type = other RNA
tggtgcggggaccagaagga

SEQ ID NO: 1120 - RNA, 20 nt, synthetic construct
  misc_feature 1..20: note = Seynthetic
  source 1..20: mol_type = other RNA
tgtatctttggaaggggctt

SEQ ID NO: 1121 - RNA, 20 nt, synthetic construct
  misc_feature 1..20: note = Seynthetic
  source 1..20: mol_type = other RNA
tgtcaagccccagaggacac

SEQ ID NO: 1122 - RNA, 20 nt, synthetic construct
  misc_feature 1..20: note = Seynthetic
  source 1..20: mol_type = other RNA
tgtcaagccccagaggccac

SEQ ID NO: 1123 - RNA, 20 nt, synthetic construct
  misc_feature 1..20: note = Seynthetic
  source 1..20: mol_type = other RNA
tgtcagataatcaatgtggg

SEQ ID NO: 1124 - RNA, 20 nt, synthetic construct
  misc_feature 1..20: note = Seynthetic
  source 1..20: mol_type = other RNA
tgtccctgtggcctctgggg

SEQ ID NO: 1125 - RNA, 20 nt, synthetic construct
  misc_feature 1..20: note = Seynthetic
  source 1..20: mol_type = other RNA
tgtccgccttcaggtgtggc

SEQ ID NO: 1126 - RNA, 20 nt, synthetic construct
  misc_feature 1..20: note = Seynthetic
  source 1..20: mol_type = other RNA
tgtcctctggggcttgacac

SEQ ID NO: 1127 - RNA, 20 nt, synthetic construct
  misc_feature 1..20: note = Seynthetic
  source 1..20: mol_type = other RNA
tgtctctgtccctgtgtcct

SEQ ID NO: 1128 - RNA, 20 nt, synthetic construct
  misc_feature 1..20: note = Seynthetic
  source 1..20: mol_type = other RNA
tgtctgcctccacaggggga

SEQ ID NO: 1129 - RNA, 20 nt, synthetic construct
  misc_feature 1..20: note = Seynthetic
  source 1..20: mol_type = other RNA
tgtgaaacaccctgggggac

SEQ ID NO: 1130 - RNA, 20 nt, synthetic construct
  misc_feature 1..20: note = Seynthetic
  source 1..20: mol_type = other RNA
tgtgaaacaccctgggtgac

SEQ ID NO: 1131 - RNA, 20 nt, synthetic construct
  misc_feature 1..20: note = Seynthetic
  source 1..20: mol_type = other RNA
tgtggcctctggggcttgac

SEQ ID NO: 1132 - RNA, 20 nt, synthetic construct
  misc_feature 1..20: note = Seynthetic
  source 1..20: mol_type = other RNA
tgtggtcacgtcaagactcc

SEQ ID NO: 1133 - RNA, 20 nt, synthetic construct
  misc_feature 1..20: note = Seynthetic
  source 1..20: mol_type = other RNA
tgtggtcacgtcaggactcc

SEQ ID NO: 1134 - RNA, 20 nt, synthetic construct
  misc_feature 1..20: note = Seynthetic
  source 1..20: mol_type = other RNA
tgtgtcctctggggcttgac

SEQ ID NO: 1135 - RNA, 20 nt, synthetic construct
  misc_feature 1..20: note = Seynthetic
  source 1..20: mol_type = other RNA
tgttatggtcacagcgggtg

SEQ ID NO: 1136 - RNA, 20 nt, synthetic construct
  misc_feature 1..20: note = Seynthetic
  source 1..20: mol_type = other RNA
ttaataaagactgcacgtga

SEQ ID NO: 1137 - RNA, 20 nt, synthetic construct
  misc_feature 1..20: note = Seynthetic
  source 1..20: mol_type = other RNA
ttaataatgactgcacgtga

SEQ ID NO: 1138 - RNA, 20 nt, synthetic construct
  misc_feature 1..20: note = Seynthetic
  source 1..20: mol_type = other RNA
ttaatccatggtgcagggac

SEQ ID NO: 1139 - RNA, 20 nt, synthetic construct
  misc_feature 1..20: note = Seynthetic
  source 1..20: mol_type = other RNA
ttaatccatggtgcggggac

SEQ ID NO: 1140 - RNA, 20 nt, synthetic construct
  misc_feature 1..20: note = Seynthetic
  source 1..20: mol_type = other RNA
ttagctaagaactttacgaa

SEQ ID NO: 1141 - RNA, 20 nt, synthetic construct
  misc_feature 1..20: note = Seynthetic
  source 1..20: mol_type = other RNA
ttatatggaaagttcaggga

SEQ ID NO: 1142 - RNA, 20 nt, synthetic construct
  misc_feature 1..20: note = Seynthetic
  source 1..20: mol_type = other RNA
ttatctgaaatttgaatgcg

SEQ ID NO: 1143 - RNA, 20 nt, synthetic construct
  misc_feature 1..20: note = Seynthetic
  source 1..20: mol_type = other RNA
ttatctgacatttgaatgcg

SEQ ID NO: 1144 - RNA, 20 nt, synthetic construct
  misc_feature 1..20: note = Seynthetic
  source 1..20: mol_type = other RNA
ttatggtcacagcaggtgta

SEQ ID NO: 1145 - RNA, 20 nt, synthetic construct
  misc_feature 1..20: note = Seynthetic
  source 1..20: mol_type = other RNA
ttatggtcacagcgggtgta

SEQ ID NO: 1146 - RNA, 20 nt, synthetic construct
  misc_feature 1..20: note = Seynthetic
  source 1..20: mol_type = other RNA
ttcaccacacccagagtccc

SEQ ID NO: 1147 - RNA, 20 nt, synthetic construct
  misc_feature 1..20: note = Seynthetic
  source 1..20: mol_type = other RNA
ttcacgttcataaagttctt

SEQ ID NO: 1148 - RNA, 20 nt, synthetic construct
  misc_feature 1..20: note = Seynthetic
  source 1..20: mol_type = other RNA
ttcacgttcgtaaagttctt

SEQ ID NO: 1149 - RNA, 20 nt, synthetic construct
  misc_feature 1..20: note = Seynthetic
  source 1..20: mol_type = other RNA
ttcagataatcaatgtgggt

SEQ ID NO: 1150 - RNA, 20 nt, synthetic construct
  misc_feature 1..20: note = Seynthetic
  source 1..20: mol_type = other RNA
ttcagggatgtccgccttca

SEQ ID NO: 1151 - RNA, 20 nt, synthetic construct
  misc_feature 1..20: note = Seynthetic
  source 1..20: mol_type = other RNA
ttcaggggtgggggacgccg

SEQ ID NO: 1152 - RNA, 20 nt, synthetic construct
  misc_feature 1..20: note = Seynthetic
  source 1..20: mol_type = other RNA
ttcaggggtgggggacgctg

SEQ ID NO: 1153 - RNA, 20 nt, synthetic construct
  misc_feature 1..20: note = Seynthetic
  source 1..20: mol_type = other RNA
ttccaaagatacagcgtccc

SEQ ID NO: 1154 - RNA, 20 nt, synthetic construct
  misc_feature 1..20: note = Seynthetic
  source 1..20: mol_type = other RNA
ttccaaagatacggcgtccc

SEQ ID NO: 1155 - RNA, 20 nt, synthetic construct
  misc_feature 1..20: note = Seynthetic
  source 1..20: mol_type = other RNA
ttcccatctcccccaccatg

SEQ ID NO: 1156 - RNA, 20 nt, synthetic construct
  misc_feature 1..20: note = Seynthetic
  source 1..20: mol_type = other RNA
ttcccatctcccccacgatg

SEQ ID NO: 1157 - RNA, 20 nt, synthetic construct
  misc_feature 1..20: note = Seynthetic
  source 1..20: mol_type = other RNA
ttccccacattgtgtttttt

SEQ ID NO: 1158 - RNA, 20 nt, synthetic construct
  misc_feature 1..20: note = Seynthetic
  source 1..20: mol_type = other RNA
ttcccccggcttttctcttc

SEQ ID NO: 1159 - RNA, 20 nt, synthetic construct
  misc_feature 1..20: note = Seynthetic
  source 1..20: mol_type = other RNA
ttccccctgcttttctcttc

SEQ ID NO: 1160 - RNA, 20 nt, synthetic construct
  misc_feature 1..20: note = Seynthetic
  source 1..20: mol_type = other RNA
ttcctcttcccatctccccc

SEQ ID NO: 1161 - RNA, 20 nt, synthetic construct
  misc_feature 1..20: note = Seynthetic
  source 1..20: mol_type = other RNA
ttcctgcccacctggcgggg

SEQ ID NO: 1162 - RNA, 20 nt, synthetic construct
  misc_feature 1..20: note = Seynthetic
  source 1..20: mol_type = other RNA
ttcgtaaagttcttagctaa

SEQ ID NO: 1163 - RNA, 20 nt, synthetic construct
  misc_feature 1..20: note = Seynthetic
  source 1..20: mol_type = other RNA
ttctaccagggaactcctgc

SEQ ID NO: 1164 - RNA, 20 nt, synthetic construct
  misc_feature 1..20: note = Seynthetic
  source 1..20: mol_type = other RNA
ttctcacgtgcagtcattat

SEQ ID NO: 1165 - RNA, 20 nt, synthetic construct
  misc_feature 1..20: note = Seynthetic
  source 1..20: mol_type = other RNA
ttctcacgtgcagtctttat

SEQ ID NO: 1166 - RNA, 20 nt, synthetic construct
  misc_feature 1..20: note = Seynthetic
  source 1..20: mol_type = other RNA
ttctggtccccgcaccatgg

SEQ ID NO: 1167 - RNA, 20 nt, synthetic construct
  misc_feature 1..20: note = Seynthetic
  source 1..20: mol_type = other RNA
ttcttagctaagaactttac

SEQ ID NO: 1168 - RNA, 20 nt, synthetic construct
  misc_feature 1..20: note = Seynthetic
  source 1..20: mol_type = other RNA
ttgacgtgaccacaggcaag

SEQ ID NO: 1169 - RNA, 20 nt, synthetic construct
  misc_feature 1..20: note = Seynthetic
  source 1..20: mol_type = other RNA
ttgcagaccaaggggctgcc

SEQ ID NO: 1170 - RNA, 20 nt, synthetic construct
  misc_feature 1..20: note = Seynthetic
  source 1..20: mol_type = other RNA
ttgcattaatccatggtgca

SEQ ID NO: 1171 - RNA, 20 nt, synthetic construct
  misc_feature 1..20: note = Seynthetic
  source 1..20: mol_type = other RNA
ttgcattaatccatggtgcg

SEQ ID NO: 1172 - RNA, 20 nt, synthetic construct
  misc_feature 1..20: note = Seynthetic
  source 1..20: mol_type = other RNA
ttgcctgtggtcacgtcaag

SEQ ID NO: 1173 - RNA, 20 nt, synthetic construct
  misc_feature 1..20: note = Seynthetic
  source 1..20: mol_type = other RNA
ttgcctgtggtcacgtcagg

SEQ ID NO: 1174 - RNA, 20 nt, synthetic construct
  misc_feature 1..20: note = Seynthetic
  source 1..20: mol_type = other RNA
ttgctcatgcttctaccagg

SEQ ID NO: 1175 - RNA, 20 nt, synthetic construct
  misc_feature 1..20: note = Seynthetic
  source 1..20: mol_type = other RNA
ttgtctctgtccctgtggcc

SEQ ID NO: 1176 - RNA, 20 nt, synthetic construct
  misc_feature 1..20: note = Seynthetic
  source 1..20: mol_type = other RNA
ttgtctctgtccctgtgtcc

SEQ ID NO: 1177 - RNA, 20 nt, synthetic construct
  misc_feature 1..20: note = Seynthetic
  source 1..20: mol_type = other RNA
ttgtgaaacaccctggggga

SEQ ID NO: 1178 - RNA, 20 nt, synthetic construct
  misc_feature 1..20: note = Seynthetic
  source 1..20: mol_type = other RNA
ttgtgaaacaccctgggtga

SEQ ID NO: 1179 - RNA, 20 nt, synthetic construct
  misc_feature 1..20: note = Seynthetic
  source 1..20: mol_type = other RNA
tttattaagtgtgccgacgc

SEQ ID NO: 1180 - RNA, 20 nt, synthetic construct
  misc_feature 1..20: note = Seynthetic
  source 1..20: mol_type = other RNA
tttcaggggtgggggacgcc

SEQ ID NO: 1181 - RNA, 20 nt, synthetic construct
  misc_feature 1..20: note = Seynthetic
  source 1..20: mol_type = other RNA
tttcaggggtgggggacgct

SEQ ID NO: 1182 - RNA, 20 nt, synthetic construct
  misc_feature 1..20: note = Seynthetic
  source 1..20: mol_type = other RNA
tttcccgcagctgggactcc

SEQ ID NO: 1183 - RNA, 20 nt, synthetic construct
  misc_feature 1..20: note = Seynthetic
  source 1..20: mol_type = other RNA
tttgtctctgtccctgtggc

SEQ ID NO: 1184 - RNA, 20 nt, synthetic construct
  misc_feature 1..20: note = Seynthetic
  source 1..20: mol_type = other RNA
ttttaatggagcgggggttt

SEQ ID NO: 1185 - RNA, 20 nt, synthetic construct
  misc_feature 1..20: note = Seynthetic
  source 1..20: mol_type = other RNA
tttttaatggagcgggggtt

SEQ ID NO: 1186 - RNA, 20 nt, synthetic construct
  misc_feature 1..20: note = Seynthetic
  source 1..20: mol_type = other RNA
ttttttaatggagcgggggt

SEQ ID NO: 1187 - RNA, 20 nt, synthetic construct
  misc_feature 1..20: note = Seynthetic
  source 1..20: mol_type = other RNA
tttttttaatggagcggggg

SEQ ID NO: 1188 - RNA, 20 nt, synthetic construct
  misc_feature 1..20: note = Seynthetic
  source 1..20: mol_type = other RNA
tttttttttaatggagcggg

SEQ ID NO: 1189 - RNA, 20 nt, synthetic construct
  misc_feature 1..20: note = Seynthetic
  source 1..20: mol_type = other RNA
ttttttttttaatggagcgg

SEQ ID NO: 1190 - RNA, 20 nt, synthetic construct
  misc_feature 1..20: note = Seynthetic
  source 1..20: mol_type = other RNA
ctctgtccctgtggcctctg

SEQ ID NO: 1191 - RNA, 20 nt, synthetic construct
  misc_feature 1..20: note = Seynthetic
  source 1..20: mol_type = other RNA
aaatgtcagataatcaatgt

SEQ ID NO: 1192 - RNA, 20 nt, synthetic construct
  misc_feature 1..20: note = Seynthetic
  source 1..20: mol_type = other RNA
ttgcagctggaacatcgtgg

SEQ ID NO: 1193 - RNA, 20 nt, synthetic construct
  misc_feature 1..20: note = Seynthetic
  source 1..20: mol_type = other RNA
accaaggctcagggcgttgg

SEQ ID NO: 1194 - RNA, 20 nt, synthetic construct
  misc_feature 1..20: note = Seynthetic
  source 1..20: mol_type = other RNA
cctgttgctgcagtccgggc

SEQ ID NO: 1195 - RNA, 20 nt, synthetic construct
  misc_feature 1..20: note = Seynthetic
  source 1..20: mol_type = other RNA
ccagcccggactgcagcaac

SEQ ID NO: 1196 - RNA, 20 nt, synthetic construct
  misc_feature 1..20: note = Seynthetic
  source 1..20: mol_type = other RNA
tccctcctagggtctagcca

SEQ ID NO: 1197 - RNA, 20 nt, synthetic construct
  misc_feature 1..20: note = Seynthetic
  source 1..20: mol_type = other RNA
agtccgggctgggagcgggt

SEQ ID NO: 1198 - RNA, 20 nt, synthetic construct
  misc_feature 1..20: note = Seynthetic
  source 1..20: mol_type = other RNA
atgtttattgtgccagatgc

SEQ ID NO: 1199 - RNA, 20 nt, synthetic construct
  misc_feature 1..20: note = Seynthetic
  source 1..20: mol_type = other RNA
gtgggcagctgaggtgaccc

SEQ ID NO: 1200 - RNA, 20 nt, synthetic construct
  misc_feature 1..20: note = Seynthetic
  source 1..20: mol_type = other RNA
cacccacactctccagcatc

SEQ ID NO: 1201 - RNA, 17 nt, synthetic construct
  misc_feature 1..17: note = Seynthetic
  source 1..17: mol_type = other RNA
aaaacacaatgtgggga

SEQ ID NO: 1202 - RNA, 17 nt, synthetic construct
  misc_feature 1..17: note = Seynthetic
  source 1..17: mol_type = other RNA
aaaaacacaatgtgggg

SEQ ID NO: 1203 - RNA, 17 nt, synthetic construct
  misc_feature 1..17: note = Seynthetic
  source 1..17: mol_type = other RNA
aaaaaacacaatgtggg

SEQ ID NO: 1204 - RNA, 17 nt, synthetic construct
  misc_feature 1..17: note = Seynthetic
  source 1..17: mol_type = other RNA
aaaaaaacacaatgtgg

(19) United States
(12) Patent Application Publication
(10) Pub. No.: US 2026/0078415 A1
(43) Pub. Date: Mar. 19, 2026

(54) Differential Knockout of an Allele of a Heterozygous Elane Gene

(71) Applicant: EmendoBio Inc., Wilmington, DE (US)

(72) Inventors: David BARAM, Tel Aviv (IL); Lior IZHAR, Tel Aviv (IL); Asael HERMAN, Ness-Ziona (IL); Rafi EMMANUEL, Ramla (IL); Michal GOLAN MASHIACH, Ness-Ziona (IL); Joseph GEORGESON, Rehovot (IL)

(21) Appl. No.: 19/398,868

(22) Filed: Nov. 24, 2025

Related U.S. Application Data
(63) Continuation of application No. 17/053,629, now Pat. No. 12,480,140.
(60) Provisional application No. 62/667,536, filed on May 6, 2018.
(60) Provisional application No. 62/723,941, filed on Aug. 28, 2018.
(60) Provisional application No. 62/743,309, filed on Oct. 9, 2018.

(51) Int. Cl. C12N 15/90; A61K 35/28; C12N 9/22; C12N 15/11; C12Q 1/6883

(52) U.S. Cl. CPC C12N 15/907; A61K 35/28; C12N 9/22; C12N 15/11; C12Q 1/6883; C12N 2310/20; C12N 2800/80; C12Q 2600/106

(57) ABSTRACT

Methods for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
introducing to the cell a composition comprising:
   a CRISPR nuclease or a sequence encoding the CRISPR nuclease; and
   a first RNA molecule comprising a guide sequence portion having 17-20 nucleotides,
   wherein a complex of the CRISPR nuclease and the first RNA molecule affects a double strand break in the mutant allele of the ELANE gene,
the method optionally further comprising introduction of a second RNA molecule comprising a guide sequence portion capable of complexing with a CRISPR nuclease, wherein the complex of the second RNA molecule and CRISPR nuclease affects a second double strand break in the ELANE gene.

This application is continuation of U.S. application Ser. No. 17/053,629, filed Nov. 6, 2020, which is a § 371 national stage of PCT App. PCT/US2019/03074, filed May 6, 2019, which claims the benefit of U.S. Prov. App. 62/743,309, filed Oct. 9, 2018, U.S. Prov. App. 62/723,941, filed Aug. 28, 2018, and U.S. Prov. App. No. 62/667,536, filed May 6, 2018, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide and/or amino acid sequences present in the file named “5798_0019US02-Seq_Listing.xml”, which is 1,515,887 bytes in size, and which was created on Nov. 24, 2025 in the IBM-PC machine format, having an operating system compatibility with MS-Windows, which is contained in the xml file filed Nov. 24, 2025 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders. SNPs are usually considered benign and not causing disease.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Gain-of-function mutations are generally dominant negative mutations. An example of a dominant negative mutation is haploinsufficiency where one allele is mutated and loses its function and the single wild type allele left does not generate enough protein to be sufficient for a specific cellular function. Other examples include dominant negative mutations which have a gene product that acts antagonistically to the wild-type allele.

### Neutropenia

Neutropenia is defined as a reduction in the absolute number of neutrophils in the blood circulation and commonly diagnosed by measuring the absolute neutrophil count (ANC) in peripheral blood. The severity of neutropenia is characterized as mild with an ANC of 1000-1500/μL, moderate with an ANC of 500-1000/μL, or severe with an ANC of less than 500/μL (Boxer 2012).

Neutropenia can be classified as congenital (hereditary) or acquired. The two main types of the congenital condition, commonly of autosomal dominant inheritance, are cyclic neutropenia (CyN) and severe congenital neutropenia (SCN). Cyclic neutropenia is characterized by fluctuating neutrophil counts from normal levels to zero while severe congenital neutropenia (SCN) is characterized by very low ANC (500/μL) observed at birth, maturation arrest of the myelopoiesis in bone marrow at the promyelocyte/myelocyte stage, and early onset of bacterial infections (Carlsson et al. 2012; Horwitz et al. 2013).

SCN may be diagnosed by measuring a very low ANC in the blood and examining bone marrow aspirate to identify myeloid maturation arrest (Dale 2017). SCN is usually diagnosed before age 6 months, while diagnosis for CyN is generally raised during the second year of life, or later, and the main clinical manifestation is recurrent acute stomatologic disorders. Bone marrow examination is often necessary to rule out malignant hemopathies, determine cellularity, assess myeloid maturation, and detect signs of a precise etiology, with cytogenetic bone marrow studies now crucial when SCN is suspected. Antineutrophil antibody assay, immunoglobulin assay (Ig GAM), lymphocyte immunophenotyping, pancreatic markers (serum trypsinogen and fecal elastase) and liposoluble vitamin levels (vitamins A, E and D) are also of interest in assessing SCN and CyN (See Donadieu 2011).

SCN can be autosomal-recessive (HAX1, G6PC3), autosomal-dominant (ELANE, GFI1), or X-linked (WAS) forms of inheritance or occur sporadically (Carlsson et al. 2012; Boxer 2012).

Cyclic and congenital neutropenia are most frequently caused by mutations in the “elastase, neutrophil expressed gene” (ELANE gene)—the gene for neutrophil elastase. “ELANE gene mutations are identified in 40-55% of SCN patients and males and females are equally affected (Donadieu et al. 2011; Dale 2017). Mutations in the ELANE gene are associated with autosomal-dominant and sporadic cases of SCN (Carlsson et al. 2012). To date, more than 200 different ELANE mutations have been identified, which are randomly distributed over all exons as well as in intron 3 and intron 4 (Skokowa et al. 2017). More than 120 distinct ELANE gene mutations related to CyN and SCN are now known, for example C151Y and 0214R particularly associated with a poor prognosis. (See Makaryan et al. 2012; see also Germeshausen et al. 2013 for a comprehensive list of ELANE mutations related to CyN and SCN).

ELANE encodes neutrophil elastase (NE) which is involved in the function of neutrophil extracellular traps (networks of fibers that bind pathogens). Some studies suggest that the product of mutant ELANE acts to disrupt neutrophil production in the bone marrow and cause neutropenia. These studies indicate that mutations in NE initiate the unfolded protein response (UPR) leading to cell loss in the process of neutrophil formation in the marrow (Makaryan et al. 2017).

### Current Treatments

Granulocyte colony-stimulating factor (G-CSF) is considered the first-line treatment for SCN (Connelly, Choi, and Levine 2012). G-CSF stimulates the production of more neutrophils and delays their apoptosis (Schäffer and Klein 2007). Overall survival is now estimated to exceed 80%, including patients developing malignancies, although 10% of SCN patients still die from severe bacterial infections or sepsis (Skokowa et al. 2017). Although G-CSF therapy is successful in preventing mortality from sepsis, long-term treatment was identified to be associated with an increased risk of developing myelodysplastic syndrome (MDS) or leukemia in SCN patients. The most common leukemia in SCN is AML, but acute lymphoid leukemia (ALL), juvenile myelomonocytic leukemia (JMML), chronic myelomonocytic leukemia (CMML), and bi-phenotypic leukemia are also reported in the literature (Connelly, Choi, and Levine 2012). It was previously demonstrated that patients who had a robust response to G-CSF (doses≤8 μg/kg/day) had a cumulative incidence of 15% for developing MDS/leukemia after 15 years on G-CSF, while an incidence of 34% was reported in patients with poor response to G-CSF despite high doses (Rosenberg et al. 2010).

Hematopoietic stem cell transplant (HSCT) is an alternative, curative therapy for patients who do not respond to G-CSF therapy or who develop AML/MDS. However, patients with chronic neutropenia who undergo HCT are at increased risk of developing infectious complications such as fungal and graft-versus-host disease (Skokowa et al. 2017). Moreover, HCT requires a matched related donor for successful survival but most patients will not have an available matched donor (Connelly, Choi, and Levine 2012).

### SUMMARY OF THE INVENTION

Disclosed is an approach for knocking out the expression of a dominant-mutant allele by disrupting the dominant-mutant allele or degrading the resulting mRNA.

The present disclosure provides a method for utilizing at least one naturally occurring heterozygous nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”).

Embodiments of the present invention provide methods for utilizing at least one heterozygous SNP in a gene expressing a dominant mutant allele in a given cell or subject. Tn embodiments of the present invention, the SNP utilized may or may not be associated with a disease phenotype. In embodiments of the present invention, an RNA molecule comprising a guide sequence targets the mutant allele of the gene by targeting the nucleotide base present at a heterozygous SNP in the mutant allele of the gene and therefore having a different nucleotide base from the functional allele of the gene.

In some embodiments, the method further comprises the step of knocking out expression of the mutated protein and allowing expression of the functional protein.

The present invention provides a method for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
introducing to the cell a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene.

The present invention provides for a modified cell obtained by the methods of the present invention.

The present invention provides for a modified cell lacking at least a portion of one allele of the ELANE gene.

The present invention provides for a composition comprising modified cells and a pharmaceutically acceptable carrier.

The present invention provides for an in vitro or ex vivo method of preparing a composition, comprising mixing the cells of the present invention with the pharmaceutically acceptable carrier.

The present invention provides for a method of preparing in vitro or ex vivo a composition comprising modified cells, the method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, and obtaining the cell from the subject;
b) introducing to the cells of step (a) a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally
c) culture expanding the modified cells of step (b),
wherein the modified cells are capable of engraftment and giving rise to progeny cells after engraftment.

The present invention provides for use of a composition prepared in vitro by a method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854;
b) introducing to the cells of step (a) a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c) for treating the SCN or CyN in the subject.

The present invention provides for a method of treating a subject afflicted with SCN or CyN, comprising administration of a therapeutically effective amount of the modified cells, compositions, or the compositions prepared by the methods of the instant invention

The present invention provides for a method for treating SCN or CyN in a subject with an ELANE gene mutation relating to SCN or CYN in need thereof and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising:
a) isolating HSPCs from cells obtained from the subject;
b) introducing to the cells of step (a) a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
thereby treating the SCN or CyN in the subject.

The present invention provides for a method for treating SCN or CyN in a subject with an ELANE gene mutation relating to SCN or CYN in need thereof and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
administering to the subject autologous modified cells or progeny of autologous modified cells, wherein the autologous modified cells are modified so as to have a double strand break in the mutant allele of the ELANE gene,
   wherein said double strand break results from introduction to the cells of a composition comprising a CRISPR nuclease or a sequence encoding the CRISPR nuclease and a first RNA molecule wherein a complex of the CRISPR nuclease and the first RNA molecule affects a double strand break in the mutant allele of the ELANE gene so as to inactive the mutant allele of the ELANE gene in the cell,
thereby treating the SCN or CyN in the subject.

The present invention provides for a method of selecting a subject for treatment from a pool of subjects diagnosed with SCN or CyN, comprising the steps of:
a) obtaining cells from each subject in the pool of subjects;
b) screening each subject's cells for an ELANE gene mutation related to SCN or CyN, and selecting only subjects with an ELANE gene mutation related to SCN or CyN;
c) screening by sequencing the cells of the subjects selected in step (b) for heterozygosity at one or more polymorphic sites selected from the group consisting of: rs10414837, rs3761005, rs1683564, and
d) selecting for treatment only subjects with cells heterozygous at the one of more polymorphic sites.

Embodiments of the present invention further comprise treating SCN or CyN in a selected subject, comprising:
e) obtaining hematopoetic stem and progenitor cells (HSPC) cells from the bone marrow of the subject either by aspiration or by mobilization and apheresis of peripheral blood;
f) introducing to the HSPC cells of step (e):
   one or more CRISPR nucleases or sequences encoding the one or more CRISPR nuclease;
   a first RNA molecule comprising a guide sequence portion having 17-20 nucleotides in a sequence of 17-20 contiguous nucleotides set forth in any one of SEQ ID NOs: 1-1192 targeting the nucleotide base of the heterozygous allele of the one or more polymorphic sites present on the mutant allele of the ELANE gene, and
   a second RNA molecule comprising a guide sequence portion targeting a sequence in intron 3, intron 4 or 3′ UTR of the ELANE gene,
wherein a complex of the first RNA molecule and a CRISPR nuclease affects a first double strand break in the mutant allele of the ELANE gene in one or more of the HSPC cells and a complex of the second RNA molecule and a CRISPR nuclease affect a second double strand break in intron 3, intron 4, or 3′ UTR of both alleles of the ELANE gene in the one or more HSPC cells in which the complex of the first RNA molecule and the CRISPR nuclease affected a first double strand break, thereby obtaining modified cells;
g) administering to the subject the modified cells of step (f),
thereby treating SCN or CyN in the subject.

The present invention provides an RNA molecule comprising a guide sequence portion having 17-20 nucleotides in the sequence of 17-20 contiguous nucleotides set forth in any one of SEQ ID NOs: 1-1192.

The present invention provides a method for inactivating in a cell a mutant ELANE allele, the method comprising delivering to the cell the RNA molecules or compositions of the present invention.

The present invention provides use of the RNA molecules, the compositions, or the composition prepared by the method of the present invention for inactivating in a cell a mutant ELANE allele.

The present invention provides a medicament comprising the RNA molecules, compositions, or the compositions prepared by the methods of the instant invention for use in inactivating in a cell a mutant ELANE allele, wherein the medicament is administered by delivering to the cell the RNA molecules, compositions, or the compositions prepared by the methods of the instant invention.

The present invention provides for use of the methods, the modified cells, the compositions, or the compositions prepared by the methods, or the RNA molecules of the instant invention for treating ameliorating or preventing SCN or CyN in to a subject having or at risk of having SCN or CyN.

The present invention provides for a medicament comprising the RNA molecules, compositions, compositions prepared by the methods of the instant invention, or the modified cells of the instant invention, for use in treating ameliorating or preventing SCN or CyN, wherein the medicament is administered by delivering to a subject having or at risk of having SCN or CyN the RNA molecules, compositions, compositions prepared by the methods of the instant invention, or the modified cells of the instant invention.

The present invention provides for a kit for inactivating a mutant ELANE allele in a cell, comprising the RNA molecules of the instant invention, a CRISPR nuclease or a sequence encoding the CRISPR nuclease, and/or a tracrRNA molecule or a sequence encoding the tracrRNA; and instructions for delivering the RNA molecule; CRISPR nuclease or sequence encoding the CRISPR nuclease, and/or tracrRNA molecule or sequence encoding the tracrRNA to the cell to inactivate the mutant ELANE allele in the cell.

The present invention provides for a kit for treating SCN or CyN in a subject, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease, and/or the tracrRNA to a subject having or at risk of having SCN or CyN so as to treat the SCN or CyN.

The present invention provides a kit for inactivating a mutant ELANE allele in a cell, comprising the compositions, the composition prepared by the methods of the instant invention, or the modified cells of the instant invention, and instructions for delivering the composition to the cell so as to inactivate the ELANE gene in the cell.

The present invention provides a kit for treating SCN or CyN in a subject, comprising the composition, the compositions prepared by the methods of the instant invention, or the modified cells of the instant invention, and instructions for delivering the compositions, the compositions prepared by the methods of the instant invention, or the modified cells of the instant invention, to a subject having or at risk of having SCN or CyN so as to treat SCN or CyN.

### BRIEF DESCRIPTION OF THE DRAWINGS

FIG. 1: excising the promoter region from an upstream SNP position until intron 3 or intron 4 or the 3′ UTR. In one example, a first guide sequence targets a specific sequence of a heterozygous SNP position in an upstream region of the mutant allele (strategy 1a—rs10414837, strategy 1b—rs3761005) and a second guide sequence targets a sequence in intron 4 which is common to two alleles of the gene.

FIG. 2: excising the promoter region from an upstream SNP position until intron 3 or intron 4 or the 3′ UTR. In one example, a first guide sequence targets a specific sequence of a heterozygous SNP position in an upstream region of the mutant allele (strategy 1a—rs10414837, strategy 1b—rs3761005) and a second guide sequence targets a sequence in intron 3 which is common to two alleles of the gene. In another, a first guide sequence targets a specific sequence of a heterozygous SNP position in an upstream region of the mutant allele (strategy 1a—rs10414837, strategy 1b—rs3761005) and a second guide sequence targets a sequence in 3′ UTR which is common to two alleles of the gene.

FIG. 3: excising from intron 3 or intron 4 or 3′ UTR to regions downstream to the 3′ UTR. In one example, a first guide sequence targets a specific sequence of a heterozygous SNP position in an upstream region of the mutant allele (strategy 2—rs1683564) and a second guide sequence targets a sequence in intron 4 which is common to two alleles of the gene. In another, a first guide sequence targets a specific sequence of a heterozygous SNP position in an upstream region of the mutant allele (strategy 2—rs1683564) and a second guide sequence targets a sequence in intron 3 which is common to two alleles of the gene. In a further, a first guide sequence targets a specific sequence of a heterozygous SNP position in an upstream region of the mutant allele (strategy 2—rs1683564) and a second guide sequence targets a sequence in 3′ UTR which is common to two alleles of the gene.

FIG. 4: excising from intron 3 or intron 4 or 3′ UTR to regions downstream to the 3′ UTR. The strategy is designed such as to specifically knock-out the disease-causing allele (‘mutant allele’), while leaving the healthy allele intact. Allele specific editing is achieved by using guides that target discriminating (heterozygous) SNP positions with relatively high heterozygosity frequency in the population.

FIG. 5: The expected coverage in the population based on heterozygotes frequency and overlap/linkage between select heterozygous SNPs. Designing alternative solutions for the three therapeutic strategies may enable a coverage of about 80% of the population. It is evaluated that 80% of the population bear one or more heterozygous SNPs from the list above. From which 33% bear one heterozygous SNP, 31% two heterozygous SNPs from the list, 16% bear three heterozygous SNPs. Whereas 20% don't bear any of the SNPs listed above.

FIG. 6: HeLa cells seeded into 96 well-plate (3K/well). 24 h later were co-transfected with either 65 ng of WT-Cas9 or Dead-Cas9 and 20 ng of gRNA plasmids, identified as g36 through g66, targeting the different regions and SNPs in ELANE using Turbofect reagent (Thermo Scientific). Percent of editing was calculated according the following formula: 100%−(Intensity not edited band/Intensity total bands)*100. The mean activity of each gRNA following subtraction of the Dead-Cas9 background activity±SD of three independent experiment is shown.

FIG. 7: HSCs from healthy donors were nucleofected with RNA components of spCas9-WT and gRNAs targeting either EMW1 (sgEMX1) or ELANE (g35: INT 4; g58: rs3761005; g62: rs1683564). 72 h post nucleofection gDNA was extracted and editing levels were assayed by IDAA. The mean % of editing±SD of 2 independent experiments performed in duplicates is shown.

FIG. 8: Specific knock-out of the mutated allele of the ELANE gene is mediated by excising intron 4 and exon5 of the mutant allele of the ELANE gene. This is achieved by mediating a DSB in intron 4 and utilizing SNP rs1683564 for mediating an allele specific DSB.

### DETAILED DESCRIPTION

Embodiments of the present invention provide a method for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
introducing to the cell a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene.

In embodiments of the present invention, the CRISPR nuclease and the first RNA molecule affects a double strand break in the mutant allele of the ELANE gene, which mutant allele is targeted for the double strand break based on the one or more polymorphic sites.

In embodiments of the present invention, the CRISPR nuclease and the first RNA molecule affects a double strand break in the mutant allele of the ELANE gene, which mutant allele is targeted for the double strand break based on a sequence of the mutant allele at the one or more polymorphic sites.

In embodiments of the present invention, the CRISPR nuclease and the first RNA molecule affects a double strand break in the mutant allele of the ELANE based on the nucleotide base of the one or more polymorphic sites present on the mutant allele of the ELANE gene.

Embodiments of the present invention further comprise introduction of a second RNA molecule comprising a guide sequence portion capable of complexing with a CRISPR nuclease, wherein the complex of the second RNA molecule and CRISPR nuclease affects a second double strand break in the ELANE gene.

In embodiments of the present invention, a composition may comprise 1, 2, 3 or more CRISPR nucleases or sequencing encoding the CRISPR nucleases. In embodiments of the present invention, introducing a composition to the cell may comprise introducing 1, 2, 3, or more compositions to the cell. In embodiments of the present invention, each composition may comprise a different CRISPR nuclease or sequence encoding the CRISPR nucleases or the same CRISPR nuclease or sequence encoding the CRISPR nuclease. In embodiments of the present invention involving two RNA molecules, the second RNA molecule may form a complex with the same CRISPR nuclease as the first RNA molecule, or may form a complex with another CRISPR nuclease.

In embodiments of the present invention, the second double strand break is within a non-coding region of the ELANE gene. In embodiments of the present invention, the non-coding region of the ELANE gene is selected an intron or an untranslated region (UTR). In embodiments of the present invention, the non-coding region is in intron 3 or intron 4. In an embodiments of the present invention the UTR is the 3′UTR.

In embodiments of the present invention, the guide sequence portion of the first RNA molecule comprises 17-20 contiguous nucleotides as set forth in any one of SEQ ID NOs: 1-1192.

According to embodiments of the present invention, the guide sequence portion of the second RNA molecule comprises 17-20 nucleotides in the sequence of 17-20 contiguous nucleotides set forth in any one of SEQ ID NOs: 1-1192, or any one of SEQ ID NOs 1193-2000.

In embodiments of the present invention, the second double strand break is within a non-coding region of the ELANE gene.

In embodiments of the present invention, the cell is heterozygous at rs10414837 or rs3761005 and the complex of the second RNA molecule and CRISPR nuclease affects a double strand break in intron 4 of the ELANE gene.

In embodiments of the present invention, the cell is heterozygous at rs10414837 or rs3761005 and the complex of the second RNA molecule and CRISPR nuclease affects a double strand break in intron 3 of the ELANE gene.

In embodiments of the present invention, the cell is heterozygous at rs10414837 or rs3761005 and the complex of the second RNA molecule and CRISPR nuclease affects a double strand break in the 3′ UTR region of the ELANE gene.

In embodiments of the present invention, the cell is heterozygous at rs1683564 and the complex of the second RNA molecule and CRISPR nuclease affects a double strand break in intron 4 of the ELANE gene.

In embodiments of the present invention, the cell is heterozygous at rs1683564 and the complex of the second RNA molecule and CRISPR nuclease affects a double strand break in intron 3 of the ELANE gene.

In embodiments of the present invention, the cell is heterozygous at rs1683564 and the complex of the second RNA molecule and CRISPR nuclease affects a double strand break in the 3′ UTR region of the ELANE gene.

In some embodiments, the cell is heterozygous at the polymorphic sites in the ELANE rs10414837, and a complex of a first RNA molecule comprising a guide sequence portion having 17-20 nucleotides and a CRISPR nuclease affects a double strand break in the mutant allele of the ELANE gene and not in the functional allele of the ELANE gene. In such embodiments the guide sequence portion of the first RNA molecule comprises having 17-20 nucleotides may comprise a sequence of 17-20 contiguous nucleotides as set forth in any one of SEQ ID NOs: 82, 86, 93, 94, 115, 119, 135, 173, 180, 213, 215, 224, 225, 262, 263, 307, 308, 319, 323, 351, 352, 374, 461, 462, 466, 467, 474, 477, 478, 491, 504, 505, 533, 537, 538, 550, 556, 569, 570, 583, 584, 684, 685, 714, 745, 790, 791, 845, 846, 854, 857, 858, 861, 863, 864, 880, 881, 886, 890, 891, 901, 911, 912, 936, 937, 939, 940, 960, 961, 972, 978, 979, 983, 984, 1018, 1034, 1035, 1040, 1086, 1110, 1111, 1135, 1144, and 1145.

In some embodiments, the cell is heterozygous at the polymorphic sites in the ELANE rs3761005, and a complex of a first RNA molecule comprising a guide sequence portion having 17-20 nucleotides and a CRISPR nuclease affects a double strand break in the mutant allele of the ELANE gene and not in the functional allele of the ELANE gene. In such embodiments the guide sequence portion of the first RNA molecule comprises having 17-20 nucleotides may comprise a sequence of 17-20 contiguous nucleotides as set forth in any one of SEQ ID NOs: 26, 57, 65, 66, 70, 187, 188, 191, 206, 220, 221, 243, 245, 261, 275, 356, 357, 392, 417, 418, 431, 441, 442, 447, 488, 513, 514, 545, 546, 548, 598, 599, 604, 607, 608, 612, 613, 639, 648, 658, 659, 660, 680, 681, 742, 743, 755, 756, 759, 762, 763, 767, 771,772, 773, 786, 787, 815, 816, 818, 819, 820, 831, 836, 849, 850, 870, 871, 898, 899, 907, 908, 1009, 1010, 1013, 1023, 1029, 1030, 1082, 1083, 1093, 1099, 1100, 1101, 1107, 1108, and 1182.

In some embodiments, the cell is heterozygous at the polymorphic sites in the ELANE rs1683564, and a complex of a first RNA molecule comprising a guide sequence portion having 17-20 nucleotides and a CRISPR nuclease affects a double strand break in the mutant allele of the ELANE gene and not in the functional allele of the ELANE gene. In such embodiments the guide sequence portion of the first RNA molecule comprises having 17-20 nucleotides may comprise a sequence of 17-20 contiguous nucleotides as set forth in any one of SEQ ID NOs: 52, 87, 122, 164, 175, 199, 214, 290, 326, 345, 346, 373, 404, 412, 436, 437, 451, 452, 483, 484, 517, 520, 525, 617, 618, 621, 641, 661, 676, 722, 736, 806, 855, 856, 878, 879, 888, 889, 896, 903, 905, 913, 914, 929, 933, 934, 935, 982, 998, 1021, 1022, 1026, 1046, 1047, 1053, 1097, 1121, 1122, 1124, 1126, 1127, 1131, 1134, 1175, 1176, 1183, and 1190.

Embodiments of the present invention comprise obtaining the cell with an ELANE gene mutation associated with severe congenital neutropenia (SCN) or CyN from a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854.

Embodiments of the present invention comprise first selecting a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, and obtaining the cell from the subject.

Embodiments of the present invention comprise obtaining the cell from the subject by mobilization and/or by apheresis.

Embodiments of the present invention comprise obtaining the cell from the subject by bone marrow aspiration.

In embodiments of the present invention, the cell is prestimulated prior to introducing the composition to the cell.

Embodiments of the present invention comprise culture expanding the cell to obtain cells.

In embodiments of the present invention, the cells are cultured with one or more of: stem cell factor (SCF), IL-3, and GM-CSF.

In embodiments of the present invention, the cells are cultured with at least one cytokine.

In embodiments of the present invention, the at least one cytokine is a recombinant human cytokine.

In embodiments of the present invention, the cell is among a plurality of cells, wherein the composition comprising the first RNA molecule or both the first and the second RNA molecule is introduced into at least the cell as well as other cells among the plurality of cells, and the mutant allele of the ELANE gene is inactivated in at least the cell as well as in the other cells among the plurality of cells, thereby obtaining multiple modified cells.

In embodiments of the present invention, introducing the composition comprising the first RNA molecule or introduction of the second RNA molecule comprises electroporation of the cell or cells.

Embodiments of the present invention provide for a modified cell obtained by the methods of the present invention.

In embodiments of the present invention, the modified cells are further culture expanded.

In embodiments of the present invention, the modified cells are capable of engraftment.

In embodiments of the invention, modified cells are capable of long-term engraftment when infused into a patient, giving rise to differentiated hematopoietic cells for at least 12 months after infusion, preferably at least 24 months and even, more preferably at least 30 months after infusion. In a further embodiment, the modified cells are capable of long-term engraftment when infused into an autologous subject. In a further embodiment, the modified cells are capable of long-term engraftment when infused into a subject without myeloablation. In an embodiment of the present invention, the modified cells are delivered to a subject in sufficient numbers that, when engrafted into a human subject, provide long term engraftment.

In embodiments of the present invention, the modified cell or cells are capable of giving rise to progeny cells.

In embodiments of the present invention, the modified cell or cells are capable of giving rise to progeny cells after engraftment.

In embodiments of the present invention, the modified cell or cells are capable of giving rise to progeny cells after an autologous engraftment.

In embodiments of the present invention, the modified cell or cells are capable of giving rise to progeny cells for at least 12 months or at least 24 months after engraftment.

In one embodiment, the cell or cells are stem cells. In one embodiment, the cell is an embryonic stem cell. In some embodiment, the stem cell is a hematopoietic stem/progenitor cell (HSPC).

In embodiments of the present invention, the modified cell or cells are CD34+ hematopoietic stem cells.

In embodiments of the present invention, the modified cell or cells are bone marrow cells or peripheral mononucleated cells (PMCs).

Embodiments of the present invention provide for a modified cell lacking at least a portion of one allele of the ELANE gene.

In embodiments of the present invention, the modified cell was modified from a cell heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854.

Embodiments of the present invention provide for a composition comprising modified cells and a pharmaceutically acceptable carrier.

Embodiments of the present invention provide for an in vitro or ex vivo method of preparing a composition, comprising mixing the cells of the present invention with the pharmaceutically acceptable carrier.

Embodiments of the present invention provide for a method of preparing in vitro or ex vivo a composition comprising modified cells, the method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, and obtaining the cell from the subject;
b) introducing to the cells of step (a) a composition comprising:
   a CRISPR nuclease or a sequence encoding the CRISPR nuclease; and
   a first RNA molecule comprising a guide sequence portion having 17-20 nucleotides.
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally
c) culture expanding the modified cells of step (b),
wherein the modified cells are capable of engraftment and giving rise to progeny cells after engraftment.

Embodiments of the present invention provide for use of a composition prepared in vitro by a method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854;
b) introducing to the cells of step (a) a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
for treating the SCN or CyN in the subject.

Embodiments of the present invention provide for a method of treating a subject afflicted with SCN or CyN, comprising administration of a therapeutically effective amount of the modified cells, compositions, or the compositions prepared by the methods of the instant invention

Embodiments of the present invention provide for a method for treating SCN or CyN in a subject with an ELANE gene mutation relating to SCN or CYN in need thereof and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising:
a) isolating HSPCs from cells obtained from the subject;
b) introducing to the cells of step (a) a composition comprising:
   a CRISPR nuclease or a sequence encoding the CRISPR nuclease; and
   a first RNA molecule comprising a guide sequence portion having 17-20 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
thereby treating the SCN or CyN in the subject.

Embodiments of the present invention provide for a method for treating SCN or CyN in a subject with an ELANE gene mutation relating to SCN or CYN in need thereof and which subject is heterozygous at one or more polymorphic sites selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
administering to the subject autologous modified cells or progeny of autologous modified cells, wherein the autologous modified cells are modified so as to have a double strand break in the mutant allele of the ELANE gene,
   wherein said double strand break results from introduction to the cells of a composition comprising a CRISPR nuclease or a sequence encoding the CRISPR nuclease and a first RNA molecule wherein a complex of the CRISPR nuclease and the first RNA molecule affects a double strand break in the mutant allele of the ELANE gene so as to inactive the mutant allele of the ELANE gene in the cell,
thereby treating the SCN or CyN in the subject.

Embodiments of the present invention provide for a method of selecting a subject for treatment from a pool of subjects diagnosed with SCN or CyN, comprising the steps of:
a) obtaining cells from each subject in the pool of subjects;
b) screening each subject's cells for an ELANE gene mutation related to SCN or CyN, and selecting only subjects with an ELANE gene mutation related to SCN or CyN;
c) screening by sequencing the cells of the subjects selected in step (b) for heterozygosity at one or more polymorphic sites selected from the group consisting of: rs10414837, rs3761005, rs1683564, and
d) selecting for treatment only subjects with cells heterozygous at the one of more polymorphic sites.

Embodiments of the present invention farther comprise treating SCN or CyN in a selected subject, comprising:
e) obtaining HSPC cells from the bone marrow of the subject either by aspiration or by mobilization and apheresis of peripheral blood;
f) introducing to the HSPC cells of step (e):
   one or more CRISPR nucleases or sequences encoding the one or more CRISPR nucleases
   a first RNA molecule comprising a guide sequence portion having 17-20 nucleotides in a sequence of 17-20 contiguous nucleotides set forth in any one of SEQ ID NOs: 1-1192 targeting the nucleotide base of the heterozygous allele of the one or more polymorphic sites present on the mutant allele of the ELANE gene, and
   a second RNA molecule comprising a guide sequence portion targeting a sequence in intron 3, intron 4 or 3′ UTR of the ELANE gene,
wherein a complex of the first RNA molecule and a CRISPR nuclease affects a first double strand break in the mutant allele of the ELANE gene in one or more of the HSPC cells and a complex of the second RNA molecule and a CRISPR nuclease affect a second double strand break in intron 3, intron 4, or 3′ UTR of both alleles of the ELANE gene in the one or more HSPC cells in which the complex of the first RNA molecule and the CRISPR nuclease affected a first double strand break, thereby obtaining modified cells;
g) administering to the subject the modified cells of step (f),
thereby treating SCN or CyN in the subject.

Embodiments of the present invention provide an RNA molecule comprising a guide sequence portion having 17-20 nucleotides in the sequence of 17-20 contiguous nucleotides set forth in any one of SEQ ID NOs: 1-1192.

Embodiments of the present invention further comprise a second RNA molecule comprising a guide sequence portion.

In embodiments of the present invention, the second RNA molecule targets a non-coding region of the ELANE gene.

In embodiments of the present invention, the nucleotide sequence of the guide sequence portion of the second RNA molecule is a different nucleotide sequence from the sequence of the guide sequence portion of the first RNA molecule.

In embodiments of the present invention, the first RNA molecule further comprise a portion having a sequence which binds to a CRISPR nuclease. In embodiments of the present invention, the second RNA molecule further comprise a portion having a sequence which binds to a CRISPR nuclease.

In embodiments of the present invention, the sequence which binds to a CRISPR nuclease is a tracrRNA sequence.

In embodiments of the present invention, the first RNA molecule further comprises a portion having a tracr mate sequence. In embodiments of the present invention, the second RNA molecule further comprises a portion having a tracr mate sequence.

In embodiments of the present invention, the first RNA molecule further comprises one or more linker portions. In embodiments of the present invention, the second RNA molecule further comprises one or more linker portions.

In embodiments of the present invention, the first RNA molecule is up to 300 nucleotides in length. In embodiments of the present invention, the second RNA molecule is up to 300 nucleotides in length.

In embodiments of the present invention, the composition further comprises one or more CRISPR nucleases or sequences encoding the one or more CRISPR nucleases. In embodiments of the present invention, the composition further comprises one or more tracrRNA molecules or sequences encoding the one or more tracrRNA molecules.

Embodiments of the present invention provide a method for inactivating in a cell a mutant ELANE allele, the method comprising delivering to the cell the RNA molecules or compositions of the present invention.

In embodiments of the present invention, the one or more CRISPR nuclease or sequences encoding the one or more CRISPR nucleases and the RNA molecule or RNA molecules are delivered to the subject and/or cells substantially at the same time or at different times.

In embodiments of the present invention, the tracrRNA molecules or sequences encoding the one or more tracrRNA molecules and the RNA molecule or RNA molecules are delivered to the subject and/or cells substantially at the same time or at different times.

In embodiments of the present invention, the method comprises removing an exon containing a disease-causing mutation from a mutant allele, wherein the first RNA molecule or the first and the second RNA molecules target regions flanking an entire exon or a portion of the exon.

In embodiments of the present invention, the method comprises removing multiple exons, the entire open reading frame of a gene, or removing the entire gene.

In embodiments of the present invention, the first RNA molecule or the first and the second RNA molecules target an alternative splicing signal sequence between an exon and an intron of a mutant allele.

In embodiments of the present invention, the second RNA molecule targets a sequence present in both a mutant allele and a functional allele.

In embodiments of the present invention, the second RNA molecule targets an intron.

In embodiments of the present invention, the method results in subjecting the mutant allele to insertion or deletion by an error prone non-homologous end joining (NHEJ) mechanism, generating a frameshift in the mutant allele's sequence.

In embodiments of the present invention, the frameshift results in inactivation or knockout of the mutant allele.

In embodiments of the present invention, the frameshift creates an early stop codon in the mutant allele or the frameshift results in nonsense-mediated mRNA decay of the transcript of the mutant allele.

In embodiments of the present invention, inactivating or treating results in a truncated protein encoded by the mutant allele and a functional protein encoded by the functional allele.

In embodiments of the present invention, the cells or the subject is heterozygous at rs10414837 or rs3761005 and wherein the complex of the second RNA molecule and CRISPR nuclease affects a double strand break in intron 4 of the ELANE gene.

In embodiments of the present invention, the cells or the subject is heterozygous at rs10414837 or rs3761005 and the complex of the second RNA molecule and CRISPR nuclease affects a double strand break in intron 3 of the ELANE gene.

In embodiments of the present invention, the cells or the subject is heterozygous at rs10414837 or rs3761005 and wherein the complex of the second RNA molecule and CRISPR nuclease affects a double strand break in the 3′ UTR region of the ELANE gene.

In embodiments of the present invention, the cells or the subject is heterozygous at rs1683564 and wherein the complex of the second RNA molecule and CRISPR nuclease affects a double strand break in intron 4 of the ELANE gene.

In embodiments of the present invention, the cells or the subject is heterozygous at rs1683564 and wherein the complex of the second RNA molecule and CRISPR nuclease affects a double strand break in intron 3 of the ELANE gene.

In embodiments of the present invention, the cells or the subject is heterozygous at rs1683564 and wherein the complex of the second RNA molecule and CRISPR nuclease affects a double strand break in the 3′ UTR region of the ELANE gene.

Embodiments of the present invention provide use of the RNA molecules, the compositions, or the composition prepared by the method of the present invention for inactivating in a cell a mutant ELANE allele.

Embodiments of the present invention provide a medicament comprising the RNA molecules, compositions, or the compositions prepared by the methods of the instant invention for use in inactivating in a cell a mutant ELANE allele, wherein the medicament is administered by delivering to the cell the RNA molecules, compositions, or the compositions prepared by the methods of the instant invention.

Embodiments of the present invention provide for use of the methods, the modified cells, the compositions, or the compositions prepared by the methods, or the RNA molecules of the instant invention for treating ameliorating or preventing SCN or CyN in to a subject having or at risk of having SCN or CyN.

Embodiments of the present invention provide for a medicament comprising the RNA molecules, compositions, compositions prepared by the methods of the instant invention, or the modified cells of the instant invention, for use in treating ameliorating or preventing SCN or CyN, wherein the medicament is administered by delivering to a subject having or at risk of having SCN or CyN the RNA molecules, compositions, compositions prepared by the methods of the instant invention, or the modified cells of the instant invention.

Embodiments of the present invention provide for a kit for inactivating a mutant ELANE allele in a cell, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease or sequence encoding the CRISPR nuclease, and/or the tracrRNA or sequence encoding the tracrRNA molecule to the cell to inactivate the mutant ELANE allele in the cell.

Embodiments of the present invention provide for a kit for treating SCN or CyN in a subject, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease or sequence encoding the CRISPR nuclease, and/or the tracrRNA or sequence encoding the tracrRNA molecule to a subject having or at risk of having SCN or CyN so as to treat the SCN or CyN.

Embodiments of the present invention provide for a kit for inactivating a mutant ELANE allele in a cell, comprising the compositions, the composition prepared by the methods of the instant invention, or the modified cells of the instant invention, and instructions for delivering the composition to the cell so as to inactivate the ELANE gene in the cell.

Embodiments of the present invention provide for a kit for treating SCN or CyN in a subject, comprising the composition, the compositions prepared by the methods of the instant invention, or the modified cells of the instant invention, and instructions for delivering the compositions, the compositions prepared by the methods of the instant invention, or the modified cells of the instant invention, to a subject having or at risk of having SCN or CyN so as to treat SCN or CyN.

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “heterozygous single nucleotide polymorphism” or “SNP” refers to a single nucleotide position in a genome that differs between paired chromosomes within a population. As used herein the most common or most prevalent nucleotide base at the position is referred to as the reference (REF), wild-type (WT), common, or major form. Less prevalent nucleotide bases at the position are referred to as the alternative (ALT), minor, rare, or variant forms.

The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, or 24 nucleotides in length, or approximately 17-24, 18-22, 19-22, 18-20, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence.

The term “targets” as used herein, refers to the guide sequence portion of the RNA molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

In the context targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is effected by a complex of an RNA molecule and the CRISPR nuclease as a result of hybridization with the target sequence, i.e. on-target hybridization.

In embodiments of the present invention, RNA guide molecule may target the mutant allele based on the nucleotide base present in the polymorphic site on the mutant allele.

In embodiments of the present invention, an RNA molecule comprises a guide sequence portion having 17-20 nucleotides in the sequence of 17-20 contiguous nucleotides set forth in any one of SEQ ID NOs: 1-1192; or set forth in the following group SEQ ID NOs: 6, 75, 76, 252, 253, 287, 295, 296, 311, 536, 592, 595, 596, 597, 695, 729, 737, 812, 839, 915, 947, 1048, 1049, 1070, 1071, 1169, or set forth in the following group SEQ ID NOs: 6, 75, 76, 93, 94, 97, 98, 148, 149, 171, 173, 180, 182, 183, 184, 187, 188, 213, 232, 234, 249, 252, 253, 264, 272, 287, 291, 295, 296, 305, 306, 307, 308, 311, 326, 333, 334, 337, 338, 339, 340, 351, 352, 358, 359, 378, 379, 385, 388, 399, 408, 410, 419, 420, 426, 427, 428, 429, 430, 436, 437, 449, 450, 465, 468, 476, 477, 478, 480, 495, 497, 499, 500, 508, 511, 521, 522, 523, 524, 529, 530, 532, 536, 542, 545, 546, 564, 565, 566, 573, 574, 583, 584, 591, 592, 595, 596, 597, 598, 599, 601, 602, 604, 612, 613, 616, 622, 623, 634, 644, 645, 658, 659, 661, 670, 671, 678, 680, 681, 684, 685, 688, 689, 694, 695, 714, 715, 716, 722, 723, 724, 729, 736, 737, 739, 740, 745, 755, 756, 760, 761, 769, 770, 771, 772, 775, 776, 786, 787, 806, 809, 812, 818, 819, 821, 822, 826, 829, 830, 833, 834, 839, 845, 846, 861, 862, 874, 875, 876, 877, 884, 888, 889, 890, 891, 893, 894, 911, 912, 913, 914, 915, 925, 928, 930, 931, 939, 940, 942, 946, 947, 948, 949, 950, 957, 972, 974, 982, 994, 998, 1006, 1007, 1008, 1021, 1022, 1026, 1027, 1028, 1031, 1032, 1034, 1035, 1039, 1046, 1047, 1048, 1049, 1057, 1070, 1071, 1072, 1074, 1075, 1076, 1079, 1084, 1090, 1091, 1093, 1094, 1095, 1112, 1113, 1116, 1117, 1118, 1119, 1121, 1122, 1124, 1140, 1168, 1169, 1170, 1171, 1179; or set forth in the following group SEQ ID NOs: 6, 10, 13, 14, 19, 21, 22, 23, 24, 25, 26, 29, 30, 34, 35, 36, 39, 42, 43, 44, 45, 46, 47, 48, 49, 50, 52, 53, 54, 55, 57, 58, 61, 62, 64, 65, 66, 67, 68, 69, 70, 71, 72, 73, 74, 75, 76, 82, 86, 87, 88, 89, 91, 92, 93, 94, 95, 96, 97, 98, 99, 100, 101, 102, 103, 107, 108, 109, 115, 119, 122, 123, 124, 125, 126, 127, 128, 130, 133, 134, 135, 136, 137, 138, 139, 140, 141, 142, 143, 144, 148, 149, 150, 152, 153, 155, 156, 158, 159, 160, 161, 162, 163, 164, 167, 168, 169, 170, 171, 172, 173, 175, 176, 177, 178, 179, 180, 181, 182, 183, 184, 187, 188, 189, 190, 191, 192, 195, 196, 198, 199, 201, 203, 206, 209, 211, 212, 213, 214, 215, 216, 217, 219, 220, 221, 223, 224, 225, 226, 227, 232, 233, 234, 236, 237, 238, 240, 241, 242, 243, 244, 245, 246, 247, 248, 249, 250, 251, 252, 253, 254, 255, 256, 261, 262, 263, 264, 265, 266, 267, 269, 270, 271, 272, 273, 274, 275, 276, 277, 281, 282, 285, 286, 287, 290, 291, 292, 293, 294, 295, 296, 302, 303, 305, 306, 307, 308, 310, 311, 314, 319, 323, 326, 327, 328, 329, 331, 332, 333, 334, 335, 336, 337, 338, 339, 340, 341, 343, 344, 345, 346, 349, 350, 351, 352, 353, 354, 356, 357, 358, 359, 360, 363, 364, 366, 370, 371, 372, 373, 374, 375, 376, 377, 378, 379, 380, 381, 382, 385, 386, 387, 388, 389, 390, 391, 392, 393, 394, 395, 396, 397, 399, 400, 404, 405, 406, 407, 408, 410, 411, 412, 415, 417, 418, 419, 420, 422, 423, 424, 425, 426, 427, 428, 429, 430, 431, 432, 433, 435, 436, 437, 440, 441, 442, 443, 444, 445, 446, 447, 449, 450, 451, 452, 454, 455, 456, 457, 460, 461, 462, 464, 465, 466, 467, 468, 469, 470, 471, 474, 475, 476, 477, 478, 479, 480, 483, 484, 485, 486, 488, 489, 491, 492, 493, 494, 495, 496, 497, 498, 499, 500, 501, 502, 504, 505, 506, 508, 509, 510, 511, 513, 514, 517, 518, 519, 520, 521, 522, 523, 524, 525, 526, 527, 528, 529, 530, 531, 532, 533, 536, 537, 538, 539, 540, 541, 542, 543, 544, 545, 546, 547, 548, 549, 550, 552, 553, 554, 555, 556, 557, 558, 559, 560, 561, 562, 563, 564, 565, 566, 567, 568, 569, 570, 571, 573, 574, 577, 579, 580, 582, 583, 584, 586, 587, 588, 590, 591, 592, 593, 595, 596, 597, 598, 599, 600, 601, 602, 604, 605, 606, 607, 608, 609, 610, 612, 613, 614, 615, 616, 617, 618, 619, 620.621, 622, 623, 624, 625, 628, 629, 630, 633, 634, 635, 636, 637, 638, 639, 640, 641, 644, 645, 648, 650, 651, 652, 653, 654, 655, 658, 659, 660, 661, 663, 664, 665, 667, 670, 671, 672, 673, 675, 676, 678, 680, 681, 683, 684, 685, 686, 688, 689, 690, 691, 692, 694, 695, 698, 700, 701, 702, 703, 704, 705, 706, 707, 708, 709, 712, 713, 714, 715, 716, 718, 719, 720, 722, 723, 724, 727, 728, 729, 730, 731, 732, 733, 734, 735, 736, 737, 738, 739, 740, 742, 743, 744, 745, 746, 747, 748, 749, 753, 754, 755, 756, 757, 758, 759, 760, 761, 762, 763, 764, 767, 769, 770, 771, 772, 773, 775, 776, 778, 779, 780, 781, 786, 787, 788, 789, 790, 791, 792, 794, 795, 798, 800, 801, 805, 806, 807, 808, 809, 811, 812, 813, 814, 815, 816, 818, 819, 820, 821, 822, 823, 826, 829, 830, 831, 832, 833, 834, 835, 836, 837, 838, 839, 843, 844, 845, 846, 849, 850, 852, 853, 854, 855, 856, 857, 858, 861, 862, 863, 864, 865, 866, 867, 868, 870, 871, 872, 874, 875, 876, 877, 878, 879, 880, 881, 882, 883, 884, 886, 887, 888, 889, 890, 891, 893, 894, 895, 896, 897, 898, 899, 901, 902, 903, 905, 906, 907, 908, 909, 910, 911, 912, 913, 914, 915, 916, 917, 920, 921, 925, 926, 927, 928, 929, 930, 931, 932, 933, 934, 935, 936, 937, 939, 940, 942, 943, 945, 946, 947, 948, 949, 950, 951, 952, 953, 954, 955, 956, 957, 958, 960, 961, 962, 963, 964, 965, 966, 967, 968, 969, 972, 974, 975, 976, 978, 979, 982, 983, 984, 988, 989, 990, 991, 992, 993, 994, 996, 997, 998, 1001, 1002, 1003, 1004, 1005, 1006, 1007, 1008, 1009, 1010, 1011, 1012, 1013, 1014, 1015, 1017, 1018, 1019, 1020, 1021, 1022, 1023, 1024, 1026, 1027, 1028, 1029, 1030, 1031, 1032, 1033, 1034, 1035, 1036, 1037, 1038, 1039, 1040, 1042, 1043, 1044, 1045, 1046, 1047, 1048, 1049, 1050, 1051, 1052, 1053, 1054, 1055, 1056, 1057, 1058, 1061, 1062, 1063, 1064, 1069, 1070, 1071, 1072, 1073, 1074, 1075, 1076, 1077, 1078, 1079, 1080, 1081, 1082, 1083, 1084, 1086, 1090, 1091, 1093, 1094, 1095, 1097, 1099, 1100, 1101, 1102, 1103, 1104, 1105, 1107, 1108, 1110, 1111, 1112, 1113, 1114, 1115, 1116, 1117, 1118, 1119, 1120, 1121, 1122, 1123, 1124, 1126, 1127, 1128, 1131, 1132, 1133, 1134, 1135, 1136, 1137, 1138, 1139, 1140, 1142, 1143, 1144, 1145, 1147, 1148, 1149, 1151, 1152, 1153, 1154, 1155, 1156, 1158, 1159, 1160, 1162, 1164, 1165, 1166, 1167, 1168, 1169, 1170, 1171, 1172, 1173, 1175, 1176, 1179, 1180, 1181, 1182, 1183. It is understood that in any of the embodiments of the present invention the guide sequence portion of an RNA molecule may comprise 17-20 contiguous nucleotides set forth in any single sequence of SEQ ID NOs: 1-1192, or in any single sequence from the above groups of sequences.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 20 nucleotides in length and consists of 20 nucleotides in the sequence of 20 contiguous nucleotides set forth in any one of SEQ ID NOs: 1-1192. In embodiments of the present invention, the guide sequence portion may be less than 20 nucleotides in length. For example, in embodiments of the present invention the guide sequence portion may be 17, 18, or 19 nucleotides in length. In such embodiments the guide sequence portion may consist of 17, 18, or 19 nucleotides, respectively, in the sequence of 17-20 contiguous nucleotides set forth in any one of SEQ ID NOs: 1-1192. For example, a guide sequence portion having 17 nucleotides in the sequence of 17 contiguous nucleotides set forth in SEQ ID NO: 1 may consist of any one of the following nucleotide sequences (nucleotides excluded from the contiguous sequence are marked in strike-through):

SEQ ID NO: 1
AAAAAAACACAAUGUGGGGA
17 nucleotide guide sequence 1:
(SEQ ID NO: 1201) [symbol]
AAAACACAAUGUGGGGA
17 nucleotide guide sequence 2:
(SEQ ID NO: 1202) [symbol]
AAAAACACAAUGUGGGGA [symbol]
17 nucleotide guide sequence 3:
(SEQ ID NO: 1203) [symbol]
AAAAAACACAAUGUGGG [symbol]
17 nucleotide guide sequence 4:
(SEQ ID NO: 1204)
AAAAAAACACAAUGUGG

[symbol]

In embodiments of the present invention, the guide sequence portion may be greater than 20 nucleotides in length. For example, in embodiments of the present invention the guide sequence portion may be 21, 22, 23, or 24 nucleotides in length. In such embodiments the guide sequence portion comprises 20 nucleotides in the sequence of 20 contiguous nucleotides set forth in any one of SEQ ID NOs: 1-1192 and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule.

In embodiments of the present invention, the RNA molecule may further comprise the sequence of a tracrRNA molecule. Such embodiments may be designed as a synthetic fusion of the guide portion of the RNA molecule and the trans-activating crRNA (tracrRNA). (See Jinek (2012) Science). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via base pairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

According to embodiments of the present invention, an RNA molecule may be up to 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 200 up to 300 nucleotides in length, 100 to 200 nucleotides in length, or 150 up to 250 nucleotides in length. Each possibility represents a separate embodiment.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

As used herein, the term HSPC refers to both hematopoietic stem cells and hematopoietic stem progenitor cells. Non-limiting examples of stem cells include a bone marrow cell, a myeloid progenitor cell, a multipotent progenitor cell, a lineage restricted progenitor cell.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

In embodiments of the present invention, an RNA molecule is designed to target a heterozygous polymorphic site present in the mutant allele of the ELANE gene, wherein the RNA molecule targets the nucleotide base, REF or ALT, of the heterozygous polymorphic site present in the mutant allele of the ELANE gene

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

Embodiments of the present invention provide methods for utilizing at least one heterozygous SNP in a gene expressing a dominant mutant allele in a given cell or subject. In embodiments of the present invention, the SNP utilized may or may not be associated with a disease phenotype. In embodiments of the present invention, an RNA molecule comprising a guide sequence targets the mutant allele of the gene by targeting the nucleotide base present at a heterozygous SNP in the mutant allele of the gene and therefore having a different nucleotide base in the functional allele of the gene.

According to embodiments of the present invention, the first RNA molecule targets a first heterozygous SNP present in an exon or promoter of the ELANE gene wherein the first RNA molecule targets the nucleotide base, REF or ALT, of the first SNP present in the mutant allele of the ELANE gene, and wherein the second RNA molecule targets a second heterozygous SNP present in the same or a different exon or an intron of the ELANE gene wherein the second RNA molecule targets the nucleotide base, REF or ALT, of the second SNP present in the mutant allele of the ELANE gene, or a the second RNA molecule targets a sequence in a non-coding region present in both the mutant or functional allele.

According to embodiments of the present invention, the first RNA molecule or the first and the second RNA molecules target a heterozygous SNP present in the promoter region, the start codon, or the untranslated region (UTR) of the ELANE gene wherein the RNA molecule targets the nucleotide base, REF or ALT, of the SNP present in the mutant allele of the ELANE gene.

According to embodiments of the present invention, the first RNA molecule or the first and the second RNA molecules targets at least a portion of the promoter and/or the start codon and/or a portion of the UTR of the mutant allele of the ELANE gene.

According to embodiments of the present invention, the first RNA molecule targets a portion of the promoter, a first heterozygous SNP present in the promoter of the ELANE gene, or a heterozygous SNP present upstream to the promoter of the ELANE gene and the second RNA molecule targets a second heterozygous SNP, which is present in the ELANE gene downstream of the first heterozygous SNP, and is in the promoter, in the UTR, or in an intron or in an exon of the ELANE gene, wherein the first RNA molecule targets the nucleotide base, REF or ALT, of the first SNP present in the mutant allele of the of the ELANE gene, wherein the second RNA molecule targets the nucleotide base, REF or ALT, of the second SNP present in the mutant allele of the ELANE gene.

According to embodiments of the present invention, the first RNA molecule targets a heterozygous SNP present in the promoter, upstream of the promoter, or the UTR of a the ELANE gene wherein the RNA molecule targets the nucleotide base, REF or ALT, of the SNP present in the mutant allele of the ELANE gene and the second RNA molecule is designed to target a sequence which is present in an intron of both the mutant allele and the functional allele of the ELANE gene.

According to embodiments of the present invention, the first RNA molecule targets a sequence upstream of the promotor which is present in both a mutant and functional allele of the ELANE gene and the second RNA molecule targets a heterozygous SNP present in any location of the of the ELANE gene wherein the second RNA molecule targets the nucleotide base, REF or ALT, of the SNP present in the mutant allele of the ELANE gene.

According to embodiments of the present invention, there is provided a method comprising removing an exon containing a disease-causing mutation from a mutant allele, wherein the first RNA molecule or the first and the second RNA molecules target regions flanking an entire exon or a portion of the exon.

According to embodiments of the present invention, there is provided a method comprising removing multiple exons, the entire open reading frame of a gene, or removing the entire gene.

According to embodiments of the present invention, the first RNA molecule targets a first heterozygous SNP present in an exon or promoter of the ELANE gene, and wherein the second RNA molecule targets a second heterozygous SNP present in the same or a different exon or in an intron of the ELANE gene wherein the second RNA molecule targets the nucleotide base, REF or ALT, of the second SNP present in the mutant allele of the ELANE gene, or the second RNA molecule targets a sequence in an intron present in both the mutant and functional allele of the ELANE gene.

According to embodiments of the present invention, the first RNA molecule or the first and the second RNA molecules target an alternative splicing signal sequence between an exon and an intron of a mutant allele.

According to embodiments of the present invention, the second RNA molecule targets a sequence present in both a mutant allele and a functional allele of the ELANE gene.

According to embodiments of the present invention, the second RNA molecule targets an intron.

According to embodiments of the present invention, there is provided a method comprising subjecting the mutant allele to insertion or deletion by an error prone non-homologous end joining (NHEJ) mechanism, generating a frameshift in the mutant allele's sequence.

According to embodiments of the present invention, the frameshift results in inactivation or knockout of the mutant allele.

According to embodiments of the present invention, the frameshift creates an early stop codon in the mutant allele.

According to embodiments of the present invention, the frameshift results in nonsense-mediated mRNA decay of the transcript of the mutant allele.

According to embodiments of the present invention, the inactivating or treating results in a truncated protein encoded by the mutant allele and a functional protein encoded by the functional allele.

The compositions and methods of the present disclosure may be utilized for treating, preventing, ameliorating, or slowing progression of SCN or CyN.

In some embodiments, a mutant allele is deactivated by delivering to a cell an RNA molecule which targets a heterozygous SNP present in the promoter region, the start codon, or the untranslated region (UTR) of the ELANE gene wherein the RNA molecule targets the nucleotide base, REF or ALT, of the SNP present in the mutant allele of the ELANE gene.

In some embodiments, a mutant allele is inactivated by removing at least a portion of the promoter and/or removing the start codon and/or a portion of the UTR. In some embodiments, the method of deactivating a mutant allele comprises removing at least a portion of the promoter. In such embodiments one RNA molecule may be designed for targeting a first heterozygous SNP present in the promoter or upstream to the promoter of the ELANE gene and another RNA molecule is designed to target a second heterozygous SNP, which is downstream of the first SNP, and is present in the promoter, in the UTR, or in an intron or in an exon of the ELANE gene. Alternatively, one RNA molecule may be designed for targeting a heterozygous SNP present in the promoter, or upstream of the promoter, or the UTR of the ELANE gene and another RNA molecule is designed to target a sequence which is present in an intron of both the mutant allele and the functional allele of the ELANE gene. Alternatively, one RNA molecule may be designed for targeting a sequence upstream of the promotor which is present in both the mutant and functional allele and the other guide is designed to target a heterozygous SNP present in any location of the ELANE gene e.g., in an exon, intron, UTR, or downstream of the promoter of the ELANE gene wherein the RNA molecule targets the nucleotide base, REF or ALT, of the SNP present in the mutant allele of the ELANE gene.

In some embodiments, the method of deactivating a mutant allele comprises an exon skipping step comprising removing an exon containing a disease-causing mutation from the mutant allele. Removing an exon containing a disease-causing mutation in the mutant allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. As an alternative to single exon skipping, multiple exons, the entire open reading frame or the entire gene can be excised using two RNA molecules flanking the region desired to be excised.

In some embodiments, the method of deactivating a mutant allele comprises delivering two RNA molecules to a cell, wherein one RNA molecule targets a first heterozygous SNP present in an exon or promoter of the ELANE gene wherein the RNA molecule targets the nucleotide base, REF or ALT, of the first SNP present in the mutant allele of the ELANE gene, and wherein the other RNA molecule targets a second heterozygous SNP present in the same or a different exon or in an intron of the ELANE gene wherein the RNA molecule targets the nucleotide base, REF or ALT, of the second SNP present in the mutant allele of the ELANE gene, or the second RNA molecule targets a sequence in an intron present in both the mutant or functional allele.

In some embodiments, an RNA molecule is used to target a CRISPR nuclease to an alternative splicing signal sequence between an exon and an intron of a mutant allele, thereby destroying the alternative splicing signal sequence in the mutant allele.

Any one of, or combination of, the above-mentioned strategies for deactivating a mutant allele may be used in the context of the invention.

Additional strategies may be used to deactivate a mutant allele. For example, in embodiments of the present invention, an RNA molecule is used to direct a CRISPR nuclease to an exon or a splice site of a mutant allele in order to create a double-stranded break (DSB), leading to insertion or deletion of nucleotides by an error-prone non-homologous end-joining (NHEJ) mechanism and formation of a frameshift mutation in the mutant allele. The frameshift mutation may result in: (1) inactivation or knockout of the mutant allele by generation of an early stop codon in the mutant allele, resulting in generation of a truncated protein; or (2) nonsense mediated mRNA decay of the transcript of the mutant allele. In further embodiments, one RNA molecule is used to direct a CRISPR nuclease to a promotor of a mutant allele.

In some embodiments, the method of deactivating a mutant allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA molecule which binds to/associates with and/or directs the RNA guided DNA nuclease e.g., CRISPR nuclease to a sequence comprising at least one nucleotide which differs between a mutant allele and a functional allele (e.g., heterozygous SNP) of a gene of interest (i.e., a sequence of the mutant allele which is not present in the functional allele).

In some embodiments, the method comprises the steps of: contacting a mutant allele of a gene of interest with an allele-specific RNA molecule and a CRISPR nuclease e.g., a Cas9 protein, wherein the allele-specific RNA molecule and the CRISPR nuclease e.g., Cas9 associate with a nucleotide sequence of the mutant allele of the gene of interest which differs by at least one nucleotide from a nucleotide sequence of a functional allele of the gene of interest, thereby modifying or knocking-out the mutant allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the cleaved mutant allele is further subjected to insertion or deletion (indel) by an error prone non-homologous end joining (NHEJ) mechanism, generating a frameshift in the mutant allele's sequence. In some embodiments, the generated frameshift results in inactivation or knockout of the mutant allele. In some embodiments, the generated frameshift creates an early stop codon in the mutant allele and results in generation of a truncated protein. In such embodiments, the method results in the generation of a truncated protein encoded by the mutant allele and a functional protein encoded by the functional allele. In some embodiments, a frameshift generated in a mutant allele using the methods of the invention results in nonsense-mediated mRNA decay of the transcript of the mutant allele.

In some embodiments, the mutant allele is an allele of the “elastase, neutrophil expressed” gene (ELANE gene). In some embodiments, the RNA molecule targets a heterozygous SNP of the ELANE gene which co-exists with/is genetically linked to the mutated sequence associated with SCN or CyN genetic disorder. In some embodiments, the RNA molecule targets a heterozygous SNP of the ELANE gene, wherein the heterozygosity of said SNP is highly prevalent in the population. In embodiments of the present invention, the REF nucleotide is prevalent in the mutant allele and not in the functional allele of an individual subject to be treated. In embodiments of the present invention, the ALT nucleotide is prevalent in the mutant allele and not in the functional allele of an individual subject to be treated. In some embodiments, a disease-causing mutation within a mutant ELANE allele is targeted.

In embodiments of the present invention, the heterozygous SNP may or may not be associated with an ELANE related disease phenotype. In embodiments of the present invention, the heterozygous SNP is associated with an ELANE related disease phenotype. In embodiments of the present invention, the SNP is not associated with an ELANE related disease phenotype

In some embodiments, the heterozygous SNP is within an exon of the gene of interest. In such embodiments, a guide sequence portion of an RNA molecule may be designed to target the exon of the gene of interest.

In some embodiments, a heterozygous SNP is within an intron or an exon of the gene of interest. In some embodiments, a heterozygous SNP is in a splice site between the intron and the exon. In some embodiments a heterozygous SNP is in a PAM site of the gene of interest.

A skilled artisan will appreciate that in each of the embodiments of the present invention, individually, each of the RNA molecules of the present invention are capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer. Accordingly, in embodiments of the present invention, the guide sequences and RNA molecules of the present invention may target a location 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from a PAM site.

Therefore, in embodiments of the present invention, the RNA molecules of the present invention in complex with a nuclease, e.g., a CRISPR nuclease, may affect a double strand break in an allele of a gene 1, 2, 3, 4, 5, 6, 7, 8, 9, 10, 11, 12, 13, 14, 15, 16, 17, 18, 19, 20, 21, 22, 23, 24, 25, 26, 27, 28 upstream or downstream from a target site. A skilled artisan will appreciate that where a heterozygous polymorphic site is present and is used to define the target, an RNA molecule may be designed to target and affect a double stranded break in only the REF or ALT nucleotide base of the heterozygous polymorphic site.

Where the heterozygous polymorphic site is within the PAM site, it is understood that the RNA molecule may be designed to target a sequence 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from the PAM site, and a complex of the RNA molecule and nuclease is designed to target only one of the REF or ALT nucleotide base of the heterozygous polymorphic site in the PAM site and effect a break in the PAM site, e.g. the tracrRNA is designed to target one of the REF or ALT nucleotide base of the heterozygous polymorphic site.

In embodiments of the present invention, an RNA molecule is designed to target a heterozygous polymorphic site present in the ELANE gene, wherein the RNA molecule and/or the complex of the RNA molecule and a CRISPR nuclease targets the nucleotide base, REF or ALT, of the heterozygous polymorphic site present in the mutant allele of the ELANE gene

In embodiments of the present invention, the RNA molecules, compositions, methods, cells, kits, or medicaments are utilized for treating a subject having a disease phenotype resulting from the heterozygote ELANE gene. In embodiments of the present invention, the disease is SCN or CyN. In such embodiments, the method results in improvement, amelioration or prevention of the disease phenotype.

In embodiments of the present invention, the RNA molecules, compositions, methods, cells, kits, or medicaments of the present invention are utilized in combination with a second therapy for SCN or CyN to treat the subject. In embodiments of the present invention, the RNA molecules, compositions, methods, kits, or medicaments of the present invention are administered prior to administration of the second therapy, during administration of the second therapy, and/or after administration of the second therapy.

In embodiments of the present invention, the RNA molecules, compositions, methods, cells, kits, or medicaments of the present invention are administered in combination with Granulocyte colony-stimulating factor (G-CSF) therapy.

Embodiments referred to above refer to a CRISPR nuclease, RNA molecule(s), and tracrRNA being effective in a subject or cells at the same time. The CRISPR, RNA molecule(s), and tracrRNA can be delivered substantially at the same time or can be delivered at different times but have effect at the same time. For example, this includes delivering the CRISPR nuclease to the subject or cells before the RNA molecule and/or tracr RNA is substantially extant in the subject or cells.

According to embodiments of the present invention, there is provided a method for inactivating in a cell a mutant allele of the ELANE gene, the method comprising the steps of:
a) selecting a cell with an ELANE gene mutation associated with SCN or CyN and who is heterozygous at one or more polymorphic sites in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854;
b) introducing to the cell a composition comprising:
   a CRISPR nuclease or sequence encoding the CRISPR nuclease, and
   a first RNA molecule comprising a guide sequence portion having 17-20 nucleotides,
wherein a complex of the CRISPR nuclease and the first RNA molecule affects a double strand break in the mutant allele of the ELANE gene and not in the functional allele of the ELANE gene in the cell;
thereby inactivating the mutant allele of the ELANE gene in the cell.

According to embodiments of the present invention, there is provided a method for inactivating in a cell a mutant allele of the ELANE gene, the method comprising the steps of:
a) selecting a cell with an ELANE gene mutation associated with SCN or CyN and who is heterozygous at one or more polymorphic sites in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854;
b) introducing to the cell a composition comprising:
   a CRISPR nuclease or sequence encoding the CRISPR nuclease, and
   a first RNA molecule comprising a guide sequence portion having 17-20 nucleotides,
wherein a complex of the CRISPR nuclease and the first RNA molecule affects a double strand break in the mutant allele of the ELANE gene and not in the functional allele of the ELANE gene in the cell;
and wherein the method further comprises introduction of a second RNA molecule comprising a guide sequence portion capable of complexing with a CRISPR nuclease, wherein the complex of the second RNA molecule and the CRISPR nuclease affects a second double strand break in the ELANE gene; thereby inactivating the mutant allele of the ELANE gene in the cell.

According to embodiments of the present invention, there is provided a method for inactivating in a cell a mutant allele of the ELANE gene having a mutation associated with SCN or CyN and which cell is heterozygous at one or more polymorphic sites in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising
introducing to the cell a composition comprising:
   a CRISPR nuclease or sequence encoding the CRISPR nuclease, and
   a first RNA molecule comprising a guide sequence portion having 17-20 nucleotides,
wherein a complex of the CRISPR nuclease and the first RNA molecule affects a double strand break in the mutant allele of the ELANE gene; thereby inactivating the mutant allele of the ELANE gene in the cell.

According to embodiments of the present invention, there is provided a method for inactivating in a cell a mutant allele of the ELANE gene with an ELANE gene mutation associated with SCN or CyN and heterozygous at one or more polymorphic sites in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854, the method comprising:
introducing to the cell a composition comprising:
   a CRISPR nuclease or sequence encoding the CRISPR nuclease, and
   a first RNA molecule comprising a guide sequence portion having 17-20 nucleotides,
wherein a complex of the CRISPR nuclease and the first RNA molecule affects a double strand break in the mutant allele of the ELANE gene;
and wherein the method further comprises introduction of a second RNA molecule comprising a guide sequence portion capable of complexing with a CRISPR nuclease, wherein the complex of the second RNA molecule and CRISPR nuclease affects a second double strand break in the ELANE gene; thereby inactivating the mutant allele of the ELANE gene in the cell.

In embodiments of the present invention, a complex of the CRISPR nuclease and the first RNA molecule affects a double strand break in the mutant allele of the ELANE gene and not in the functional allele of the ELANE gene in the cell.

In embodiments of the present invention, the cell is also heterozygous at least one additional polymorphic site in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854.

In embodiments of the present invention, a cell with an ELANE gene mutation associated with SCN or CyN may be from a subject with the ELANE gene mutation and/or afflicted with SCN or CyN. Accordingly, selecting a cell with an ELANE gene mutation may comprise selecting a subject with the ELA NE gene mutation. In further embodiments of the present invention, selecting a cell may comprise selecting a cell from a subject with the ELANE gene mutation. In embodiments of the present invention, introducing the compositions of the subject invention to the cell may comprise introducing the compositions of the invention to the cell of a subject afflicted with the ELANE gene mutation.

Accordingly, in embodiments of the present invention, there is provided a method for inactivating in a cell a mutant allele of the ELANE gene of a subject, the method comprising the step of selecting a subject with an ELANE gene mutation resulting in SCN or CyN and who is heterozygous at one or more polymorphic sites in the ELANE gene selected from the group consisting of: rs10414837, rs3761005, rs1683564, rs9749274, rs740021, rs201048029, rs199720952, rs28591229, rs71335276, rs58082177, rs3826946, rs10413889, rs761481944, rs3761008, rs10409474, rs3761007, rs17216649, rs10469327, rs8107095, rs10424470 and rs78302854n.

Accordingly, embodiments of the present invention encompass the screening of subjects or cells for the ELANE gene. A person having ordinary skill in the art would readily understand methods of screening for mutations within the ELANE gene in the art, by way of non-limiting examples, e.g., sequencing-by-synthesis, Sanger sequencing, karyotyping, Fluorescence In situ Hybridization, and/or microarray testing. In embodiments of the present invention, mutations within the ELANE gene are screened by exon sequencing.

In embodiments of the present invention, the subject is or has been diagnosed with SCN or CyN by measuring the absolute neutrophil count (ANC) in peripheral blood. In embodiments of the present invention, SCN is or was diagnosed before the subject reaches the age 6 months. In embodiments of the present invention, CyN is or was diagnosed between the ages of 12 and 24 months, or after the age of 24 months. In embodiments of the present invention, SCN or Cyn is diagnosed by one or more of recurrent acute stomatologic disorders. In embodiments of the present invention, SCN or CyN is diagnosed by bone marrow examination, preferably the bone marrow examination is a cytogenetic bone marrow study. In embodiments of the present invention, SCN or CyN is diagnosed by one or more of: antineutrophil antibody assay, immunoglobulin assay (Ig GAM), lymphocyte immunophenotyping, pancreatic markers (serum trypsinogen and fecal elastase) and liposoluble vitamin levels (vitamins A, E and D). It is understood that any diagnostic method may be used with any other diagnostic method.

In embodiments of the present invention, a subject diagnosed with SCN or CyN is screened by Exon sequencing to identify an ELANE pathogenic mutation in the ELANE gene. In further embodiments the subject is screened by Sanger sequencing to confirm heterozygocity of at least one SNP in Table 1. In embodiments of the present invention, the SNP is one of rs1683564, rs10414837, and rs3761005. In embodiments of the present invention, the nucleotide of the heterozygous SNP on the mutant allele of the ELANE gene determined using BAC bio. In embodiments of the present invention, appropriate guides are selected according to Table 2. In embodiments of the present invention, the guides selected are introduced to cells, e.g. PBMCs, obtained from the subject and reduction in the pathogenic ELANE mutation in the cells is measured by, e.g. Next Generation Sequencing.

It is understood that the CRISPR/Cas9 gene editing system enables targeting the nuclease to a target site in a sequence specific manner to address disease-causing mutations. Hematopoietic stem and progenitor cells (HSPCs) have therapeutic potential because of their ability to both self-renew and differentiate (Yu, Natanson, and Dunbar 2016). Accordingly, embodiments of the present invention apply genome editing to HSPCs.

In embodiments of the present invention, an autologous therapy and utilizes autologous CD34+ hematopoietic stem cells from patients diagnosed with SCN or CyN which are edited with CRISPR/Cas9. In embodiments of the present invention, CD34+ cells are isolated from bone marrow or peripheral blood mononucleated cells (PBMCs) following patient apheresis.

In the case of dominant negative (or compound heterozygous) indications, such as SCN or CyN, the strategy is to edit the mutant allele and avoid cleavage in the non-mutant allele or other off targets by targeting a heterozygous SNP sequence.

Embodiments of the present invention may include the following steps:
Selection of a patient diagnosed with SCN or CyN identified as exhibiting heterozygosity in at least one of the SNPs of Table 1 hereinbelow. In embodiments of the present invention, the subject is heterozygous at rs10414837, rs3761005, or rs1683564;
Selection of a therapeutic strategy based on the identified heterozygous SNP position of the candidate patient;
Obtaining HSPC cells from the bone marrow of the subject either by aspiration or by mobilization and apheresis of peripheral blood, optionally, the HSPC cells are processed (e.g., enriched, stimulated, both);
Introducing into the HSPC cells (e.g., by ex vivo electroporation) a composition comprising:
   a CRISPR nuclease or a sequence encoding the same (e.g., mRNA),
   a discriminatory RNA molecule that targets a particular sequence in the identified heterozygous SNP position of the mutant allele (REF/ALT sequence), and
   a non-discriminatory RNA molecule targeting a sequence in intron 3, intron 4 or 3′ UTR, which is common to both the mutant allele and the other allele,
   thereby editing the HSPC cells to knockout expression of mutant ELANE allele; and
Introducing the edited HSPC to the candidate patient.

In embodiments of the present invention, CD34+ cells may be isolated from bone marrow or peripheral blood mononucleated cells (PBMCs) following patient apheresis. Bone marrow or PBMCs may be collected from the patient by apheresis following HSPC mobilization. In embodiments of the invention the apheresis product may be washed to remove platelets and a CD34+ cell population may be enriched via purification using, e.g. a CliniMACS system (Miltenyi Biotec). In embodiments of the present invention, the selected cells may be prestimulated ex vivo, e.g. with a mixture of recombinant human cytokines. In embodiments of the present invention, the cells may undergo electroporation. In embodiments of the present invention, prior to electroporation, stimulated cells (e.g. CD34+ cells), the CRISPR nuclease mRNA and gRNA may be preincubated under defined conditions. In embodiments of the present invention, the cells are electroporated ex vivo with the CRISPR nuclease mRNA/gRNA mixture or with a preassembled RNPs (Ribonuclease protein of the CRISPR nuclease protein and gRNA), followed by cell washing. In embodiments of the present invention, the cells are suspended into a final formulation. In embodiments of the present invention, the cells may be resuspended. In embodiments of the present invention, the resuspended cells may be filled into bags for infusion. In embodiments of the present invention, the bags may be frozen using a freeze down step in a controlled rate freezer and/or stored in the vapor phase of liquid nitrogen. In embodiments of the present invention, the product may be administered by intravenous (1V) administration to a patient.

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, SCN or CyN. In some embodiments, the dominant genetic disorder is SCN or CyN. In some embodiments, the target gene is the ELANE gene (Entrez Gene, gene ID No: 335).

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising at least one nucleotide which differs between a mutant allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutant allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

In embodiments of the present invention, a type II CRISPR system utilizes a mature crRNA:tracrRNA complex directs a CRISPR nuclease, e.g. Cas9, to the target DNA via Watson-Crick base-pairing between the crRNA and the protospacer on the target DNA next to the protospacer adjacent motif (PAM), an additional requirement for target recognition. The CRISPR nuclease then mediates cleavage of target DNA to create a double-stranded break within the protospacer. A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTTV for Cpf1. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015-0211023, incorporated herein by reference.

CRISPR systems that may be used in the practice of the invention vary greatly. CRISPR systems can be a type I, a type II, type 111, or type V system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas12a, Cas12b, Cas12c, Cas12d, Cas12d, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966. (See, e.g., Koonin 2017).

In some embodiments, the RNA-guided DNA nuclease is a CRISPR nuclease derived from a type IT CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium dificile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina. Francisella cf novicida Fx1, Alicyclobacillus acidoterrestris, Oleiphilus sp., Bacterium CG09_39_24, Deltaproteobacteria bacterium, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., spCas9 D1135E variant, spCas9 VQR variant, spCas9 EQR variant, or spCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al. (2015) Cell.).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to one or more of: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-nethylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-O-methyl (M), 3′-phosphorothioate (MS), 3′-thioPACE (MSP), pseudouridine, or 1-methyl pseudo-uridine. Each possibility represents a separate embodiment of the present invention.

Further non-limiting examples of suitable chemical modifications include: m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladeno sine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); mt⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2G (N²,N²-dimethylguanosine); m²Gm (N^{2,2}′-O-dimethylguanosine); m²₂Gm (N²,N², 2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqucuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s2U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmmm⁵s²U (5-carboxymethylaminomethyl-2-thiouridine); dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m²¹⁷G (N²,7-dimethylguanosine); m2,2,7G (N²,N2,7-trimethylguanosine); m³Um (3,2′-O-dimethyluridine); m⁵D (5-methyldihydrouridine); f⁵Cm (5-formyl-2′-O-methylcytidine); m¹ Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine). Each possibility represents a separate embodiment of the present invention. (See e.g. U.S. Pat. No. 9,750,824).

Guide Sequences which Specifically Target a Mutant Allele

A given gene may contain thousands of SNPs. Utilizing a 24 base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated protein, inactivating a mutant ELANE gene allele, and treating SCN or CyN.

The present disclosure provides guide sequences capable of specifically targeting a mutant allele for inactivation while leaving the functional allele unmodified. The guide sequences of the present invention are designed to, and are most likely to, specifically differentiate between a mutant allele and a functional allele. Of all possible guide sequences which target a mutant allele desired to be inactivated, the specific guide sequences disclosed herein are specifically effective to function with the disclosed embodiments.

Briefly, the guide sequences may have properties as follows: (1) target a heterozygous SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutant allele: (1) Knockout strategy using one RNA molecule—one RNA molecule is utilized to direct a CRISPR nuclease to a mutant allele and create a double-strand break (DSB) leading to formation of a frameshift mutation in an exon or in a splice site region of the mutant allele; (2) Knockout strategy using two RNA molecules—two RNA molecules are utilized. A first RNA molecule targets a region in the promoter or an upstream region of a mutant allele and another RNA molecule targets downstream of the first RNA molecule in a promoter, exon, or intron of the mutant allele; (3) Exon(s) skipping strategy—one RNA molecule may be used to target a CRISPR nuclease to a splice site region, either at the 5′end of an intron (donor sequence) or the 3′ end of an intron (acceptor sequence), in order to destroy the splice site. Alternatively, two RNA molecules may be utilized such that a first RNA molecule targets an upstream region of an exon and a second RNA molecule targets a region downstream of the first RNA molecule, thereby excising the exon(s). Based on the locations of identified SNPs/insertions/deletions/indels for each mutant allele, any one of, or a combination of, the above-mentioned methods to deactivate the mutant allele may be utilized.

When only one RNA molecule is used is that the location of the SNP is in an exon or in close proximity (e.g., within 20 basepairs) to a splice site between the intron and the exon. When two RNA molecules are used, guide sequences may target two SNPs such that the first SNP is upstream of exon 1 e.g., within the 5′ untranslated region, or within the promoter or within the first 2 kilobases 5′ of the transcription start site, and the second SNP is downstream of the first SNP e.g., within the first 2 kilobases 5′ of the transcription start site, or within intron 1, 2 or 3, or within exon 1, exon 2, or exon 3.

Guide sequences of the present invention may target a SNP in the upstream portion of the targeted gene, preferably upstream of the last exon of the targeted gene. Guide sequences may target a SNP upstream to exon 1, for example within the 5′ untranslated region, or within the promoter or within the first 4-5 kilobases 5′ of the transcription start site.

Guide sequences of the present invention may also target a SNP within close proximity (e.g., within 50 basepairs, more preferably with 20 basepairs) to a known protospacer adjacent motif (PAM) site.

Guide sequences of the present invention also may target: (1) a heterozygous SNP for the targeted gene; (2) a heterozygous SNPs upstream and downstream of the gene; (3) a SNPs with a prevalence of the SNP/insertion/deletion/indel in the general population, in a specific ethnic population, or in a patient population above 1%; (4) have a guanine-cytosine content of greater than 30% and less than 85%; (5) have no repeat of 4 or more thymine/uracil or 8 or more guanine, cytosine, or adenine; (6) having no off-target identified by off-target analysis; and (7) preferably target Exons over Introns or be upstream of a SNP rather than downstream of a SNP.

In embodiments of the present invention, the SNP may be upstream or downstream of the gene. In embodiments of the present invention, the SNP is within 4,000 base pairs upstream or downstream of the gene.

The at least one nucleotide which differs between the mutant allele and the functional allele, may be upstream, downstream or within the sequence of the disease-causing mutation of the gene of interest. The at least one nucleotide which differs between the mutant allele and the functional allele, may be within an exon or within an intron of the gene of interest. In some embodiments, the at least one nucleotide which differs between the mutant allele and the functional allele is within an exon of the gene of interest. In some embodiments, the at least one nucleotide which differs between the mutant allele and the functional allele is within an intron or an exon of the gene of interest, in close proximity to a splice site between the intron and the exon e.g., 1, 2, 3, 4, 5, 6, 7, 8, 9, 10, 11, 12, 13, 14, 15, 16, 17, 18, 19, or 20 nucleotides upstream or downstream to the splice site.

In some embodiments, the at least one nucleotide is a single nucleotide polymorphisms (SNPs). In some embodiments, each of the nucleotide variants of the SNP may be expressed in the mutant allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutant allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutant allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

Guide sequences of the present invention may satisfy any one of the above criteria and are most likely to differentiate between a mutant allele from its corresponding functional allele.

In some embodiments the RNA molecule targets a heterozygous SNP present in the ELANE gene from the SNPs as shown in Table 1 below. The SNP details are indicated in the 1^{st} column and include: SNP ID No. (based on NCBI's 2018 database of Single Nucleotide Polymorphisms (dbSNP)). For variants with no available rs number variants characteristic are indicated based on gnomAD 2018 browser database. The 2^{nd} column indicates an assigned identifier for each SNP. The 3^{rd} column indicates the location of each SNP on the ELANE gene.

TABLE 1
ELANE gene SNPs

| RSID | SNP No. | SNP location in the gene |
|---|---|---|
| rs9749274 | s1 | downstream +972 bp |
| rs740021 | s2 | upstream −198 bp |
| rs201048029 | s3 | upstream −2614 bp |
| rs199720952 | s4 | downstream +173 bp |
| rs28591229 | s5 | downstream +2053 bp |
| rs71335276 | s6 | downstream +3125 bp |
| rs58082177 | s7 | upstream −2840 bp |
| rs3826946 | s8 | upstream −2103 bp |
| rs10413889 | s9 | upstream −2003 bp |
| rs3761005 | s10 | upstream −1509 bp |
| rs761481944 | s11 | downstream +2824 bp |
| rs3761008 | s12 | upstream −2279 bp |
| rs10409474 | s13 | upstream −1569 bp |
| rs3761007 | s14 | upstream −1728 bp |
| rs1683564 | s15 | downstream +2971 bp |
| rs17216649 | s16 | Exon_5 of 5 |
| rs10469327 | s17 | downstream +2133 bp |
| rs8107095 | s18 | downstream +3588 bp |
| rs10414837 | s19 | upstream −2684 bp |
| rs10424470 | s20 | upstream −3504 bp |
| rs78302854 | s21 | downstream +862 bp |

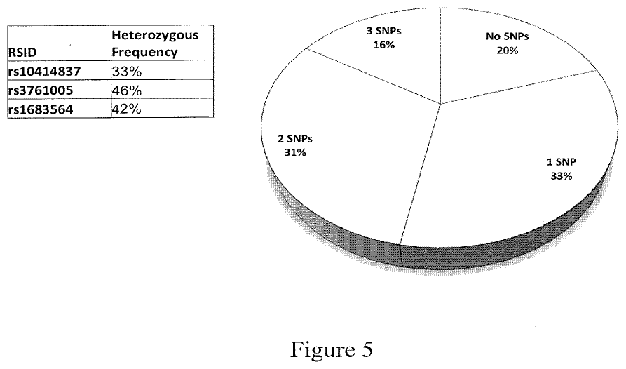

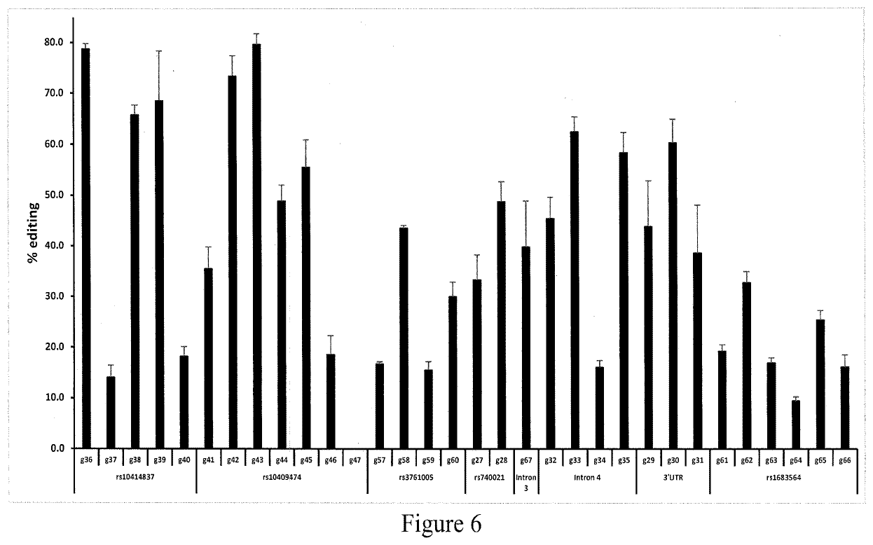

FIG. 5 discloses the heterogenicity of given selections of SNPs from Table 1 in the human population.

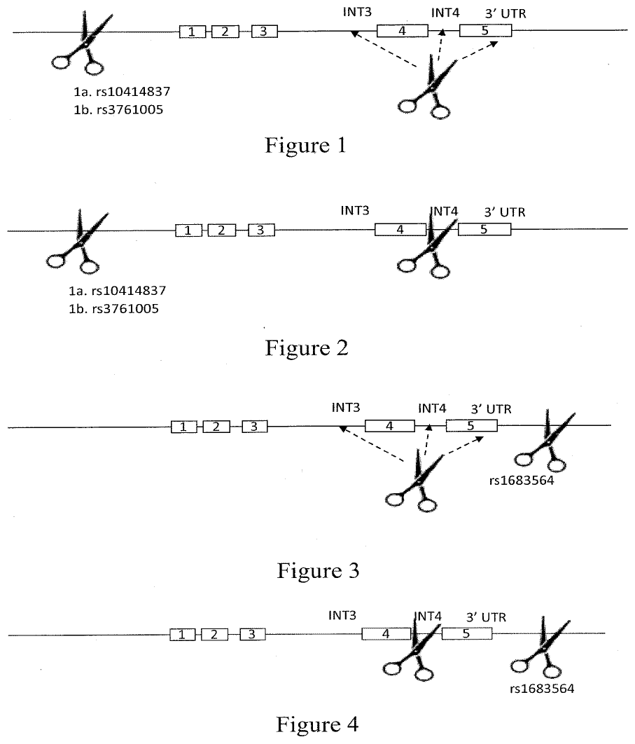

Embodiments of the present invention may include excising the promoter region from an upstream SNP position until intron 3 or intron 4 or the 3′ UTR. In an embodiment, a first guide sequence targets a specific sequence of a heterozygous SNP position in an upstream region of the mutant allele (strategy 1a—rs10414837, strategy 1b—rs3761005) and a second guide sequence targets a sequence in intron 4 which is common to two alleles of the gene. (FIG. 1). In a further embodiment, a first guide sequence targets a specific sequence of a heterozygous SNP position in an upstream region of the mutant allele (strategy 1a—rs10414837, strategy 1b—rs3761005) and a second guide sequence targets a sequence in intron 3 which is common to two alleles of the gene. In another, a first guide sequence targets a specific sequence of a heterozygous SNP position in an upstream region of the mutant allele (strategy 1a—rs10414837, strategy 1b—rs3761005) and a second guide sequence targets a sequence in 3′ UTR which is common to two alleles of the gene. (FIG. 2).

Embodiments of the present invention may include excising from intron 3 or intron 4 or 3′ UTR to regions downstream to the 3′ UTR. In an embodiment, a first guide sequence targets a specific sequence of a heterozygous SNP position in an upstream region of the mutant allele (strategy 2—rs1683564) and a second guide sequence targets a sequence in intron 4 which is common to two alleles of the gene. In a further embodiment, a first guide sequence targets a specific sequence of a heterozygous SNP position in an upstream region of the mutant allele (strategy 2—rs1683564) and a second guide sequence targets a sequence in intron 3 which is common to two alleles of the gene. In a further, a first guide sequence targets a specific sequence of a heterozygous SNP position in an upstream region of the mutant allele (strategy 2—rs1683564) and a second guide sequence targets a sequence in 3′ UTR which is common to two alleles of the gene. (FIG. 3).

Embodiments of the present invention excising from intron 3 or intron 4 or 3′ UTR to regions downstream to the 3′ UTR. The strategy is designed such as to specifically knock-out the disease-causing allele (‘mutant allele’), while leaving the healthy allele intact. Allele specific editing is achieved by using guides that target discriminating (heterozygous) SNP positions with relatively high heterozygosity frequency in the population. (FIG. 4).

### Delivery to Cells

It is understood that in the methods embodied, the RNA molecules and compositions described herein may be delivered to a target cell or subject by any suitable means. The following embodiments provide non-limiting examples of methods of delivery of the RNA molecules and composition of the present invention.

In some embodiments, RNA molecule compositions of the present invention may be targeted to any cell which contains and/or expresses a dominant negative allele, including any mammalian or plant cell. For example, in one embodiment the RNA molecule specifically targets a mutant ELANE allele and the target cell is a hepatocyte cell.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada et al. (1995) in Current Topics in Microbiology and Immunology Doerfler and Bohm (eds.); and Yu et al. (1994) Gene Therapy 1:13-26.

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizobium meliloti, Mesorhizobium loti, tobacco mosaic virus, potato virus X, cauliflower mosaic virus and cassava vein mosaic virus). (See, e.g., Chung et al. (2006) Trends Plant Sci. 11(1):1-4). Sonoporation using, e.g., the Sonitron 2000 system (Rich-Mar), can also be used for delivery of nucleic acids. Cationic-lipid mediated delivery of proteins and/or nucleic acids is also contemplated as an in vivo or in vitro delivery method. (See Zuris et al. (2015) Nat. Biotechnol. 33(1):73-80; see also Coelho et al. (2013) N. Engl. J. Med. 369, 819-829; Judge et al. (2006) Mol. Ther. 13, 494-505; and Basha et al. (2011) Mol. Ther. 19, 2186-2200).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al (2009) Nature Biotechnology 27(7):643).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; PCT/US94/05700).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94:22 12133-12138). PA317/pLASN was the first therapeutic vector used in a gene therapy trial. (Blaese et al. (1995). Transduction efficiencies of 50% or greater have been observed for MFG-S packaged vectors. (Ellem et al. (1997) Immunol Immunother. 44(1):10-20; Dranoff et al. (1997) Hum. Gene Ther. 1:111-2).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney et al. (1994) Culture of Animal Cells, A Manual of Basic Technique, 3rd ed, and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-K1, CHO-DG44, CHO-DUXB1l, CHO-DUKX, CHOK1SV), VERO, MDCK, W138, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodoptera frugiperda (Sf), or fungal cells such as Saccharomyces, Pichia and Schizosaccharomyces. In certain embodiments, the cell line is a CHO-K 1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), OR-1 (granulocytes), and lad (differentiated antigen presenting cells) (as a non-limiting example see Inaba et al. (1992) J. Exp. Med. 176:1693-1702). Stem cells that have been modified may also be used in some embodiments.

Typically, the cells are administered in a pharmaceutical composition comprising at least one pharmaceutically-acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically-acceptable material, composition or vehicle, such as a liquid or solid filler, diluent, excipient, or solvent encapsulating material.

Any one of the RNA molecule compositions described herein is suitable for genome editing in post-mitotic cells or any cell which is not actively dividing, e.g., arrested cells. Examples of post-mitotic cells which may be edited using an RNA molecule composition of the present invention include, but are not limited to, a hepatocyte cell.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009-0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between a mutant allele and a functional allele (e.g., SNP) of a gene of interest (i.e., a sequence of the mutant allele which is not present in the functional allele). The sequence may be within the disease associated mutation. The sequence may be upstream or downstream to the disease associated mutation. Any sequence difference between the mutant allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for Several Embodiments Disclosed Herein

Mutations in ELANE that were demonstrated to lead to SCN or CyN, mediate translation from alternative in frame ORF (open reading frame) that generate truncated N-terminus protein thus causing ER and protein misfolding stress.

Without being bound by any theory or mechanism, the instant invention may be utilized to apply a CRISPR nuclease to process the mutated pathologic ELANE allele and not the functional ELANE allele, such as to prevent expression of the mutated pathologic allele or to produce a truncated non-pathologic peptide from the mutated pathologic allele, or to repair/correct the mutated pathologic ELANE allele in order to prevent ameliorate or treat SCN or CyN.

Several alternative editing strategies utilizing SNPs located upstream and downstream to the ORF may be applied. The strategies include exclusion of the whole gene, truncation of the gene to exclude the C-terminus of the gene, and attenuation of the expression of the gene.

In some embodiments, two guides (e.g., guides disclosed in Table 2) may be utilized to remove the entire gene (i.e., exons 1, 2, 3, 4, and 5) to knockout the mutant protein. In some embodiments, a first guide RNA is utilized to mediate an allele specific DSB by targeting a SNP/WT sequence located upstream to the ORF of the mutated allele of the ELANE gene, and a second guide RNA may be utilized to mediate DSB in a SNP/WT sequence located in exon 5 or downstream to the mutated allele of the ELANE gene, or a sequence located in intron 4, 3′ UTR or downstream to the alleles of the ELANE gene, or a SNP/WT sequence located in intron 4 3′UTR or downstream to the alleles of the ELANE gene.

There are records of healthy individuals harboring frameshift mutation that result in gain of stop codon located till exon 3. Therefore, a potential strategy may be to truncate the mutated allele such that to include at most exons 1 till 3. In some embodiments, two guides (e.g., guides disclosed in Table 2) may be utilized to truncate the c-terminus of the mutated allele of the ELANE gene. In some embodiments, a first guide RNA may be utilized to mediate an allele specific DSB by targeting a SNP/WT sequence in exon 5 or downstream of the mutated allele, and a second guide RNA may be utilized to mediate DSB in a sequence located in intron 1, 2 or 3 of the ELANE gene, or a SNP/WT sequence. A peptide/protein encoded by the truncated mutated allele may exhibit no pathological effect. Alternatively, a nonsense-mediated mRNA decay may be triggered resulting in knockout of the expression of the mutated allele. Results may be verified by examining mRNA and protein expression.

In some embodiments, the expression of the mutated allele may be attenuated by excising elements from the proximal promoter and enhancer regions using the SNPs located upstream to the ORF. In a non-limiting example, a significant reduction may be achieved by excising most of the enhancer region by targeting a SNP.

Examples of RNA Guide Sequences which Specifically Target Mutant Alleles of ELANE Gene

Although a large number of guide sequences can be designed to target a mutant allele, the nucleotide sequences described in Tables 2 identified by SEQ ID NOs: 1-1192 below were specifically selected to effectively implement the methods set forth herein and to effectively discriminate between alleles.

Referring to columns 1-4, each of SEQ ID NOs: 1-1192 indicated in column 1 corresponds to an engineered guide sequence. The corresponding SNP details are indicated in column 2. The SNP details indicated in the 2nd column include the assigned identifier for each SNP corresponding to a SNP ID indicated in Table 1. Column 3 indicates whether the target of each guide sequence is the ELANE gene polymorph or wild type sequence where indicated. Column 4 indicates the guanine-cytosine content of each guide sequence where indicated.

Table 2 shows guide sequences designed for use as described in the embodiments above to associate with different SNPs within a sequence of a mutant ELANE allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 2
Guide sequences designed to associate
with specific SNPs of the ELANE gene

| SEQ ID NO: | SNP ID (Table 1) | Target (ALT/REF) | % GC |
|---|---|---|---|
| 1 | s1 | REF | 35% |
| 2 | s1 | ALT | 30% |
| 3 | s1 | REF | 35% |
| 4 | s1 | ALT | 30% |
| 5 | s1 | REF | 40% |
| 6 | s2 | BOTH | 30% |
| 7 | s3 | BOTH | 30% |
| 8 | s1 | ALT | 35% |
| 9 | s1 | REF | 40% |
| 10 | s2 | REF | 30% |
| 11 | s4 | BOTH | 45% |
| 12 | s3 | BOTH | 30% |
| 13 | s5 | ALT | 55% |
| 14 | s5 | REF | 60% |
| 15 | s1 | ALT | 35% |
| 16 | s1 | REF | 45% |
| 17 | s6 | REF | 60% |
| 18 | s6 | ALT | 55% |
| 19 | s2 | BOTH | 35% |
| 20 | s7 | ALT | 30% |
| 21 | s8 | ALT | 45% |
| 22 | s9 | ALT | 55% |
| 23 | s9 | REF | 60% |
| 24 | s5 | ALT | 60% |
| 25 | s5 | REF | 65% |
| 26 | s10 | REF | 55% |
| 27 | s11 | REF | 55% |
| 28 | s1 | ALT | 40% |
| 29 | s12 | ALT | 30% |
| 30 | s2 | ALT | 35% |
| 31 | s1 | REF | 45% |
| 32 | s6 | REF | 65% |
| 33 | s6 | ALT | 60% |
| 34 | s13 | REF | 60% |
| 35 | s13 | ALT | 60% |
| 36 | s2 | BOTH | 35% |
| 37 | s4 | BOTH | 45% |
| 38 | s6 | ALT | 50% |
| 39 | s12 | ALT | 30% |
| 40 | s1 | REF | 30% |
| 41 | s3 | BOTH | 35% |
| 42 | s12 | ALT | 35% |
| 43 | s12 | REF | 30% |
| 44 | s14 | ALT | 65% |
| 45 | s8 | ALT | 45% |
| 46 | s5 | ALT | 50% |
| 47 | s5 | REF | 55% |
| 48 | s9 | ALT | 60% |
| 49 | s9 | REF | 65% |
| 50 | s5 | ALT | 60% |
| 51 | s11 | REF | 60% |
| 52 | s15 | REF | 70% |
| 53 | s9 | ALT | 45% |
| 54 | s9 | REF | 50% |
| 55 | s5 | REF | 65% |
| 56 | s11 | REF | 55% |
| 57 | s10 | REF | 60% |
| 58 | s16 | REF | 75% |
| 59 | s1 | BOTH | 45% |
| 60 | s11 | REF | 55% |
| 61 | s8 | ALT | 40% |
| 62 | s8 | REF | 40% |
| 63 | s1 | ALT | 40% |
| 64 | s17 | REF | 60% |
| 65 | s10 | ALT | 60% |
| 66 | s10 | REF | 60% |
| 67 | s2 | REF | 35% |
| 68 | s2 | ALT | 30% |
| 69 | s8 | REF | 45% |
| 70 | s10 | ALT | 55% |
| 71 | s2 | REF | 30% |
| 72 | s12 | REF | 30% |
| 73 | s12 | ALT | 35% |
| 74 | s2 | ALT | 35% |
| 75 | s2 | REF | 35% |
| 76 | s2 | ALT | 30% |
| 77 | s1 | REF | 50% |
| 78 | s7 | ALT | 35% |
| 79 | s6 | ALT | 65% |
| 80 | s6 | REF | 70% |
| 81 | s18 | ALT | 70% |
| 82 | s19 | REF | 55% |
| 83 | s6 | REF | 70% |
| 84 | s6 | ALT | 65% |
| 85 | s11 | REF | 60% |
| 86 | s19 | ALT | 50% |
| 87 | s15 | REF | 50% |
| 88 | s5 | REF | 65% |
| 89 | s5 | ALT | 60% |
| 90 | s18 | BOTH | 70% |
| 91 | s8 | ALT | 35% |
| 92 | s8 | REF | 35% |
| 93 | s19 | ALT | 55% |
| 94 | s19 | REF | 60% |
| 95 | s9 | ALT | 70% |
| 96 | s16 | REF | 75% |
| 97 | s13 | REF | 60% |
| 98 | s13 | ALT | 60% |
| 99 | s8 | REF | 45% |
| 100 | s8 | ALT | 45% |
| 101 | s2 | REF | 40% |
| 102 | s16 | REF | 70% |
| 103 | s16 | ALT | 65% |
| 104 | s6 | ALT | 60% |
| 105 | s6 | REF | 65% |
| 106 | s20 | REF | 65% |
| 107 | s13 | ALT | 55% |
| 108 | s2 | ALT | 35% |
| 109 | s2 | REF | 40% |
| 110 | s6 | ALT | 65% |
| 111 | s6 | REF | 70% |
| 112 | s6 | ALT | 50% |
| 113 | s4 | BOTH | 45% |
| 114 | s18 | ALT | 70% |
| 115 | s19 | REF | 55% |
| 116 | s6 | REF | 70% |
| 117 | s6 | ALT | 65% |
| 118 | s11 | REF | 60% |
| 119 | s19 | ALT | 50% |
| 120 | s18 | REF | 70% |
| 121 | s18 | ALT | 75% |
| 122 | s15 | REF | 50% |
| 123 | s12 | ALT | 30% |
| 124 | s5 | REF | 60% |
| 125 | s5 | ALT | 55% |
| 126 | s13 | REF | 55% |
| 127 | s9 | REF | 55% |
| 128 | s9 | ALT | 50% |
| 129 | s18 | BOTH | 75% |
| 130 | s9 | REF | 75% |
| 131 | s18 | ALT | 75% |
| 132 | s18 | REF | 70% |
| 133 | s14 | ALT | 60% |
| 134 | s14 | REF | 65% |
| 135 | s19 | BOTH | 65% |
| 136 | s8 | REF | 35% |
| 137 | s8 | ALT | 35% |
| 138 | s12 | REF | 35% |
| 139 | s12 | ALT | 40% |
| 140 | s17 | BOTH | 40% |
| 141 | s8 | BOTH | 45% |
| 142 | s16 | REF | 70% |
| 143 | s8 | ALT | 35% |
| 144 | s8 | REF | 35% |
| 145 | s6 | BOTH | 55% |
| 146 | s1 | ALT | 35% |
| 147 | s1 | REF | 40% |
| 148 | s14 | BOTH | 55% |
| 149 | s12 | ALT | 30% |
| 150 | s12 | BOTH | 30% |
| 151 | s1 | REF | 35% |
| 152 | s5 | ALT | 55% |
| 153 | s5 | REF | 60% |
| 154 | s3 | BOTH | 40% |
| 155 | s12 | ALT | 35% |
| 156 | s12 | REF | 30% |
| 157 | s1 | BOTH | 45% |
| 158 | s16 | REF | 70% |
| 159 | s14 | ALT | 70% |
| 160 | s8 | ALT | 45% |
| 161 | s5 | ALT | 55% |
| 162 | s5 | REF | 60% |
| 163 | s14 | BOTH | 70% |
| 164 | s15 | REF | 60% |
| 165 | s6 | ALT | 55% |
| 166 | s6 | REF | 60% |
| 167 | s9 | ALT | 65% |
| 168 | s9 | REF | 70% |
| 169 | s5 | REF | 70% |
| 170 | s5 | ALT | 65% |
| 171 | s5 | BOTH | 65% |
| 172 | s5 | ALT | 65% |
| 173 | s19 | ALT | 60% |
| 174 | s11 | REF | 65% |
| 175 | s15 | ALT | 65% |
| 176 | s9 | ALT | 50% |
| 177 | s9 | REF | 55% |
| 178 | s5 | REF | 70% |
| 179 | s14 | REF | 70% |
| 180 | s19 | REF | 65% |
| 181 | s9 | ALT | 65% |
| 182 | s8 | BOTH | 45% |
| 183 | s12 | ALT | 40% |
| 184 | s12 | REF | 35% |
| 185 | s20 | ALT | 55% |
| 186 | s20 | ALT | 70% |
| 187 | s10 | REF | 60% |
| 188 | s10 | ALT | 60% |
| 189 | s13 | REF | 60% |
| 190 | s13 | ALT | 60% |
| 191 | s10 | BOTH | 70% |
| 192 | s14 | REF | 70% |
| 193 | s20 | ALT | 60% |
| 194 | s20 | REF | 55% |
| 195 | s12 | ALT | 30% |
| 196 | s2 | BOTH | 45% |
| 197 | s6 | BOTH | 50% |
| 198 | s16 | ALT | 65% |
| 199 | s15 | REF | 60% |
| 200 | s11 | REF | 55% |
| 201 | s14 | REF | 60% |
| 202 | s20 | REF | 65% |
| 203 | s17 | ALT | 60% |
| 204 | s20 | ALT | 70% |
| 205 | s11 | REF | 60% |
| 206 | s10 | REF | 65% |
| 207 | s20 | REF | 60% |
| 208 | s18 | REF | 75% |
| 209 | s5 | ALT | 65% |
| 210 | s20 | ALT | 70% |
| 211 | s9 | REF | 70% |
| 212 | s9 | ALT | 65% |
| 213 | s19 | BOTH | 55% |
| 214 | s15 | BOTH | 65% |
| 215 | s19 | ALT | 65% |
| 216 | s5 | REF | 60% |
| 217 | s5 | ALT | 55% |
| 218 | s6 | ALT | 55% |
| 219 | s8 | REF | 40% |
| 220 | s10 | REF | 65% |
| 221 | s10 | ALT | 65% |
| 222 | s6 | REF | 60% |
| 223 | s14 | REF | 60% |
| 224 | s19 | REF | 60% |
| 225 | s19 | ALT | 55% |
| 226 | s14 | ALT | 55% |
| 227 | s8 | ALT | 40% |
| 228 | s1 | ALT | 45% |
| 229 | s1 | REF | 50% |
| 230 | s11 | REF | 55% |
| 231 | s20 | REF | 50% |
| 232 | s8 | ALT | 40% |
| 233 | s12 | ALT | 30% |
| 234 | s8 | REF | 40% |
| 235 | s1 | ALT | 45% |
| 236 | s9 | ALT | 65% |
| 237 | s9 | REF | 70% |
| 238 | s8 | ALT | 40% |
| 239 | s11 | REF | 40% |
| 240 | s5 | BOTH | 40% |
| 241 | s17 | ALT | 60% |
| 242 | s17 | REF | 65% |
| 243 | s10 | ALT | 65% |
| 244 | s17 | BOTH | 60% |
| 245 | s10 | REF | 65% |
| 246 | s17 | REF | 65% |
| 247 | s17 | ALT | 60% |
| 248 | s2 | REF | 35% |
| 249 | s13 | REF | 60% |
| 250 | s13 | ALT | 55% |
| 251 | s13 | REF | 55% |
| 252 | s2 | ALT | 30% |
| 253 | s2 | REF | 35% |
| 254 | s12 | ALT | 30% |
| 255 | s8 | REF | 45% |
| 256 | s2 | ALT | 35% |
| 257 | s11 | ALT | 65% |
| 258 | s20 | REF | 50% |
| 259 | s20 | ALT | 55% |
| 260 | s11 | REF | 45% |
| 261 | s10 | ALT | 60% |
| 262 | s19 | ALT | 50% |
| 263 | s19 | REF | 55% |
| 264 | s5 | REF | 50% |
| 265 | s17 | ALT | 60% |
| 266 | s17 | REF | 65% |
| 267 | s2 | REF | 35% |
| 268 | s11 | REF | 60% |
| 269 | s17 | ALT | 45% |
| 270 | s17 | REF | 50% |
| 271 | s2 | REF | 30% |
| 272 | s8 | REF | 45% |
| 273 | s12 | REF | 30% |
| 274 | s12 | ALT | 35% |
| 275 | s10 | ALT | 65% |
| 276 | s8 | REF | 40% |
| 277 | s8 | ALT | 40% |
| 278 | s20 | ALT | 45% |
| 279 | s6 | REF | 55% |
| 280 | s6 | ALT | 50% |
| 281 | s2 | ALT | 30% |
| 282 | s9 | BOTH | 65% |
| 283 | s4 | REF | 30% |
| 284 | s4 | BOTH | 50% |
| 285 | s9 | ALT | 55% |
| 286 | s9 | REF | 60% |
| 287 | s2 | REF | 30% |
| 288 | s6 | ALT | 55% |
| 289 | s11 | REF | 65% |
| 290 | s15 | REF | 70% |
| 291 | s9 | REF | 50% |
| 292 | s16 | REF | 75% |
| 293 | s2 | REF | 40% |
| 294 | s2 | ALT | 35% |
| 295 | s2 | REF | 40% |
| 296 | s2 | ALT | 35% |
| 297 | s1 | REF | 50% |
| 298 | s7 | ALT | 35% |
| 299 | s6 | ALT | 65% |
| 300 | s6 | REF | 70% |
| 301 | s11 | REF | 60% |
| 302 | s5 | REF | 70% |
| 303 | s5 | ALT | 65% |
| 304 | s18 | BOTH | 70% |
| 305 | s8 | ALT | 35% |
| 306 | s8 | REF | 35% |
| 307 | s19 | ALT | 60% |
| 308 | s19 | REF | 65% |
| 309 | s7 | ALT | 30% |
| 310 | s16 | REF | 75% |
| 311 | s2 | REF | 30% |
| 312 | s6 | ALT | 65% |
| 313 | s6 | REF | 70% |
| 314 | s13 | ALT | 55% |
| 315 | s6 | ALT | 65% |
| 316 | s6 | REF | 70% |
| 317 | s6 | ALT | 50% |
| 318 | s18 | REF | 70% |
| 319 | s19 | REF | 55% |
| 320 | s6 | REF | 70% |
| 321 | s6 | ALT | 65% |
| 322 | s11 | REF | 65% |
| 323 | s19 | ALT | 50% |
| 324 | s18 | REF | 70% |
| 325 | s18 | ALT | 75% |
| 326 | s15 | BOTH | 55% |
| 327 | s5 | REF | 65% |
| 328 | s5 | ALT | 60% |
| 329 | s13 | REF | 55% |
| 330 | s18 | BOTH | 75% |
| 331 | s14 | ALT | 60% |
| 332 | s14 | REF | 65% |
| 333 | s8 | REF | 40% |
| 334 | s8 | ALT | 40% |
| 335 | s12 | REF | 35% |
| 336 | s12 | ALT | 40% |
| 337 | s17 | BOTH | 45% |
| 338 | s8 | ALT | 40% |
| 339 | s8 | REF | 40% |
| 340 | s14 | BOTH | 60% |
| 341 | s12 | BOTH | 35% |
| 342 | s1 | BOTH | 50% |
| 343 | s16 | REF | 70% |
| 344 | s16 | ALT | 65% |
| 345 | s15 | ALT | 60% |
| 346 | s15 | REF | 65% |
| 347 | s6 | ALT | 55% |
| 348 | s6 | REF | 60% |
| 349 | s5 | REF | 70% |
| 350 | s5 | ALT | 65% |
| 351 | s19 | ALT | 60% |
| 352 | s19 | REF | 65% |
| 353 | s9 | ALT | 70% |
| 354 | s8 | BOTH | 50% |
| 355 | s3 | BOTH | 45% |
| 356 | s10 | REF | 60% |
| 357 | s10 | ALT | 60% |
| 358 | s13 | REF | 65% |
| 359 | s13 | ALT | 65% |
| 360 | s14 | REF | 70% |
| 361 | s20 | ALT | 60% |
| 362 | s20 | REF | 55% |
| 363 | s14 | REF | 65% |
| 364 | s14 | ALT | 60% |
| 365 | s20 | REF | 70% |
| 366 | s17 | ALT | 65% |
| 367 | s20 | ALT | 75% |
| 368 | s11 | REF | 65% |
| 369 | s20 | REF | 65% |
| 370 | s5 | ALT | 65% |
| 371 | s9 | REF | 75% |
| 372 | s9 | ALT | 70% |
| 373 | s15 | BOTH | 65% |
| 374 | s19 | ALT | 65% |
| 375 | s8 | REF | 40% |
| 376 | s8 | ALT | 40% |
| 377 | s12 | REF | 30% |
| 378 | s17 | REF | 65% |
| 379 | s17 | ALT | 60% |
| 380 | s13 | REF | 60% |
| 381 | s13 | ALT | 60% |
| 382 | s13 | REF | 60% |
| 383 | s20 | REF | 50% |
| 384 | s20 | ALT | 55% |
| 385 | s5 | REF | 50% |
| 386 | s17 | ALT | 60% |
| 387 | s17 | REF | 65% |
| 388 | s13 | ALT | 60% |
| 389 | s17 | ALT | 50% |
| 390 | s17 | REF | 55% |
| 391 | s8 | REF | 45% |
| 392 | s10 | ALT | 65% |
| 393 | s8 | REF | 45% |
| 394 | s8 | ALT | 45% |
| 395 | s2 | REF | 40% |
| 396 | s9 | ALT | 60% |
| 397 | s9 | REF | 65% |
| 398 | s6 | ALT | 55% |
| 399 | s9 | BOTH | 50% |
| 400 | s16 | REF | 75% |
| 401 | s6 | ALT | 65% |
| 402 | s6 | REF | 70% |
| 403 | s11 | REF | 60% |
| 404 | s15 | REF | 55% |
| 405 | s16 | REF | 75% |
| 406 | s2 | ALT | 30% |
| 407 | s2 | REF | 35% |
| 408 | s13 | ALT | 55% |
| 409 | s18 | REF | 75% |
| 410 | s13 | REF | 55% |
| 411 | s14 | BOTH | 60% |
| 412 | s15 | ALT | 60% |
| 413 | s6 | ALT | 60% |
| 414 | s6 | REF | 65% |
| 415 | s8 | BOTH | 55% |
| 416 | s3 | BOTH | 50% |
| 417 | s10 | REF | 60% |
| 418 | s10 | ALT | 60% |
| 419 | s14 | REF | 65% |
| 420 | s14 | ALT | 60% |
| 421 | s20 | REF | 70% |
| 422 | s17 | REF | 65% |
| 423 | s17 | ALT | 60% |
| 424 | s13 | ALT | 60% |
| 425 | s13 | REF | 60% |
| 426 | s5 | ALT | 45% |
| 427 | s5 | REF | 50% |
| 428 | s17 | ALT | 60% |
| 429 | s17 | REF | 65% |
| 430 | s13 | ALT | 60% |
| 431 | s10 | ALT | 65% |
| 432 | s2 | REF | 40% |
| 433 | s13 | ALT | 60% |
| 434 | s18 | REF | 75% |
| 435 | s13 | REF | 60% |
| 436 | s15 | ALT | 65% |
| 437 | s15 | REF | 70% |
| 438 | s6 | ALT | 65% |
| 439 | s6 | REF | 70% |
| 440 | s8 | BOTH | 60% |
| 441 | s10 | REF | 65% |
| 442 | s10 | ALT | 65% |
| 443 | s17 | REF | 70% |
| 444 | s17 | ALT | 65% |
| 445 | s13 | ALT | 60% |
| 446 | s13 | REF | 60% |
| 447 | s10 | ALT | 70% |
| 448 | s1 | REF | 40% |
| 449 | s13 | ALT | 65% |
| 450 | s13 | REF | 65% |
| 451 | s15 | ALT | 65% |
| 452 | s15 | REF | 70% |
| 453 | s6 | REF | 55% |
| 454 | s17 | REF | 70% |
| 455 | s17 | ALT | 65% |
| 456 | s13 | ALT | 65% |
| 457 | s13 | REF | 65% |
| 458 | s6 | REF | 55% |
| 459 | s4 | BOTH | 45% |
| 460 | s5 | REF | 60% |
| 461 | s19 | REF | 70% |
| 462 | s19 | ALT | 65% |
| 463 | s18 | ALT | 75% |
| 464 | s17 | REF | 55% |
| 465 | s5 | REF | 60% |
| 466 | s19 | REF | 70% |
| 467 | s19 | ALT | 65% |
| 468 | s5 | ALT | 55% |
| 469 | s9 | ALT | 55% |
| 470 | s9 | REF | 60% |
| 471 | s17 | REF | 55% |
| 472 | s18 | ALT | 75% |
| 473 | s4 | ALT | 35% |
| 474 | s19 | REF | 60% |
| 475 | s13 | BOTH | 70% |
| 476 | s5 | REF | 55% |
| 477 | s19 | REF | 70% |
| 478 | s19 | ALT | 65% |
| 479 | s17 | ALT | 50% |
| 480 | s5 | ALT | 50% |
| 481 | s6 | REF | 70% |
| 482 | s6 | ALT | 65% |
| 483 | s15 | REF | 70% |
| 484 | s15 | ALT | 65% |
| 485 | s9 | ALT | 50% |
| 486 | s9 | REF | 55% |
| 487 | s18 | REF | 70% |
| 488 | s10 | REF | 70% |
| 489 | s17 | REF | 50% |
| 490 | s18 | ALT | 75% |
| 491 | s19 | REF | 60% |
| 492 | s16 | REF | 75% |
| 493 | s16 | REF | 75% |
| 494 | s13 | BOTH | 70% |
| 495 | s5 | REF | 50% |
| 496 | s5 | REF | 70% |
| 497 | s9 | REF | 55% |
| 498 | s16 | BOTH | 65% |
| 499 | s13 | REF | 60% |
| 500 | s13 | ALT | 60% |
| 501 | s16 | ALT | 70% |
| 502 | s16 | REF | 75% |
| 503 | s18 | REF | 75% |
| 504 | s19 | REF | 65% |
| 505 | s19 | ALT | 60% |
| 506 | s13 | REF | 65% |
| 507 | s11 | REF | 65% |
| 508 | s14 | REF | 60% |
| 509 | s16 | ALT | 70% |
| 510 | s17 | ALT | 50% |
| 511 | s5 | ALT | 45% |
| 512 | s18 | REF | 75% |
| 513 | s10 | ALT | 65% |
| 514 | s10 | REF | 65% |
| 515 | s6 | REF | 70% |
| 516 | s6 | ALT | 65% |
| 517 | s15 | REF | 70% |
| 518 | s14 | ALT | 55% |
| 519 | s14 | REF | 60% |
| 520 | s15 | ALT | 65% |
| 521 | s9 | ALT | 50% |
| 522 | s9 | REF | 55% |
| 523 | s17 | REF | 65% |
| 524 | s17 | ALT | 60% |
| 525 | s15 | REF | 55% |
| 526 | s12 | ALT | 30% |
| 527 | s5 | REF | 65% |
| 528 | s5 | ALT | 60% |
| 529 | s13 | REF | 60% |
| 530 | s17 | REF | 45% |
| 531 | s2 | REF | 35% |
| 532 | s9 | REF | 60% |
| 533 | s19 | REF | 60% |
| 534 | s11 | REF | 55% |
| 535 | s18 | REF | 75% |
| 536 | s16 | REF | 75% |
| 537 | s19 | REF | 65% |
| 538 | s19 | ALT | 60% |
| 539 | s8 | ALT | 40% |
| 540 | s8 | REF | 40% |
| 541 | s16 | REF | 70% |
| 542 | s13 | BOTH | 65% |
| 543 | s9 | REF | 75% |
| 544 | s5 | REF | 50% |
| 545 | s10 | REF | 60% |
| 546 | s10 | ALT | 60% |
| 547 | s17 | REF | 70% |
| 548 | s10 | BOTH | 70% |
| 549 | s5 | REF | 70% |
| 550 | s19 | REF | 70% |
| 551 | s18 | REF | 75% |
| 552 | s12 | ALT | 35% |
| 553 | s9 | REF | 50% |
| 554 | s14 | ALT | 65% |
| 555 | s14 | REF | 70% |
| 556 | s19 | BOTH | 65% |
| 557 | s8 | ALT | 45% |
| 558 | s8 | REF | 45% |
| 559 | s8 | REF | 40% |
| 560 | s8 | ALT | 40% |
| 561 | s13 | REF | 65% |
| 562 | s12 | ALT | 40% |
| 563 | s16 | REF | 65% |
| 564 | s13 | REF | 55% |
| 565 | s13 | ALT | 55% |
| 566 | s17 | BOTH | 45% |
| 567 | s12 | ALT | 40% |
| 568 | s12 | REF | 35% |
| 569 | s19 | REF | 55% |
| 570 | s19 | ALT | 50% |
| 571 | s8 | BOTH | 50% |
| 572 | s6 | BOTH | 45% |
| 573 | s8 | REF | 40% |
| 574 | s8 | ALT | 40% |
| 575 | s20 | REF | 50% |
| 576 | s20 | ALT | 55% |
| 577 | s16 | REF | 75% |
| 578 | s18 | REF | 70% |
| 579 | s13 | ALT | 65% |
| 580 | s13 | REF | 65% |
| 581 | s20 | ALT | 55% |
| 582 | s16 | REF | 70% |
| 583 | s19 | REF | 65% |
| 584 | s19 | ALT | 60% |
| 585 | s3 | BOTH | 40% |
| 586 | s13 | ALT | 65% |
| 587 | s13 | REF | 65% |
| 588 | s2 | ALT | 35% |
| 589 | s11 | REF | 60% |
| 590 | s2 | REF | 40% |
| 591 | s14 | REF | 60% |
| 592 | s16 | ALT | 70% |
| 593 | s17 | ALT | 45% |
| 594 | s18 | REF | 75% |
| 595 | s16 | REF | 75% |
| 596 | s16 | BOTH | 70% |
| 597 | s16 | ALT | 70% |
| 598 | s10 | REF | 60% |
| 599 | s10 | ALT | 60% |
| 600 | s5 | ALT | 45% |
| 601 | s13 | REF | 60% |
| 602 | s13 | ALT | 60% |
| 603 | s18 | REF | 75% |
| 604 | s10 | ALT | 70% |
| 605 | s14 | REF | 60% |
| 606 | s14 | ALT | 55% |
| 607 | s10 | ALT | 60% |
| 608 | s10 | REF | 60% |
| 609 | s14 | REF | 70% |
| 610 | s14 | ALT | 65% |
| 611 | s6 | REF | 70% |
| 612 | s10 | ALT | 60% |
| 613 | s10 | REF | 60% |
| 614 | s17 | REF | 65% |
| 615 | s17 | ALT | 60% |
| 616 | s9 | ALT | 50% |
| 617 | s15 | REF | 70% |
| 618 | s15 | REF | 65% |
| 619 | s14 | ALT | 55% |
| 620 | s14 | REF | 60% |
| 621 | s15 | ALT | 60% |
| 622 | s8 | ALT | 40% |
| 623 | s8 | REF | 40% |
| 624 | s12 | ALT | 35% |
| 625 | s12 | REF | 30% |
| 626 | s6 | ALT | 55% |
| 627 | s6 | REF | 60% |
| 628 | s9 | ALT | 50% |
| 629 | s9 | REF | 55% |
| 630 | s13 | REF | 60% |
| 631 | s1 | REF | 40% |
| 632 | s20 | REF | 55% |
| 633 | s17 | REF | 65% |
| 634 | s14 | ALT | 55% |
| 635 | s14 | ALT | 55% |
| 636 | s14 | REF | 60% |
| 637 | s12 | ALT | 30% |
| 638 | s8 | ALT | 45% |
| 639 | s10 | REF | 65% |
| 640 | s12 | ALT | 30% |
| 641 | s15 | REF | 55% |
| 642 | s1 | REF | 40% |
| 643 | s1 | ALT | 35% |
| 644 | s5 | ALT | 60% |
| 645 | s5 | REF | 65% |
| 646 | s6 | REF | 60% |
| 647 | s6 | ALT | 55% |
| 648 | s10 | REF | 60% |
| 649 | s11 | REF | 55% |
| 650 | s12 | ALT | 30% |
| 651 | s2 | ALT | 35% |
| 652 | s13 | REF | 60% |
| 653 | s12 | ALT | 35% |
| 654 | s12 | REF | 30% |
| 655 | s5 | REF | 55% |
| 656 | s11 | REF | 60% |
| 657 | s1 | BOTH | 50% |
| 658 | s10 | ALT | 60% |
| 659 | s10 | REF | 60% |
| 660 | s10 | ALT | 60% |
| 661 | s15 | ALT | 50% |
| 662 | s11 | REF | 45% |
| 663 | s16 | REF | 70% |
| 664 | s9 | REF | 55% |
| 665 | s9 | ALT | 50% |
| 666 | s18 | REF | 75% |
| 667 | s16 | REF | 70% |
| 668 | s1 | ALT | 40% |
| 669 | s1 | REF | 45% |
| 670 | s5 | ALT | 55% |
| 671 | s5 | REF | 60% |
| 672 | s5 | BOTH | 65% |
| 673 | s14 | BOTH | 75% |
| 674 | s20 | ALT | 55% |
| 675 | s12 | BOTH | 35% |
| 676 | s15 | REF | 65% |
| 677 | s18 | REF | 75% |
| 678 | s5 | REF | 60% |
| 679 | s6 | ALT | 55% |
| 680 | s10 | REF | 65% |
| 681 | s10 | ALT | 65% |
| 682 | s6 | REF | 60% |
| 683 | s14 | REF | 60% |
| 684 | s19 | REF | 60% |
| 685 | s19 | ALT | 55% |
| 686 | s14 | ALT | 55% |
| 687 | s20 | REF | 50% |
| 688 | s9 | ALT | 70% |
| 689 | s9 | REF | 75% |
| 690 | s5 | BOTH | 40% |
| 691 | s12 | ALT | 30% |
| 692 | s2 | ALT | 40% |
| 693 | s11 | REF | 60% |
| 694 | s13 | REF | 60% |
| 695 | s2 | REF | 30% |
| 696 | s6 | REF | 55% |
| 697 | s6 | ALT | 50% |
| 698 | s9 | BOTH | 65% |
| 699 | s4 | BOTH | 55% |
| 700 | s2 | REF | 40% |
| 701 | s2 | ALT | 35% |
| 702 | s5 | REF | 75% |
| 703 | s5 | ALT | 70% |
| 704 | s8 | ALT | 40% |
| 705 | s8 | REF | 40% |
| 706 | s16 | REF | 75% |
| 707 | s5 | BOTH | 70% |
| 708 | s13 | REF | 65% |
| 709 | s13 | ALT | 65% |
| 710 | s20 | ALT | 60% |
| 711 | s20 | REF | 55% |
| 712 | s17 | ALT | 65% |
| 713 | s5 | ALT | 70% |
| 714 | s19 | ALT | 65% |
| 715 | s8 | REF | 40% |
| 716 | s8 | ALT | 40% |
| 717 | s11 | ALT | 45% |
| 718 | s17 | ALT | 50% |
| 719 | s17 | REF | 55% |
| 720 | s9 | BOTH | 55% |
| 721 | s11 | REF | 65% |
| 722 | s15 | REF | 55% |
| 723 | s14 | REF | 70% |
| 724 | s14 | ALT | 65% |
| 725 | s18 | ALT | 75% |
| 726 | s18 | REF | 75% |
| 727 | s9 | ALT | 55% |
| 728 | s9 | REF | 60% |
| 729 | s16 | REF | 75% |
| 730 | s5 | REF | 75% |
| 731 | s9 | REF | 60% |
| 732 | s13 | REF | 60% |
| 733 | s13 | ALT | 60% |
| 734 | s16 | ALT | 70% |
| 735 | s16 | REF | 75% |
| 736 | s15 | REF | 70% |
| 737 | s16 | ALT | 70% |
| 738 | s14 | REF | 75% |
| 739 | s14 | ALT | 60% |
| 740 | s14 | REF | 65% |
| 741 | s11 | REF | 55% |
| 742 | s10 | REF | 65% |
| 743 | s10 | ALT | 65% |
| 744 | s17 | REF | 70% |
| 745 | s19 | REF | 70% |
| 746 | s8 | ALT | 45% |
| 747 | s8 | REF | 45% |
| 748 | s12 | ALT | 40% |
| 749 | s12 | REF | 35% |
| 750 | s20 | BOTH | 55% |
| 751 | s20 | ALT | 55% |
| 752 | s3 | BOTH | 45% |
| 753 | s16 | REF | 75% |
| 754 | s16 | ALT | 70% |
| 755 | s10 | REF | 65% |
| 756 | s10 | ALT | 65% |
| 757 | s13 | REF | 65% |
| 758 | s13 | ALT | 65% |
| 759 | s10 | BOTH | 75% |
| 760 | s14 | REF | 65% |
| 761 | s14 | ALT | 60% |
| 762 | s10 | ALT | 65% |
| 763 | s10 | REF | 65% |
| 764 | s9 | ALT | 55% |
| 765 | s20 | REF | 55% |
| 766 | s20 | ALT | 60% |
| 767 | s10 | REF | 60% |
| 768 | s11 | REF | 55% |
| 769 | s13 | REF | 60% |
| 770 | s13 | ALT | 60% |
| 771 | s10 | ALT | 60% |
| 772 | s10 | REF | 60% |
| 773 | s10 | ALT | 60% |
| 774 | s11 | REF | 50% |
| 775 | s9 | REF | 55% |
| 776 | s9 | ALT | 50% |
| 777 | s18 | REF | 75% |
| 778 | s16 | ALT | 70% |
| 779 | s14 | REF | 75% |
| 780 | s16 | REF | 75% |
| 781 | s5 | BOTH | 65% |
| 782 | s20 | ALT | 60% |
| 783 | s20 | ALT | 70% |
| 784 | s18 | REF | 75% |
| 785 | s20 | ALT | 75% |
| 786 | s10 | REF | 65% |
| 787 | s10 | ALT | 65% |
| 788 | s16 | REF | 70% |
| 789 | s14 | REF | 60% |
| 790 | s19 | REF | 60% |
| 791 | s19 | ALT | 55% |
| 792 | s14 | ALT | 55% |
| 793 | s20 | REF | 55% |
| 794 | s12 | BOTH | 30% |
| 795 | s2 | BOTH | 45% |
| 796 | s11 | REF | 60% |
| 797 | s6 | BOTH | 55% |
| 798 | s9 | BOTH | 65% |
| 799 | s4 | BOTH | 60% |
| 800 | s8 | ALT | 40% |
| 801 | s8 | REF | 40% |
| 802 | s20 | ALT | 65% |
| 803 | s20 | REF | 60% |
| 804 | s11 | ALT | 50% |
| 805 | s9 | BOTH | 60% |
| 806 | s15 | REF | 60% |
| 807 | s14 | REF | 70% |
| 808 | s14 | ALT | 65% |
| 809 | s13 | BOTH | 60% |
| 810 | s11 | REF | 55% |
| 811 | s9 | REF | 70% |
| 812 | s16 | REF | 75% |
| 813 | s14 | REF | 65% |
| 814 | s14 | ALT | 60% |
| 815 | s10 | BOTH | 70% |
| 816 | s10 | REF | 60% |
| 817 | s20 | REF | 65% |
| 818 | s10 | ALT | 65% |
| 819 | s10 | REF | 65% |
| 820 | s10 | ALT | 60% |
| 821 | s9 | REF | 55% |
| 822 | s9 | ALT | 50% |
| 823 | s16 | REF | 75% |
| 824 | s20 | ALT | 70% |
| 825 | s11 | REF | 65% |
| 826 | s9 | BOTH | 65% |
| 827 | s20 | ALT | 70% |
| 828 | s20 | REF | 65% |
| 829 | s14 | REF | 70% |
| 830 | s14 | ALT | 65% |
| 831 | s10 | BOTH | 70% |
| 832 | s17 | REF | 65% |
| 833 | s9 | REF | 60% |
| 834 | s9 | ALT | 55% |
| 835 | s16 | REF | 75% |
| 836 | s10 | BOTH | 70% |
| 837 | s9 | REF | 65% |
| 838 | s9 | ALT | 60% |
| 839 | s16 | ALT | 75% |
| 840 | s6 | REF | 70% |
| 841 | s20 | ALT | 75% |
| 842 | s20 | REF | 70% |
| 843 | s9 | REF | 75% |
| 844 | s9 | ALT | 70% |
| 845 | s19 | ALT | 55% |
| 846 | s19 | REF | 60% |
| 847 | s20 | ALT | 70% |
| 848 | s20 | REF | 65% |
| 849 | s10 | ALT | 60% |
| 850 | s10 | REF | 60% |
| 851 | s6 | ALT | 65% |
| 852 | s9 | REF | 70% |
| 853 | s9 | ALT | 65% |
| 854 | s19 | REF | 70% |
| 855 | s15 | ALT | 65% |
| 856 | s15 | REF | 70% |
| 857 | s19 | ALT | 55% |
| 858 | s19 | REF | 60% |
| 859 | s20 | ALT | 70% |
| 860 | s20 | REF | 65% |
| 861 | s19 | BOTH | 55% |
| 862 | s17 | BOTH | 55% |
| 863 | s19 | ALT | 55% |
| 864 | s19 | REF | 60% |
| 865 | s14 | ALT | 60% |
| 866 | s14 | REF | 65% |
| 867 | s17 | REF | 60% |
| 868 | s17 | ALT | 55% |
| 869 | s18 | REF | 75% |
| 870 | s10 | ALT | 60% |
| 871 | s10 | REF | 60% |
| 872 | s5 | REF | 60% |
| 873 | s6 | ALT | 60% |
| 874 | s17 | ALT | 60% |
| 875 | s17 | REF | 65% |
| 876 | s9 | REF | 65% |
| 877 | s9 | ALT | 60% |
| 878 | s15 | ALT | 65% |
| 879 | s15 | REF | 70% |
| 880 | s19 | ALT | 55% |
| 881 | s19 | REF | 60% |
| 882 | s5 | REF | 60% |
| 883 | s5 | ALT | 55% |
| 884 | s12 | ALT | 30% |
| 885 | s20 | REF | 65% |
| 886 | s19 | REF | 50% |
| 887 | s17 | BOTH | 55% |
| 888 | s15 | ALT | 60% |
| 889 | s15 | REF | 65% |
| 890 | s19 | ALT | 55% |
| 891 | s19 | REF | 60% |
| 892 | s6 | ALT | 55% |
| 893 | s14 | ALT | 60% |
| 894 | s14 | REF | 65% |
| 895 | s2 | REF | 40% |
| 896 | s15 | BOTH | 65% |
| 897 | s8 | REF | 40% |
| 898 | s10 | REF | 70% |
| 899 | s10 | ALT | 70% |
| 900 | s6 | REF | 60% |
| 901 | s19 | BOTH | 65% |
| 902 | s17 | ALT | 50% |
| 903 | s15 | REF | 75% |
| 904 | s18 | ALT | 75% |
| 905 | s15 | ALT | 65% |
| 906 | s14 | REF | 65% |
| 907 | s10 | ALT | 60% |
| 908 | s10 | REF | 60% |
| 909 | s8 | ALT | 40% |
| 910 | s8 | REF | 40% |
| 911 | s19 | REF | 60% |
| 912 | s19 | ALT | 55% |
| 913 | s15 | REF | 65% |
| 914 | s15 | ALT | 60% |
| 915 | s16 | BOTH | 70% |
| 916 | s14 | ALT | 60% |
| 917 | s8 | ALT | 40% |
| 918 | s6 | REF | 60% |
| 919 | s6 | ALT | 55% |
| 920 | s5 | ALT | 55% |
| 921 | s5 | REF | 60% |
| 922 | s6 | ALT | 55% |
| 923 | s1 | ALT | 45% |
| 924 | s1 | REF | 50% |
| 925 | s17 | ALT | 60% |
| 926 | s8 | REF | 35% |
| 927 | s8 | ALT | 35% |
| 928 | s17 | REF | 65% |
| 929 | s15 | REF | 65% |
| 930 | s9 | REF | 60% |
| 931 | s9 | ALT | 55% |
| 932 | s14 | REF | 60% |
| 933 | s15 | ALT | 60% |
| 934 | s15 | REF | 65% |
| 935 | s15 | ALT | 60% |
| 936 | s19 | ALT | 50% |
| 937 | s19 | REF | 55% |
| 938 | s11 | ALT | 40% |
| 939 | s19 | ALT | 50% |
| 940 | s19 | REF | 55% |
| 941 | s11 | REF | 60% |
| 942 | s12 | REF | 30% |
| 943 | s5 | REF | 55% |
| 944 | s20 | REF | 50% |
| 945 | s13 | REF | 60% |
| 946 | s12 | ALT | 35% |
| 947 | s16 | REF | 65% |
| 948 | s13 | REF | 55% |
| 949 | s13 | ALT | 55% |
| 950 | s17 | BOTH | 45% |
| 951 | s8 | ALT | 40% |
| 952 | s12 | ALT | 30% |
| 953 | s12 | ALT | 35% |
| 954 | s12 | REF | 30% |
| 955 | s8 | ALT | 40% |
| 956 | s8 | REF | 40% |
| 957 | s17 | REF | 60% |
| 958 | s8 | REF | 40% |
| 959 | s1 | ALT | 45% |
| 960 | s19 | REF | 50% |
| 961 | s19 | ALT | 45% |
| 962 | s9 | ALT | 70% |
| 963 | s8 | REF | 45% |
| 964 | s8 | ALT | 45% |
| 965 | s12 | ALT | 30% |
| 966 | s9 | REF | 75% |
| 967 | s8 | BOTH | 45% |
| 968 | s12 | ALT | 35% |
| 969 | s12 | REF | 30% |
| 970 | s6 | BOTH | 45% |
| 971 | s20 | REF | 65% |
| 972 | s19 | REF | 50% |
| 973 | s11 | REF | 35% |
| 974 | s17 | BOTH | 55% |
| 975 | s2 | ALT | 30% |
| 976 | s2 | REF | 35% |
| 977 | s11 | REF | 40% |
| 978 | s19 | ALT | 50% |
| 979 | s19 | REF | 55% |
| 980 | s3 | BOTH | 30% |
| 981 | s11 | BOTH | 60% |
| 982 | s15 | REF | 65% |
| 983 | s19 | ALT | 55% |
| 984 | s19 | REF | 60% |
| 985 | s6 | ALT | 60% |
| 986 | s6 | REF | 65% |
| 987 | s6 | ALT | 50% |
| 988 | s14 | ALT | 55% |
| 989 | s14 | REF | 60% |
| 990 | s8 | REF | 35% |
| 991 | s8 | ALT | 35% |
| 992 | s12 | REF | 30% |
| 993 | s12 | ALT | 35% |
| 994 | s14 | REF | 70% |
| 995 | s11 | REF | 60% |
| 996 | s9 | REF | 75% |
| 997 | s9 | ALT | 70% |
| 998 | s15 | ALT | 60% |
| 999 | s20 | REF | 50% |
| 1000 | s20 | ALT | 55% |
| 1001 | s8 | REF | 40% |
| 1002 | s9 | ALT | 55% |
| 1003 | s9 | REF | 60% |
| 1004 | s16 | REF | 70% |
| 1005 | s14 | BOTH | 60% |
| 1006 | s5 | BOTH | 45% |
| 1007 | s17 | ALT | 60% |
| 1008 | s17 | REF | 65% |
| 1009 | s10 | REF | 65% |
| 1010 | s10 | ALT | 65% |
| 1011 | s13 | ALT | 60% |
| 1012 | s13 | REF | 60% |
| 1013 | s10 | ALT | 65% |
| 1014 | s17 | REF | 65% |
| 1015 | s17 | ALT | 60% |
| 1016 | s6 | REF | 55% |
| 1017 | s5 | REF | 55% |
| 1018 | s19 | BOTH | 65% |
| 1019 | s17 | REF | 50% |
| 1020 | s17 | ALT | 45% |
| 1021 | s15 | REF | 70% |
| 1022 | s15 | ALT | 65% |
| 1023 | s10 | REF | 65% |
| 1024 | s16 | REF | 70% |
| 1025 | s18 | ALT | 75% |
| 1026 | s15 | ALT | 65% |
| 1027 | s13 | REF | 60% |
| 1028 | s14 | REF | 60% |
| 1029 | s10 | ALT | 60% |
| 1030 | s10 | REF | 60% |
| 1031 | s17 | REF | 65% |
| 1032 | s17 | ALT | 60% |
| 1033 | s2 | REF | 35% |
| 1034 | s19 | REF | 60% |
| 1035 | s19 | ALT | 55% |
| 1036 | s8 | ALT | 40% |
| 1037 | s8 | REF | 40% |
| 1038 | s12 | ALT | 35% |
| 1039 | s13 | REF | 60% |
| 1040 | s19 | REF | 55% |
| 1041 | s3 | BOTH | 35% |
| 1042 | s8 | REF | 40% |
| 1043 | s8 | ALT | 40% |
| 1044 | s13 | ALT | 60% |
| 1045 | s13 | REF | 60% |
| 1046 | s15 | REF | 60% |
| 1047 | s15 | ALT | 55% |
| 1048 | s2 | ALT | 30% |
| 1049 | s2 | REF | 35% |
| 1050 | s16 | BOTH | 65% |
| 1051 | s17 | REF | 65% |
| 1052 | s17 | ALT | 60% |
| 1053 | s15 | ALT | 60% |
| 1054 | s12 | ALT | 35% |
| 1055 | s13 | ALT | 60% |
| 1056 | s13 | REF | 60% |
| 1057 | s14 | ALT | 55% |
| 1058 | s8 | ALT | 40% |
| 1059 | s6 | REF | 60% |
| 1060 | s6 | ALT | 55% |
| 1061 | s12 | ALT | 30% |
| 1062 | s2 | ALT | 30% |
| 1063 | s5 | ALT | 50% |
| 1064 | s5 | REF | 55% |
| 1065 | s11 | REF | 55% |
| 1066 | s6 | ALT | 55% |
| 1067 | s1 | ALT | 40% |
| 1068 | s1 | REF | 45% |
| 1069 | s16 | ALT | 65% |
| 1070 | s2 | REF | 40% |
| 1071 | s2 | ALT | 35% |
| 1072 | s17 | ALT | 40% |
| 1073 | s16 | REF | 70% |
| 1074 | s13 | REF | 60% |
| 1075 | s13 | ALT | 60% |
| 1076 | s17 | ALT | 60% |
| 1077 | s8 | REF | 35% |
| 1078 | s8 | ALT | 35% |
| 1079 | s17 | ALT | 45% |
| 1080 | s14 | ALT | 55% |
| 1081 | s14 | REF | 60% |
| 1082 | s10 | REF | 60% |
| 1083 | s10 | ALT | 60% |
| 1084 | s17 | REF | 65% |
| 1085 | s20 | BOTH | 55% |
| 1086 | s19 | ALT | 55% |
| 1087 | s20 | REF | 55% |
| 1088 | s20 | ALT | 60% |
| 1089 | s11 | REF | 50% |
| 1090 | s13 | REF | 55% |
| 1091 | s13 | ALT | 55% |
| 1092 | s18 | REF | 70% |
| 1093 | s10 | BOTH | 65% |
| 1094 | s14 | REF | 60% |
| 1095 | s14 | ALT | 55% |
| 1096 | s21 | ALT | 30% |
| 1097 | s15 | REF | 65% |
| 1098 | s18 | ALT | 75% |
| 1099 | s10 | ALT | 60% |
| 1100 | s10 | REF | 60% |
| 1101 | s10 | ALT | 65% |
| 1102 | s14 | REF | 70% |
| 1103 | s14 | ALT | 65% |
| 1104 | s9 | REF | 60% |
| 1105 | s9 | ALT | 55% |
| 1106 | s6 | REF | 65% |
| 1107 | s10 | ALT | 55% |
| 1108 | s10 | REF | 55% |
| 1109 | s6 | ALT | 60% |
| 1110 | s19 | ALT | 55% |
| 1111 | s19 | REF | 60% |
| 1112 | s14 | ALT | 55% |
| 1113 | s14 | REF | 60% |
| 1114 | s17 | REF | 60% |
| 1115 | s17 | ALT | 55% |
| 1116 | s5 | ALT | 50% |
| 1117 | s5 | REF | 55% |
| 1118 | s17 | ALT | 60% |
| 1119 | s17 | REF | 65% |
| 1120 | s9 | ALT | 45% |
| 1121 | s15 | ALT | 60% |
| 1122 | s15 | REF | 65% |
| 1123 | s2 | REF | 40% |
| 1124 | s15 | REF | 70% |
| 1125 | s11 | REF | 65% |
| 1126 | s15 | ALT | 60% |
| 1127 | s15 | ALT | 55% |
| 1128 | s16 | BOTH | 65% |
| 1129 | s6 | REF | 60% |
| 1130 | s6 | ALT | 55% |
| 1131 | s15 | REF | 65% |
| 1132 | s14 | ALT | 55% |
| 1133 | s14 | REF | 60% |
| 1134 | s15 | ALT | 60% |
| 1135 | s19 | REF | 55% |
| 1136 | s8 | ALT | 35% |
| 1137 | s8 | REF | 35% |
| 1138 | s17 | ALT | 50% |
| 1139 | s17 | REF | 55% |
| 1140 | s12 | ALT | 30% |
| 1141 | s11 | BOTH | 35% |
| 1142 | s2 | ALT | 30% |
| 1143 | s2 | REF | 35% |
| 1144 | s19 | ALT | 45% |
| 1145 | s19 | REF | 50% |
| 1146 | s6 | REF | 60% |
| 1147 | s12 | REF | 30% |
| 1148 | s12 | ALT | 35% |
| 1149 | s2 | ALT | 35% |
| 1150 | s11 | REF | 55% |
| 1151 | s9 | REF | 75% |
| 1152 | s9 | ALT | 70% |
| 1153 | s9 | ALT | 50% |
| 1154 | s9 | REF | 55% |
| 1155 | s13 | ALT | 60% |
| 1156 | s13 | REF | 60% |
| 1157 | s1 | REF | 35% |
| 1158 | s5 | REF | 55% |
| 1159 | s5 | ALT | 50% |
| 1160 | s13 | REF | 60% |
| 1161 | s18 | ALT | 75% |
| 1162 | s12 | ALT | 30% |
| 1163 | s20 | REF | 55% |
| 1164 | s8 | REF | 40% |
| 1165 | s8 | ALT | 40% |
| 1166 | s17 | REF | 65% |
| 1167 | s12 | ALT | 30% |
| 1168 | s14 | ALT | 55% |
| 1169 | s16 | REF | 65% |
| 1170 | s17 | ALT | 40% |
| 1171 | s17 | REF | 45% |
| 1172 | s14 | ALT | 55% |
| 1173 | s14 | REF | 60% |
| 1174 | s20 | BOTH | 50% |
| 1175 | s15 | REF | 60% |
| 1176 | s15 | ALT | 55% |
| 1177 | s6 | REF | 55% |
| 1178 | s6 | ALT | 50% |
| 1179 | s8 | ALT | 45% |
| 1180 | s9 | REF | 70% |
| 1181 | s9 | ALT | 65% |
| 1182 | s10 | REF | 65% |
| 1183 | s15 | REF | 55% |
| 1184 | s4 | BOTH | 45% |
| 1185 | s4 | BOTH | 45% |
| 1186 | s4 | BOTH | 45% |
| 1187 | s4 | BOTH | 45% |
| 1188 | s4 | BOTH | 35% |
| 1189 | s4 | ALT | 30% |
| 1190 | s15 | REF | 65% |
| 1191 | s2 | REF | 25% |
| 1192 | s13 | REF | 55% |

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

Further, the examples herein below disclose methods utilizing SpCas9 and guide sequences suitable to target the SpCas9 to the disclosed SNP positions. The examples demonstrate the feasibility of the strategies disclosed. A person having ordinary skill in the art would understand that the same guides sequences may be used with different CRISPR nuclease to target the disclosed SNPs to apply each of the specified strategies. Further, different guide sequences that target other CRISPR nucleases to the same SNPs may be used together with the other nucleases to apply each of the specified strategies.

### EXPERIMENTAL DETAILS

### Example 1: Screening Guide Sequences Suitable to Work in Conjunction with SpCas9 and Targeting SNPs and Sequences Complying with the Disclosed Strategies

HeLa cells were seeded into 96 well-plate (3K/well). 24 h later, cells were co-transfected with either 65 ng of WT-Cas9 or Dead-Cas9 and 20 ng of gRNA plasmids, identified as g36 through g66, targeting the different regions and SNPs in ELANE using Turbofect reagent (Thermo Scientific). 12 hours later, fresh media was added, and 72 hours post transfection, genomic DNA were extracted, and the expected region targeted by the Cas9 was amplified and the product size was analyzed by capillary electrophoreses with a DNA ladder. The intensity of the bands was analyzed using the Peak Scanner software v1.0. The percent of editing was calculated according the following formula: 100%−(Intensity not edited band/Intensity total bands)*100. FIG. 6 represents the mean activity of each gRNA following subtraction of the Dead-Cas9 background activity±SD of three independent experiment.

TABLE 3
guides sg36 through sg66 of Example 1 as
identified by SEQ ID NO.

| | Example | SEQ ID | |
|---|---|---|---|
| Guide sequence | 1 gID | NO: | SNP location |
| UAGGGGUGUUAUGGUCACAG | g36 | 972 | upstream −2590 bp |
| CACAGCGGGUGUAGACUCCG | g37 | 308 | upstream −2590 bp |
| ACAGCGGGUGUAGACUCCGA | g38 | 94 | upstream −2590 bp |
| CAGCGGGUGUAGACUCCGAG | g39 | 352 | upstream −2590 bp |
| AGCGGGUGUAGACUCCGAGG | g40 | 180 | upstream −2590 bp |
| CCGUUGCAGCUGGAACAUCG | g41 | 499 | upstream −1475 bp |
| CGUUGCAGCUGGAACAUCGU | g42 | 564 | upstream −1475 bp |
| GUUGCAGCUGGAACAUCGUG | g43 | 948 | upstream −1475 bp |
| UUGCAGCUGGAACAUCGUGG | g44 | 1192 | upstream −1475 bp |
| CUGGAACAUCGUGGGGGAGA | g45 | 601 | upstream −1475 bp |
| UGGAACAUCGUGGGGGAGAU | g46 | 1090 | upstream −1475 bp |
| AUCGUGGGGGAGAUGGGAAG | g47 | 249 | upstream −1475 bp |
| GGAGUCCCAGCUGCGGGAAA | g57 | 786 | upstream −1415 bp |
| GCUGCGGGAAAGGGAUUCCC | g58 | 755 | upstream −1415 bp |
| GGGAAUCCCUUUCCCGCAGC | g59 | 819 | upstream −1415 bp |
| GGAAUCCCUUUCCCGCAGCU | g60 | 772 | upstream −1415 bp |
| CAAAUGUCAGAUAAUCAAUG | g27 | 287 | upstream |
| AAAUGUCAGAUAAUCAAUGU | g28 | 1191 | upstream |
| ACCAAGGCUCAGGGCGUUGG | g67 | 1193 | Int3 |
| CCUGUUGCUGCAGUCCGGGC | g32 | 1194 | Int4 |
| CCAGCCCGGACUGCAGCAAC | g33 | 1195 | Int4 |
| UCCCUCCUAGGGUCUAGCCA | g34 | 1196 | Int4 |
| AGUCCGGGCUGGGAGCGGGU | g35 | 1197 | Int4 |
| AUGUUUAUUGUGCCAGAUGC | g29 | 1198 | 3UTR |
| GUGGGCAGCUGAGGUGACCC | g30 | 1199 | 3UTR |
| CACCCACACUCUCCAGCAUC | g31 | 1200 | 3UTR |
| UGUCAAGCCCCAGAGGCCAC | g61 | 1122 | downstream +2968 bp |
| GUCAAGCCCCAGAGGCCACA | g62 | 889 | downstream +2968 bp |
| GUCUCUGUCCCUGUGGCCUC | g63 | 913 | downstream +2968 bp |
| UCUCUGUCCCUGUGGCCUCU | g64 | 1046 | downstream +2968 bp |
| CUCUGUCCCUGUGGCCUCUG | g65 | 1190 | downstream +2968 bp |
| UGUCAAGCCCCAGAGGCCAC | g66 | 1122 | downstream +2968 bp |

### Example 2: Demonstrating the Feasibility of the Excision Strategies

HeLa cells were co-transfected with spCas9-WT and RNA pairs; sg35 (INT 4) with either g39 (rs10414837), g58 (rs3761005) or g62 (rs1683564) for strategies 1a, 1b and 2, respectively. 72 h post-transfection, gDNA was extracted and excision efficiency was assessed by measuring the decrease in copy number of exon 1 (strategy 1) or exon 5 (strategy 2), using droplet digital PCR (ddPCR) kits (10042958 and 10031228, Bio-Rad Laboratories). In addition, exon 1 was used to normalize the excision rate of strategy 2 while exon 5 was used to normalize the excision rate of strategy 1. The results disclosed in Table 4 represent the mean % excision±SD (standard deviation) of 2 independent experiments.

TABLE 4
Tested excision rate for each strategy

| Strategy | Guide-RNA pair | Excision rate (%) |
|---|---|---|
| 1.a | g39 ± g35 | 49 ± 2 |
| 1.b | g58 ± g35 | 45 ± 9.6 |
| 2 | g62 ± g35 | 41 ± 12.6 |

### Example 3: Assessing Allele Discriminating Editing with the Different sgRNAs

Ribonucleoprotein complexes (RNPs) were assembled from the relevant gRNAs, targeting the reference sequence, and WT-Cas9 (#1081058) or HiFi Cas9 (#1081060) purchased from Integrated DNA Technology (IDT) according to the manufacturer instructions. The RNPs were then nucleofected into iPSCs harboring the relevant SNPs using the 4D-Nucleofecor® System (Lonza). 72 h later, gDNA is extracted and the SNPs regions were amplified and sent to NGS analysis. Allele discrimination were assessed according to % of editing detected in the reference and the alternative alleles. The indels frequency in each site was calculated using Cas-Analyzer software. Results are summarized in Table 5.

TABLE 5
% Editing using indicated guide sequences

| | % of Editing | | | | | |
|---|---|---|---|---|---|---|
| | rs10414837 (g39) | | rs3761005 (g58) | | rs1683564 (g62) | |
| spCas9 | Reference | Alternative | Reference | Alternative | Reference | Alternative |
| Varaint | Allele | Allele | Allele | Allele | Allele | Allele |
| WT-Cas9 | 65.3 | 34.7 | 50 | 50 | 100 | 0 |
| HiFi-Cas9 | 94.3 | 5.7 | 49 | 51 | 100 | 0 |

### Example 4: Editing Efficiency in HSCs

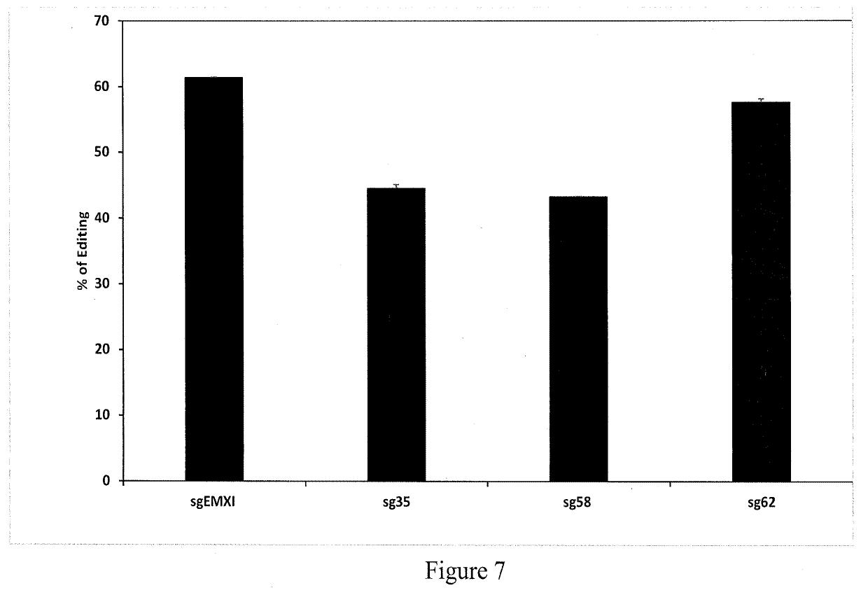

HSCs from healthy donors were nucleofected with RNA components of spCas9-WT and gRNAs targeting either EMX1 (sgEMX1) or ELANE (g35: INT 4; g58: rs3761005; g62: rs1683564). 72 h post nucleofection gDNA was extracted and editing levels were assayed by IDAA. FIG. 7 represents the mean % of editing±SD of 2 independent experiments performed in duplicates.

### Example 5: Functional Maturation Assay

To prove the rescue of the phenotype in corrected cells, a maturation assay starting from patient-derived induced pluripotent cells (iPSC), is prepared from reprogrammed somatic cells. The cells provide a renewable source of patient-derived cells and are shown to accurately replicate the disease phenotype. Briefly, patient PBMCs are transfected with episomal constructs expressing the reprogramming genes, Oct4, Sox2, Nanog, Lin28, L-Myc, Klf4, and SV40LT. Patient-derived iPSC and normal iPSCs harboring the same SNPs genotype are differentiated into hematopoietic progenitor cells using a commercial kit (STEMdiff™ Hematopoietic Kit, STEMCELL Technologies). After 12 days the differentiation efficiency is estimated by analyzing the cells for their expression of progenitor markers CD34 and CD45, by Flow cytometry analysis. Normal and SCN differentiated progenitor cells are be subjected to gene editing and are grown for 5 days in conditioned media containing stem cell factor (SCF), IL-3, and GM-CSF that promotes the differentiation into neutrophils. Normal unedited and edited cells, SCN edited cells differentiate into neutrophils while unedited SCN-derived cells arrest at earlier differentiation stages. The efficiency of differentiation into neutrophils is measured by detecting neutrophils surface markers (e.g. upregulation of CD16, CD66b and the pan myeloid marker CD33) by Flow cytometry.

### Example 6: In Vivo Pilot Dose-Range Finding and Biodistribution Study in Immunodeficient Mice

The dosing schedule, dose range, and route of administration, are studied to determine the presence and number of HSCs with self-renewal and multilineage capacity in immunodeficient NSG mice, following G-CSF administration. A repopulation assay is conducted to determine the presence and number of HSCs that are able to regenerate a functional immune system to establish long term engraftment. For such verification, the NSG strain is used, which is highly supportive of human engraftment and hematopoietic repopulation. The NSG mouse is a NOD SCID mouse lacking mature T cells, B cells, and natural killer (NK) cells, in addition to being deficient in multiple cytokine signaling pathways and having many defects in innate immunity. Engraftment is assessed 16 weeks after primary transplantation (analysis after 12 weeks post-transplant).

The 16-week pilot biodistribution study in NSG mice investigates dose and maximum duration and is conducted at several cell dose levels. The study includes three dose levels plus a group of mice receiving unedited SCN cells. Duration of the pilot study is 16 weeks; however, one of two high dose groups continues for up to 6 months. Mice survive for 6 months in the pilot study, and the duration of the pivotal biodistribution study is 6 months. During the study, persistence of expression via qPCR and immune histochemistry is studied.

### Example 7: Pivotal Biodistribution Study in Immunodeficient Mice

The pivotal biodistribution study utilizes NSG mice and follows the pilot dose-range findings of Example 6. This study is conducted in compliance with Good Laboratory Practice (GLP) and is a pivotal nonclinical pharmacokinetics, pharmacodynamics, and toxicology study. Assessment of toxicity is based on mortality, clinical observations, body and organ weights, and clinical and anatomic pathology following HSC infusion.

Transplantation of gene-edited CD34+ cells into NSG mice requires conditioning in order to provide depletion of endogenous bone marrow and to allow the engraftment of donor cells. Accordingly, Busulfan-conditioning is employed to mimic the clinical situation. Three groups (gene edited cells, unedited cells and busulfan vehicle only controls) of 10 male and female mice per group are utilized.

Although the NSG mouse model is not completely similar to the human neutrophil depleted situation, this does not affect the validity of the model for use in the biodistribution study of the gene-edited CD34+ cells, since the mice at any rate are treated with busulfan, depleting the bone marrow function, before the in vivo injection of ex vivo gene edited cells. An available neutrophil-depleted mouse strain, myeloid cell leukemia 1 (Mcl-1) antiapoptotic protein in Lyz2^{Cre/Cre}Mcl-1^{flox/flox} (Mcl-1^{ΔMyelo}) shows that myeloid-specific deletion of Mcl-1 lead to very severe neutropenia (Csepregi et al. 2018). Mcl-1^{ΔMyelo} mice are able to breed and their survival is close to normal both under specific pathogen-free and conventional housing conditions. However, in contrast to the NSG mouse, there is limited experience with the Mcl-1^{ΔMyelo} mouse model.

### Example 8: A Correction Analysis

Guide sequences comprising 17-20 nucleotides in the sequences of 17-20 contiguous nucleotides set forth in SEQ ID NOs: 1-1192 are screened for high on target activity. On target activity is determined by DNA capillary electrophoresis analysis.

According to DNA capillary electrophoresis analysis, guide sequences comprising 17-20 nucleotides in the sequences of 17-20 contiguous nucleotides set forth in SEQ ID NOs: 1-1192 are found to be suitable for correction of the ELANE gene.

### Example 9: Efficacy of Allele Specific Knockout of ELANE

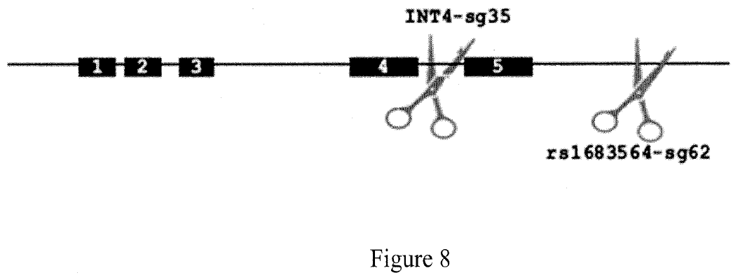

Specific knock-out of the mutated allele of the ELANE gene is mediated by excising intron 4 and exon5 of the mutant allele of the ELANE gene. This is achieved by mediating a DSB in intron 4 and utilizing SNP rs1683564 for mediating an allele specific DSB as described in FIG. 8. To demonstrate that this strategy effectively enables HSCs to differentiate into mature and functional neutrophils, healthy donors are nucleofected with HSCs (Lonza) with RNPs containing g35 and g62 targeting the respective SNP of intron 4 (See Table 1). Unedited cells are used as a positive control. Forty-eight (48) hours following nucleofection, HSCs are differentiated towards neutrophils according to a published protocol (Zhenwang Jie, et al. PlosOne 2017). The differentiation efficiency of the edited and unedited cells is measured by FACS following staining with the neutrophils specific markers CD66b and CD177. To assess the function of the HSCs-mediated neutrophils, the following assays are performed:
1. Phagocytosis: is assayed using the EZCell™ Phagocytosis Assay Kit (BioVision). The kit utilizes pre-labeled Zymosan particles as a tool for rapid and accurate detection and quantification of in vitro phagocytosis by flow cytometry.
2. A killing assay is conducted by incubating the HSCs-derived neutrophils with E. coli, with the bacteria then seeded on agar plate for colonies formation. Untreated bacteria or bacteria that were incubated with undifferentiated HSCs are used as controls. The killing efficiency is calculated as follows: (# of colonies_{Neutrophils}/# of colonies_{Control})×100.
3. Chemotaxis is assayed using the EZCell™ Cell Migration/Chemotaxis Assay Kit (Biovision).

### Example 10: Subject Selection for Treatment

Step 1: Four patients A-D diagnosed with SCN or CyN are screened by Exon sequencing to identify an ELANE pathogenic mutation in the ELANE gene. Step 2: Subjects with an identified mutation are then screened by Sanger sequencing to confirm heterozygocity of at least one of rs1683564, rs10414837 and rs3761005. Step 3: For each subject determined to be heterozygous at at least one of rs1683564, rs10414837 and rs3761005, the nucleotide of the heterozygous SNP on the mutant allele of the ELANE gene is determined using BAC bio. Step 4: Appropriate guides are selected according to Table 6.

TABLE 6
Guides Designed for Discriminating SNPs and used for the Editing Strategy

| | SEQ ID | Target | | | |
|---|---|---|---|---|---|
| gRNA | NO: | SNP | DNA Sequence | Location | Mechanism |
| g39 | 352 | rs10414837 | CAGCGGGTGTAGACTCC | Promoter | Excision, |
| REF | | | GAG | region | allele knock |
| ALT | 351 | | CAGCAGGTGTAGACTCC | | out |
| | | | GAG | | |
| g58 | 755 | rs3761005 | GCTGCGGGAAAGGGATT | Promoter | Excision, |
| REF | | | CCC | region | allele knock |
| ALTt | 756 | | GCTGCGGGAATGGGATT | | out |
| | | | CCC | | |
| g62 | 889 | rs1683564 | GTCAAGCCCCAGAGGCC | Downstream | Excision, |
| REF | | | ACA | to | allele knock |
| ALT | 888 | | GTCAAGCCCCAGAGGAC | 3′UTR | out |
| | | | ACA | | |
| g35 | 1197 | | AGTCCGGGCTGGGAGCG | Intron 4 | Excision, |
| | | | GGT | | allele knock |
| | | | | | out |

Step 5: The guides selected are introduced to PBMCs obtained from each respective subject and reduction in the pathogenic ELANE mutation in the PBMCs is verified by Next Generation Sequencing. The methodology for patients A-D is illustrated below:
1. Patient A is screened according to step 1 and found to have a known pathogenic mutation in his ELANE gene, in agreement with his phenotype and clinical condition. Patient A is screened according to step 2, around the SNPs of interest, and is found to be homozygous for all three SNP—rs10414837, rs1683564 and rs3761005. Patient A is determined to not eligible for treatment.
2. Patient B is verified for a known pathogenic ELANE mutation. Patient B is screened according to step 2 and is found to be homozygous for SNPs rs1683564 and rs10414837. Patient B is found to be heterozygous for rs3761005 in the promotor region. Patient B is determined to be eligible for treatment. According to step 3, the nucleotide of the SNP residing on the same allele as the pathogenic ELANE mutation (linkage determination) is determined. Patient B is found to have the reference nucleotide base in the rs3761005 SNP position on the same allele as the ELANE pathogenic mutation. g58ref is fully complementary to this SNP presentation and is selected in combination with g35 directed at the non-coding region of intron 4. According to step 4, the chosen guide composition includes a pair of guides g58ref and g35. Successful excision of the mutated allele using the selected pair of guides is verified according to step 5 on the patient PMBCs using NGS readout.
3. Patient C suffers from Severe Neutropenia since early childhood. Screening according to step A, no pathogenic mutation is found in his ELANE gene. Patient C is determined to not eligible for treatment.
4. Patient D is verified to have a known pathogenic mutation in ELANE gene, and when genotyping his SNPs according to step 2, he is found to be heterozygous at 2 out of 3 SNPs—both rs10414837 and rs1683564 are found to be heterozygous while rs3761005 is found to be homozygous. A selection between two possible SNPs to use for the gene manipulation is made. In order to make the selection, step 3 is performed to determine the linkage between the SNPs and the pathogenic mutation, i.e. determination of which nucleotide of the SNP (reference or alternative) resides on the same allele as the ELANE pathogenic mutation (SNP presentation). Patient D is determined to have the reference presentation of rs10414837 and sg39ref is determined to be appropriate for use. Referring to the rs1683564 SNP, the alternative presentation is found to be linked to the ELANE pathogenic mutation, sg62alt is determined to be the appropriate guide for use. Each of these guides is used in combination with g35. According to Step 4, two pairs of possible guides compositions are identified: sg39ref+g35, and g62alt+g35. To determine which of the guide pairs is preferable, a database of editing properties and characterization of each of the guides and guide pairs is assessed to determine off-target and editing efficiencies A guide pair is selected based on the database assessment, and is utilized according to step 5 on PBCS providing an NGS readout.

### REFERENCES

1. Ahmad and Allen (1992) “Antibody-mediated Specific Binging and Cytotoxicity of Liposome-entrapped Doxorubicin to Lung Cancer Cells in Vitro”, Cancer Research 52:4817-20
2. Anders (1992) “Human gene therapy”, Science 256:808-13
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200
4. Behr (1994) Gene transfer with synthetic cationic amphiphiles: Prospects for gene therapy”, Bioconjuage Chem 5:382-89
5. Blaese (1995) “Vectors in cancer therapy: how will they deliver”, Cancer Gene Ther. 2:291-97
6. Blaese et al. (1995) “T lymphocyte-directed gene therapy for ADA-SCID: initial trial results after 4 years”, Science 270(5235):475-80
7. Boxer, L. A. (2012) “How to approach neutropenia”, Hematology Am Soc Hematol Educ Program 2012: 174-182
8. Buchschacher and Panganiban (1992) “Human immunodeficiency virus vectors for inducible expression of foreign genes”, J. Virol. 66:2731-39
9. Burstein et al. (2017) “New CRISPR-Cas systems from uncultivated microbes”, Nature 542:237-41
10. Carlsson, G et al. (2012) “Incidence of severe congenital neutropenia in Sweden and risk of evolution to myelodysplastic syndrome/leukaemia”, Br J Haematol 158(3): 363-369
11. Chung et al. (2006) “Agrobacterium is not alone: gene transfer to plants by viruses and other bacteria”, Trends Plant Sci. 11(1):1-4
12. Connelly, J. A. et al. (2012) “Hematopoietic stem cell transplantation for severe congenital neutropenia”, Curr Opin Hematol 19(1): 44-51
13. Crystal (1995) “Transfer of genes to humans: early lessons and obstacles to success”, Science 270(5235):404-10
14. Dale, D. C. (2017) “How I manage children with neutropenia”, Br J Haematol 178(3): 351-363
15. Dillon (1993) “Regulation gene expression in gene therapy” Trends in Biotechnology 11(5):167-173
16. Donadieu, J. et al. (2011) “Congenital neutropenia: diagnosis, molecular bases and patient management”, Orphanet J Rare Dis 6: 26.
17. Dranoff et al. (1997) “A phase I study of vaccination with autologous, irradiated melanoma cells engineered to secrete human granulocyte macrophage colony stimulating factor”, Hum. Gene Ther. 8(1):111-23
18. Donadieu et al. (2011) “Congenital neutropenia: diagnosis, molecular bases and patient management”, Orphanet J Rare Dis. 6:26
19. Dunbar et al. (1995) “Retrovirally marked CD34-enriched peripheral blood and bone marrow cells contribute to long-term engraftment after autologous transplantation”, Blood 85:3048-57
20. Ellem et al. (1997) “A case report: immune responses and clinical course of the first human use of ganulocyte/macrophage-colony-stimulating-factor-transduced autologous melanoma cells for immunotherapy”, Cancer Immunol Immunother 44:10-20
21. Gao and Huang (1995) “Cationic liposome-mediated gene transfer” Gene Ther. 2(10):710-22
22. Germeshausen et al. (2013) “The spectrum of ELANE mutations and their implications in severe congenital and cyclic neutropenia”, Hum Mutat. 34(6):905-14
23. Haddada et al. (1995) “Gene Therapy Using Adenovirus Vectors”, in: The Molecular Repertoire of Adenoviruses III: Biology and Pathogenesis, ed. Doerfler and Böhm, pp. 297-306
24. Han et al. (1995) “Ligand-directed retro-viral targeting of human breast cancer cells”, Proc Natl Acad Sci U.S.A. 92(21):9747-51
25. Horwitz, M. S. et al. (2013) “ELANE mutations in cyclic and severe congenital neutropenia: genetics and pathophysiology”, Hematol Oncol Clin North Am 27(1): 19-41, vii.
26. Inaba et al. (1992) “Generation of large numbers of dendritic cells from mouse bone marrow cultures supplemented with granulocyte/macrophage colony-stimulating factor”, J Exp Med. 176(6):1693-702
27. Jinek et al. (2012) “A programmable dual-RNA-guided DNA endonuclease in adaptive bacterial immunity,” Science 337(6096):816-21
28. Johan et al. (1992) “GLVR1, a receptor for gibbon ape leukemia virus, is homologous to a phosphate permease of Neurospora crassa and is expressed at high levels in the brain and thymus”, J Virol 66(3):1635-40
29. Judge et al. (2006) “Design of noninflammatory synthetic siRNA mediating potent gene silencing in vivo”, Mol Ther. 13(3):494-505
30. Kohn et al. (1995) “Engraftment of gene-modified umbilical cord blood cells in neonates with adnosine deaminase deficiency”, Nature Medicine 1:1017-23
31. Koonen et al. (2017) “Diversity, classification and evolution of CRIPR-Cas systems”, Current Opinion in Microbiology 37:67-78
32. Kremer and Perricaudet (1995) “Adenovirus and adeno-associated virus mediated gene transfer”, Br. Med. Bull. 51(1):31-44
33. Macdiarmid et al. (2009) “Sequential treatment of drug-resistant tumors with targeted minicells containing siRNA or a cytotoxic drug”, Nat Biotechnol. 27(7):643-51
34. Malech et al. (1997) “Prolonged production of NADPH oxidase-corrected granulocytes after gene therapy of chronic granulomatous disease”, PNAS 94(22):12133-38
35. Makaran et al. (2015) “The diversity of mutations and clinical outcomes for ELANE-associated neutropenia”, Curr Opin Hematol. 22(1):3-11
36. Miller et al. (1991) “Construction and properties of retrovirus packaging cells based on gibbon ape leukemia virus”, J Virol. 65(5):2220-24
37. Miller (1992) “Human gene therapy comes of age”, Nature 357:455-60
38. Mitani and Caskey (1993) “Delivering therapeutic genes—matching approach and application”, Trends in Biotechnology 11(5):162-66
39. Nabel and Felgner (1993) “Direct gene transfer for immunotherapy and immunization”, Trends in Biotechnology 11(5):211-15
40. Remy et al. (1994) “Gene Transfer with a Series of Lipphilic DNA-Binding Molecules”, Bioconjugate Chem. 5(6):647-54
41. Rosenberg, P. S. et al. (2010) “Stable long-term risk of leukaemia in patients with severe congenital neutropenia maintained on G-CSF therapy”, Br J Haematol 150(2): 196-199.
42. Schaffer, A et al. (2007) “Genetic heterogeneity in severe congenital neutropenia: how many aberrant pathways can kill a neutrophil?” Curr Opin Allergy Clin Immunol 7(6): 481-494
43. Sentmanat et al. (2018) “A Survey of Validation Strategies for CRISPR-Cas9 Editing”, Scientific Reports 8:888, doi:10.1038/s41598-018-19441-8
44. Skokowa, J. et al. (2017) “Severe congenital neutropenias”, Nat Rev Dis Primers 3: 17032
45. Sommerfelt et al. (1990) “Localization of the receptor gene for type D simian retroviruses on human chromosome 19”, J. Virol. 64(12):6214-20
46. Van Brunt (1988) “Molecular framing: transgenic animals as bioactors” Biotechnology 6:1149-54
47. Vigne et al. (1995) “Third-generation adenovectors for gene therapy”, Restorative Neurology and Neuroscience 8(1,2): 35-36
48. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78
49. Yu et al. (1994) “Progress towards gene therapy for HIV infection”, Gene Ther. 1(1):13-26
50. Yu, K et al. (2016) “Gene Editing of Human Hematopoietic Stem and Progenitor Cells: Promise and Potential Hurdles”, Hum Gene Ther 27(10): 729-740
51. Zetsche et al. (2015) “Cpf1 is a single RNA-guided endonuclease of a class 2 CRIPSR-Cas system” Cell 163(3):759-71
52. Zhenwang et al. (2017) “Large-scale ex vivo generation of human neutrophils from cord blood CD34⁺ cells”, PLOS ONE, available at <https://doi.org/10.1371/journal.pone.0180832>
53. Zuris et al. (2015) “Cationic lipid-mediated delivery of proteins enables efficient protein based genome editing in vitro and in vivo” Nat Biotechnol. 33(1):73-80

1. A method for inactivating in a cell a mutant allele of the elastase, neutrophil expressed gene (ELANE) gene having a mutation associated with severe congenital neutropenia (SCN) or cyclic neutropenia (CyN), the method comprising
(a) delivering to an isolated human cell that comprises a mutant ELANE allele and a functional ELANE allele a composition comprising:
a CRISPR nuclease or a sequence encoding the CRISPR nuclease; and
an isolated guide RNA molecule (gRNA) that targets the mutant ELANE allele, wherein the gRNA comprises a CRISPR RNA (crRNA) comprising a nucleic acid sequence of 17-20 nucleotides which comprise 17-20 contiguous nucleotides set forth in SEQ ID NOs: 913, 914, 1046, 1047, 1121, 1122, or 1190; and,
(b) culturing the cell obtained in step (a) such that the mutant ELANE allele is inactivated and the functional ELANE allele remains intact.

2. The method of claim 1, further comprising introduction of an isolated second gRNA, wherein the gRNA comprises a CRISPR RNA (crRNA) that complexes with a CRISPR nuclease to affect a second double strand break in the ELANE gene, wherein the second double strand break is within intron 4 of the ELANE gene.

3. The method of claim 2, wherein the isolated second gRNA molecule comprises a nucleic acid sequence of 17-20 nucleotides which comprise 17-20 contiguous nucleotides set forth in SEQ ID NO: 1197.

4. The method of claim 1, wherein the isolated human cell is from a subject with an ELANE gene mutation related to SCN or CyN and/or suffering from SCN or CyN and wherein the subject has a mutant ELANE allele and a functional ELANE allele.

5. The method of claim 4, comprising obtaining the isolated human cell from the subject by mobilization and/or by apheresis, or by bone marrow aspiration.

6. The method of claim 4, wherein the isolated human cell is prestimulated prior to introducing the composition to the cell.

7. The method of claim 4, further comprising culture expanding the isolated human cell to obtain multiple isolated human cells.

8. The method of claim 7, wherein the isolated human cells are cultured with one or more of: stem cell factor (SCF), IL-3, and GM-CSF;
wherein the cells are cultured with at least one cytokine; and/or
wherein the at least one cytokine is a recombinant human cytokine.

9. A composition comprising a cell obtained by the method of claim 1, wherein the mutant ELANE allele is inactivated and the functional ELANE allele remains intact (“the modified cell”) and a pharmaceutically acceptable carrier.

10. The composition of claim 9, wherein the modified cell is a hematopoietic stem cell and/or progenitor cell (HSPC);
wherein the modified cell is a CD34+ hematopoietic stem cell; or
wherein the modified cell is a bone marrow cell or peripheral mononucleated cell (PMC).

11. The composition of claim 9, wherein the modified cell is lacking at least a portion of one allele of the ELANE gene.

12. An isolated gRNA that targets a mutant ELANE allele, wherein the gRNA comprises a crRNA consisting of the nucleic acid sequence of SEQ ID NOs: 913, 914, 1046, 1047, 1121, 1122, or 1190.

13. The isolated gRNA of claim 12, wherein the gRNA comprises a crRNA consisting of the nucleic acid sequence of SEQ ID NOs: 913 or 914.

14. The isolated gRNA of claim 12, wherein the gRNA comprises a crRNA consisting of the nucleic acid sequence of SEQ ID NOs: 1046 or 1047.

15. The isolated gRNA of claim 12, wherein the gRNA comprises a crRNA consisting of the nucleic acid sequence of SEQ ID NOs: 1121 or 1122.

16. The isolated gRNA of claim 12, wherein the gRNA comprises a crRNA consisting of the nucleic acid sequence of SEQ ID NO. 1190.

17. The composition of claim 9, wherein the wherein the gRNA comprises crRNA) comprising a nucleic acid sequence of 17-20 nucleotides which comprise 17-20 contiguous nucleotides set forth in SEQ ID NOs: 913 or 914.

18. The composition of claim 9, wherein the wherein the gRNA comprises crRNA) comprising a nucleic acid sequence of 17-20 nucleotides which comprise 17-20 contiguous nucleotides set forth in SEQ ID NOs: 1046 or 1047.

19. The composition of claim 9, wherein the wherein the gRNA comprises crRNA) comprising a nucleic acid sequence of 17-20 nucleotides which comprise 17-20 contiguous nucleotides set forth in SEQ ID NOs: 1121 or 1122.

20. The composition of claim 9, wherein the wherein the gRNA comprises crRNA) comprising a nucleic acid sequence of 17-20 nucleotides which comprise 17-20 contiguous nucleotides set forth in SEQ ID NO. 1190.
